(19) United States
(12) Patent Application Publication
(10) Pub. No.: US 2009/0286791 A1
(43) Pub. Date: Nov. 19, 2009

(54) Amide Compounds

(75) Inventors: Shuji Kitamura, Osaka (JP); Thomas Daniel Aicher, Superior, CO (US); Steve Gonzales, Media, PA (US); Yvan Le Huerou, Boulder, CO (US); Scott Alan Pratt, Longmont, CO (US); Tim Turner, Longmont, CO (US); Yoshihisa Nakada, Osaka (JP)

Correspondence Address: WENDEROTH, LIND & PONACK, L.L.P., 1030 15th Street, N.W.,, Suite 400 East, Washington, DC, 20005-1503, US

(73) Assignee: TAKEDA PHARMACEUTICAL COMPANY LIMITED, OSAKA (JP)

(21) Appl. No.: 12/309,493

(22) PCT Filed: Jul. 20, 2007
(86) PCT No.: PCT/US2007/016425

§ 371 Date: Apr. 14, 2009

Related U.S. Application Data
(60) Provisional application No. 60/832,115, filed on Jul. 21, 2006.

(30) Foreign Application Priority Data
Nov. 27, 2001 (EP) 01127442.0

(51) Int. Cl. A61K 31/4155; C07D 401/12; C07D 403/12; C07D 413/14; C07D 231/14; A61P 3/04; A61P 3/10; A61K 31/4439; A61K 31/4184; A61K 31/498; A61K 31/497; A61K 31/5377; A61K 31/506

(52) U.S. Cl. 514/236.5; 546/275.4; 548/306.1; 544/405; 544/355; 544/131; 546/275.7; 546/273.4; 548/374.1; 514/341; 514/394; 514/249; 514/255.05; 514/273; 514/406

(57) ABSTRACT

The present invention provides compounds represented by the formula (Ia):

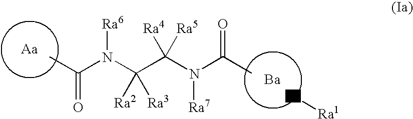

the formula (Ib):

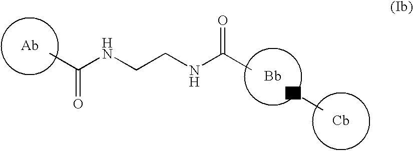

the formula (Ic):

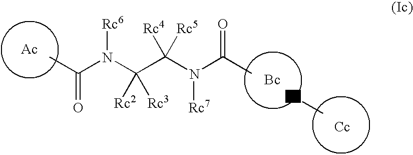

and the formula (Id):

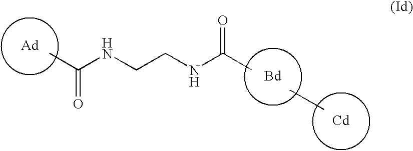

wherein each symbol is as defined in the specification.

According to the present invention, these compounds have a DGAT inhibitory activity and are useful for the prophylaxis, treatment or improvement of diseases or pathologies caused by high expression or high activation of DGAT.

### TECHNICAL FIELD OF THE INVENTION

The present invention relates to a novel amide compound having a diacylglycerol acyl transferase (hereinafter sometimes to be abbreviated as DGAT in the present specification) inhibitory activity, which is useful for the treatment of obesity, hyperlipidemia, diabetes and the like.

### BACKGROUND OF THE INVENTION

Obesity is a state of excess accumulation of fat, mainly triglyceride, in the body, and is deeply involved in the progression into the pathology such as arteriosclerosis, diabetes, hypertension and the like. Therefore, the development of a drug for the prophylaxis or treatment thereof has been desired. In mammals, two major triglyceride synthesis pathways have been biochemically clarified. One is the glycelophosphoric acid pathway present in all tissues, and the other pathway is a monoglyceride pathway. In any pathway, fatty acid in the cell is converted to acyl coenzyme A by an acyl coenzyme A synthetase and introduced into triglyceride through the both pathways. As the enzyme involved in the final stage of the intracellular or intraorgan triglyceride synthesis process, DGAT has been known. As DGAT, DGAT1 and DGAT2 have been cloned. DGAT1 knockout mice have been created and analyzed. As a result, the mice did not become obese easily with high fat diet and showed promoted energy consumption and insulin sensitivity, as compared to wild-type mice. In a mating test of DGAT1 knockout mice and Ay/a mice, moreover, body weight gain was suppressed with a normal diet and a phenotype of promoted insulin sensitivity and elimination of leptin resistance was shown. Thus, DGAT1 inhibitors are expected to be antiobesity drugs.

DGAT is an enzyme (EC2.3.1.20) also designated as acyl coenzyme A:diacylglycerol acyl transferase. cDNA cloning of DGAT1 is reported in Proc. Natl. Acad. Sci. USA. 95, 13018-13023, 1998, and cDNA cloning of DGAT2 is reported in The Journal of Biological Chemistry, 276, 42, 38862-38869, 2001 and The Journal of Biological Chemistry, 276, 42, 38870-38876, 2001. Since the enzyme molecule of DGAT was not clarified for a long time, there is not much finding relating to the DGAT activity. Since the DGAT activity is detected in the endoplasmic reticulum membrane fraction, it was considered to be an endoplasmic reticulum membrane protein. However, ever since cDNA cloning of DGAT was reported, the properties thereof have been rapidly elucidated. For example, it has been reported to be a protein forming a tetramer in Biochem. Journal, 359, 707-714, 2001. A knockout mouse of DGAT1 (DGAT1 defective mouse) was created and its phenotype was reported in Nature Genetics, 25, 87-90, 2000, The Journal of Clinical Investigation, 109, 175-181, 2002 and The Journal of Clinical Investigation, 109, 1049-1055, 2002. From these reports, the DGAT1 inhibitors have been suggested to show an antiobesity action, an anti-insulin resistance action, and an anti-leptin resistance action, and DGAT1 inhibitors are expected to become pharmaceutical products.

In addition, DGAT2 knockout mice were also created and their phenotype is reported in The Journal of Biological Chemistry, 279, 11767-11776 (2004). As a result, DGAT2 was clarified to be an enzyme that plays a key role in the synthesis of triglyceride in the liver. The Journal of Biological Chemistry, 274, 35577-35582, 1999 reports that there are a DGAT activity involved in a storage-type triglyceride synthesis in the cytoplasm side of the endoplasmic reticulum membrane and a DGAT activity that supplies triglyceride to be mobilized for lipoprotein secretion in the lumen side, which suggests that DGAT1 and DGAT2 play different roles as triglyceride synthases and further that DGAT2 inhibitors are effective for hypertriglyceridemia.

In addition, since DGAT expression is promoted in various pathologies and diseases such as obesity, diabetes, insulin-resistant diabetes, leptin resistance, arteriosclerosis, hypertriglyceridemia, hypercholesterolemia, arteriosclerosis, hypertension and the like, high expression or hyper activation of DGAT is suggested to be involved in the excess accumulation of triglyceride in the cell, tissue or organ, and closely involved in the onset and aggravation of these diseases.

In, for example, fat organs and adipocytes, expression of DGAT is regulated by hormones such as insulin, leptin and the like, and DGAT is suggested to be deeply involved in the pathologies such as insulin resistance, leptin resistance and the like. Therefrom it is considered that a compound having a DGAT inhibitory activity is effective for the treatment of obesity, insulin resistant diabetes, hyperorexia or obesity based on leptin resistance.

As amide compounds, the following compounds are known.

(1) Amide compounds useful as leukemia therapeutic agents (Journal of Medicinal Chemistry (2005), 48(24), 7906-7910).
(2) A compound represented by the following formula (I), which is useful as an orexin receptor antagonist (WO01/96302):

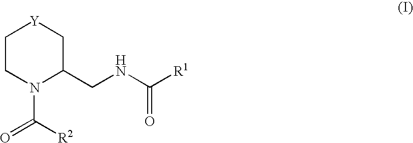

wherein:

Y represents a group (CH₂), wherein n represents 0, 1 or 2;

R¹ is phenyl, naphthyl, a mono or bicyclic heteroaryl group; or a group NR³R⁴, wherein one of R³ and R⁴ is hydrogen or optionally substituted (C₁₋₄)alkyl and the other is phenyl, naphthyl or a mono or bicyclic heteroaryl group, or R³ and R⁴ together with the N atom to which they are attached form a 5 to 7-membered cyclic amine which has an optionally fused phenyl ring; any of which R¹ groups may be optionally substituted;

R² represents phenyl or a 5- or 6-membered heteroaryl group, wherein the phenyl or heteroaryl group is substituted by R⁵, and further optional substituents; or R² represents an optionally substituted bicyclic aromatic or bicyclic heteroaromatic group;

R⁵ represents an optionally substituted C₁₋₄ alkoxy, halo, optionally substituted C₁₋₆ alkyl, optionally substituted phenyl, or an optionally substituted 5- or 6-membered heterocyclic ring.

(3) A compound represented by the following formula, which is useful as a herbicide (WO01/46152):

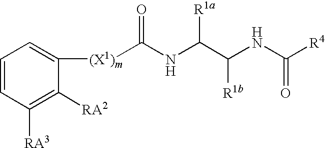

wherein:

one of R^{1a} and R^{1b} is a methyl, hydroxymethyl or monohalomethyl group and the other is hydrogen;

X¹ is a methylene, oxy or thio linkage;

m is 0 or 1;

RA² is a hydrogen, halogen or methyl group;

RA³ is a halogen or halomethyl group; and

R⁴ is an α-halo- or α,α-dihalo-(C₁₋₃)alkyl group or a group having the formula -(X²)ₙ-R⁵ where X² is a methylene, oxy or thio linkage, n is 0 or 1, and R⁵ is an optionally substituted 5- or 6-member aromatic or heterocyclic ring.

(4) A compound represented by the following formula (I), which is useful as a phosphodiesterase 4 (PDE4) inhibitor (WO2005/116009):

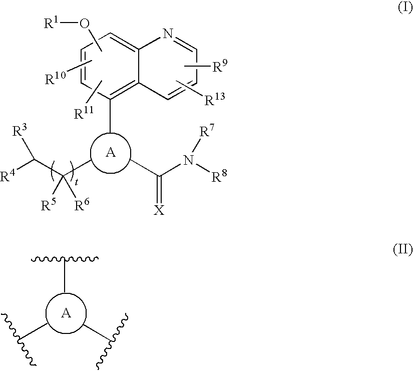

wherein:

the formula (II) is a 5-membered heteroaryl;

X is S or O;

R¹ is H, alkyl, cycloalkyl, cycloalkylalkyl-, or the like;

R³ and R⁴ are each independently H, alkyl, hydroxyalkyl or —C(O)—O-alkyl;

R⁵ and R⁶ are each independently H, alkyl, hydroxyalkyl, alkoxyalkyl, mercaptoalkyl, or the like;

R⁷ is H, alkyl, alkenyl, hydroxyalkyl, cycloalkyl, alkoxyalkyl, aminoalkyl, (R¹⁷-phenyl) alkyl or —CH₂—C(O)—O-alkyl; and R⁸ is alkyl, heteroaryl, phenyl, cycloalkyl, or heterocycloalkyl, all optionally substituted, or a cycloalkyl- or heterocycloalkyl-substituted amide; or R⁷ and R⁸ and the nitrogen to which they are attached together form an optionally substituted ring;

R⁹ is H, halo, alkyl, cycloalkyl, or the like;

R¹⁰, R¹¹, and R¹³ are each independently H or halo;

R¹⁷ is 1 to 3 substituents independently selected from the group consisting of H, halo, cycloalkyl, and the like.

(5) Amide compounds useful as farnesyltransferase inhibitors (Journal of Combinatorial Chemistry (2004), 6(3), 407-413).
(6) A compound represented by the following formula (I), which is useful as an adenosine receptor ligand (WO2003/045385):

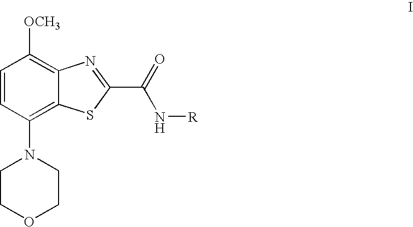

wherein:

R is hydrogen, —(CH₂)ₙ-phenyl optionally substituted, —(CH₂)ₙ-pyridinyl optionally substituted, —(CH₂)ₙ—C₃₋₆-cycloalkyl optionally substituted, —(CH₂), —N(R′)—C₃₋₆-cycloalkyl optionally substituted, —(CH₂)ₙ-benzo[1,3]-dioxolyl, —(CR₁₂)ₙ-thiophenyl optionally substituted, —(CR₁₂)ₙ-thiazolyl optionally substituted, —(CH₂)ₙ—C(O)-thiophenyl optionally substituted, —(CH₂)ₙ-furanyl optionally substituted, —(CH₂)ₙ—C(O)—(CH₂)ₙ-thiophenyl, —(CHR′)ₙ-benzofuran-2-yl, —(CH₂)ₙ-benzo[b]thiophenyl optionally substituted, —(CH₂)ₙ—N(R′)—C(O)-phenyl optionally substituted, —(CH₂)ₙ—C(O)-phenyl optionally substituted, (CH₂)ₙ—C(O)-2,3-dihydro-benzo[1,4]dioxin-6-yl, —(CH₂)ₙ—N(R′)—C(O)-pyridinyl, —(CH₂)ₙ-tetrahydrofuranyl, —CH-bi-phenyl, —CH (phenyl)-pyridinyl, —(CH₂)ₙ-1-oxo-1,3-dihydro-isoindol-2-yl, —(CH₂)ₙ-1,3-dioxo-1,3-dihydro-isoindol-2-yl, —(CH₂)ₙ—CH(phenyl)-tetrahydropyranyl, —(CH₂)ₙ-1-oxo-1,2,3,4-tetrahydro-isoquinolin-3-yl or (CH₂)ₙ—S—[1,3,4]thiazol-2-yl optionally substituted;

R′ is hydrogen or lower alkyl, independently from each other in case R′₂; and

n is 0, 1, 2, 3 or 4.

(7) Amide compounds useful as synthetic intermediates for tripodal ligands (Helvetica Chimica Acta (1998), 81(2), 207-218).
(8) Amide compounds useful as radical scavengers (Tetrahedron (2004), 60(39), 8729-8738).

However, none of the above-mentioned prior art reports on the compound of the present invention.

### DISCLOSURE OF THE INVENTION

There is a demand on the development of a novel compound having a superior DGAT inhibitory activity and superior in properties (stability, solubility etc.), oral absorbability, migration to target organs and the like.

The present inventors have searched for a compound having a DGAT inhibitory activity, and found that the compounds represented by the below-mentioned formulas (Ia), (Ib), (Ic) and (Id) have a superior DGAT inhibitory activity, and are superior in the properties as a pharmaceutical product, such as stability and the like, which resulted in the completion of the present invention.

Accordingly, the present invention relates to

[1] A compound represented by formula (Ia):

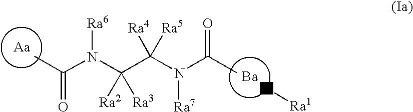

wherein

ring Ba is a 5-membered nitrogen-containing aromatic heterocycle optionally condensed with an aromatic ring, which is optionally further substituted;

Ra¹ is a hydrogen atom or a substituent;

ring Aa is an optionally substituted aromatic heterocycle; and

Ra², Ra³, Ra⁴, Ra⁵, Ra⁶ and Ra⁷ are each independently a hydrogen atom or a substituent;

provided that

1) when ring Ba is pyrazole which is optionally further substituted, then ring Ba does not have optionally substituted tetrahydrofurylmethoxy as a substituent other than Ra¹;

2) when ring Ba is imidazole which is optionally further substituted, then ring Ba does not have optionally substituted quinolyl as a substituent other than Ra¹;

3) when ring Ba is pyrazole which is optionally further substituted, then Ra¹ is not optionally substituted quinolyl; and

4) ring Aa is not the same as ring Ba; or a salt thereof (hereinafter to be abbreviated as compound (Ia));

[1A] a compound represented by formula (IaA):

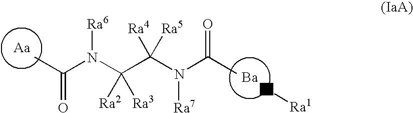

wherein

ring Ba is a 5-membered nitrogen-containing aromatic heterocycle optionally condensed with an aromatic ring, which is optionally further substituted;

Ra¹ is a hydrogen atom or a substituent;

ring Aa is an optionally substituted aromatic heterocycle; and

Ra², Ra³, Ra⁴, Ra⁵, Ra⁶ and Ra⁷ are each independently a hydrogen atom or a substituent;

provided that

1) ring Ba is not oxadiazole which is optionally further substituted;

2) when ring Ba is pyrazole which is optionally further substituted, then ring Ba does not have optionally substituted tetrahydrofurylmethoxy as a substituent other than Ra¹;

3) when ring Ba is imidazole which is optionally further substituted, then ring Ba does not have optionally substituted quinolyl as a substituent other than Ra¹;

4) when ring Ba is pyrazole which is optionally further substituted, then Ra¹ is not optionally substituted quinolyl; and

5) ring Aa is not the same as ring Ba; or a salt thereof;

[2] The compound of above-mentioned [1], wherein ring Ba is pyrazole, benzimidazole, indole or indazole, each of which is optionally further substituted;
[3] The compound of above-mentioned [1], wherein Ra¹ is a hydrogen atom, an optionally substituted hydrocarbon group, an optionally substituted heterocyclic group or an acyl group;
[4] The compound of above-mentioned [1], wherein ring Aa is an aromatic heterocycle optionally substituted by 1 to 3 substituents selected from an optionally substituted hydrocarbon group, an optionally substituted heterocyclic group, an optionally substituted hydroxy group, an optionally substituted amino group, an optionally substituted mercapto group, a cyano group, an acyl group and a halogen atom;
[5] The compound of above-mentioned [1], wherein Ra², Ra³, Ra⁴, Ra⁵, Ra⁶ and Ra⁷ are both hydrogen atoms;
[6] A compound represented by formula (Ib):

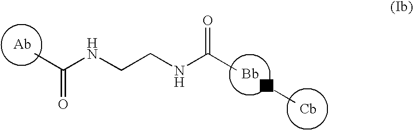

wherein

ring Bb is a 5-membered nitrogen-containing aromatic heterocycle optionally condensed with an aromatic ring, which is optionally further substituted;

ring Cb is an optionally substituted aromatic heterocycle; and

ring Ab is an optionally substituted aromatic hydrocarbon;

provided that when ring Bb is pyrazole which is optionally further substituted, then ring Cb is not optionally substituted quinoline;

or a salt thereof (hereinafter to be abbreviated as compound (Ib));
[7] The compound of above-mentioned [6], wherein ring Bb is pyrazole, benzimidazole, indole or indazole, each of which is optionally further substituted;
[8] The compound of above-mentioned [6], wherein ring Cb is an aromatic heterocycle optionally substituted by 1 to 3 substituents selected from a halogen atom, a hydroxy group, a C₁₋₆ alkyl group and a C₁₋₆ alkoxy group;
[9] The compound of above-mentioned [6], wherein ring Ab is an aromatic hydrocarbon optionally substituted by 1 to 3 substituents selected from an optionally substituted hydrocarbon group, an optionally substituted heterocyclic group, an optionally substituted hydroxy group, a cyano group, an acyl group and a halogen atom;
[10] A compound represented by formula (Ic):

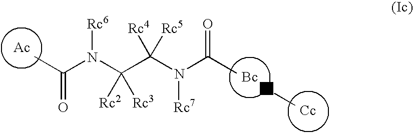

wherein

ring Bc is a 5-membered nitrogen-containing aromatic heterocycle optionally condensed with an aromatic ring, which is optionally further substituted;

ring Cc is an optionally substituted aromatic ring;

ring Ac is an optionally substituted aromatic hydrocarbon; and

Rc², Rc³, Rc⁴, Rc⁵, Rc⁶ and Rc⁷ are each independently a hydrogen atom or a substituent, or any two of Rc², Rc³, Rc⁴, Rc⁵, Rc⁶ and Rc⁷ are optionally bonded to each other to form a non-aromatic ring;

provided that

1) ring Bc is not pyrazol-5-yl and 2H-1,2,3-triazol-4-yl, each of which is optionally further substituted;

2) ring Cc is not optionally substituted quinoline;

3) a compound wherein Rc², Rc³, Rc⁴, Rc⁵, Rc⁶ and Rc⁷ are hydrogen atoms is excluded; and

4) when Rc⁶ and Rc⁷ are bonded, then they do not form piperazine;

or a salt thereof (hereinafter to be abbreviated as compound (Ic));
[11] The compound of above-mentioned [10], wherein ring Bc is pyrazole, benzimidazole, indole or indazole, each of which is optionally further substituted;
[12] The compound of above-mentioned [10], wherein ring Cc is an aromatic hydrocarbon optionally substituted by 1 to 3 substituents selected from a halogen atom, a hydroxy group, a C₁₋₆ alkyl group and a C₁₋₆ alkoxy group;
[13] The compound of above-mentioned [10], wherein ring Ac is an aromatic hydrocarbon optionally substituted by 1 to 3 substituents selected from an optionally substituted hydrocarbon group, an optionally substituted heterocyclic group, an optionally substituted hydroxy group, a cyano group, an acyl group and a halogen atom;
[14] The compound of above-mentioned [10], wherein Rc² and Rc³ are each independently a hydrogen atom, an acyl group or an optionally substituted hydrocarbon group, or Rc² or Rc³ is bonded to Rc⁴ or Rc⁵ to form a non-aromatic ring, bonded to Rc⁶ to form a non-aromatic heterocycle, or bonded to Rc⁷ to form a non-aromatic heterocycle;
[15] The compound of above-mentioned [10], wherein Rc⁴ and Rc⁵ are each independently a hydrogen atom, an acyl group or an optionally substituted hydrocarbon group, or Rc⁴ or Rc⁵ is bonded to Rc² or Rc³ to form a non-aromatic ring, bonded to Rc⁶ to form a non-aromatic heterocycle, or bonded to Rc⁷ to form a non-aromatic heterocycle;
[16] The compound of above-mentioned [10], wherein Rc⁶ is a hydrogen atom or an optionally substituted hydrocarbon group, or Rc⁶ is bonded to Rc² or Rc³ to form a non-aromatic heterocycle, or bonded to Rc⁴ or Rc⁵ to form a non-aromatic heterocycle;
[17] The compound of above-mentioned [10], wherein Rc⁷ is a hydrogen atom or an optionally substituted hydrocarbon group, or Rc⁷ is bonded to Rc² or Rc³ to form a non-aromatic heterocycle, or bonded to Rc⁴ or Rc⁵ to form a non-aromatic heterocycle;
[18] A compound represented by formula (Id):

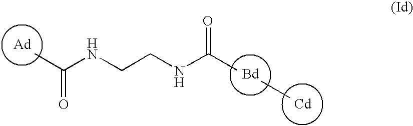

wherein

ring Bd is an aromatic heterocycle which is optionally further substituted;

ring Cd is an optionally substituted aromatic ring; and

ring Ad is an optionally substituted aromatic hydrocarbon;

provided that

1) ring Bd is not pyrazol-4-yl and pyrrol-3-yl, each of which is optionally further substituted;

2) ring Cd is not optionally substituted quinoline;

3) when ring Bd is pyridine or quinoline, each of which is optionally further substituted, then ring Bd has substituent(s) besides ring Cd; and

4) when ring Bd is a 5-membered nitrogen-containing aromatic heterocycle optionally condensed with an aromatic ring, which is optionally further substituted, then ring Bd does not have an optionally substituted aromatic heterocyclic group as a substituent other than ring Cd and ring Cd is an optionally substituted aromatic hydrocarbon;

or a salt thereof (hereinafter to be abbreviated as compound (Id));
[19] The compound of above-mentioned [18], wherein ring Bd is pyridine, pyrazole, triazole or indole, each of which is optionally further substituted;
[20] The compound of above-mentioned [18], wherein ring Cd is an aromatic hydrocarbon optionally substituted by 1 to 3 substituents selected from a halogen atom, a hydroxy group, a C₁₋₆ alkyl group and a C₁₋₆ alkoxy group;
[21] The compound of above-mentioned [18], wherein ring Ad is an aromatic hydrocarbon optionally substituted by 1 to 3 substituents selected from an optionally substituted hydrocarbon group, an optionally substituted heterocyclic group, an optionally substituted hydroxy group, a cyano group, an acyl group and a halogen atom;
[22] N-(2-(1-phenyl-3-(trifluoromethyl)-1H-pyrazole-4-carboxamido)ethyl)-6-(2,2,2-trifluoroethoxy)nicotinamide (Example A27);
6-(cyclopropylmethoxy)-N-(2-(1-phenyl-3-(trifluoromethyl)-1H-pyrazole-4-carboxamido)ethyl)nicotinamide (Example A35);
N-(2-(1-phenyl-3-(trifluoromethyl)-1H-pyrazole-4-carboxamido)ethyl)-6-(3,3,3-trifluoropropoxy)nicotinamide (Example A42);
6-(2-(ethylsulfonyl)ethoxy)-N-(2-(1-phenyl-3-(trifluoromethyl)-1H-pyrazole-4-carboxamido)ethyl)nicotinamide (Example A43);
N-(2-(1-phenyl-3-(trifluoromethyl)-1H-pyrazole-4-carboxamido)ethyl)-6-propylnicotinamide (Example A47);
1-phenyl-N-(2-(6-(2,2,2-trifluoroethoxy)nicotinamido)ethyl)-1H-indole-3-carboxamide (Example A53);
N-(2-(1-phenyl-3-(trifluoromethyl)-1H-pyrazole-4-carboxamido)ethyl)-6-o-tolylnicotinamide (Example A73);
1-benzoyl-N-(2-(6-(2,2,2-trifluoroethoxy)nicotinamido)ethyl)-1H-indole-3-carboxamide (Example A82);
6-(5-isopropyl-1,2,4-oxadiazol-3-yl)-N-(2-(1-phenyl-3-(trifluoromethyl)-1H-pyrazole-4-carboxamido)ethyl)nicotinamide (Example A103); or
N-(2-(4-ethoxybenzamido)ethyl)-1-(pyridin-2-yl)-3-(trifluoromethyl)-1H-pyrazole-4-carboxamide (Example B3);
   or a salt thereof;
   [23] A prodrug of the compound of above-mentioned [1], [6], [10] or [18];
   [24] A pharmaceutical agent comprising the compound of above-mentioned [1], [6], [10] or [18], or a prodrug thereof;
   [25] The pharmaceutical agent of above-mentioned [24], which is an agent for the prophylaxis or treatment of obesity, hyperlipidemia or diabetes;
   [26] A DGAT inhibitor comprising the compound of above-mentioned [1], [6], [10] or [18], or a prodrug thereof;
   [27] Use of the compound of above-mentioned [1], [6], [10] or [18], or a prodrug thereof for the production of an agent for the prophylaxis or treatment of obesity, hyperlipidemia or diabetes;
   [28] Use of the compound of above-mentioned [1], [6], [10] or [18], or a prodrug thereof for the production of a DGAT inhibitor;
   [29] A method for the prophylaxis or treatment of obesity, hyperlipidemia or diabetes in a mammal, which comprises administering the compound of above-mentioned [1], [6], [10] or [18], or a prodrug thereof to the mammal;
   [30] A method of inhibiting DGAT in a mammal, which comprises administering the compound of above-mentioned [1], [6], [10] or [18], or a prodrug thereof to the mammal;
   and the like.

The compound (Ia), compound (Ib), compound (Ic) and compound (Id) (these are also collectively referred to as the compound of the present invention in this specification) have a DGAT inhibitory activity and are useful for the prophylaxis, treatment or amelioration of diseases or pathologies caused by high expression or high activation of DGAT (sometimes to be abbreviated as DGAT-related diseases in this specification).

### DETAILED DESCRIPTION OF THE INVENTION

In the present specification, unless otherwise specified, the “halogen atom” means fluorine atom, chlorine atom, bromine atom or iodine atom.

In the present specification, unless otherwise specified, the “C₁₋₃ alkylenedioxy group” means methylenedioxy, ethylenedioxy, trimethylenedioxy or the like.

In the present specification, unless otherwise specified, the “C₁₋₆ alkyl group” means methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl or the like.

In the present specification, unless otherwise specified, the “C₁₋₆ alkoxy group” means methoxy, ethoxy, propoxy, isopropoxy, butoxy, isobutoxy, sec-butoxy, tert-butoxy or the like.

In the present specification, unless otherwise specified, the “C₁₋₆ alkoxy-carbonyl group” means methoxycarbonyl, ethoxycarbonyl, propoxycarbonyl, tert-butoxycarbonyl or the like.

In the present specification, unless otherwise specified, the “C₁₋₆ alkyl-carbonyl group” means acetyl, propanoyl, butanoyl, isobutanoyl, pentanoyl, isopentanoyl, hexanoyl or the like.

Each symbol in the formula (Ia) is described in detail in the following.

Ra¹, Ra², Ra³, Ra⁴, Ra⁵, Ra⁶ and Ra⁷ are each independently a hydrogen atom or a substituent.

As the “substituent” for Ra¹, Ra², Ra³, Ra⁴, Ra⁵, Ra⁶ or Ra⁷, an “optionally substituted hydrocarbon group”, an “optionally substituted heterocyclic group”, an “optionally substituted hydroxy group”, an “optionally substituted amino group”, an “optionally substituted mercapto group”, a “cyano group”, a “nitro group”, an “acyl group”, a “halogen atom” and the like can be mentioned.

As the “hydrocarbon group” of the aforementioned “optionally substituted hydrocarbon group”, for example, a C₁₋₁₀ alkyl group, a C₂₋₁₀ alkenyl group, a C₂₋₁₀ alkynyl group, a C₃₋₁₀ cycloalkyl group, a C₃₋₁₀ cycloalkenyl group, a C₄₋₁₀ cycloalkadienyl group, a C₆₋₁₄ aryl group, a C₇₋₁₃ aralkyl group, a C₈₋₁₃ arylalkenyl group, a C₃₋₁₀ cycloalkyl-C₁₋₆ alkyl group and the like can be mentioned.

Here, as the C₁₋₁₀ alkyl group, for example, methyl, ethyl, propyl, isopropyl, butyl, isobutyl, sec-butyl, tert-butyl, pentyl, isopentyl, neopentyl, 1-ethylpropyl, hexyl, isohexyl, 1,1-dimethylbutyl, 2,2-dimethylbutyl, 3,3-dimethylbutyl, 2-ethylbutyl, heptyl, octyl, nonyl, decyl and the like can be mentioned.

As the C₂₋₁₀ alkenyl group, for example, ethenyl, 1-propenyl, 2-propenyl, 2-methyl-1-propenyl, 1-butenyl, 2-butenyl, 3-butenyl, 3-methyl-2-butenyl, 1-pentenyl, 2-pentenyl, 3-pentenyl, 4-pentenyl, 4-methyl-3-pentenyl, 1-hexenyl, 3-hexenyl, 5-hexenyl, 1-heptenyl, 1-octenyl and the like can be mentioned.

As the C₂₋₁₀ alkynyl group, for example, ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl, 1-heptynyl, 1-octynyl and the like can be mentioned.

As the C₃₋₁₀ cycloalkyl group, for example, cyclopropyl, cyclobutyl, cyclopentyl, cyclohexyl, cycloheptyl, cyclooctyl and the like can be mentioned.

As the C₃₋₁₀ cycloalkenyl group, for example, 2-cyclopenten-1-yl, 3-cyclopenten-1-yl, 2-cyclohexen-1-yl, 3-cyclohexen-1-yl and the like can be mentioned.

As the C₄₋₁₀ cycloalkadienyl group, for example, 2,4-cyclopentadien-1-yl, 2,4-cyclohexadien-1-yl, 2,5-cyclohexadien-1-yl and the like can be mentioned.

The above-mentioned C₃₋₁₀ cycloalkyl group, C₃₋₁₀ cycloalkenyl group and C₄₋₁₀ cycloalkadienyl group are each optionally condensed with a benzene ring, and as such a fused ring group, for example, indanyl, dihydronaphthyl, tetrahydronaphthyl, fluorenyl and the like can be mentioned.

The above-mentioned C₃₋₁₀ cycloalkyl group, C₃₋₁₀ cycloalkenyl group and C₄₋₁₀ cycloalkadienyl group each may be a C₇₋₁₀ crosslinked hydrocarbon group. As the C₇₋₁₀ crosslinked hydrocarbon group, bicyclo[2.2.1]heptyl (norbornyl), bicyclo[2.2.2]octyl, bicyclo[3.2.1]octyl, bicyclo[3.2.2]nonyl, bicyclo[3.3.1]nonyl, bicyclo[4.2.1]nonyl, bicyclo[4.3.1]decyl, adamantyl and the like can be mentioned.

The above-mentioned C₃₋₁₀ cycloalkyl group, C₃₋₁₀ cycloalkenyl group and C₄₋₁₀ cycloalkadienyl group each optionally form, together with a C₃₋₁₀ cycloalkane, a C₃₋₁₀ cycloalkene or a C₄₋₁₀ cycloalkadiene, a spiro ring group. Here, as the C₃₋₁₀ cycloalkane, C₃₋₁₀ cycloalkene and C₄₋₁₀ cycloalkadiene, rings corresponding to the above-mentioned C₃₋₁₀ cycloalkyl group, C₃₋₁₀ cycloalkenyl group and C₄₋₁₀ cycloalkadienyl group can be mentioned. As such a spiro ring group, spiro[4.5]decan-8-yl and the like can be mentioned.

As the C₆₋₁₄ aryl group, for example, phenyl, naphthyl, anthryl, phenanthryl, acenaphthylenyl, biphenylyl and the like can be mentioned.

As the C₇₋₁₃ aralkyl group, for example, benzyl, phenethyl, naphthylmethyl, biphenylylmethyl and the like can be mentioned.

As the C₈₋₁₃ arylalkenyl group, for example, styryl and the like can be mentioned.

As the C₃₋₁₀ cycloalkyl-C₁₋₆ alkyl group, for example, cyclohexylmethyl and the like can be mentioned.

The C₁₋₁₀ alkyl group, C₂₋₁₀ alkenyl group and C₂₋₁₀ alkynyl group, which are exemplarily recited as the aforementioned “hydrocarbon group”, each optionally have 1 to 3 substituents at substitutable position(s).

As such substituents, for example,

(1) a C₃₋₁₀ cycloalkyl group (e.g., cyclopropyl, cyclohexyl);
(2) a C₆₋₁₄ aryl group (e.g., phenyl, naphthyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms,

(d) a halogen atom, and

(e) a cyano group;

(3) an aromatic heterocyclic group (e.g., thienyl, furyl, pyridyl, pyrazolyl, imidazolyl, tetrazolyl, oxazolyl, thiazolyl, oxadiazolyl, thiadiazolyl, benzothiazolyl, pyrazinyl, quinolyl, indolyl, pyrimidinyl, triazolyl, isoxazolyl) optionally substituted by 1 to 3 substituents selected from

(a) a halogen atom,

(b) a hydroxy group,

(c) a C₁₋₆ alkyl group optionally substituted by 1 to 3 substituents selected from
(i) a halogen atom,
(ii) a C₁₋₆ alkoxy group optionally substituted by 1 to 3 halogen atoms,
(iii) a C₁₋₆ alkoxy-carbonyl group, and
(iv) an amino group optionally mono- or di-substituted by C₁₋₆ alkyl group(s),

(d) a C₁₋₆ alkoxy group optionally substituted by 1 to 3 halogen atoms,

(e) a C₆₋₁₄ aryl group (e.g., phenyl) optionally substituted by 1 to 3 substituents selected from
(i) a halogen atom,
(ii) a C₁₋₆ alkyl group optionally substituted by 1 to 3 halogen atoms, and
(iii) a C₁₋₆ alkylsulfonyl group (e.g., methylsulfonyl),

(f) a C₇₋₁₃ aralkyl group (e.g., benzyl),

(g) a C₃₋₁₀ cycloalkyl group (e.g., cyclopropyl) optionally substituted by 1 to 3 halogen atoms,

(h) an aromatic heterocyclic group (e.g., pyridyl, thienyl, pyrimidinyl), and

(i) a non-aromatic heterocyclic group (e.g., tetrahydropyranyl);

(4) a non-aromatic heterocyclic group (e.g., tetrahydrofuryl, morpholinyl, thiomorpholinyl, piperidinyl, pyrrolidinyl, piperazinyl, dioxolyl, dioxolanyl, 1,3-dihydro-2-benzofuranyl, thiazolidinyl, tetrahydropyranyl, dihydrooxadiazolyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group optionally substituted by 1 to 3 halogen atoms,

(d) an oxo group, and

(e) a halogen atom;

(5) an amino group optionally mono- or di-substituted by substituent(s) selected from

(a) a C₁₋₆ alkyl group optionally substituted by 1 to 3 substituents selected from
(i) a hydroxyl group,
(ii) a C₁₋₆ alkoxy group optionally substituted by 1 to 3 halogen atoms,
(iii) an amino group optionally mono- or di-substituted by C₁₋₆ alkyl group(s) optionally substituted by 1 to 3 halogen atoms, and
(iv) a halogen atom,

(b) a C₁₋₆ alkyl-carbonyl group optionally substituted by 1 to 3 halogen atoms,

(c) a C₁₋₆ alkoxy-carbonyl group optionally substituted by 1 to 3 halogen atoms,

(d) a C₆₋₁₄ aryl-carbonyl group (e.g., benzoyl) optionally substituted by 1 to 3 C₁₋₆ alkyl groups optionally substituted by 1 to 3 halogen atoms,

(e) a C₇₋₁₃ aralkyl-carbonyl group (e.g., benzylcarbonyl) optionally substituted by 1 to 3 halogen atoms,

(f) a C₃₋₁₀ cycloalkyl-carbonyl group (e.g., cyclopropylcarbonyl, cyclohexylcarbonyl) optionally substituted by 1 to 3 halogen atoms,

(g) an aromatic heterocyclylcarbonyl group (e.g., pyrazolylcarbonyl, pyrazinylcarbonyl, isoxazolylcarbonyl, pyridylcarbonyl) optionally substituted by 1 to 3 C₁₋₆ alkyl groups optionally substituted by 1 to 3 halogen atoms,

(h) a non-aromatic heterocyclylcarbonyl group (e.g., tetrahydrofurylcarbonyl, tetrahydrothiopyranylcarbonyl),

(i) a C₁₋₆ alkylsulfonyl group (e.g., methylsulfonyl),

(j) a C₆₋₁₄ arylsulfonyl group (e.g., benzenesulfonyl),

(k) an aromatic heterocyclylsulfonyl group (e.g., thienylsulfonyl),

(l) a C₃₋₁₀ cycloalkyl group (e.g., cyclopropyl) optionally substituted by 1 to 3 halogen atoms,

(m) a C₆₋₁₄ aryl group (e.g., phenyl) optionally substituted by 1 to 3 C₁₋₆ alkyl groups optionally substituted by 1 to 3 halogen atoms, and

(n) an aromatic heterocyclic group (e.g., pyrazolyl, pyrazinyl, isoxazolyl, pyridyl) optionally substituted by 1 to 3 C₁₋₆ alkyl groups optionally substituted by 1 to 3 halogen atoms;

(6) an amidino group;
(7) a C₁₋₆ alkyl-carbonyl group optionally substituted by 1 to 3 halogen atoms;
(8) a C₁₋₆ alkoxy-carbonyl group optionally substituted by 1 to 3 substituents selected from

(a) a halogen atom, and

(b) a C₁₋₆ alkoxy group;

(9) a C₁₋₆ alkylsulfonyl group (e.g., methylsulfonyl, ethylsulfonyl, isopropylsulfonyl) optionally substituted by 1 to 3 halogen atoms;
(10) a C₃₋₁₀ cycloalkylsulfonyl group (e.g., cyclopropylsulfonyl);
(11) a C₆₋₁₄ arylsulfonyl group (e.g., benzenesulfonyl) optionally substituted by 1 to 3 substituents selected from

(a) a C₁₋₆ alkyl group optionally substituted by 1 to 3 halogen atoms, and

(b) a C₁₋₆ alkoxy group optionally substituted by 1 to 3 halogen atoms;

(12) an aromatic heterocyclylsulfonyl group (e.g., imidazolylsulfonyl, pyridylsulfonyl) optionally substituted by 1 to 3 C₁₋₆ alkyl groups;
(13) a carbamoyl group optionally mono- or di-substituted by substituent(s) selected from

(a) a C₁₋₆ alkyl group optionally substituted by 1 to 3 substituents selected from
(i) a halogen atom,
(ii) an aromatic heterocyclic group (e.g., pyridyl, furyl) optionally substituted by 1 to 3 C₁₋₆ alkyl groups, and
(iii) a C₁₋₆ alkylsulfonyl group (e.g., methylsulfonyl),

(b) a C₆₋₁₄ aryl group (e.g., phenyl),

(c) a C₇₋₁₃ aralkyl group (e.g., benzyl),

(d) an aromatic heterocyclic group (e.g., pyridyl, thiadiazolyl, oxadiazolyl) optionally substituted by 1 to 3 C₁₋₆ alkyl groups optionally substituted by 1 to 3 halogen atoms, and

(e) a non-aromatic heterocyclic group (e.g., 1,1-dioxidotetrahydrothienyl);

(14) a thiocarbamoyl group optionally mono- or di-substituted by C₁₋₆ alkyl group(s) optionally substituted by 1 to 3 halogen atoms;
(15) a sulfamoyl group optionally mono- or di-substituted by C₁₋₆ alkyl group(s) optionally substituted by 1 to 3 halogen atoms;
(16) a carboxy group;
(17) a hydroxy group;
(18) a C₁₋₆ alkoxy group optionally substituted by 1 to 3 substituents selected from

(a) a halogen atom,

(b) a carboxy group,

(c) hydroxyl group,

(d) a C₁₋₆ alkoxy group,

(e) a C₁₋₆ alkoxy-carbonyl group,

(f) an amino group optionally mono- or di-substituted by substituent(s) selected from a C₁₋₆ alkyl group and a C₁₋₆ alkoxy-carbonyl group,

(g) a C₃₋₁₀ cycloalkyl group (e.g., cyclopropyl, cyclopentyl),

(h) a C₁₋₆ alkylsulfonyl group (e.g., methylsulfonyl, ethylsulfonyl),

(i) an aromatic heterocyclic group (e.g., imidazolyl) optionally substituted by 1 to 3 C₁₋₆ alkyl groups, and

(j) a non-aromatic heterocyclic group (e.g., morpholinyl);

(19) a C₂₋₆ alkenyloxy group (e.g., ethenyloxy) optionally substituted by 1 to 3 halogen atoms;
(20) a C₃₋₁₀ cycloalkyloxy group (e.g., cyclohexyloxy, cyclopentyloxy);
(21) a C₇₋₁₃ aralkyloxy group (e.g., benzyloxy);
(22) a C₆₋₁₄ aryloxy group (e.g., phenyloxy, naphthyloxy);
(23) a non-aromatic heterocyclyloxy group (e.g., tetrahydropyranyloxy, tetrahydrothiopyranyloxy, 1,1-dioxidotetrahydrothiopyranyloxy);
(24) a C₁₋₆ alkyl-carbonyloxy group (e.g., acetyloxy, tert-butylcarbonyloxy);
(25) a C₃₋₁₀ cycloalkyl-oxycarbonyl group (e.g., cyclopentyloxycarbonyl);
(26) a C₆₋₁₄ aryl-carbonyl group (e.g., benzoyl) optionally substituted by 1 to 3 substituents selected from

(a) a halogen atom, and

(b) a C₁₋₆ alkyl group optionally substituted by 1 to 3 halogen atoms;

(27) a non-aromatic heterocyclylcarbonyl group (e.g., pyrrolidinylcarbonyl, morpholinylcarbonyl, 1,1-dioxidothiomorpholinylcarbonyl) optionally substituted by 1 to 3 substituents selected from

(a) a C₆₋₁₄ aryl group (e.g., phenyl), and

(b) a C₁₋₆ alkyl group optionally substituted by 1 to 3 halogen atoms;

(28) a mercapto group;
(29) a C₁₋₆ alkylthio group (e.g., methylthio, ethylthio) optionally substituted by 1 to 3 halogen atoms;
(30) a C₁₋₁₃ aralkylthio group (e.g., benzylthio);
(31) a C₆₋₁₄ arylthio group (e.g., phenylthio, naphthylthio);
(32) a sulfo group;
(33) a cyano group;
(34) an azido group;
(35) a nitro group;
(36) a nitroso group;
(37) a halogen atom;
(38) a C₁₋₆ alkylsulfinyl group (e.g., methylsulfinyl);
(39) a C₁₋₃ alkylenedioxy group;
(40) an aromatic heterocyclylcarbonyl group (e.g., pyrazolylcarbonyl, pyrazinylcarbonyl, isoxazolylcarbonyl, pyridylcarbonyl, thiazolylcarbonyl) optionally substituted by 1 to 3 C₁₋₆ alkyl groups optionally substituted by 1 to 3 halogen atoms;
(41) a hydroxyimino group;
and the like can be mentioned.

The C₃₋₁₀ cycloalkyl group, C₃₋₁₀ cycloalkenyl group, C₄₋₁₀ cycloalkadienyl group, C₆₋₁₄ aryl group, C₇₋₁₃ aralkyl group, C₈₋₁₃ arylalkenyl group and C₃₋₁₀ cycloalkyl-C₁₋₆ alkyl group, which are exemplarily recited as the aforementioned “hydrocarbon group”, each optionally have 1 to 3 substituents at substitutable position(s).

As such substituents, for example,

(1) those exemplarily recited as the substituents of the aforementioned C₁₋₁₀ alkyl group and the like;
(2) a C₁₋₆ alkyl group optionally substituted by 1 to 3 substituents selected from

(a) a halogen atom,

(b) a carboxy group,

(c) a hydroxy group,

(d) a C₁₋₆ alkoxy-carbonyl group,

(e) a C₁₋₆ alkyl-carbonyloxy group (e.g., acetyloxy),

(f) a carbamoyl group,

(g) a cyano group,

(h) an amino group,

(i) a hydroxyimino group, and

(j) a non-aromatic heterocyclic group (e.g., morpholinyl, pyrrolidinyl);

(3) a C₂₋₆ alkenyl group (e.g., ethenyl, 1-propenyl) optionally substituted by 1 to 3 substituents selected from

(a) a halogen atom,

(b) a carboxy group,

(c) a C₁₋₆ alkoxy-carbonyl group, and

(d) a carbamoyl group;

(4) a C₇₋₁₃ aralkyl group (e.g., benzyl) optionally substituted by 1 to 3 substituents selected from

(a) a C₁₋₆ alkyl group optionally substituted by 1 to 3 halogen atoms,

(b) a hydroxy group,

(c) a C₁₋₆ alkoxy group, and

(d) a halogen atom;

(5) an oxo group;
and the like can be mentioned.

As the “heterocyclic group” of the aforementioned “optionally substituted heterocyclic group”, an aromatic heterocyclic group and a non-aromatic heterocyclic group can be mentioned.

Here, as the aromatic heterocyclic group, for example, a 5- to 7-membered monocyclic aromatic heterocyclic group containing, as a ring-constituting atom besides carbon atoms, 1 to 4 heteroatoms selected from an oxygen atom, a sulfur atom and a nitrogen atom, and a fused aromatic heterocyclic group can be mentioned. As the fused aromatic heterocyclic group, for example, a group derived from a fused ring wherein a ring constituting such 5- to 7-membered monocyclic aromatic heterocyclic group, and 1 or 2 rings selected from a 5- or 6-membered aromatic heterocycle containing 1 or 2 nitrogen atoms (e.g., pyrrole, imidazole, pyrazole, pyrazine, pyridine, pyrimidine), a 5-membered aromatic heterocycle containing one sulfur atom (e.g., thiophene) and a benzene ring are condensed, and the like can be mentioned.

As preferable examples of the aromatic heterocyclic group,

monocyclic aromatic heterocyclic groups such as furyl (e.g., 2-furyl, 3-furyl), thienyl (e.g., 2-thienyl, 3-thienyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl, 4-pyridazinyl), pyrazinyl (e.g., 2-pyrazinyl), pyrrolyl (e.g., 1-pyrrolyl, 2-pyrrolyl, 3-pyrrolyl), imidazolyl (e.g., 1-imidazolyl, 2-imidazolyl, 4-imidazolyl, 5-imidazolyl), pyrazolyl (e.g., 1-pyrazolyl, 3-pyrazolyl, 4-pyrazolyl), thiazolyl (e.g., 2-thiazolyl, 4-thiazolyl, 5-thiazolyl), isothiazolyl (e.g., 4-isothiazolyl), oxazolyl (e.g., 2-oxazolyl, 4-oxazolyl, 5-oxazolyl), isoxazolyl, oxadiazolyl (e.g., 1,2,4-oxadiazol-5-yl, 1,3,4-oxadiazol-2-yl), thiadiazolyl (e.g., 1,3,4-thiadiazol-2-yl), triazolyl (e.g., 1,2,4-triazol-1-yl, 1,2,4-triazol-3-yl, 1,2,3-triazol-1-yl, 1,2,3-triazol-2-yl, 1,2,3-triazol-4-yl), tetrazolyl (e.g., tetrazol-1-yl, tetrazol-5-yl), triazinyl (e.g., 1,2,4-triazin-1-yl, 1,2,4-triazin-3-yl, 1,3,5-triazin-1-yl,) and the like; fused aromatic heterocyclic groups such as quinolyl (e.g., 2-quinolyl, 3-quinolyl, 4-quinolyl, 6-quinolyl), isoquinolyl (e.g., 3-isoquinolyl), quinazolyl (e.g., 2-quinazolyl, 4-quinazolyl), quinoxalyl (e.g., 2-quinoxalyl, 6-quinoxalyl), benzofuranyl (e.g., 2-benzofuranyl, 3-benzofuranyl), benzothiophenyl (e.g., 2-benzothiophenyl, 3-benzothiophenyl), benzoxazolyl (e.g., 2-benzoxazolyl), benzisoxazolyl (e.g., 7-benzisoxazolyl), benzothiazolyl (e.g., 2-benzothiazolyl), benzimidazolyl (e.g., benzimidazol-1-yl, benzimidazol-2-yl, benzimidazol-5-yl), benzotriazolyl (e.g., 1H-1,2,3-benzotriazol-5-yl), indolyl (e.g., indol-1-yl, indol-2-yl, indol-3-yl, indol-5-yl), indazolyl (e.g., 1H-indazol-3-yl), pyrrolopyrazinyl (e.g., 1H-pyrrolo[2,3-b]pyrazin-2-yl, 1H-pyrrolo[2,3-b]pyrazin-6-yl), imidazopyridinyl (e.g., 1H-imidazo[4,5-b]pyridin-2-yl, 1H-imidazo[4,5-c]pyridin-2-yl, 2H-imidazo[1,2-a]pyridin-3-yl, 2H-imidazo[1,2-a]pyridin-6-yl), imidazopyrazinyl (e.g., 1H-imidazo[4,5-b]pyrazin-2-yl), pyrazolopyridinyl (e.g., 1H-pyrazolo[4,3-c]pyridin-3-yl), pyrazolothienyl (e.g., 2H-pyrazolo[3,4-b]thiophen-2-yl), pyrazolotriazinyl (e.g., pyrazolo[5,1-c][1,2,4]triazin-3-yl) and the like;
and the like can be mentioned.

In the present specification, the “heteroaryl group” has the same meaning as the aromatic heterocyclic group described above.

As the non-aromatic heterocyclic group, for example, a 5- to 7-membered monocyclic non-aromatic heterocyclic group containing, as a ring-constituting atom besides carbon atoms, 1 to 4 heteroatoms selected from an oxygen atom, a sulfur atom and a nitrogen atom, and a fused non-aromatic heterocyclic group can be mentioned. As the fused non-aromatic heterocyclic group, for example, a group derived from a fused ring wherein a ring constituting such 5- to 7-membered monocyclic non-aromatic heterocyclic group, and 1 or 2 rings selected from a 5- or 6-membered aromatic or non-aromatic heterocycle containing 1 or 2 nitrogen atoms (e.g., pyrrole, imidazole, pyrazole, pyrazine, pyridine, pyrimidine), a 5-membered aromatic or non-aromatic heterocycle containing one sulfur atom (e.g., thiophene) and a benzene ring are condensed, a group obtained by partial saturation of said group, and the like can be mentioned.

As preferable examples of the non-aromatic heterocyclic group, tetrahydrofuryl (e.g., 2-tetrahydrofuryl), pyrrolidinyl (e.g., 1-pyrrolidinyl), 1,1-dioxidotetrahydrothienyl (e.g., 1,1-dioxidotetrahydro-3-thienyl), piperidinyl (e.g., piperidino), morpholinyl (e.g., morpholino), thiomorpholinyl (e.g., thiomorpholino), 1,1-dioxidothiomorpholinyl (e.g., 1,1-dioxidothiomorpholino), piperazinyl (e.g., 1-piperazinyl), hexamethyleneiminyl (e.g., hexamethyleneimin-1-yl), oxazolidinyl (e.g., oxazolidin-3-yl), thiazolidinyl (e.g., thiazolidin-3-yl), imidazolidinyl (e.g., imidazolidin-3-yl), dihydroisoindolyl (e.g., 1,3-dihydro-2H-isoindol-2-yl), dioxolyl (e.g., 1,3-dioxol-4-yl), dioxolanyl (e.g., 1,3-dioxolan-4-yl), dihydrooxadiazolyl (e.g., 4,5-dihydro-1,2,4-oxadiazol-3-yl), thioxooxazolidinyl (e.g., 2-thioxo-1,3-oxazolidin-5-yl), tetrahydropyranyl (e.g., 4-tetrahydropyranyl), tetrahydrothiopyranyl (e.g., 4-tetrahydrothiopyranyl), 1,1-dioxidotetrahydrothiopyranyl (e.g., 1,1-dioxidotetrahydrothiopyran-4-yl), dihydrobenzofuranyl (e.g., 2,3-dihydro-1-benzofuran-5-yl), dihydrobenzodioxinyl (e.g., 2,3-dihydro-1,4-benzodioxin-2-yl), dihydrobenzodioxepinyl (e.g., 3,4-dihydro-2H-1,5-benzodioxepin-2-yl), tetrahydrobenzofuranyl (e.g., 4,5,6,7-tetrahydro-1-benzofuran-3-yl), tetrahydrobenzothiazolyl (e.g., 4,5,6,7-tetrahydro-1-benzothiazol-2-yl), tetrahydrobenzoxazolyl (e.g., 4,5,6,7-tetrahydro-1-benzoxazol-2-yl), chromenyl (e.g., 4H-chromen-2-yl, 2H-chromen-3-yl), dihydroquinolinyl (e.g., 1,2-dihydroquinolin-2-yl), tetrahydroquinolinyl (e.g., 1,2,3,4-tetrahydroquinolin-2-yl), dihydroisoquinolinyl (e.g., 1,2-dihydroisoquinolin-2-yl), tetrahydroisoquinolinyl (e.g., 1,2,3,4-tetrahydroisoquinolin-4-yl, 1,2,3,4-tetrahydroisoquinolin-2-yl), dihydrophthalazinyl (e.g., 1,4-dihydrophthalazin-4-yl), pyrazolidinyl (e.g., pyrazolidin-1-yl), tetrahydroindazolyl (e.g., 4,5,6,7-tetrahydro-2H-indazol-2-yl), tetrahydroquinazolinyl (e.g., 5,6,7,8-tetrahydroquinazolin-6-yl), tetrahydrothiazolopyridinyl (e.g., 4,5,6,7-tetrahydrothiazolo [5.4-c]pyridin-6-yl), tetrahydroimidazopyridinyl (e.g., 1,2,3,4-tetrahydroimidazo[4.5-c]pyridin-2-yl), tetrahydropyrazolopyridinyl (e.g., 1,2,3,4-tetrahydropyrazolo[3.4-c]pyridin-2-yl), tetrahydrotriazolopyrazinyl (e.g., 1,2,3,4-tetrahydrotriazolo[4.3-a]pyrazin-2-yl), tetrahydroimidazopyrazinyl (e.g., 1,2,3,4-tetrahydroimidazo[1.2-a]pyrazin-2-yl, 1,2,3,4-tetrahydroimidazo[3.4-a]pyrazin-2-yl), tetrahydropyridopyrimidinyl (e.g., 5,6,7,8-tetrahydropyrido [5.4-c]pyrimidin-6-yl) and the like can be mentioned.

The above-mentioned non-aromatic heterocyclic group may be a heterospiro ring group. For example, the 5- to 7-membered monocyclic non-aromatic heterocyclic group and the fused non-aromatic heterocyclic group optionally form, together with a C₃₋₁₀ cycloalkane, a C₃₋₁₀ cycloalkene, a C₄₋₁₀ cycloalkadiene or a non-aromatic heterocycle, a spiro ring group. Here, as the C₃₋₁₀ cycloalkane, C₃₋₁₀ cycloalkene and C₄₋₁₀ cycloalkadiene, rings corresponding to the C₃₋₁₀ cycloalkyl group, C₃₋₁₀ cycloalkenyl group and C₄₋₁₀ cycloalkadienyl group, which are exemplarily recited as the “hydrocarbon group” of the above-mentioned “optionally substituted hydrocarbon group”, can be mentioned. As the non-aromatic heterocycle, a ring corresponding to the above-mentioned non-aromatic heterocyclic group can be mentioned. As such a spiro ring group, 2,8-diazaspiro[4.5]decan-8-yl and the like can be mentioned.

The above-mentioned non-aromatic heterocyclic group may be a crosslinked non-aromatic heterocyclic group. As the crosslinked non-aromatic heterocyclic group, 2,5-diazabicyclo[2.2.1]heptan-2-yl and the like can be mentioned.

The “heterocyclic group” of the aforementioned “optionally substituted heterocyclic group” optionally has 1 to 3 substituents at substitutable position(s). As such substituents, for example, those exemplarily recited as the substituents of the C₃₋₁₀ cycloalkyl group and the like exemplarily recited as the “hydrocarbon group” of the aforementioned “optionally substituted hydrocarbon group” can be mentioned.

As the aforementioned “optionally substituted hydroxy group”, for example, a hydroxy group optionally substituted by a substituent selected from a C₁₋₁₀ alkyl group, a C₂₋₁₀ alkenyl group, a C₃₋₁₀ cycloalkyl group, a C₃₋₁₀ cycloalkenyl group, a C₆₋₁₄ aryl group, a C₇₋₁₃ aralkyl group, a C₈₋₁₃ arylalkenyl group, a C₁₋₆ alkyl-carbonyl group, a heterocyclic group and the like, each of which is optionally substituted, can be mentioned.

Here, as the C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group and C₈₋₁₃ arylalkenyl group, those exemplarily recited as the “hydrocarbon group” of the aforementioned “optionally substituted hydrocarbon group” can be mentioned.

As the heterocyclic group, the “aromatic heterocyclic group” and “non-aromatic heterocyclic group”, which are exemplarily recited as the “heterocyclic group” of the aforementioned “optionally substituted heterocyclic group”, can be mentioned.

The aforementioned C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group, C₈₋₁₃ arylalkenyl group, C₁₋₆ alkyl-carbonyl group and heterocyclic group each optionally have 1 to 5 (preferably 1 to 3) substituents at substitutable position(s).

As the substituents of the C₁₋₁₀ alkyl group, C₂₋₁₀ alkenyl group and C₁₋₆ alkyl-carbonyl group, those exemplarily recited as the substituents of the C₁₋₁₀ alkyl group and the like exemplarily recited as the “hydrocarbon group” of the aforementioned “optionally substituted hydrocarbon group” can be mentioned.

As the substituents of the C₃₋₁₀ cycloalkyl group, C₃₋₁₀ cycloalkenyl group, C₆₋₁₄ aryl group, C₇₋₁₃ aralkyl group, C₈₋₁₃ arylalkenyl group and heterocyclic group, those exemplarily recited as the substituents of the C₃₋₁₀ cycloalkyl group and the like exemplarily recited as the “hydrocarbon group” of the aforementioned “optionally substituted hydrocarbon group” can be mentioned.

As the aforementioned “optionally substituted mercapto group”, for example, a mercapto group optionally substituted by a substituent selected from a C₁₋₁₀ alkyl group, a C₂₋₁₀ alkenyl group, a C₃₋₁₀ cycloalkyl group, a C₃₋₁₀ cycloalkenyl group, a C₆₋₁₄ aryl group, a C₇₋₁₃ aralkyl group, a C₈₋₁₃ arylalkenyl group, a C₁₋₆ alkyl-carbonyl group, a heterocyclic group and the like, each of which is optionally substituted, can be mentioned.

As the substituents, those exemplarily recited as the substituents of the aforementioned “optionally substituted hydroxy group” can be mentioned.

As the aforementioned “optionally substituted amino group”, for example, an amino group optionally mono- or di-substituted by substituent(s) selected from a C₁₋₁₀ alkyl group, a C₂₋₁₀ alkenyl group, a C₃₋₁₀ cycloalkyl group, a C₃₋₁₀ cycloalkenyl group, a C₆₋₁₄ aryl group, a C₇₋₁₃ aralkyl group, a C₈₋₁₃ arylalkenyl group and a heterocyclic group, each of which is optionally substituted; an acyl group and the like, can be mentioned.

Here, as the C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group and C₈₋₁₃ arylalkenyl group, those exemplarily recited as the “hydrocarbon group” of the aforementioned “optionally substituted hydrocarbon group” can be mentioned.

As the heterocyclic group, the “aromatic heterocyclic group” and “non-aromatic heterocyclic group”, which are exemplarily recited as the “heterocyclic group” of the aforementioned “optionally substituted heterocyclic group”, can be mentioned. Of these, a 5- to 7-membered monocyclic aromatic heterocyclic group is preferable.

The aforementioned C₁₋₁₀ alkyl group, C₂₋₁₀ alkenyl group, C₃₋₁₀ cycloalkyl group, C₃₋₁₀ cycloalkenyl group, C₆₋₁₄ aryl group, C₇₋₁₃ aralkyl group, C₈₋₁₃ arylalkenyl group and heterocyclic group each optionally have 1 to 3 substituents at substitutable positions(s).

As the substituents of the C₁₋₁₀ alkyl group and C₂₋₁₀ alkenyl group, those exemplarily recited as the substituents of the C₁₋₁₀ alkyl group and the like exemplarily recited as the “hydrocarbon group” of the aforementioned “optionally substituted hydrocarbon group” can be mentioned.

As the substituents of the C₃₋₁₀ cycloalkyl group, C₃₋₁₀ cycloalkenyl group, C₆₋₁₄ aryl group, C₇₋₁₃ aralkyl group, C₈₋₁₃ arylalkenyl group and heterocyclic group, those exemplarily recited as the substituents of the C₃₋₁₀ cycloalkyl group and the like exemplarily recited as the “hydrocarbon group” of the aforementioned “optionally substituted hydrocarbon group” can be mentioned.

As the “acyl group” exemplarily recited as the substituent of the “optionally substituted amino group”, those exemplarily recited as “acyl group” below, which is exemplarily recited as the “substituent” for Ra¹, Ra², Ra³, Ra⁴, Ra⁵, Ra⁶ or Ra⁷, can be mentioned.

As the “acyl group” which is exemplarily recited as the “substituent” for Ra¹, Ra², Ra³, Ra⁴, Ra⁵, Ra⁶ or Ra⁷, for example, a group represented by the formula: —COR^{a}, —CO—OR^{a}, —SO₃R^{a}, —SO₂R^{a}, —SOR^{a}, —CO—NR^{a}′R^{b}′, —CS—NR^{a}′R^{b}′ or —SO₂NR^{a}′R^{b}′ wherein R^{a} is a hydrogen atom, an optionally substituted hydrocarbon group or an optionally substituted heterocyclic group, and R^{a}′ and R^{b}′ are the same or different and each is a hydrogen atom, an optionally substituted hydrocarbon group or an optionally substituted heterocyclic group, or R^{a}′ and R^{b}′ optionally form, together with the adjacent nitrogen atom, an optionally substituted nitrogen-containing heterocycle, and the like can be mentioned.

As the “optionally substituted hydrocarbon group” and “optionally substituted heterocyclic group” for R^{a}, R^{a}′ or R^{b}′, those exemplarily recited as the “optionally substituted hydrocarbon group” and “optionally substituted heterocyclic group”, which are those exemplarily recited as the “substituent” for Ra¹, Ra², Ra³, Ra⁴, Ra⁵, Ra⁶ or Ra⁷, can be mentioned.

As the “nitrogen-containing heterocycle” of the “optionally substituted nitrogen-containing heterocycle” formed by R^{a}′ and R^{b}′ together with the adjacent nitrogen atom, for example, a 5- to 7-membered nitrogen-containing heterocycle containing, as a ring-constituting atom besides carbon atoms, at least one nitrogen atom and optionally further containing one or two heteroatoms selected from an oxygen atom, a sulfur atom and a nitrogen atom can be mentioned. As preferable examples of the nitrogen-containing heterocycle, pyrrolidine, imidazolidine, pyrazolidine, piperidine, piperazine, morpholine, thiomorpholine, oxopiperazine and the like can be mentioned.

The nitrogen-containing heterocycle optionally has 1 to 3 (preferably 1 or 2) substituents at substitutable position(s). As such substituents, those exemplarily recited as the substituents of the C₃₋₁₀ cycloalkyl group and the like exemplarily recited as the “hydrocarbon group” of the aforementioned “optionally substituted hydrocarbon group” can be mentioned.

As preferable examples of the “acyl group”,

(1) a formyl group;
(2) a carboxy group;
(3) a C₁₋₆ alkyl-carbonyl group optionally substituted by 1 to 3 halogen atoms;
(4) a C₁₋₆ alkoxy-carbonyl group (e.g., methoxycarbonyl, ethoxycarbonyl, propoxycarbonyl, tert-butoxycarbonyl) optionally substituted by 1 to 3 substituents selected from

(a) a halogen atom,

(b) a carboxy group,

(c) a carbamoyl group,

(d) a thiocarbamoyl group,

(e) a C₁₋₆ alkoxy group,

(f) a C₁₋₆ alkoxy-carbonyl group, and

(g) a C₁₋₆ alkyl-carbonyloxy group;

(5) a C₃₋₁₀ cycloalkyl-carbonyl group (e.g., cyclopropylcarbonyl, cyclopentylcarbonyl, cyclohexylcarbonyl);
(6) a C₃₋₁₀ cycloalkyl-oxycarbonyl group (e.g., cyclopentyloxycarbonyl);
(7) a C₆₋₁₄ aryl-carbonyl group (e.g., benzoyl, 1-naphthoyl, 2-naphthoyl) optionally substituted by 1 to 3 substituents selected from

(a) a halogen atom,

(b) a cyano group,

(c) a C₁₋₆ alkyl group optionally substituted by 1 to 3 halogen atoms,

(d) a C₁₋₆ alkoxy group,

(e) a carboxy group,

(f) a C₁₋₆ alkoxy-carbonyl group,

(g) an aromatic heterocyclic group (e.g., tetrazolyl, oxadiazolyl),

(h) a non-aromatic heterocyclic group optionally substituted by 1 to 3 oxo groups (e.g., oxooxadiazolinyl), and

(i) a carbamoyl group;

(8) a C₆₋₁₄ aryloxy-carbonyl group (e.g., phenyloxycarbonyl, naphthyloxycarbonyl) optionally substituted by 1 to 3 substituents selected from

(a) a carboxy group,

(b) a C₁₋₆ alkoxy-carbonyl group, and

(c) a carbamoyl group;

(9) a C₇₋₁₃ aralkyloxy-carbonyl group (e.g., benzyloxycarbonyl, phenethyloxycarbonyl) optionally substituted by 1 to 3 substituents selected from

(a) a carboxy group,

(b) a carbamoyl group,

(c) a thiocarbamoyl group,

(d) a C₁₋₆ alkoxy-carbonyl group,

(e) a halogen atom,

(f) a cyano group,

(g) a nitro group,

(h) a C₁₋₆ alkoxy group,

(i) a C₁₋₆ alkylsulfonyl group, and

(j) a C₁₋₆ alkyl group;

(10) a carbamoyl group optionally mono- or di-substituted by substituent(s) selected from

(a) a C₁₋₆ alkyl group optionally substituted by 1 to 3 substituents selected from
(i) a halogen atom,
(ii) an aromatic heterocyclic group (e.g., pyridyl, furyl) optionally substituted by 1 to 3 C₁₋₆ alkyl groups, and
(iii) a C₁₋₆ alkylsulfonyl group (e.g., methylsulfonyl),

(b) a C₆₋₁₄ aryl group (e.g., phenyl) optionally substituted by 1 to 3 substituents selected from
(i) a halogen atom,
(ii) an aromatic heterocyclic group (e.g., pyridyl, furyl) optionally substituted by 1 to 3 C₁₋₆ alkyl groups, and
(iii) a C₁₋₆ alkylsulfonyl group (e.g., methylsulfonyl),

(c) a C₇₋₁₃ aralkyl group (e.g., benzyl),

(d) an aromatic heterocyclic group (e.g., pyridyl, thiadiazolyl, oxadiazolyl) optionally substituted by 1 to 3 C₁₋₆ alkyl groups optionally substituted by 1 to 3 halogen atoms, and

(e) a non-aromatic heterocyclic group (e.g., 1,1-dioxidotetrahydrothienyl);

(11) a C₁₋₆ alkylsulfonyl group (e.g., methylsulfonyl, ethylsulfonyl, isopropylsulfonyl) optionally substituted by 1 to 3 substituents selected from

(a) a halogen atom,

(b) a carboxy group,

(c) a carbamoyl group, and

(d) a C₁₋₆ alkoxy-carbonyl group;

(12) a C₃₋₁₀ cycloalkylsulfonyl group (e.g., cyclopropylsulfonyl);
(13) a C₆₋₁₄ arylsulfonyl group (e.g., benzenesulfonyl) optionally substituted by 1 to 3 substituents selected from

(a) a C₁₋₆ alkyl group optionally substituted by 1 to 3 halogen atoms, and

(b) a C₁₋₆ alkoxy group optionally substituted by 1 to 3 halogen atoms;

(14) an aromatic heterocyclylsulfonyl group (e.g., thienylsulfonyl, imidazolylsulfonyl, pyridylsulfonyl) optionally substituted by 1 to 3 C₁₋₆ alkyl groups;
(15) a sulfamoyl group;
(16) a C₁₋₆ alkylsulfinyl group (e.g., methylsulfinyl);
(17) a thiocarbamoyl group;
(18) a C₇₋₁₃ aralkyl-carbonyl group (e.g., benzylcarbonyl, phenethylcarbonyl) optionally substituted by 1 to 3 halogen atoms;
(19) an aromatic heterocyclylcarbonyl group (e.g., furylcarbonyl, thienylcarbonyl, thiazolylcarbonyl, pyrazolylcarbonyl, isoxazolylcarbonyl, pyridylcarbonyl, pyrazinylcarbonyl, benzofurylcarbonyl, benzothienylcarbonyl, quinoxalinylcarbonyl, imidazolylcarbonyl) optionally substituted by 1 to 3 substituents selected from

(a) a C₁₋₆ alkyl group optionally substituted by 1 to 3 halogen atoms,

(b) a C₆₋₁₄ aryl group,

(c) a C₇₋₁₃ aralkyl group,

(d) a C₁₋₆ alkoxy group,

(e) a carboxy group,

(f) a C₁₋₆ alkoxy-carbonyl group, and

(g) a carbamoyl group;

(20) a non-aromatic heterocyclylcarbonyl group (e.g., tetrahydrofurylcarbonyl, tetrahydrothiopyranylcarbonyl, pyrrolidinylcarbonyl, morpholinylcarbonyl, 1,1-dioxidothiomorpholinylcarbonyl) optionally substituted by 1 to 3 substituents selected from

(a) a C₆₋₁₄ aryl group (e.g., phenyl), and

(b) a C₁₋₆ alkyl group optionally substituted by 1 to 3 halogen atoms;

and the like can be mentioned.

Ra¹ is preferably a hydrogen atom, an optionally substituted hydrocarbon group, an optionally substituted heterocyclic group, an acyl group and the like, more preferably a hydrogen atom, an optionally substituted C₁₋₁₀ alkyl group (preferably, C₁₋₆ alkyl group), an optionally substituted C₆₋₁₄ aryl group, an optionally substituted C₇₋₁₃ aralkyl group, an optionally substituted aromatic heterocyclic group, an optionally substituted C₆₋₁₄ aryl-carbonyl group, an optionally substituted C₆₋₁₄ arylsulfonyl group and the like.

Ra¹ is further preferably

(1) a hydrogen atom;
(2) a C₁₋₆ alkyl group optionally substituted by 1 to 3 aromatic heterocyclic groups (e.g., pyridyl);
(3) a C₆₋₁₄ aryl group (e.g., phenyl) optionally substituted by 1 to 3 substituents selected from

(a) a halogen atom,

(b) a hydroxy group,

(c) a C₁₋₆ alkyl group, and

(d) a C₁₋₆ alkoxy group;

(4) a C₇₋₁₃ aralkyl group (e.g., benzyl) optionally substituted by 1 to 3 substituents selected from

(a) a halogen atom,

(b) a hydroxy group,

(c) a C₁₋₆ alkyl group, and

(d) a C₁₋₆ alkoxy group;

(5) an aromatic heterocyclic group (e.g., pyrimidinyl) optionally substituted by 1 to 3 substituents selected from

(a) a halogen atom,

(b) a hydroxy group,

(c) a C₁₋₆ alkyl group, and

(d) a C₁₋₆ alkoxy group;

(6) a C₆₋₁₄ aryl-carbonyl group (e.g., benzoyl) optionally substituted by 1 to 3 substituents selected from

(a) a halogen atom,

(b) a hydroxy group,

(c) a C₁₋₆ alkyl group, and

(d) a C₁₋₆ alkoxy group;

(7) a C₆₋₁₄ arylsulfonyl group (e.g., benzenesulfonyl) optionally substituted by 1 to 3 substituents selected from

(a) a halogen atom,

(b) a hydroxy group,

(c) a C₁₋₆ alkyl group, and

(d) a C₁₋₆ alkoxy group;

and the like.

Ra² and Ra³ are preferably both hydrogen atoms.

Ra⁴ and Ra⁵ are preferably both hydrogen atoms.

Ra⁶ is preferably a hydrogen atom.

Ra⁷ is preferably a hydrogen atom.

Ring Aa is an optionally substituted aromatic heterocycle.

As the “aromatic heterocycle” of the “optionally substituted aromatic heterocycle” for ring Aa, a ring corresponding to the aromatic heterocyclic group exemplarily recited as the “substituent” for Ra¹, Ra², Ra³, Ra⁴, Ra⁵, Ra⁶ or Ra⁷ can be mentioned. The aromatic heterocycle can be bonded to a carbon atom of the adjacent carbonyl group at any bondable position.

As the “aromatic heterocycle” of the “optionally substituted aromatic heterocycle” for ring Aa, pyridine, pyrazine, pyrimidine, benzimidazole, quinoxaline, indazole, indole, imidazopyridine, and pyridazine are preferable.

The “aromatic heterocycle” of the “optionally substituted aromatic heterocycle” for ring Aa optionally has 1 to 3 substituents at substitutable position(s). As such substituents, for example, those exemplarily recited as the “substituent” for Ra¹, Ra², Ra³, Ra⁴, Ra⁵, Ra⁶ or Ra⁷ can be mentioned.

As the substituents of ring Aa,

an optionally substituted hydrocarbon group;
an optionally substituted heterocyclic group;
an optionally substituted hydroxy group;
an optionally substituted amino group;
an optionally substituted mercapto group;
a cyano group;
an acyl group;
a halogen atom;
and the like are preferable.

As the substituents of ring Aa,

(1) a halogen atom;
(2) a carboxy group;
(3) a cyano group;
(4) a C₁₋₆ alkyl group optionally substituted by 1 to 3 substituents selected from

(a) a halogen atom,

(b) a hydroxy group,

(c) a non-aromatic heterocyclic group (e.g., pyrrolidinyl),

(d) an amino group, and

(e) a hydroxyimino group;

(5) a C₆₋₁₄ aryl group (e.g., phenyl) optionally substituted by 1 to 3 C₁₋₆ alkyl groups;
(6) a C₇₋₁₃ aralkyl group (e.g., benzyl, 2-phenethyl);
(7) an aromatic heterocyclic group (e.g., pyrazolyl, thiazolyl, oxadiazolyl) optionally substituted by 1 to 3 C₁₋₆ alkyl groups optionally substituted by 1 to 3 halogen atoms;
(8) a non-aromatic heterocyclic group (e.g., pyrrolidinyl, morpholinyl);
(9) a C₁₋₆ alkoxy group optionally substituted by 1 to 5 (preferably 1 to 3) substituents selected from

(a) a halogen atom,

(b) a C₁₋₆ alkoxy group,

(c) a C₃₋₁₀ cycloalkyl group (e.g., cyclopropyl, cyclopentyl),

(d) a C₁₋₆ alkylsulfonyl group (e.g., methylsulfonyl, ethylsulfonyl), and

(e) an aromatic heterocyclic group (e.g., imidazolyl) optionally substituted by 1 to 3 C₁₋₆ alkyl groups;

(10) a C₃₋₁₀ cycloalkyloxy group (e.g., cyclopentyloxy);
(11) a C₆₋₁₄ aryloxy group (e.g., phenoxy);
(12) a C₇₋₁₃ aralkyloxy group (e.g., benzyloxy);
(13) a non-aromatic heterocyclyloxy group (e.g., tetrahydropyranyloxy, tetrahydrothiopyranyloxy, 1,1-dioxidotetrahydrothiopyranyloxy);
(14) a C₁₋₆ alkyl-carbonyl group;
(15) a C₁₋₆ alkoxy-carbonyl group;
(16) a C₁₋₆ alkylthio group (e.g., methylthio, ethylthio);
(17) a C₆₋₁₄ arylthio group (e.g., phenylthio);
(18) a C₁₋₆ alkylsulfonyl group (e.g., methylsulfonyl, ethylsulfonyl);
(19) a C₆₋₁₄ arylsulfonyl group (e.g., benzenesulfonyl);
(20) an amino group optionally mono- or di-substituted by substituent(s) selected from

(a) a C₁₋₆ alkyl group optionally substituted by 1 to 3 substituents selected from (i) a hydroxy group,
(ii) a C₁₋₆ alkoxy group, and
(iii) an amino group optionally mono- or di-substituted by C₁₋₆ alkyl group(s),

(b) a C₁₋₆ alkyl-carbonyl group, and

(c) a C₁₋₆ alkoxy-carbonyl group;

(21) a carbamoyl group optionally mono- or di-substituted by C₁₋₆ alkyl group(s);
(22) a hydroxy group;
and the like are more preferable.

Ring Aa is preferably an aromatic heterocycle (preferably, pyridine, pyrazine, pyrimidine, benzimidazole, quinoxaline, indazole, indole, imidazopyridine, pyridazine) optionally substituted by 1 to 3 substituents selected from

an optionally substituted hydrocarbon group;
an optionally substituted heterocyclic group;
an optionally substituted hydroxy group;
an optionally substituted amino group;
an optionally substituted mercapto group;
a cyano group;
an acyl group; and
a halogen atom.

Ring Aa is more preferably an aromatic heterocycle (preferably, pyridine, pyrazine, pyrimidine, benzimidazole, quinoxaline, indazole, indole, imidazopyridine, pyridazine) optionally substituted by 1 to 3 substituents selected from

(1) a halogen atom;
(2) a carboxy group;
(3) a cyano group;
(4) a C₁₋₆ alkyl group optionally substituted by 1 to 3 substituents selected from

(a) a halogen atom,

(b) a hydroxy group,

(c) a non-aromatic heterocyclic group (e.g., pyrrolidinyl),

(d) an amino group, and

(e) a hydroxyimino group;

(5) a C₆₋₁₄ aryl group (e.g., phenyl) optionally substituted by 1 to 3 C₁₋₆ alkyl groups;
(6) a C₇₋₁₃ aralkyl group (e.g., benzyl, 2-phenethyl);
(7) an aromatic heterocyclic group (e.g., pyrazolyl, thiazolyl, oxadiazolyl) optionally substituted by 1 to 3 C₁₋₆ alkyl groups optionally substituted by 1 to 3 halogen atoms;
(8) a non-aromatic heterocyclic group (e.g., pyrrolidinyl, morpholinyl);
(9) a C₁₋₆ alkoxy group optionally substituted by 1 to 5 (preferably 1 to 3) substituents selected from

(a) a halogen atom,

(b) a C₁₋₆ alkoxy group,

(c) a C₃₋₁₀ cycloalkyl group (e.g., cyclopropyl, cyclopentyl),

(d) a C₁₋₆ alkylsulfonyl group (e.g., methylsulfonyl, ethylsulfonyl), and

(e) an aromatic heterocyclic group (e.g., imidazolyl) optionally substituted by 1 to 3 C₁₋₆ alkyl groups;

(10) a C₃₋₁₀ cycloalkyloxy group (e.g., cyclopentyloxy);
(11) a C₆₋₁₄ aryloxy group (e.g., phenoxy);
(12) a C₇₋₁₃ aralkyloxy group (e.g., benzyloxy);
(13) a non-aromatic heterocyclyloxy group (e.g., tetrahydropyranyloxy, tetrahydrothiopyranyloxy, 1,1-dioxidotetrahydrothiopyranyloxy);
(14) a C₁₋₆ alkyl-carbonyl group;
(15) a C₁₋₆ alkoxy-carbonyl group;
(16) a C₁₋₆ alkylthio group (e.g., methylthio, ethylthio);
(17) a C₆₋₁₄ arylthio group (e.g., phenylthio);
(18) a C₁₋₆ alkylsulfonyl group (e.g., methylsulfonyl, ethylsulfonyl);
(19) a C₆₋₁₄ arylsulfonyl group (e.g., benzenesulfonyl);
(20) an amino group optionally mono- or di-substituted by substituent(s) selected from

(a) a C₁₋₆ alkyl group optionally substituted by 1 to 3 substituents selected from
(i) a hydroxy group,
(ii) a C₁₋₆ alkoxy group, and
(iii) an amino group optionally mono- or di-substituted by C₁₋₆ alkyl group(s),

(b) a C₁₋₆ alkyl-carbonyl group, and

(c) a C₁₋₆ alkoxy-carbonyl group;

(21) a carbamoyl group optionally mono- or di-substituted by C₁₋₆ alkyl group(s); and
(22) a hydroxy group.

Ring Ba is a 5-membered nitrogen-containing aromatic heterocycle optionally condensed with an aromatic ring, which is optionally further substituted.

As the “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Ba, a ring corresponding to the 5-membered nitrogen-containing aromatic heterocyclic group, and a ring corresponding to the 5-membered nitrogen-containing aromatic heterocyclic group condensed with an aromatic ring selected from a 5- or 6-membered aromatic heterocycle containing 1 or 2 nitrogen atoms (e.g., pyrrole, imidazole, pyrazole, pyrazine, pyridine, pyrimidine), a 5-membered aromatic heterocycle containing one sulfur atom (e.g., thiophene) and a benzene ring, can be mentioned, from among the aromatic heterocyclic groups exemplarily recited as the “substituent” for Ra¹, Ra², Ra³, Ra⁴, Ra⁵, Ra⁶ or Ra⁷. The 5-membered nitrogen-containing aromatic heterocycle optionally condensed with an aromatic ring can be bonded to a carbon atom of the adjacent carbonyl group at any bondable position of the 5-membered ring thereof.

As the “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Ba, pyrazole, benzimidazole, indole and indazole are preferable, and pyrazole and indole are particularly preferable.

The “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Ba optionally further has 1 to 3 substituents, besides Ra¹, at substitutable position(s). As such substituents, for example, those (except an oxo group) exemplarily recited as the substituents of the C₃₋₁₀ cycloalkyl group and the like exemplarily recited as the “substituent” for Ra¹, Ra², Ra³, Ra⁴, Ra⁵, Ra⁶ or Ra⁷ can be mentioned.

As the substituents other than Ra¹ of ring Ba,

(1) a C₁₋₆ alkyl group optionally substituted by 1 to 3 halogen atoms;
(2) a C₆₋₁₄ aryl group;
(3) a C₁₋₆ alkoxy group;
(4) a C₇₋₁₃ aralkyloxy group (e.g., benzyloxy);
(5) an amino group optionally mono- or di-substituted by substituent(s) selected from

(a) a C₁₋₆ alkyl group, and

(b) a C₁₋₆ alkoxy-carbonyl group;

and the like are preferable (a C₁₋₆ alkyl group optionally substituted by 1 to 3 halogen atoms is particularly preferable).

Ring Ba is preferably pyrazole, benzimidazole, indole or indazole (particularly preferably, pyrazole or indole), each of which is substituted by Ra¹ and optionally further substituted.

Ring Ba is more preferably pyrazole, benzimidazole, indole or indazole (particularly preferably, pyrazole or indole), each of which is substituted by Ra¹ and optionally further substituted by 1 to 3 substituents selected from

(1) a C₁₋₆ alkyl group optionally substituted by 1 to 3 halogen atoms;
(2) a C₆₋₁₄ aryl group;
(3) a C₁₋₆ alkoxy group;
(4) a C₇₋₁₃ aralkyloxy group (e.g., benzyloxy); and
(5) an amino group optionally mono- or di-substituted by substituent(s) selected from

(a) a C₁₋₆ alkyl group, and

(b) a C₁₋₆ alkoxy-carbonyl group

(particularly preferably, a C₁₋₆ alkyl group optionally substituted by 1 to 3 halogen atoms).

In compound (Ia),

1) when ring Ba is pyrazole which is optionally further substituted, then ring Ba does not have optionally substituted tetrahydrofurylmethoxy as a substituent other than Ra¹;

2) when ring Ba is imidazole which is optionally further substituted, then ring Ba does not have optionally substituted quinolyl as a substituent other than Ra¹;

3) when ring Ba is pyrazole which is optionally further substituted, then Ra¹ is not optionally substituted quinolyl; and

4) ring Aa is not the same as ring Ba.

As preferable examples of compound (Ia), the following compounds can be mentioned.

### [Compound Ia-A]

A compound wherein

ring Ba is pyrazole, benzimidazole, indole or indazole (particularly preferably, pyrazole or indole), each of which is substituted by Ra¹ and optionally further substituted [ring Ba is preferably pyrazole, benzimidazole, indole or indazole (particularly preferably, pyrazole or indole), each of which is substituted by Ra¹ and optionally further substituted by 1 to 3 substituents selected from

(1) a C₁₋₆ alkyl group optionally substituted by 1 to 3 halogen atoms;
(2) a C₆₋₁₄ aryl group;
(3) a C₁₋₆ alkoxy group;
(4) a C₇₋₁₃ aralkyloxy group (e.g., benzyloxy); and
(5) an amino group optionally mono- or di-substituted by substituent(s) selected from

(a) a C₁₋₆ alkyl group, and

(b) a C₁₋₆ alkoxy-carbonyl group

(particularly preferably, a C₁₋₆ alkyl group optionally substituted by 1 to 3 halogen atoms)];

Ra¹ is a hydrogen atom, an optionally substituted hydrocarbon group, an optionally substituted heterocyclic group or an acyl group

[Ra¹ is preferably a hydrogen atom, an optionally substituted C₁₋₁₀ alkyl group (preferably, a C₁₋₆ alkyl group), an optionally substituted C₆₋₁₄ aryl group, an optionally substituted C₇₋₁₃ aralkyl group, an optionally substituted aromatic heterocyclic group, an optionally substituted C₆₋₁₄ aryl-carbonyl group or an optionally substituted C₆₋₁₄ arylsulfonyl group.
Ra¹ is more preferably
(1) a hydrogen atom;
(2) a C₁₋₆ alkyl group optionally substituted by 1 to 3 aromatic heterocyclic groups (e.g., pyridyl);
(3) a C₆₋₁₄ aryl group (e.g., phenyl) optionally substituted by 1 to 3 substituents selected from

(a) a halogen atom,

(b) a hydroxy group,

(c) a C₁₋₆ alkyl group, and

(d) a C₁₋₆ alkoxy group;

(4) a C₇₋₁₃ aralkyl group (e.g., benzyl) optionally substituted by 1 to 3 substituents selected from

(a) a halogen atom,

(b) a hydroxy group,

(c) a C₁₋₆ alkyl group, and

(d) a C₁₋₆ alkoxy group;

(5) an aromatic heterocyclic group (e.g., pyrimidinyl) optionally substituted by 1 to 3 substituents selected from

(a) a halogen atom,

(b) a hydroxy group,

(c) a C₁₋₆ alkyl group, and

(d) a C₁₋₆ alkoxy group;

(6) a C₆₋₁₄ aryl-carbonyl group (e.g., benzoyl) optionally substituted by 1 to 3 substituents selected from

(a) a halogen atom,

(b) a hydroxy group,

(c) a C₁₋₆ alkyl group, and

(d) a C₁₋₆ alkoxy group; or

(7) a C₆₋₁₄ arylsulfonyl group (e.g., benzenesulfonyl) optionally substituted by 1 to 3 substituents selected from

(a) a halogen atom,

(b) a hydroxy group,

(c) a C₁₋₆ alkyl group, and

(d) a C₁₋₆ alkoxy group];

ring Aa is an aromatic heterocycle (preferably, pyridine, pyrazine, pyrimidine, benzimidazole, quinoxaline, indazole, indole) optionally substituted by 1 to 3 substituents selected from

an optionally substituted hydrocarbon group;
an optionally substituted heterocyclic group;
an optionally substituted hydroxy group;
an optionally substituted amino group;
an optionally substituted mercapto group;
a cyano group;
an acyl group; and
a halogen atom
[ring Aa is preferably an aromatic heterocycle (preferably, pyridine, pyrazine, pyrimidine, benzimidazole, quinoxaline, indazole, indole) optionally substituted by 1 to 3 substituents selected from
(1) a halogen atom;
(2) a carboxy group;
(3) a cyano group;
(4) a C₁₋₆ alkyl group optionally substituted by 1 to 3 substituents selected from

(a) a halogen atom,

(b) a hydroxy group,

(c) a non-aromatic heterocyclic group (e.g., pyrrolidinyl),

(d) an amino group, and

(e) a hydroxyimino group;

(5) a C₆₋₁₄ aryl group (e.g., phenyl) optionally substituted by 1 to 3 C₁₋₆ alkyl groups;
(6) a C₇₋₁₃ aralkyl group (e.g., benzyl, 2-phenethyl);
(7) an aromatic heterocyclic group (e.g., pyrazolyl, thiazolyl, oxadiazolyl) optionally substituted by 1 to 3 C₁₋₆ alkyl groups optionally substituted by 1 to 3 halogen atoms;
(8) a non-aromatic heterocyclic group (e.g., pyrrolidinyl, morpholinyl);
(9) a C₁₋₆ alkoxy group optionally substituted by 1 to 5 (preferably 1 to 3) substituents selected from

(a) a halogen atom,

(b) a C₁₋₆ alkoxy group,

(c) a C₃₋₁₀ cycloalkyl group (e.g., cyclopropyl, cyclopentyl),

(d) a C₁₋₆ alkylsulfonyl group (e.g., methylsulfonyl, ethylsulfonyl), and

(e) an aromatic heterocyclic group (e.g., imidazolyl) optionally substituted by 1 to 3 C₁₋₆ alkyl groups;

(10) a C₃₋₁₀ cycloalkyloxy group (e.g., cyclopentyloxy);
(11) a C₆₋₁₄ aryloxy group (e.g., phenoxy);
(12) a C₇₋₁₃ aralkyloxy group (e.g., benzyloxy);
(13) a non-aromatic heterocyclyloxy group (e.g., tetrahydropyranyloxy, tetrahydrothiopyranyloxy, 1,1-dioxidotetrahydrothiopyranyloxy);
(14) a C₁₋₆ alkyl-carbonyl group;
(15) a C₁₋₆ alkoxy-carbonyl group;
(16) a C₁₋₆ alkylthio group (e.g., methylthio, ethylthio);
(17) a C₆₋₁₄ arylthio group (e.g., phenylthio);
(18) a C₁₋₆ alkylsulfonyl group (e.g., methylsulfonyl, ethylsulfonyl);
(19) a C₆₋₁₄ arylsulfonyl group (e.g., benzenesulfonyl);
(20) an amino group optionally mono- or di-substituted by substituent(s) selected from

(a) a C₁₋₆ alkyl group optionally substituted by 1 to 3 substituents selected from
(i) a hydroxy group,
(ii) a C₁₋₆ alkoxy group, and
(iii) an amino group optionally mono- or di-substituted by C₁₋₆ alkyl group(s),

(b) a C₁₋₆ alkyl-carbonyl group, and

(c) a C₁₋₆ alkoxy-carbonyl group; and

(21) a carbamoyl group optionally mono- or di-substituted by C₁₋₆ alkyl group(s)];

Ra² and Ra³ are both hydrogen atoms;

Ra⁴ and Ra⁵ are both hydrogen atoms;

Ra⁶ is a hydrogen atom; and

Ra⁷ is a hydrogen atom.

### [Compound Ia-B]

A compound wherein

ring Ba is pyrazole, benzimidazole, indole or indazole (particularly preferably, pyrazole or indole), each of which is substituted by Ra¹ and optionally further substituted [ring Ba is preferably pyrazole, benzimidazole, indole or indazole (particularly preferably, pyrazole or indole), each of which is substituted by Ra¹ and optionally further substituted by 1 to 3 substituents selected from

(1) a C₁₋₆ alkyl group optionally substituted by 1 to 3 halogen atoms;
(2) a C₆₋₁₄ aryl group;
(3) a C₁₋₆ alkoxy group;
(4) a C₇₋₁₃ aralkyloxy group (e.g., benzyloxy); and
(5) an amino group optionally mono- or di-substituted by substituent(s) selected from

(a) a C₁₋₆ alkyl group, and

(b) a C₁₋₆ alkoxy-carbonyl group

(particularly preferably, a C₁₋₆ alkyl group optionally substituted by 1 to 3 halogen atoms)];

Ra¹ is a hydrogen atom, an optionally substituted hydrocarbon group, an optionally substituted heterocyclic group or an acyl group

[Ra¹ is preferably a hydrogen atom, an optionally substituted C₁₋₁₀ alkyl group (preferably, a C₁₋₆ alkyl group), an optionally substituted C₆₋₁₄ aryl group, an optionally substituted C₇₋₁₃ aralkyl group, an optionally substituted aromatic heterocyclic group, an optionally substituted C₆₋₁₄ aryl-carbonyl group or an optionally substituted C₆₋₁₄ arylsulfonyl group.

Ra¹ is more preferably

(1) a hydrogen atom;
(2) a C₁₋₆ alkyl group optionally substituted by 1 to 3 aromatic heterocyclic groups (e.g., pyridyl);
(3) a C₆₋₁₄ aryl group (e.g., phenyl) optionally substituted by 1 to 3 substituents selected from

(a) a halogen atom,

(b) a hydroxy group,

(c) a C₁₋₆ alkyl group, and

(d) a C₁₋₆ alkoxy group;

(4) a C₇₋₁₃ aralkyl group (e.g., benzyl) optionally substituted by 1 to 3 substituents selected from

(a) a halogen atom,

(b) a hydroxy group,

(c) a C₁₋₆ alkyl group, and

(d) a C₁₋₆ alkoxy group;

(5) an aromatic heterocyclic group (e.g., pyrimidinyl) optionally substituted by 1 to 3 substituents selected from

(a) a halogen atom,

(b) a hydroxy group,

(c) a C₁₋₆ alkyl group, and

(d) a C₁₋₆ alkoxy group;

(6) a C₆₋₁₄ aryl-carbonyl group (e.g., benzoyl) optionally substituted by 1 to 3 substituents selected from

(a) a halogen atom,

(b) a hydroxy group,

(c) a C₁₋₆ alkyl group, and

(d) a C₁₋₆ alkoxy group; or

(7) a C₆₋₁₄ arylsulfonyl group (e.g., benzenesulfonyl) optionally substituted by 1 to 3 substituents selected from

(a) a halogen atom,

(b) a hydroxy group,

(c) a C₁₋₆ alkyl group, and

(d) a C₁₋₆ alkoxy group];

ring Aa is an aromatic heterocycle (preferably, pyridine, pyrazine, pyrimidine, benzimidazole, quinoxaline, indazole, indole, imidazopyridine, pyridazine) optionally substituted by 1 to 3 substituents selected from an optionally substituted hydrocarbon group;

an optionally substituted heterocyclic group;
an optionally substituted hydroxy group;
an optionally substituted amino group;

an optionally substituted mercapto group;

a cyano group;

an acyl group; and

a halogen atom
[ring Aa is preferably an aromatic heterocycle (preferably, pyridine, pyrazine, pyrimidine, benzimidazole, quinoxaline, indazole, indole, imidazopyridine, pyridazine) optionally substituted by 1 to 3 substituents selected from
(1) a halogen atom;
(2) a carboxy group;
(3) a cyano group;
(4) a C₁₋₆ alkyl group optionally substituted by 1 to 3 substituents selected from

(a) a halogen atom,

(b) a hydroxy group,

(c) a non-aromatic heterocyclic group (e.g., pyrrolidinyl),

(d) an amino group, and

(e) a hydroxyimino group;

(5) a C₆₋₁₄ aryl group (e.g., phenyl) optionally substituted by 1 to 3 C₁₋₆ alkyl groups;
(6) a C₇₋₁₃ aralkyl group (e.g., benzyl, 2-phenethyl);
(7) an aromatic heterocyclic group (e.g., pyrazolyl, thiazolyl, oxadiazolyl) optionally substituted by 1 to 3 C₁₋₆ alkyl groups optionally substituted by 1 to 3 halogen atoms;
(8) a non-aromatic heterocyclic group (e.g., pyrrolidinyl, morpholinyl);
(9) a C₁₋₆ alkoxy group optionally substituted by 1 to 5 (preferably 1 to 3) substituents selected from

(a) a halogen atom,

(b) a C₁₋₆ alkoxy group,

(c) a C₃₋₁₀ cycloalkyl group (e.g., cyclopropyl, cyclopentyl),

(d) a C₁₋₆ alkylsulfonyl group (e.g., methylsulfonyl, ethylsulfonyl), and

(e) an aromatic heterocyclic group (e.g., imidazolyl) optionally substituted by 1 to 3 C₁₋₆ alkyl groups;

(10) a C₃₋₁₀ cycloalkyloxy group (e.g., cyclopentyloxy);
(11) a C₆₋₁₄ aryloxy group (e.g., phenoxy);
(12) a C₇₋₁₃ aralkyloxy group (e.g., benzyloxy);
(13) a non-aromatic heterocyclyloxy group (e.g., tetrahydropyranyloxy, tetrahydrothiopyranyloxy, 1,1-dioxidotetrahydrothiopyranyloxy);
(14) a C₁₋₆ alkyl-carbonyl group;
(15) a C₁₋₆ alkoxy-carbonyl group;
(16) a C₁₋₆ alkylthio group (e.g., methylthio, ethylthio);
(17) a C₆₋₁₄ arylthio group (e.g., phenylthio);
(18) a C₁₋₆ alkylsulfonyl group (e.g., methylsulfonyl, ethylsulfonyl);
(19) a C₆₋₁₄ arylsulfonyl group (e.g., benzenesulfonyl);
(20) an amino group optionally mono- or di-substituted by substituent(s) selected from

(a) a C₁₋₆ alkyl group optionally substituted by 1 to 3 substituents selected from
(i) a hydroxy group,
(ii) a C₁₋₆ alkoxy group, and
(iii) an amino group optionally mono- or di-substituted by C₁₋₆ alkyl group(s),

(b) a C₁₋₆ alkyl-carbonyl group, and

(c) a C₁₋₆ alkoxy-carbonyl group;

(21) a carbamoyl group optionally mono- or di-substituted by C₁₋₆ alkyl group(s); and
(22) a hydroxy group];

Ra² and Ra³ are both hydrogen atoms;

Ra⁴ and Ra⁵ are both hydrogen atoms;

Ra⁶ is a hydrogen atom; and

Ra⁷ is a hydrogen atom.

Each symbol in the formula (Ib) is described in detail in the following.

Ring Ab is an optionally substituted aromatic hydrocarbon.

As the “aromatic hydrocarbon” of the “optionally substituted aromatic hydrocarbon” for ring Ab, a ring corresponding to the C₆₋₁₄ aryl group exemplarily recited as the “substituent” for Ra¹, Ra², Ra³, Ra⁴, Ra⁵, Ra⁶ or Ra⁷ can be mentioned. The aromatic hydrocarbon can be bonded to a carbon atom of the adjacent carbonyl group at any bondable position.

As the “aromatic hydrocarbon” of the “optionally substituted aromatic hydrocarbon” for ring Ab, benzene is preferable.

The “aromatic hydrocarbon” of the “optionally substituted aromatic hydrocarbon” for ring Ab optionally has 1 to 3 substituents at substitutable position(s). As such substituents, for example, those exemplarily recited as the “substituent” for Ra¹, Ra², Ra³, Ra⁴, Ra⁵, Ra⁶ or Ra⁷ can be mentioned.

As the substituents of ring Ab,

an optionally substituted hydrocarbon group;
an optionally substituted heterocyclic group;
an optionally substituted hydroxy group;
a cyano group;
an acyl group;
a halogen atom;
and the like are preferable.

As the substituents of ring Ab,

(1) a C₁₋₆ alkoxy group optionally substituted by 1 to 3 substituents selected from a hydroxy group and a halogen atom,
(2) a hydroxy group,
(3) a halogen atom,
(4) a C₁₋₆ alkyl group,
(5) an aromatic heterocyclic group (e.g., imidazolyl, pyrazolyl),
(6) a sulfamoyl group,
(7) a cyano group
and the like are more preferable, and a halogen atom and a C₁₋₆ alkoxy group optionally substituted by 1 to 3 halogen atoms are particularly preferable.

Ring Ab is preferably an aromatic hydrocarbon (preferably, benzene) optionally substituted by 1 to 3 substituents selected from

an optionally substituted hydrocarbon group;
an optionally substituted heterocyclic group;
an optionally substituted hydroxy group;
a cyano group;
an acyl group; and
a halogen atom.

Ring Ab is more preferably an aromatic hydrocarbon (preferably, benzene) optionally substituted by 1 to 3 substituents selected from

(1) C₁₋₆ alkoxy group optionally substituted by 1 to 3 substituents selected from a hydroxy group and a halogen atom,
(2) a hydroxy group,
(3) a halogen atom,
(4) a C₁₋₆ alkyl group,
(5) an aromatic heterocyclic group (e.g., imidazolyl, pyrazolyl),
(6) a sulfamoyl group, and
(7) a cyano group,
particularly preferably an aromatic hydrocarbon (preferably, benzene) optionally substituted by 1 to 3 substituents selected from a halogen atom and a C₁₋₆ alkoxy group optionally substituted by 1 to 3 halogen atoms.

Ring Bb is a 5-membered nitrogen-containing aromatic heterocycle optionally condensed with an aromatic ring, which is optionally further substituted.

As the “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Bb, a ring corresponding to the 5-membered nitrogen-containing aromatic heterocyclic group, and a ring corresponding to the 5-membered nitrogen-containing aromatic heterocyclic group condensed with an aromatic ring selected from a 5- or 6-membered aromatic heterocycle containing 1 or 2 nitrogen atoms (e.g., pyrrole, imidazole, pyrazole, pyrazine, pyridine, pyrimidine), a 5-membered aromatic heterocycle containing one sulfur atom (e.g., thiophene) and a benzene ring, can be mentioned, from among the aromatic heterocyclic groups exemplarily recited as the “substituent” for Ra¹, Ra², Ra³, Ra⁴, Ra⁵, Ra⁶ or Ra⁷. The 5-membered nitrogen-containing aromatic heterocycle optionally condensed with an aromatic ring can be bonded to a carbon atom of the adjacent carbonyl group at any bondable position of the 5-membered ring thereof.

As the “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Bb, pyrazole, benzimidazole, indole and indazole are preferable, and pyrazole and indole are particularly preferable.

The “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Bb optionally further has 1 to 3 substituents, besides ring Cb, at substitutable position(s). As such substituents, for example, those (except an oxo group) exemplarily recited as the substituents of the C₃₋₁₀ cycloalkyl group and the like exemplarily recited as the “substituent” for Ra¹, Ra², Ra³, Ra⁴, Ra⁵, Ra⁶ or Ra⁷ can be mentioned.

As the substituents other than ring Cb of ring Bb,

(1) a C₁₋₆ alkyl group optionally substituted by 1 to 3 halogen atoms;
(2) a C₆₋₁₄ aryl group;
(3) a C₁₋₆ alkoxy group;
(4) a C₇₋₁₃ aralkyloxy group (e.g., benzyloxy);
(5) an amino group optionally mono- or di-substituted by substituent(s) selected from

(a) a C₁₋₆ alkyl group, and

(b) a C₁₋₆ alkoxy-carbonyl group;

and the like are preferable (a C₁₋₆ alkyl group optionally substituted by 1 to 3 halogen atoms is particularly preferable).

Ring Bb is preferably pyrazole, benzimidazole, indole or indazole (particularly preferably, pyrazole or indole), each of which is substituted by ring Cb and optionally further substituted.

Ring Bb is more preferably pyrazole, benzimidazole, indole or indazole (particularly preferably, pyrazole or indole), each of which is substituted by ring Cb and optionally further substituted by 1 to 3 substituents selected from

(1) a C₁₋₆ alkyl group optionally substituted by 1 to 3 halogen atoms;
(2) a C₆₋₁₄ aryl group;
(3) a C₁₋₆ alkoxy group;
(4) a C₇₋₁₃ aralkyloxy group (e.g., benzyloxy); and
(5) an amino group optionally mono- or di-substituted by substituent(s) selected from

(a) a C₁₋₆ alkyl group, and

(b) a C₁₋₆ alkoxy-carbonyl group

(particularly preferably, a C₁₋₆ alkyl group optionally substituted by 1 to 3 halogen atoms).

Ring Cb is an optionally substituted aromatic heterocycle.

As the “aromatic heterocycle” of the “optionally substituted aromatic heterocycle” for ring Cb, a ring corresponding to the aromatic heterocyclic group exemplarily recited as the “substituent” for Ra¹, Ra², Ra³, Ra⁴, Ra⁵, Ra⁶ or Ra⁷ can be mentioned.

As the “aromatic heterocycle” of the “optionally substituted aromatic heterocycle” for ring Cb, indole, pyridine and pyrimidine are preferable.

The “aromatic heterocycle” of the “optionally substituted aromatic heterocycle” for ring Cb optionally has 1 to 3 substituents at substitutable position(s). As such substituents, for example, those (except an oxo group) exemplarily recited as the substituents of the C₃₋₁₀ cycloalkyl group and the like exemplarily recited as the “substituent” for Ra¹, Ra², Ra³, Ra⁴, Ra⁵, Ra⁶ or Ra⁷ can be mentioned.

As the substituents of ring Cb,

(1) a halogen atom,
(2) a hydroxy group,
(3) a C₁₋₆ alkyl group,
(4) a C₁₋₆ alkoxy group,
and the like are preferable.

Ring Cb is preferably an aromatic heterocycle

(preferably, indole, pyridine, pyrimidine) optionally substituted by 1 to 3 substituents selected from

(1) a halogen atom,
(2) a hydroxy group,
(3) a C₁₋₆ alkyl group, and
(4) a C₁₋₆ alkoxy group.

In compound (Ib), when ring Bb is pyrazole which is optionally further substituted, then ring Cb is not optionally substituted quinoline.

As preferable examples of compound (Ib), the following compounds can be mentioned.

### [Compound Ib-A]

A compound wherein

ring Bb is pyrazole, benzimidazole, indole or indazole (particularly preferably, pyrazole or indole), each of which is substituted by ring Cb and optionally further substituted [ring Bb is preferably pyrazole, benzimidazole, indole or indazole (particularly preferably, pyrazole or indole), each of which is substituted by ring Cb and optionally further substituted by 1 to 3 substituents selected from

(1) a C₁₋₆ alkyl group optionally substituted by 1 to 3 halogen atoms;
(2) a C₆₋₁₄ aryl group;
(3) a C₁₋₆ alkoxy group;
(4) a C₇₋₁₃ aralkyloxy group (e.g., benzyloxy); and
(5) an amino group optionally mono- or di-substituted by substituent(s) selected from

(a) a C₁₋₆ alkyl group, and

(b) a C₁₋₆ alkoxy-carbonyl group

(particularly preferably, a C₁₋₆ alkyl group optionally substituted by 1 to 3 halogen atoms)];

ring Cb is an aromatic heterocycle (preferably, indole, pyridine, pyrimidine) optionally substituted by 1 to 3 substituents selected from

(1) a halogen atom,
(2) a hydroxy group,
(3) a C₁₋₆ alkyl group, and
(4) a C₁₋₆ alkoxy group; and

ring Ab is an aromatic hydrocarbon (preferably, benzene) optionally substituted by 1 to 3 substituents selected from an optionally substituted hydrocarbon group;

an optionally substituted heterocyclic group;
an optionally substituted hydroxy group;
a cyano group;
an acyl group; and
a halogen atom
[ring Ab is preferably an aromatic hydrocarbon (preferably, benzene) optionally substituted by 1 to 3 substituents selected from
(1) a C₁₋₆ alkoxy group optionally substituted by 1 to 3 substituents selected from a hydroxy group and a halogen atom,
(2) a hydroxy group,
(3) a halogen atom,
(4) a C₁₋₆ alkyl group,
(5) an aromatic heterocyclic group (e.g., imidazolyl, pyrazolyl),
(6) a sulfamoyl group, and
(7) a cyano group,
more preferably an aromatic hydrocarbon (preferably, benzene) optionally substituted by 1 to 3 substituents selected from a halogen atom and a C₁₋₆ alkoxy group optionally substituted by 1 to 3 halogen atoms].

Each symbol in the formula (Ic) is described in detail in the following.

In the following explanation, a moiety in the formula (Ic), which is represented by

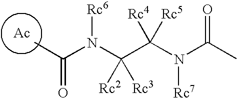

wherein each symbol is as defined in the formula (Ic), is sometimes to be referred to as substituent C.

ring Ac is an optionally substituted aromatic hydrocarbon.

As the “aromatic hydrocarbon” of the “optionally substituted aromatic hydrocarbon” for ring Ac, a ring corresponding to the C₆₋₁₄ aryl group exemplarily recited as the “substituent” for Ra¹, Ra², Ra³, Ra⁴, Ra⁵, Ra⁶ or Ra⁷ can be mentioned. The aromatic hydrocarbon can be bonded to a carbon atom of the adjacent carbonyl group at any bondable position.

As the “aromatic hydrocarbon” of the “optionally substituted aromatic hydrocarbon” for ring Ac, benzene is preferable.

The “aromatic hydrocarbon” of the “optionally substituted aromatic hydrocarbon” for ring Ac optionally has 1 to 3 substituents at substitutable position(s). As such substituents, for example, those exemplarily recited as the “substituent” for Ra¹, Ra², Ra³, Ra⁴, Ra⁵, Ra⁶ or Ra⁷ can be mentioned.

As the substituents of ring Ac,

an optionally substituted hydrocarbon group;
an optionally substituted heterocyclic group;
an optionally substituted hydroxy group;
a cyano group;
an acyl group;
a halogen atom;
and the like are preferable.

As the substituents of ring Ac,

(1) a C₁₋₆ alkoxy group optionally substituted by 1 to 3 substituents selected from a hydroxy group and a halogen atom,
(2) a hydroxy group,
(3) a halogen atom,
(4) a C₁₋₆ alkyl group,
(5) an aromatic heterocyclic group (e.g., imidazolyl, pyrazolyl),
(6) a sulfamoyl group,
(7) a cyano group
and the like are more preferable, and a halogen atom and a C₁₋₆ alkoxy group optionally substituted by 1 to 3 halogen atoms are particularly preferable.

Ring Ac is preferably an aromatic hydrocarbon (preferably, benzene) optionally substituted by 1 to 3 substituents selected from

an optionally substituted hydrocarbon group;
an optionally substituted heterocyclic group;
an optionally substituted hydroxy group;
a cyano group;
an acyl group; and
a halogen atom.

Ring Ac is more preferably an aromatic hydrocarbon (preferably, benzene) optionally substituted by 1 to 3 substituents selected from

(1) a C₁₋₆ alkoxy group optionally substituted by 1 to 3 substituents selected from a hydroxy group and a halogen atom,
(2) a hydroxy group,
(3) a halogen atom,
(4) a C₁₋₆ alkyl group,
(5) an aromatic heterocyclic group (e.g., imidazolyl, pyrazolyl),
(6) a sulfamoyl group, and
(7) a cyano group,
particularly preferably an aromatic hydrocarbon (preferably, benzene) optionally substituted by 1 to 3 substituents selected from a halogen atom and a C₁₋₆ alkoxy group optionally substituted by 1 to 3 halogen atoms.

Ring Bc is a 5-membered nitrogen-containing aromatic heterocycle optionally condensed with an aromatic ring, which is optionally further substituted.

As the “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Bc, a ring corresponding to the 5-membered nitrogen-containing aromatic heterocyclic group, and a ring corresponding to the 5-membered nitrogen-containing aromatic heterocyclic group condensed with an aromatic ring selected from a 5- or 6-membered aromatic heterocycle containing 1 or 2 nitrogen atoms (e.g., pyrrole, imidazole, pyrazole, pyrazine, pyridine, pyrimidine), a 5-membered aromatic heterocycle containing one sulfur atom (e.g., thiophene) and a benzene ring, can be mentioned, from among the aromatic heterocyclic groups exemplarily recited as the “substituent” for Ra¹, Ra², Ra³, Ra⁴, Ra⁵, Ra⁶ or Ra⁷. The 5-membered nitrogen-containing aromatic heterocycle optionally condensed with an aromatic ring can be bonded to a carbon atom of the adjacent carbonyl group at any bondable position of the 5-membered ring thereof.

As the “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Bc, pyrazole, benzimidazole, indole and indazole are preferable, and pyrazole and indole are particularly preferable.

The “5-membered nitrogen-containing aromatic heterocycle optionally condensed with an aromatic ring” of the “5-membered nitrogen-containing aromatic heterocycle optionally condensed with an aromatic ring, which is optionally further substituted” for ring Bc optionally further has 1 to 3 substituents, besides ring Cc, at substitutable position(s). As such substituents, for example, those (except an oxo group) exemplarily recited as the substituents of the C₃₋₁₀ cycloalkyl group and the like exemplarily recited as the “substituent” for Ra¹, Ra², Ra³, Ra⁴, Ra⁵, Ra⁶ or Ra⁷ can be mentioned.

As the substituents other than ring Cc of ring Bc,

(1) a C₁₋₆ alkyl group optionally substituted by 1 to 3 halogen atoms;
(2) a C₆₋₁₄ aryl group;
(3) a C₁₋₆ alkoxy group;
(4) a C₇₋₁₃ aralkyloxy group (e.g., benzyloxy);
(5) an amino group optionally mono- or di-substituted by substituent(s) selected from

(a) a C₁₋₆ alkyl group, and

(b) a C₁₋₆ alkoxy-carbonyl group and the like are preferable (a C₁₋₆ alkyl group optionally substituted by 1 to 3 halogen atoms is particularly preferable).

Ring Bc is preferably pyrazole, benzimidazole, indole or indazole (particularly preferably, pyrazole or indole), each of which is substituted by ring Cc and optionally further substituted.

Ring Bc is more preferably pyrazole, benzimidazole, indole or indazole (particularly preferably, pyrazole or indole), each of which is substituted by ring Cc and optionally further substituted by 1 to 3 substituents selected from

(1) a C₁₋₆ alkyl group optionally substituted by 1 to 3 halogen atoms;
(2) a C₆₋₁₄ aryl group;
(3) a C₁₋₆ alkoxy group;
(4) a C₇₋₁₃ aralkyloxy group (e.g., benzyloxy); and
(5) an amino group optionally mono- or di-substituted by substituent(s) selected from

(a) a C₁₋₆ alkyl group, and

(b) a C₁₋₆ alkoxy-carbonyl group

(particularly preferably, a C₁₋₆ alkyl group optionally substituted by 1 to 3 halogen atoms).

Ring Cc is an optionally substituted aromatic ring.

As the “aromatic ring” of the “optionally substituted aromatic ring” for ring Cc, an aromatic hydrocarbon and an aromatic heterocycle can be mentioned.

Here, as the aromatic hydrocarbon, a ring corresponding to the C₆₋₁₄ aryl group exemplarily recited as the “substituent” for Ra¹, Ra², Ra³, Ra⁴, Ra⁵, Ra⁶ or Ra⁷ can be mentioned.

As the aromatic heterocycle, a ring corresponding to the aromatic heterocyclic group exemplarily recited as the “substituent” for Ra¹, Ra², Ra³, Ra⁴, Ra⁵, Ra⁶ or Ra⁷ can be mentioned.

As the “aromatic ring” of the “optionally substituted aromatic ring” for ring Cc, an aromatic hydrocarbon is preferable, and benzene is particularly preferable.

The “aromatic ring” of the “optionally substituted aromatic ring” for ring Cc optionally has 1 to 3 substituents at substitutable position(s). As such substituents, for example, those (except an oxo group) exemplarily recited as the substituents of the C₃₋₁₀ cycloalkyl group and the like exemplarily recited as the “substituent” for Ra¹, Ra², Ra³, Ra⁴Ra⁵, Ra⁶ or Ra⁷ can be mentioned.

As the substituents of ring Cc,

(1) a halogen atom,
(2) a hydroxy group,
(3) a C₁₋₆ alkyl group,
(4) a C₁₋₆ alkoxy group
and the like are preferable.

Ring Cc is preferably an aromatic hydrocarbon (preferably, benzene) optionally substituted by 1 to 3 substituents selected from

(1) a halogen atom,
(2) a hydroxy group,
(3) a C₁₋₆ alkyl group, and
(4) a C₁₋₆ alkoxy group.

Rc², Rc³, Rc⁴, Rc⁵, Rc⁶ and Rc⁷ are each independently a hydrogen atom or a substituent, or any two of Rc², Rc³, Rc⁴, Rc⁵, Rc⁶ and Rc⁷ are optionally bonded to each other to form a non-aromatic ring.

As the “substituent” for Rc², Rc³, Rc⁴, Rc⁵, Rc⁶ and Rc⁷, those exemplarily recited as the “substituent” for Ra¹, Ra²Ra³, Ra⁴, Ra⁵, Ra⁶ or Ra⁷ can be mentioned.

As the “non-aromatic ring” formed by any two of Rc², Rc³, Rc⁴, Rc⁵, Rc⁶ and Rc⁷ bonded to each other, a non-aromatic cyclic hydrocarbon and a non-aromatic heterocycle can be mentioned.

Here, as the non-aromatic cyclic hydrocarbon, for example, a C₃₋₁₀ cycloalkane, C₃₋₁₀ cycloalkene, C₄₋₁₀ cycloalkadiene and the like, each of which is optionally condensed with a benzene ring, can be mentioned. As the C₃₋₁₀ cycloalkane, C₃₋₁₀ cycloalkene and C₄₋₁₀ cycloalkadiene, rings corresponding to the C₃₋₁₀ cycloalkyl group, C₃₋₁₀ cycloalkenyl group and C₄₋₁₀ cycloalkadienyl group, which are exemplarily recited as the “substituent” for Ra¹, Ra², Ra³, Ra⁴, Ra⁵, Ra⁶ or Ra⁷, can be mentioned.

As the non-aromatic heterocycle, a ring corresponding to the non-aromatic heterocyclic group, which is exemplarily recited as the “substituent” for Ra¹, Ra², Ra³, Ra⁴, Ra⁵, Ra⁶ or Ra⁷, can be mentioned.

Rc² and Rc³ are preferably each independently a hydrogen atom, an acyl group or an optionally substituted hydrocarbon group, or Rc² or Rc³ is bonded to Rc⁴ or Rc⁵ to form a non-aromatic ring, bonded to Rc⁶ to form a non-aromatic heterocycle, or bonded to Rc⁷ to form a non-aromatic heterocycle.

Rc² and Rc³ are more preferably each independently a hydrogen atom, an acyl group or an optionally substituted C₁₋₁₀ alkyl group (preferably, C₁₋₆ alkyl group), or Rc² or Rc³ is bonded to Rc⁴ or Rc⁵ to form a non-aromatic hydrocarbon, bonded to Rc⁶ to form a non-aromatic heterocycle, or bonded to Rc⁷ to form a non-aromatic heterocycle.

Rc² and Rc³ are particularly preferably each independently

(1) a hydrogen atom;
(2) a carboxy group;
(3) a C₁₋₆ alkoxy-carbonyl group; or
(4) a C₁₋₆ alkyl group optionally substituted by 1 to 3 hydroxy groups; or
(5) Rc² or Rc³ is bonded to Rc⁴ or Rc⁵ to form a C₃₋₁₀ cycloalkane (e.g., cyclohexane);
(6) Rc² or Rc³ is bonded to Rc⁶ to form a non-aromatic heterocycle (e.g., piperidine, pyrrolidine); or
(7) Rc² or Rc³ is bonded to Rc⁷ to form a non-aromatic heterocycle (e.g., piperidine).

Rc⁴ and Rc⁵ are preferably each independently a hydrogen atom, an acyl group or an optionally substituted hydrocarbon group, or Rc⁴ or Rc⁵ is bonded to Rc² or Rc³ to form a non-aromatic ring, bonded to Rc⁶ to form a non-aromatic heterocycle, or bonded to Rc⁷ to form a non-aromatic heterocycle.

Rc⁴ and Rc⁵ are more preferably each independently a hydrogen atom, an acyl group or an optionally substituted C₁₋₁₀ alkyl group (preferably, C₁₋₆ alkyl group), or Rc⁴ or Rc⁵ is bonded to Rc² or Rc³ to form a non-aromatic hydrocarbon, bonded to Rc⁶ to form a non-aromatic heterocycle, or bonded to Rc⁷ to form a non-aromatic heterocycle.

Rc⁴ and Rc⁵ are particularly preferably each independently

(1) a hydrogen atom;
(2) a carboxy group;
(3) a C₁₋₆ alkoxy-carbonyl group; or
(4) a C₁₋₆ alkyl group optionally substituted by 1 to 3 hydroxy groups; or
(5) Rc⁴ or Rc⁵ is bonded to Rc² or Rc³ to form a C₃₋₁₀ cycloalkane (e.g., cyclohexane);
(6) Rc⁴ or Rc⁵ is bonded to Rc⁶ to form a non-aromatic heterocycle (e.g., piperidine); or
(7) Rc⁴ or Rc⁵ is bonded to Rc⁷ to form a non-aromatic heterocycle (e.g., piperidine, pyrrolidine).

Rc⁶ is preferably a hydrogen atom or an optionally substituted hydrocarbon group, or Rc⁶ is bonded to Rc² or Rc³ to form a non-aromatic heterocycle, or bonded to Rc⁴ or Rc⁵ to form a non-aromatic heterocycle.

Rc⁶ is more preferably a hydrogen atom or an optionally substituted C₁₋₁₀ alkyl group (preferably, C₁₋₆ alkyl group), or Rc⁶ is bonded to Rc² or Rc³ to form a non-aromatic heterocycle, or bonded to Rc⁴ or Rc⁵ to form a non-aromatic heterocycle.

Rc⁶ is particularly preferably

(1) a hydrogen atom; or
(2) a C₁₋₆ alkyl group; or
(3) Rc⁶ is bonded to Rc² or Rc³ to form a non-aromatic heterocycle (e.g., piperidine, pyrrolidine); or
(4) Rc⁶ is bonded to Rc⁴ or Rc⁵ to form a non-aromatic heterocycle (e.g., piperidine).

Rc⁷ is preferably a hydrogen atom or an optionally substituted hydrocarbon group, or Rc⁷ is bonded to Rc² or Rc³ to form a non-aromatic heterocycle, or bonded to Rc⁴ or Rc⁵ to form a non-aromatic heterocycle.

Rc⁷ is more preferably a hydrogen atom or an optionally substituted C₁₋₁₀ alkyl group (preferably, C₁₋₆ alkyl group), or Rc⁷ is bonded to Rc² or Rc³ to form a non-aromatic heterocycle, or bonded to Rc⁴ or Rc⁵ to form a non-aromatic heterocycle.

Rc⁷ is particularly preferably

(1) a hydrogen atom; or
(2) a C₁₋₆ alkyl group; or
(3) Rc⁷ is bonded to Rc² or Rc³ to form a non-aromatic heterocycle (e.g., piperidine); or
(4) Rc⁷ is bonded to Rc⁴ or Rc⁵ to form a non-aromatic heterocycle (e.g., piperidine, pyrrolidine).

In compound (Ic)

1) ring Bc is not pyrazol-5-yl and 2H-1,2,3-triazol-4-yl, each of which is optionally further substituted (i.e., ring Bc is not pyrazole having substituent C at the 5-position, and 2H-1,2,3-triazole having substituent C at the 4-position, each of which is optionally further substituted);

2) ring Cc is not optionally substituted quinoline;

3) a compound wherein Rc², Rc³, Rc⁴, Rc⁵, Rc⁶ and Rc⁷ are hydrogen atoms is excluded; and

4) when Rc⁶ and Rc⁷ are bonded, then they do not form piperazine.

As preferable examples of compound (Ic), the following compounds can be mentioned.

### [Compound Ic-A]

A compound wherein

ring Bc is pyrazole, benzimidazole, indole or indazole (particularly preferably, pyrazole or indole), each of which is substituted by ring Cc and optionally further substituted [ring Bc is preferably pyrazole, benzimidazole, indole or indazole (particularly preferably, pyrazole or indole), each of which is substituted by ring Cc and optionally further substituted by 1 to 3 substituents selected from

(1) a C₁₋₆ alkyl group optionally substituted by 1 to 3 halogen atoms;
(2) a C₆₋₁₄ aryl group;
(3) a C₁₋₆ alkoxy group;
(4) a C₇₋₁₃ aralkyloxy group (e.g., benzyloxy); and
(5) an amino group optionally mono- or di-substituted by substituent(s) selected from

(a) a C₁₋₆ alkyl group, and

(b) a C₁₋₆ alkoxy-carbonyl group;

(particularly preferably, a C₁₋₆ alkyl group optionally substituted by 1 to 3 halogen atoms)];

ring Cc is an aromatic hydrocarbon (preferably, benzene) optionally substituted by 1 to 3 substituents selected from

(1) a halogen atom,
(2) a hydroxy group,
(3) a C₁₋₆ alkyl group, and
(4) a C₁₋₆ alkoxy group;

ring Ac is an aromatic hydrocarbon (preferably, benzene) optionally substituted by 1 to 3 substituents selected from

an optionally substituted hydrocarbon group;
an optionally substituted heterocyclic group;
an optionally substituted hydroxy group;
a cyano group;
an acyl group; and
a halogen atom
[ring Ac is preferably an aromatic hydrocarbon (preferably, benzene) optionally substituted by 1 to 3 substituents selected from
(1) a C₁₋₆ alkoxy group optionally substituted by 1 to 3 substituents selected from a hydroxy group and a halogen atom,
(2) a hydroxy group,
(3) a halogen atom,
(4) a C₁₋₆ alkyl group,
(5) an aromatic heterocyclic group (e.g., imidazolyl, pyrazolyl),
(6) a sulfamoyl group, and
(7) a cyano group,
more preferably an aromatic hydrocarbon (preferably, benzene) optionally substituted by 1 to 3 substituents selected from a halogen atom and a C₁₋₆ alkoxy group optionally substituted by 1 to 3 halogen atoms];

Rc² and Rc³ are each independently a hydrogen atom, an acyl group or an optionally substituted hydrocarbon group, or Rc² or Rc³ is bonded to Rc⁴ or Rc⁵ to form a non-aromatic ring, bonded to Rc⁶ to form a non-aromatic heterocycle, or bonded to Rc⁷ to form a non-aromatic heterocycle

[Rc² and Rc³ are preferably each independently a hydrogen atom, an acyl group or an optionally substituted C₁₋₁₀ alkyl group (preferably, C₁₋₆ alkyl group), or Rc² or Rc³ is bonded to Rc⁴ or Rc⁵ to form a non-aromatic hydrocarbon, bonded to Rc⁶ to form a non-aromatic heterocycle, or bonded to Rc⁷ to form a non-aromatic heterocycle.
Rc² and Rc³ are more preferably each independently
(1) a hydrogen atom;
(2) a carboxy group;
(3) a C₁₋₆ alkoxy-carbonyl group; or
(4) a C₁₋₆ alkyl group optionally substituted by 1 to 3 hydroxy groups; or
(5) Rc² or Rc³ is bonded to Rc⁴ or Rc⁵ to form a C₃₋₁₀ cycloalkane (e.g., cyclohexane);
(6) Rc² or Rc³ is bonded to Rc⁶ to form a non-aromatic heterocycle (e.g., piperidine, pyrrolidine); or
(7) Rc² or Rc³ is bonded to Rc⁷ to form a non-aromatic heterocycle (e.g., piperidine)];

Rc⁴ and Rc⁵ are each independently a hydrogen atom, an acyl group or an optionally substituted hydrocarbon group, or Rc⁴ or Rc⁵ is bonded to Rc² or Rc³ to form a non-aromatic ring, bonded to Rc⁶ to form a non-aromatic heterocycle, or bonded to Rc⁷ to form a non-aromatic heterocycle

[Rc⁴ and Rc⁵ are preferably each independently a hydrogen atom, an acyl group or an optionally substituted C₁₋₁₀ alkyl group (preferably, C₁₋₆ alkyl group), or Rc⁴ or Rc⁵ is bonded to Rc² or Rc³ to form a non-aromatic hydrocarbon, bonded to Rc⁶ to form a non-aromatic heterocycle, or bonded to Rc⁷ to form a non-aromatic heterocycle.
Rc⁴ and Rc⁵ are more preferably each independently
(1) a hydrogen atom;
(2) a carboxy group;
(3) a C₁₋₆ alkoxy-carbonyl group; or
(4) a C₁₋₆ alkyl group optionally substituted by 1 to 3 hydroxy groups; or
(5) Rc⁴ or Rc⁵ is bonded to Rc² or Rc³ to form a C₃₋₁₀ cycloalkane (e.g., cyclohexane);
(6) Rc⁴ or Rc⁵ is bonded to Rc⁶ to form a non-aromatic heterocycle (e.g., piperidine); or
(7) Rc⁴ or Rc⁵ is bonded to Rc⁷ to form a non-aromatic heterocycle (e.g., piperidine, pyrrolidine)];

Rc⁶ is a hydrogen atom or an optionally substituted hydrocarbon group, or Rc⁶ is bonded to Rc² or Rc³ to form a non-aromatic heterocycle, or bonded to Rc⁴ or Rc⁵ to form a non-aromatic heterocycle

[Rc⁶ is preferably a hydrogen atom or an optionally substituted C₁₋₁₀ alkyl group (preferably, C₁₋₆ alkyl group), or Rc⁶ is bonded to Rc² or Rc³ to form a non-aromatic heterocycle, or bonded to Rc⁴ or Rc⁵ to from a non-aromatic heterocycle. Rc⁶ is more preferably
(1) a hydrogen atom; or
(2) a C₁₋₆ alkyl group; or
(3) Rc⁶ is bonded to Rc² or Rc³ to form a non-aromatic heterocycle (e.g., piperidine, pyrrolidine); or
(4) Rc⁶ is bonded to Rc⁴ or Rc⁵ to form a non-aromatic heterocycle (e.g., piperidine)]; and

Rc⁷ is a hydrogen atom or an optionally substituted hydrocarbon group, or Rc⁷ is bonded to Rc² or Rc³ to form a non-aromatic heterocycle, or bonded to Rc⁴ or Rc⁵ to form a non-aromatic heterocycle

[Rc⁷ is preferably a hydrogen atom or an optionally substituted C₁₋₁₀ alkyl group (preferably, C₁₋₆ alkyl group), or Rc⁷ is bonded to Rc² or Rc³ to form a non-aromatic heterocycle, or bonded to Rc⁴ or Rc⁵ to form a non-aromatic heterocycle.
Rc⁷ is more preferably
(1) a hydrogen atom; or
(2) a C₁₋₆ alkyl group; or
(3) Rc⁷ is bonded to Rc² or Rc³ to form a non-aromatic heterocycle (e.g., piperidine); or
(4) Rc⁷ is bonded to Rc⁴ or Rc⁵ to form a non-aromatic heterocycle (e.g., piperidine, pyrrolidine)].

Each symbol in the formula (Id) is described in detail in the following.

In the following explanation, a moiety in the formula (Id), which is represented by

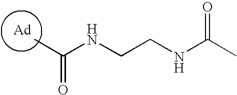

wherein each symbol is as defined in the formula (Id), is sometimes to be referred to as substituent D.

Ring Ad is an optionally substituted aromatic hydrocarbon.

As the “aromatic hydrocarbon” of the “optionally substituted aromatic hydrocarbon” for ring Ad, a ring corresponding to the C₆₋₁₄ aryl group exemplarily recited as the “substituent” for Ra¹, Ra², Ra³, Ra⁴, Ra⁵, Ra⁶ or Ra⁷ can be mentioned. The aromatic hydrocarbon can be bonded to a carbon atom of the adjacent carbonyl group at any bondable position.

As the “aromatic hydrocarbon” of the “optionally substituted aromatic hydrocarbon” for ring Ad, benzene is preferable.

The “aromatic hydrocarbon” of the “optionally substituted aromatic hydrocarbon” for ring Ad optionally has 1 to 3 substituents at substitutable position(s). As such substituents, for example, those exemplarily recited as the “substituent” for Ra¹, Ra², Ra³, Ra⁴, Ra⁵, Ra⁶ or Ra⁷ can be mentioned.

As the substituents of ring Ad,

an optionally substituted hydrocarbon group;
an optionally substituted heterocyclic group;
an optionally substituted hydroxy group;
a cyano group;
an acyl group;
a halogen atom;
and the like are preferable.

As the substituents of ring Ad,

(1) a C₁₋₆ alkoxy group optionally substituted by 1 to 3 substituents selected from a hydroxy group and a halogen atom,
(2) a hydroxy group,
(3) a halogen atom,
(4) a C₁₋₆ alkyl group,
(5) an aromatic heterocyclic group (e.g., imidazolyl, pyrazolyl),
(6) a sulfamoyl group,
(7) a cyano group
and the like are more preferable, and a halogen atom and a C₁₋₆ alkoxy group optionally substituted by 1 to 3 halogen atoms are particularly preferable.

Ring Ad is preferably an aromatic hydrocarbon (preferably, benzene) optionally substituted by 1 to 3 substituents selected from an optionally substituted hydrocarbon group;

an optionally substituted heterocyclic group;
an optionally substituted hydroxy group;
a cyano group;
an acyl group; and
a halogen atom.

Ring Ad is more preferably an aromatic hydrocarbon (preferably, benzene) optionally substituted by 1 to 3 substituents selected from

(1) a C₁₋₆ alkoxy group optionally substituted by 1 to 3 substituents selected from a hydroxy group and a halogen atom,
(2) a hydroxy group,
(3) a halogen atom,
(4) a C₁₋₆ alkyl group,
(5) an aromatic heterocyclic group (e.g., imidazolyl, pyrazolyl),
(6) a sulfamoyl group, and
(7) a cyano group,
particularly preferably an aromatic hydrocarbon (preferably, benzene) optionally substituted by 1 to 3 substituents selected from a halogen atom and a C₁₋₆ alkoxy group optionally substituted by 1 to 3 halogen atoms.

Ring Bd is an aromatic heterocycle which is optionally further substituted.

As the “aromatic heterocycle” of the “aromatic heterocycle which is optionally further substituted” for ring Bd, a ring corresponding to the aromatic heterocyclic group exemplarily recited as the “substituent” for Ra¹, Ra², Ra³, Ra⁴Ra⁵, Ra⁶ or Ra⁷ can be mentioned. The aromatic heterocycle can be bonded to a carbon atom of the adjacent carbonyl group at any bondable position.

As the “aromatic heterocycle” of the “aromatic heterocycle which is optionally further substituted” for ring Bd, pyridine, pyrazole, triazole and indole are preferable.

The “aromatic heterocycle” of the “aromatic heterocycle which is optionally further substituted” for ring Bd optionally further has 1 to 3 substituents, besides ring Cd, at substitutable position(s). As such substituents, for example, those (except an oxo group) exemplarily recited as the substituents of the C₃₋₁₀ cycloalkyl group and the like exemplarily recited as the “substituent” for Ra¹, Ra², Ra³, Ra⁴, Ra⁵, Ra⁶ or Ra⁷ can be mentioned.

As the substituents other than ring Cd of ring Bd,

(1) a C₁₋₆ alkyl group optionally substituted by 1 to 3 halogen atoms;
(2) a C₆₋₁₄ aryl group;
(3) a C₁₋₆ alkoxy group;
(4) a C₇₋₁₃ aralkyloxy group (e.g., benzyloxy);
(5) an amino group optionally mono- or di-substituted by substituent(s) selected from
(a) a C₁₋₆ alkyl group, and
(b) a C₁₋₆ alkoxy-carbonyl group; and the like are preferable, and
   (1) a C₁₋₆ alkyl group optionally substituted by 1 to 3 halogen atoms;
   (2) a C₆₋₁₄ aryl group and the like are more preferable.

Ring Bd is preferably pyridine, pyrazole, triazole or indole, each of which is substituted by ring Cd and optionally further substituted.

Ring Bd is more preferably pyridine, pyrazole, triazole or indole, each of which is substituted by ring Cd and optionally further substituted by 1 to 3 substituents selected from

(1) a C₁₋₆ alkyl group optionally substituted by 1 to 3 halogen atoms;
(2) a C₆₋₁₄ aryl group;
(3) a C₁₋₆ alkoxy group;
(4) a C₇₋₁₃ aralkyloxy group (e.g., benzyloxy); and
(5) an amino group optionally mono- or di-substituted by substituent(s) selected from

(a) a C₁₋₆ alkyl group, and

(b) a C₁₋₆ alkoxy-carbonyl group; particularly preferably pyridine, pyrazole, triazole or indole, each of which is substituted by ring Cd and optionally further substituted by 1 to 3 substituents selected from

(1) a C₁₋₆ alkyl group optionally substituted by 1 to 3 halogen atoms; and
(2) a C₆₋₁₄ aryl group.

Ring Cd is an optionally substituted aromatic ring.

As the “aromatic ring” of the “optionally substituted aromatic ring” for ring Cd, an aromatic hydrocarbon and an aromatic heterocycle can be mentioned.

Here, as the aromatic hydrocarbon, a ring corresponding to the C₆₋₁₄ aryl group exemplarily recited as the “substituent” for Ra¹, Ra², Ra³, Ra⁴, Ra⁵, Ra⁶ or Ra⁷ can be mentioned.

As the aromatic heterocycle, a ring corresponding to the aromatic heterocyclic group exemplarily recited as the “substituent” for Ra¹, Ra², Ra³, Ra⁴, Ra⁵, Ra⁶ or Ra⁷ can be mentioned.

As the “aromatic ring” of the “optionally substituted aromatic ring” for ring Cd, an aromatic hydrocarbon is preferable, and benzene is particularly preferable.

The “aromatic ring” of the “optionally substituted aromatic ring” for ring Cd optionally has 1 to 3 substituents at substitutable position(s). As such substituents, for example, those (except an oxo group) exemplarily recited as the substituents of the C₃₋₁₀ cycloalkyl group and the like exemplarily recited as the “substituent” for Ra¹, Ra², Ra³, Ra⁴, Ra⁵, Ra⁶ or Ra⁷ can be mentioned.

As the substituents of ring Cd,

(1) a halogen atom,
(2) a hydroxy group,
(3) a C₁₋₆ alkyl group,
(4) a C₁₋₆ alkoxy group and the like are preferable.

Ring Cd is preferably an aromatic hydrocarbon (preferably, benzene) optionally substituted by 1 to 3 substituents selected from

(1) a halogen atom,
(2) a hydroxy group,
(3) a C₁₋₆ alkyl group, and
(4) a C₁₋₆ alkoxy group.

In compound (Id),

1) ring Bd is not pyrazol-4-yl and pyrrol-3-yl, each of which is optionally further substituted (i.e., ring Bd is not pyrazole having substituent D at the 4-position, and pyrrole having substituent D at the 3-position, each of which is optionally further substituted);

2) ring Cd is not optionally substituted quinoline;

3) when ring Bd is pyridine or quinoline, each of which is optionally further substituted, then ring Bd has substituent(s) besides ring Cd; and

4) when ring Bd is a 5-membered nitrogen-containing aromatic heterocycle optionally condensed with an aromatic ring, which is optionally further substituted, then ring Bd does not have an optionally substituted aromatic heterocyclic group as a substituent other than ring Cd and ring Cd is an optionally substituted aromatic hydrocarbon.

As preferable examples of compound (Id), the following compounds can be mentioned.

### [Compound Id-A]

A compound wherein

ring Bd is pyridine, pyrazole, triazole or indole, each of which is substituted by ring Cd and optionally further substituted

[ring Bd is preferably pyridine, pyrazole, triazole or indole, each of which is substituted by ring Cd and optionally further substituted by 1 to 3 substituents selected from
(1) a C₁₋₆ alkyl group optionally substituted by 1 to 3 halogen atoms;
(2) a C₆₋₁₄ aryl group;
(3) a C₁₋₆ alkoxy group;
(4) a C₇₋₁₃ aralkyloxy group (e.g., benzyloxy); and
(5) an amino group optionally mono- or di-substituted by substituent(s) selected from

(a) a C₁₋₆ alkyl group, and

(b) a C₁₋₆ alkoxy-carbonyl group;

more preferably pyridine, pyrazole, triazole or indole, each of which is substituted by ring Cd and optionally further substituted by 1 to 3 substituents selected from
(1) a C₁₋₆ alkyl group optionally substituted by 1 to 3 halogen atoms; and
(2) a C₆₋₁₄ aryl group];

ring Cd is an aromatic hydrocarbon (preferably, benzene) optionally substituted by 1 to 3 substituents selected from

(1) a halogen atom,
(2) a hydroxy group,
(3) a C₁₋₆ alkyl group, and
(4) a C₁₋₆ alkoxy group; and

ring Ad is an aromatic hydrocarbon (preferably, benzene) optionally substituted by 1 to 3 substituents selected from

an optionally substituted hydrocarbon group;
an optionally substituted heterocyclic group;
an optionally substituted hydroxy group;
a cyano group;
an acyl group; and
a halogen atom
[ring Ad is preferably an aromatic hydrocarbon (preferably, benzene) optionally substituted by 1 to 3 substituents selected from
(1) a C₁₋₆ alkoxy group optionally substituted by 1 to 3 substituents selected from a hydroxy group and a halogen atom,
(2) a hydroxy group,
(3) a halogen atom,
(4) a C₁₋₆ alkyl group,
(5) an aromatic heterocyclic group (e.g., imidazolyl, pyrazolyl),
(6) a sulfamoyl group, and
(7) a cyano group,
more preferably an aromatic hydrocarbon (preferably, benzene) optionally substituted by 1 to 3 substituents selected from a halogen atom and a C₁₋₆ alkoxy group optionally substituted by 1 to 3 halogen atoms].

As the other preferable examples of compounds (Ia), (Ib), (Ic) and (Id), the following compounds can be mentioned.

### [Compound Iz]

N-(2-(1-phenyl-3-(trifluoromethyl)-1H-pyrazole-4-carboxamido)ethyl)-6-(2,2,2-trifluoroethoxy)nicotinamide (Example A27);
6-(cyclopropylmethoxy)-N-(2-(1-phenyl-3-(trifluoromethyl)-1H-pyrazole-4-carboxamido)ethyl)nicotinamide (Example A35);
N-(2-(1-phenyl-3-(trifluoromethyl)-1H-pyrazole-4-carboxamido)ethyl)-6-(3,3,3-trifluoropropoxy)nicotinamide (Example A42);
6-(2-(ethylsulfonyl)ethoxy)-N-(2-(1-phenyl-3-(trifluoromethyl)-1H-pyrazole-4-carboxamido)ethyl)nicotinamide (Example A43);
N-(2-(1-phenyl-3-(trifluoromethyl)-1H-pyrazole-4-carboxamido)ethyl)-6-propylnicotinamide (Example A47);
1-phenyl-N-(2-(6-(2,2,2-trifluoroethoxy)nicotinamido)ethyl)-1H-indole-3-carboxamide (Example A53);
N-(2-(1-phenyl-3-(trifluoromethyl)-1H-pyrazole-4-carboxamido)ethyl)-6-o-tolylnicotinamide (Example A73);
1-benzoyl-N-(2-(6-(2,2,2-trifluoroethoxy)nicotinamido)ethyl)-1H-indole-3-carboxamide (Example A82);
6-(5-isopropyl-1,2,4-oxadiazol-3-yl)-N-(2-(1-phenyl-3-(trifluoromethyl)-1H-pyrazole-4-carboxamido)ethyl)nicotinamide (Example A103); or
N-(2-(4-ethoxybenzamido)ethyl)-1-(pyridin-2-yl)-3-(trifluoromethyl)-1H-pyrazole-4-carboxamide (Example B3);
   or a salt thereof.

As a salt of the compound of the present invention, a pharmacologically acceptable salt is preferable. Examples of such a salt include a salt with inorganic base, a salt with organic base, a salt with inorganic acid, a salt with organic acid, a salt with basic or acidic amino acid and the like.

Preferable examples of the salt with inorganic base include alkali metal salts such as sodium salt, potassium salt and the like; alkaline earth metal salts such as calcium salt, magnesium salt and the like; aluminum salt; ammonium salt and the like.

Preferable examples of the salt with organic base include a salt with trimethylamine, triethylamine, pyridine, picoline, ethanolamine, diethanolamine, triethanolamine, tromethamine[tris(hydroxymethyl)methylamine], tert-butylamine, cyclohexylamine, benzylamine, dicyclohexylamine, N,N-dibenzylethylenediamine or the like.

Preferable examples of the salt with inorganic acid include a salt with hydrochloric acid, hydrobromic acid, nitric acid, sulfuric acid, phosphoric acid or the like.

Preferable examples of the salt with organic acid include a salt with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid or the like.

Preferable examples of the salt with basic amino acid include a salt with arginine, lysine, ornithine or the like.

Preferable examples of the salt with acidic amino acid include a salt with aspartic acid, glutamic acid or the like.

A prodrug of the compound of the present invention is a compound that converts to the compound of the present invention due to the reaction by enzyme, gastric acid and the like under the physiological conditions in the body; that is, a compound that converts to the compound of the present invention by enzymatic oxidation, reduction, hydrolysis and the like, and a compound that converts to the compound of the present invention by hydrolysis and the like by gastric acid and the like. Examples of a prodrug of the compound of the present invention include a compound wherein an amino group of the compound of the present invention is acylated, alkylated or phosphorylated (e.g., a compound where amino group of the compound of the present invention is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated or tert-butylated); a compound wherein a hydroxy group of the compound of the present invention is acylated, alkylated, phosphorylated or borated (e.g., a compound where a hydroxy group of the compound of the present invention is acetylated, palmitoylated, propanoylated, pivaloylated, succinylated, fumarylated, alanylated or dimethylaminomethylcarbonylated); a compound wherein a carboxyl group of the compound of the present invention is esterified or amidated (e.g., a compound where a carboxyl group of the compound of the present invention is ethyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified or methylamidated) and the like. These compounds can be produced from the compound of the present invention according to a method known per se.

A prodrug of the compound of the present invention may be a compound that converts to the compound of the present invention under physiological conditions as described in Development of Pharmaceutical Products, vol. 7, Molecule Design, pp. 163-198, Hirokawa Shoten (1990).

The compound of the present invention may be labeled with an isotope (e.g., ³H, ¹⁴C, ³⁵S, ¹²⁵I and the like) and the like.

The compound of the present invention may be an anhydride or a hydrate.

The compound of the present invention and a prodrug thereof (hereinafter sometimes to be simply referred to as the compound of the present invention) show low toxicity and can be used as an agent for the prophylaxis or treatment of various diseases to be mentioned later for mammals (e.g., human, mouse, rat, rabbit, dog, cat, cattle, horse, swine, simian) as they are or by admixing with a pharmacologically acceptable carrier and the like to give a pharmaceutical composition.

Here, various organic or inorganic carriers conventionally used as materials for pharmaceutical preparations are used as a pharmacologically acceptable carrier, which are added as an excipient, a lubricant, a binder, a disintegrant and the like for solid preparations; and a solvent, a dissolution aid, a suspending agent, an isotonicity agent, a buffer, a soothing agent and the like for liquid preparations. Where necessary, an additive for pharmaceutical preparations such as a preservative, an antioxidant, a coloring agent, a sweetening agent and the like can be used.

Preferable examples of the excipient include lactose, sucrose, D-mannitol, D-sorbitol, starch, pregelatinized starch, dextrin, crystalline cellulose, low-substituted hydroxypropyl cellulose, sodium carboxymethylcellulose, powdered acacia, pullulan, light anhydrous silicic acid, synthetic aluminum silicate, magnesium aluminate metasilicate and the like.

Preferable examples of the lubricant include magnesium stearate, calcium stearate, talc, colloidal silica and the like.

Preferable examples of the binder include pregelatinized starch, saccharose, gelatin, powdered acacia, methylcellulose, carboxymethylcellulose, sodium carboxymethylcellulose, crystalline cellulose, sucrose, D-mannitol, trehalose, dextrin, pullulan, hydroxypropyl cellulose, hydroxypropyl methylcellulose, polyvinylpyrrolidone and the like.

Preferable examples of the disintegrant include lactose, sucrose, starch, carboxymethylcellulose, calcium carboxymethylcellulose, sodium croscarmellose, sodium carboxymethyl starch, light anhydrous silicic acid, low-substituted hydroxypropyl cellulose and the like.

Preferable examples of the solvent include water for injection, physiological brine, Ringer's solution, alcohol, propylene glycol, polyethylene glycol, sesame oil, corn oil, olive oil, cottonseed oil and the like.

Preferable examples of the dissolution aid include polyethylene glycol, propylene glycol, D-mannitol, trehalose, benzyl benzoate, ethanol, trisaminomethane, cholesterol, triethanolamine, sodium carbonate, sodium citrate, sodium salicylate, sodium acetate and the like.

Preferable examples of the suspending agent include surfactants such as stearyltriethanolamine, sodium lauryl sulfate, lauryl aminopropionate, lecithin, benzalkonium chloride, benzethonium chloride, glycerol monostearate and the like; hydrophilic polymers such as polyvinyl alcohol, polyvinylpyrrolidone, sodium carboxymethylcellulose, methylcellulose, hydroxymethylcellulose, hydroxyethylcellulose, hydroxypropyl cellulose and the like; polysorbates, polyoxyethylene hydrogenated castor oil, and the like.

Preferable examples of the isotonicity agent include sodium chloride, glycerol, D-mannitol, D-sorbitol, glucose and the like.

Preferable examples of the buffer include phosphate buffer, acetate buffer, carbonate buffer, citrate buffer and the like.

Preferable examples of the soothing agent include benzyl alcohol and the like.

Preferable examples of the preservative include p-oxybenzoates, chlorobutanol, benzyl alcohol, phenethyl alcohol, dehydroacetic acid, sorbic acid and the like.

Preferable examples of the antioxidant include sulfite, ascorbate and the like.

Preferable examples of the coloring agent include water-soluble edible tar pigments (e.g., foodcolors such as Food Color Red Nos. 2 and 3, Food Color Yellow Nos. 4 and 5, Food Color Blue Nos. 1 and 2 and the like), water insoluble lake pigments (e.g., aluminum salt of the aforementioned water-soluble edible tar pigment), natural pigments (e.g., beta carotene, chlorophil, red iron oxide) and the like.

Preferable examples of the sweetening agent include saccharin sodium, dipotassium glycyrrhizinate, aspartame, stevia and the like.

The dosage form of the aforementioned pharmaceutical composition is, for example, an oral agent such as tablets (inclusive of sublingual tablets and orally disintegrable tablets), capsules (inclusive of soft capsules and microcapsules), granules, powders, troches, syrups, emulsions, suspensions and the like; or a parenteral agent such as injections (e.g., subcutaneous injections, intravenous injections, intramuscular injections, intraperitoneal injections, drip infusions), external agents (e.g., transdermal preparations, ointments), suppositories (e.g., rectal suppositories, vaginal suppositories), pellets, nasal preparations, pulmonary preparations (inhalations), ophthalmic preparations and the like. These may be administered safely via an oral or parenteral route.

These agents may be controlled-release preparations such as rapid-release preparations and sustained-release preparations (e.g., sustained-release microcapsules).

The pharmaceutical composition can be produced according to a method conventionally used in the field of pharmaceutical preparation, such as the method described in Japan Pharmacopoeia and the like. Concrete production methods of preparations are described in detail in the following.

While the content of the compound of the present invention in the pharmaceutical composition varies depending on the dosage form, dose of the compound of the present invention and the like, it is, for example, about 0.1-100 wt %.

Where necessary, the aforementioned oral agents may be coated with a coating base for the purpose of masking taste, enteric property or sustained release.

Examples of the coating base include a sugar-coating base, a water-soluble film coating base, an enteric film coating base, a sustained-release film coating base and the like.

As the sugar-coating base, sucrose may be used, if necessary, along with one or more species selected from talc, precipitated calcium carbonate, gelatin, powdered acacia, pullulan, carnauba wax and the like.

As the water-soluble film coating base, for example, cellulose polymers such as hydroxypropyl cellulose, hydroxypropyl methylcellulose, hydroxyethylcellulose, methylhydroxyethylcellulose and the like; synthetic polymers such as polyvinyl acetal diethylaminoacetate, aminoalkyl methacrylate copolymer E [Eudragit E, trade name, Roehm Pharma], polyvinylpyrrolidone and the like; polysaccharides such as pullulan and the like; and the like are used.

As the enteric film coating base, for example, cellulose polymers such as hydroxypropyl methylcellulose phthalate, hydroxypropyl methylcellulose acetate succinate, carboxymethylethylcellulose, cellulose acetate phthalate and the like; acrylic acid polymers such as methacrylic acid copolymer L [Eudragit L, trade name, Roehm Pharma], methacrylic acid copolymer LD [Eudragit L-30D55, trade name, Roehm Pharma], methacrylic acid copolymer S [Eudragit S, trade name, Roehm Pharma] and the like; natural products such as shellac and the like; and the like are used.

As the sustained-release film coating base, for example, cellulose polymers such as ethylcellulose and the like; acrylic acid polymers such as aminoalkyl methacrylate copolymer RS [Eudragit RS, trade name, Roehm Pharma], ethyl acrylate-methyl methacrylate copolymer suspension [Eudragit NE, trade name, Roehm Pharma] and the like; and the like are used.

Two or more kinds of the above-mentioned coating bases may be mixed in an appropriate ratio for use. In addition, a light shielding agent such as titanium oxide, ferric oxide and the like may be used during coating.

The compound of the present invention shows low toxicity (e.g., acute toxicity, chronic toxicity, genetic toxicity, reproductive toxicity, cardiotoxicity, carcinogenic), causes fewer side effects and can be used as an agent for the prophylaxis or treatment or diagnosis of various diseases for mammals (e.g., human, cattle, horse, dog, cat, simian, mouse, rat, especially human).

The compound of the present invention has a DGAT (DGAT1 or DGAT2 or both) inhibitory action, and is useful for the prophylaxis, treatment or amelioration of DGAT-related diseases.

As the DGAT-related diseases, for example, obesity, diabetes (e.g., type 1 diabetes, type 2 diabetes, gestational diabetes), insulin resistance, leptin resistance, arteriosclerosis, hyperlipidemia (e.g., hypertriglyceridemia, hypercholesterolemia, hypo-HDL-cholesterolemia, postprandial hyperlipemia), arteriosclerosis, hypertension, cardiac failure, metabolic syndrome and the like can be mentioned.

For diagnostic criteria of diabetes, Japan Diabetes Society reported new diagnostic criteria in 1999.

According to this report, diabetes is a condition showing any of a fasting blood glucose level (glucose concentration of intravenous plasma) of not less than 126 mg/dl, a 75 g oral glucose tolerance test (75 g OGTT) 2 h level (glucose concentration of intravenous plasma) of not less than 200 mg/dl, and a non-fasting blood glucose level (glucose concentration of intravenous plasma) of not less than 200 mg/dl. A condition not falling under the above-mentioned diabetes and different from “a condition showing a fasting blood glucose level (glucose concentration of intravenous plasma) of less than 110 mg/dl or a 75 g oral glucose tolerance test (75 g OGTT) 2 h level (glucose concentration of intravenous plasma) of less than 140 mg/dl” (normal type) is called a “borderline type”.

In addition, ADA (American Diabetes Association) reported new diagnostic criteria of diabetes in 1997 and WHO in 1998.

According to these reports, diabetes is a condition showing a fasting blood glucose level (glucose concentration of intravenous plasma) of not less than 126 mg/dl and a 75 g oral glucose tolerance test 2 h level (glucose concentration of intravenous plasma) of not less than 200 mg/dl.

According to the above-mentioned reports, impaired glucose tolerance is a condition showing a fasting blood glucose level (glucose concentration of intravenous plasma) of less than 126 mg/dl and a 75 g oral glucose tolerance test 2 h level (glucose concentration of intravenous plasma) of not less than 140 mg/dl and less than 200 mg/dl. According to the report of ADA, a condition showing a fasting blood glucose level (glucose concentration of intravenous plasma) of not less than 110 mg/dl and less than 126 mg/dl is called IFG (Impaired Fasting Glucose). According to the report of WHO, among the IFG (Impaired Fasting Glucose), a condition showing a 75 g oral glucose tolerance test 2 h level (glucose concentration of intravenous plasma) of less than 140 mg/dl is called IFG (Impaired Fasting Glycemia).

The compound of the present invention can be also used as an agent for the prophylaxis or treatment of diabetes, borderline type, impaired glucose tolerance, IFG (Impaired Fasting Glucose) and IFG (Impaired Fasting Glycemia), as determined according to the above-mentioned new diagnostic criteria. Moreover, the compound of the present invention can prevent progress of borderline type, impaired glucose tolerance, IFG (Impaired Fasting Glucose) or IFG (Impaired Fasting Glycemia) into diabetes.

The compound of the present invention can be also used as an agent for the prophylaxis or treatment of, for example, diabetic complications [e.g., neuropathy, nephropathy, retinopathy, cataract, macroangiopathy, osteopenia, hyperosmolar diabetic coma, infectious disease (e.g., respiratory infection, urinary tract infection, gastrointestinal infection, dermal soft tissue infection, inferior limb infection), diabetic gangrene, xerostomia, hypacusis, cerebrovascular disorder, peripheral blood circulation disorder], osteoporosis, cachexia (e.g., cancerous cachexia, tuberculous cachexia, diabetic cachexia, blood disease cachexia, endocrine disease cachexia, infectious disease cachexia or cachexia due to acquired immunodeficiency syndrome), fatty liver, polycystic ovary syndrome, kidney disease (e.g., diabetic nephropathy, glomerular nephritis, glomerulosclerosis, nephrotic syndrome, hypertensive nephrosclerosis, end stage kidney disease), muscular dystrophy, myocardial infarction, angina pectoris, cerebrovascular accident (e.g., cerebral infarction, cerebral apoplexy), Alzheimer's disease, Parkinson's syndrome, anxiety, dementia, insulin resistance syndrome, Syndrome X, hyperinsulinemia, hyperinsulinemia-induced sensory disorder, tumor (e.g., leukemia, breast cancer, prostatic cancer, skin cancer), irritable bowel syndrome, acute or chronic diarrhea, inflammatory diseases (e.g., chronic rheumatoid arthritis, spondylitis deformans, osteoarthritis, lumbago, gout, postoperative or traumatic inflammation, swelling, neuralgia, pharyngolaryngitis, cystitis, hepatitis (inclusive of nonalcoholic steatohepatitis), pneumonia, pancreatitis, enteritis, inflammatory bowel diseases (including inflammatory disease of large intestine), ulcerative colitis, gastric mucosal injury (inclusive of gastric mucosal injury caused by aspirin)), small intestine mucous membrane trauma, malabsorption, testis function disorder, visceral obesity syndrome and the like.

The compound of the present invention can also be used for the secondary prophylaxis or suppression of the progression of the above-mentioned various diseases (e.g., cardiovascular events such as cardiac infarction and the like).

While the dose of the compound of the present invention varies depending on the administration subject, administration route, target disease, condition and the like, the compound of the present invention is generally given in a single dose of about 0.01-100 mg/kg body weight, preferably 0.05-30 mg/kg body weight, more preferably 0.1-10 mg/kg body weight, in the case of, for example, oral administration to adult diabetic patients. This dose is desirably given 1 to 3 times a day.

The compound of the present invention can be used in combination with drugs such as a therapeutic agent for diabetes, a therapeutic agent for diabetic complications, an antihyperlipemic agent, an antihypertensive agent, an antiobestic agent, a diuretic, an antithrombotic agent and the like (hereinafter to be referred to as a combination drug), with the aim of enhancing the action of the compound, reducing the dose of the compound and the like. In this case, the timing of administration of the compound of the present invention and a combination drug is not limited. These may be simultaneously administered to an administration subject or administered in a staggered manner. Moreover, the compound of the present invention and a combination drug may be administered as two kinds of preparations each containing an active ingredient, or may be administered as a single preparation containing both active ingredients.

The dose of the combination drug can be determined as appropriate based on the dose clinically employed. The proportion of the compound of the present invention and the combination drug can be appropriately determined depending on the administration subject, administration route, target disease, condition, combination and the like. When, for example, the administration subject is human, the combination drug is used in an amount of 0.01-100 parts by weight per 1 part by weight of the compound of the present invention.

As the therapeutic agent for diabetes, insulin preparations (e.g., animal insulin preparations extracted from the pancreas of bovine or pig; human insulin preparations genetically synthesized using Escherichia coli or yeast; zinc insulin; protamine zinc insulin; fragment or derivative of insulin (e.g., INS-1), oral insulin preparation), insulin sensitizers (e.g., pioglitazone or a salt thereof (preferably hydrochloride), rosiglitazone or a salt thereof (preferably maleate), Reglixane (JTT-501), Netoglitazone (MCC-555), DRF-2593, KRP-297, R-119702, Rivoglitazone (CS-011), FK-614, compounds described in WO99/58510 (e.g., (E)-4-[4-(5-methyl-2-phenyl-4-oxazolylmethoxy)benzyloxyimino]-4-phenylbutyric acid), compounds described in WO01/38325, Tesaglitazar (AZ-242), Ragaglitazar (N,N-622), Muraglitazar (BMS-298585), ONO-5816, Edaglitazone (BM-13-1258), LM-4156, MBX-102, Naveglitazar (LY-519818), MX-6054, LY-510929, Balaglitazone (N,N-2344), T-131 or a salt thereof, THR-0921), PPARγ agonists, PPARγ antagonists, PPARγ/α dual agonists, α-glucosidase inhibitors (e.g., voglibose, acarbose, miglitol, emiglitate), biguanides (e.g., phenformin, metformin, buformin or salts thereof (e.g., hydrochloride, fumarate, succinate)), insulin secretagogues [sulfonylureas (e.g., tolbutamide, glibenclamide, gliclazide, chlorpropamide, tolazamide, acetohexamide, glyclopyramide, glimepiride, glipizide, glybuzole), repaglinide, senaglinide, nateglide, mitiglinide or calcium salt hydrate thereof], GPR40 agonists, GLP-1 receptor agonists [e.g., GLP-1, GLP-1MR, N,N-2211, AC-2993 (exendin-4), BIM-51077, Aib(8,35) hGLP-1 (7,37)NH₂, CJC-1131], amylin agonists (e.g., pramlintide), phosphotyrosine phosphatase inhibitors (e.g., sodium vanadate), dipeptidyl peptidase IV inhibitors (e.g., NVP-DPP-728, PT-100, P32/98, Vidagliptin (LAF-237), P93/01, TS-021, Sitagliptin (MK-431), Saxagliptin (BMS-477118), T-6666), β3 agonists (e.g., AJ-9677, AZ40140), gluconeogenesis inhibitors (e.g., glycogen phosphorylase inhibitors, glucose-6-phosphatase inhibitors, glucagon antagonists), SGLT (sodium-glucose cotransporter) inhibitors (e.g., T-1095), 11β-hydroxysteroid dehydrogenase inhibitors (e.g., BVT-3498), adiponectin or agonists thereof, IKK inhibitors (e.g., AS-2868), leptin resistance improving drugs, somatostatin receptor agonists (compounds described in WO01/25228, WO03/42204, WO98/44921, WO98/45285 and WO99/22735) and glucokinase activators (e.g., Ro-28-1675) can be mentioned.

Examples of the therapeutic agent for diabetic complications include aldose reductase inhibitors (e.g., Tolrestat, Epalrestat, Zenarestat, Zopolrestat, Minalrestat, Fidarestat, CT-112, Ranirestat), neurotrophic factors and increasing drugs thereof (e.g., NGF, NT-3, BDNF, neurotrophin production-secretion promoters described in WO01/14372 (e.g., 4-(4-chlorophenyl)-2-(2-methyl-1-imidazolyl)-5-[3-(2-methylphenoxy)propyl]oxazole)), neuranagenesis stimulators (e.g., Y-128), PKC inhibitors (e.g., ruboxistaurin mesylate), AGE inhibitors (e.g., ALT946, pimagedine, pyratoxanthine, N-phenacylthiazolium bromide (ALT766), ALT-711, EXO-226, Pyridorin, Pyridoxamine), reactive oxygen scavengers (e.g., thioctic acid), cerebral vasodilators (e.g., tiapride, mexiletine), somatostatin receptor agonists (e.g., BIM23190) and apoptosis signal regulating kinase-1 (ASK-1) inhibitors.

Examples of the antihyperlipemic agent include HMG-CoA reductase inhibitors (e.g., pravastatin, simvastatin, lovastatin, atorvastatin, fluvastatin, itavastatin, rosuvastatin, pitavastatin and salts thereof (e.g., sodium salt, calcium salt)), squalene synthase inhibitors (e.g., compounds described in WO97/10224, such as N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]-piperidine-4-acetic acid), fibrate compounds (e.g., bezafibrate, clofibrate, simfibrate, clinofibrate), ACAT inhibitors (e.g., Avasimibe, Eflucimibe), anion exchange resins (e.g., colestyramine), probucol, nicotinic acid drugs (e.g., nicomol, niceritrol), ethyl icosapentate and plant sterols (e.g., soysterol, γ-oryzanol).

Examples of the antihypertensive agent include angiotensin converting enzyme inhibitors (e.g., captopril, enalapril, delapril), angiotensin II receptor antagonists (e.g., candesartan cilexetil, losartan, eprosartan, valsartan, telmisartan, irbesartan, tasosartan, 1-[[2′-(2,5-dihydro-5-oxo-4H-1,2,4-oxadiazol-3-yl)biphenyl-4-yl]methyl]-2-ethoxy-1H-benzimidazole-7-carboxylic acid), calcium antagonists (e.g., manidipine, nifedipine, amlodipine, efonidipine, nicardipine), potassium channel openers (e.g., levcromakalim, L-27152, AL 0671, NIP-121) and Clonidine.

Examples of the antiobestic agent include antiobestic agents acting on the central nervous system (e.g., Dexfenfluramine, fenfluramine, phentermine, Sibutramine, amfepramone, dexamphetamine, Mazindol, phenylpropanolamine, clobenzorex; MCH receptor antagonists (e.g., SB-568849; SNAP-7941; compounds encompassed in WO01/82925 and WO01/87834); neuropeptide Y antagonists (e.g., CP-422935); cannabinoid receptor antagonists (e.g., SR-141716, SR-147778); ghrelin antagonists; 11β-hydroxysteroid dehydrogenase inhibitors (e.g., BVT-3498)), pancreatic lipase inhibitors (e.g., orlistat, ATL-962), β3 agonists (e.g., AJ-9677, AZ40140), peptidic anorexiants (e.g., leptin, CNTF (Ciliary Neurotropic Factor)), cholecystokinin agonists (e.g., lintitript, FPL-15849) and feeding deterrents (e.g., P-57).

Examples of the diuretic include xanthine derivatives (e.g., sodium salicylate and theobromine, calcium salicylate and theobromine), thiazide preparations (e.g., ethiazide, cyclopenthiazide, trichloromethiazide, hydrochlorothiazide, hydroflumethiazide, benzylhydrochlorothiazide, penflutizide, polythiazide, methyclothiazide), antialdosterone preparations (e.g., spironolactone, triamterene), carbonate dehydratase inhibitors (e.g., acetazolamide), chlorobenzenesulfonamide preparations (e.g., chlortalidone, mefruside, indapamide), azosemide, isosorbide, etacrynic acid, piretanide, bumetanide and furosemide.

Examples of the antithrombotic agent include heparins (e.g., heparin sodium, heparin calcium, dalteparin sodium), warfarins (e.g., warfarin potassium), anti-thrombin drugs (e.g., aragatroban), thrombolytic agents (e.g., urokinase, tisokinase, alteplase, nateplase, monteplase, pamiteplase), platelet aggregation inhibitors (e.g., ticlopidine hydrochloride, cilostazol, ethyl icosapentate, beraprost sodium, sarpogrelate hydrochloride) and the like.

Hereinafter the production methods of the compound of the present invention are explained.

The compounds of this invention may possess one or more asymmetric centers; such compounds can therefore be produced as individual (R) or (S)-stereoisomers or as mixtures thereof. Unless indicated otherwise, the description or naming of a particular compound in the specification and claims is intended to include both individual enantiomers and diastereomers, and mixtures, racemate or otherwise, thereof. Accordingly, this invention also includes all such isomers, including diastereomeric mixtures, enantiomeric mixture, diastereomers and pure enantiomers of the compounds of this invention. The term “enantiomer” refers to two stereoisomers of a compound which are non-superimposable mirror images of one another. The term “diastereomer” refers to a pair of optical isomers which are not mirror images of one another.

Diastereomers have different physical properties, e.g. melting points, boiling points, spectral properties, and reactivities.

The compounds of the present invention may also exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

In the structures shown herein, where the stereochemistry of any particular chiral atom is not specified, then all stereoisomers are contemplated and included as the compounds of the invention. Where stereochemistry is specified by a solid wedge or a hashed wedge representing a particular configuration, then that stereoisomer is so specified and defined. When stereochemistry is specified by a solid line or a hashed line representing a relative conformation such as cis and trans, then that conformation is so specified and defined.

In the present specification, the following abbreviations may be used:

### DCM: Dichloromethane

### MeCN: Acetonitrile

### THF: Tetrahydrofuran

### EtOH: Ethanol

### MeOH: Methanol

iPrOH: Isopropanol

### CHCl₃: Chloroform

### DCE: Dichloroethane

DMSO: Dimethyl sulfoxide

### DMF: Dimethylformamide

### DMA: Dimethylacetamide

AcOEt: Ethyl acetate
Et₂O: Diethyl ether

### CH₃CN: Acetonitrile

### H₂O: Water

HATU: O-(7-Azabenzotriazol-1-yl)-N,N,N′,N′-tetramethyluronium hexafluorophosphate
BOP-Cl: Benzotriazol-1-yl-oxytris(dimethylamino)phosphonium hexafluorophosphate
EDAC.HCl: 1-Ethyl-3-(3-dimethylaminopropyl)carbodiimide hydrochloride
HOBt.H₂O: Hydroxybenzotriazole monohydrate
NaOH: Sodium hydroxide
KOH: Potassium hydroxide
K₂CO₃: Potassium carbonate
Cs₂CO₃: Cesium carbonate
Na₂CO₃: Sodium carbonate
K₃PO₄: Potassium phosphate
KF: Potassium fluoride

### TEA: Triethylamine

### DIPEA: Diisopropylethylamine

### Py: Pyridine

NaH: Sodium hydride
LDA: Lithium diisopropylamide
NaHCO₃: Sodium hydrogencarbonate
H₂SO₄: Sulfuric acid
HCl: Hydrochloric acid
HBr: Hydrobromic acid
NH₄Cl: Ammonium chloride
TFA: Trifluoroacetic acid
AcOH: Acetic acid
TFAA: Trifluoroacetic anhydride
Na₂SO₄: Sodium sulfate
MgSO₄: Magnesium sulfate

### Pd(PPh₃)₄: Tetrakis(triphenylphosphine)palladium

Pd(OAc)₂: Palladium acetate
PdCl₂(dppf): Dichloro[1,1′-bis(diphenylphosphino)ferrocene]palladium

### PdCl₂(PPh₃)₂: Dichlorobis(triphenylphosphine)palladium

Pd₂ (dba) 3: Tris(dibenzylideneacetone) dipalladium
PdCl₂(dppp): Dichloro[1,3-Bis(diphenylphosphino)propane]palladium
Xantphos: (9,9-Dimethyl-9H-xanthene-4,5-diyl)bis(diphenylphosphine)
(BINAP)PdCl₂: (2,2′-Bis(diphenylphosphino)-1,1′-binaphthyl)palladium (II) chloride
TsCl: p-Toluenesulfonyl chloride
MCPBA: m-Chloroperbenzoic acid
KMnO₄: Potassium permanganate

### CDI: N,N-Carbonyldiimidazole

PtO₂: Platinum oxide
Pd/C: Palladium on carbon
DMF-DMA: Dimethylformamide dimethylacetal
POCl₃: Phosphorus oxychloride
TFFH: Tetramethylfluoroformamidinium hexafluorophosphate
NaCN: Sodium cyanide
KCN: Potassium cyanide
CuCN: Copper cyanide
Zn(CN)₂: Zinc cyanide
Boc: tert-Butoxycarbonyl

### Cbz: Benzyloxycarbonyl

For illustrative purposes, Schemes 1-35 show general methods for preparing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

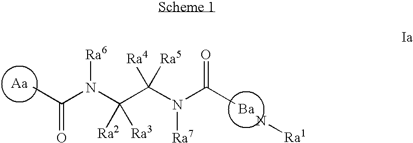

Compounds of formula Ia of this invention can be prepared by several methods generally known in the art of organic chemistry.

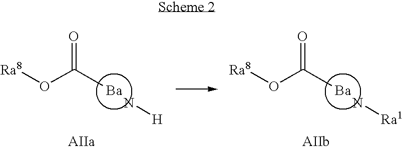

wherein Ra⁸ is a C₁-C₄ alkyl or benzyl group and other symbols are as defined above.

Intermediate esters AIIb, which are suitable for use in preparing compounds Ia and Ia-I can be prepared under various conditions depending on the nature of the Ra¹ substituent.

In the case wherein Ra¹ is an optionally substituted aryl or heteroaryl group, esters AIIb can be prepared according to one of the following references: Tetrahedron Lett. 1998, 39, 2941-2944; Eur. J. Org. Chem. 2004, 695-709; J. Am. Chem. Soc. 2001, 123, 7727-7729; J. Am. Chem. Soc. 2002, 124, 11684-11688; J. Org. Chem. 2004, 69, 5578. Typically, the N-arylation or N-heteroarylation of the Ba ring is performed with an aryl or hetero aryl halide (preferably iodide) in the presence of copper catalyst such as copper iodide or copper oxide, in the presence of a ligand such as substituted ethylene diamines, salicylaldoximes or other ligands reported in Eur. J. Org. Chem. 2004, 695-709. The reaction requires a base such as potassium phosphate or alkali carbonates (potassium carbonate, sodium carbonate or cesium carbonate) and is performed in a degassed solvent such as acetonitrile, toluene or DMF at a temperature of 20° C. to 150° C. for 24 to 48 hours under inert atmosphere. Preferably, the N-arylation or N-heteroarylation is conducted according to the method described in J. Org. Chem. 2004, 69, 5578, in toluene with 1 equivalent of AIIa, 1.1-10 equivalents of aryl or heteroaryl halide, 2 equivalents of diamine ligand, 2-3 equivalents of base and 0.05 equivalents of copper(I) iodide, or according to the method described in Eur. J. Org. Chem. 2004, 695-709, in DMF with 1 equivalent of AIIa, 1.5-10 equivalents of aryl or heteroaryl halide, 0.2-0.4 equivalents of oxime ligand, 2-3 equivalents of base and 0.05 equivalents of copper(II) oxide.

In the case where Ra¹ is an optionally substituted non-aromatic hydrocarbon group or an optionally substituted non-aromatic heterocyclic group, esters AIIb can be prepared by direct alkylation with the corresponding halide or the corresponding sulfonate in the presence of a base such as alkali carbonates or hydrides (sodium hydride or potassium hydride) in a solvent such as DMF at a temperature ranging from 20° C. to 130° C. for 24 to 48 hours. In the case of hindered or poorly reactive halides, the corresponding halide may be used as the solvent at a temperature ranging from 20° C. to 130° C. for 10 to 48 hours. Alternatively, esters AIIb can be prepared from the amine AIIa by opening of the corresponding epoxide in the presence of a base such as alkali carbonates in a solvent such as halogenated hydrochlorides (DCM or CHCl₃) or neat at a temperature from 20° C. to 100° C. for 1 to 48 hours. Preferably, the alkylation is run in DMF or halogenated hydrocarbons with 1 equivalent of AIIa, 1.1-10 equivalents of halide, sulfonate or epoxide and 1-5 equivalents of base.

In the case where Ra¹ is an acyl group, esters AIIb can be prepared with the corresponding acid halides or sulfonyl halides in the presence of a base such as sodium hydride, alkali carbonates, sodium hydroxide or triethylamine in a solvent such as DMF, acetone or halogenated hydrocarbons at a temperature ranging from 0° C. to 130° C. for 10 to 24 hours. Preferably, this reaction is run in DMF or halogenated hydrocarbons with 1 equivalent of AIIa, 1.1-2 equivalents of acid halide or sulfonyl halide and 1-5 equivalents of base.

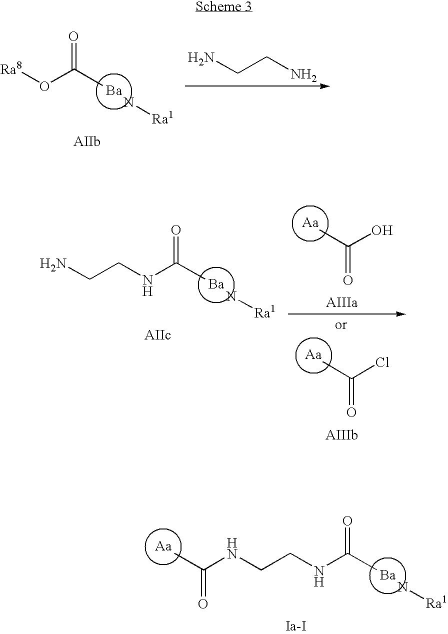

wherein the symbols are as defined above.

Compounds Ia-I can be prepared according to the sequence shown in Scheme 3. Esters AIIb, where Ra⁸ is preferably methyl or ethyl group, can be treated with ethylenediamine at refluxing temperature to produce amines AIIc. Compounds Ia-I can then be prepared from acids AIIIa and amines AIIc or their salts by reacting both intermediates in the presence of various condensing reagents. Known condensing reagents that effect amide bond formation include, but are not limited to, N,N-carbonyldiimidazole, halopyridine salts, 2,4,6-trichlorobenzoyl chloride, HATU, BOP-Cl or EDAC.HCl/HOBt.H₂O. In the present invention, the preferred reagent is either HATU or EDAC.HCl/HOBt.H₂O. The reaction can be conducted in aprotic solvents such as tetrahydrofuran, halogenated hydrocarbons, acetonitrile, dimethylformamide, or a mixture of these solvents, at a temperature from 0° C. to 130° C., preferably 20° C. to 70° C., for a time ranging from 1 to 48 hours, preferably 10 to 20 hours. A base such as triethylamine or diisopropylethylamine may be used especially if the reacting amine is in a salt form. While the amount of reagent varies depending on the coupling reagent used, the following amounts are used preferably with HATU or EDAC.HCl/HOBt.H₂O: amine or its salt (1 equivalent), acid (1 equivalent), HATU or EDAC.HCl/HOBt.H₂O (1 to 2 equivalents), base (1 to 3 equivalents if salt form of amine is used). Compounds Ia-I can also be prepared from acid chlorides AIIIb and amines AIIc in the presence of a base such as triethylamine, diisopropylethylamine or pyridine in an aprotic solvent such as THF, benzene, halogenated hydrocarbons at temperatures from 20° C. to 90° C. for 0.5 to 24 hours.

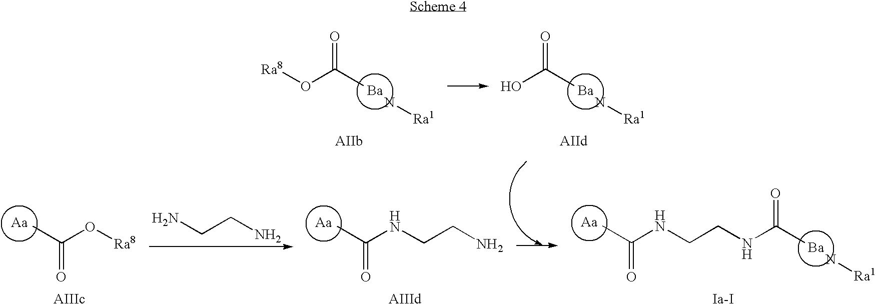

wherein the symbols are as defined above.

Alternatively, compounds Ia-I can be prepared following the sequence described in Scheme 4. Esters AIIIc, where Ra⁸ is preferably methyl or ethyl group, can be treated with ethylenediamine at refluxing temperature to produce amines AIIId. Compounds Ia-I can be prepared under the conditions mentioned in Scheme 3 using an amine AIIId or its salt, and an acid AIId. Acids AIId can be prepared from the corresponding esters AIIb by using a base such as lithium hydroxide, sodium hydroxide, alkali carbonates in a polar protic solvent such as methanol, ethanol, water or in mixtures of solvents including the mentioned polar protic solvent or other aproptic solvents. Typically, the hydrolysis is performed in an alcohol (methanol or ethanol) or in a 1:1 mixture of alcohol/THF, with water in the presence of sodium hydroxide (1-10 equivalents) at a temperature ranging from 20° C. to 100° C. for 4 to 24 hours. Acids AIId can also be prepared from the corresponding esters AIIb by acid hydrolysis using an acid such as TFA, HCl, H₂SO₄, AcOH or in a mixture of these acids in neat or aqueous condition at a temperature ranging from 20° C. to 100° C. for 0.5 to 24 hours. In the case where Ra⁸ is benzyl group, acids AIId can be prepared from the corresponding esters AIIb by hydrogenolysis using catalysts such as palladium on carbon or palladium hydroxide in a protic solvent such as EtOH or aprotic solvent such as EtOAc, under hydrogen atmosphere at a pressure of 15 to 150 psi, at a temperature from 20° C. to 100° C. for 1 to 48 hours. Additional conditions for the hydrolysis of ester groups can be found in T. W. Green, Protective Groups in Organic Synthesis, John Wiley and Sons, Inc., 1981.

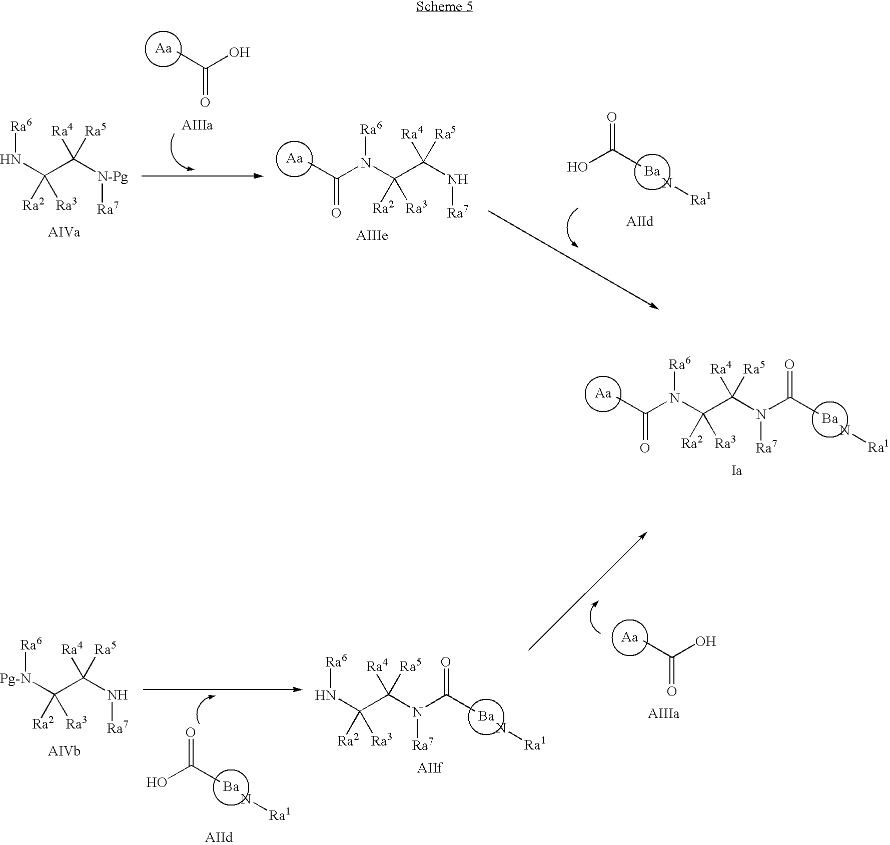

wherein Pg is a protecting group an other symbols are as defined above.

Compounds Ia can be prepared according to Scheme 5.

Compounds AIIIe can be the result of an amide coupling between a suitably protected amine AIVa, where Pg is preferably Boc or Cbz group, and an acid AIIIa in conditions commonly employed to form amide bonds (mentioned previously), followed by the deprotection of the amino group. In the preferred case wherein Pg is Boc group, the deprotection is conveniently performed in the presence of acids such as TFA or HCl, neat or in a solvent such as ethyl ether or dioxane at a temperature from 0° C. to 100° C. for 5 minutes to 24 hours. Additional conditions for the deprotection of amines can be found in T. W. Green, Protective Groups in Organic Synthesis, John Wiley and Sons, Inc., 1981. In the present invention, the preferred deprotection method for Boc-protected amines consists in treating the protected amine in 4N HCl in dioxanes (1-10 equivalents) at 20° C. for 10 minutes to 3 hours. Compounds AIIIe can be further coupled to acids AIId in conditions commonly employed to form amide bonds to produce compounds Ia.

Alternatively, compounds AIIf can be the result of the coupling between a suitably protected amine AIVb, where Pg is preferably Boc or Cbz group, and an acid AIId under conditions commonly employed to form amide bonds, followed by deprotection of the amino group. Compounds Ia can be produced by further coupling the amine AIIf with an acid AIIIa under conditions commonly employed to form amide bonds.

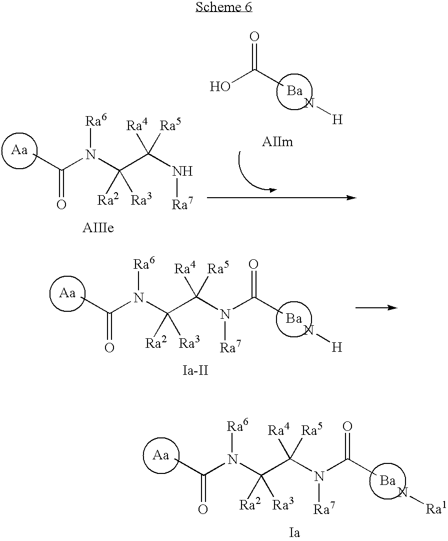

wherein the symbols are as defined above.

Scheme 6 shows an alternative method for the preparation of compounds Ia. The amine AIIIe prepared according to Scheme can be coupled to an acid AIIm under conditions commonly employed to form amide bonds. The resulting amine Ia-II can be further functionalized to compounds Ia, wherein Ra¹ is an optionally substituted hydrocarbon group, an optionally substituted heterocyclic group or an acyl group, under conditions similar to those described in Scheme 2.

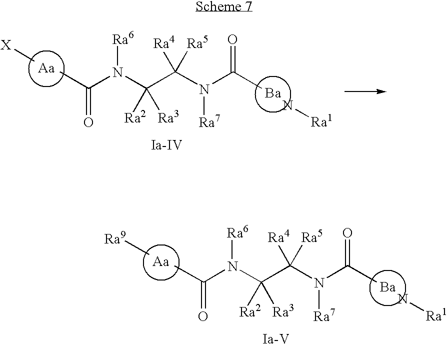

wherein X is a halogen atom (preferably chloride, bromide or iodide), Ra⁹ is an optionally substituted amino group and other symbols are as defined above.

Scheme 7 shows a method for preparing compounds of formula Ia-V. In the preferred case where Aa is an optionally substituted 2-halo-pyridine ring, compounds Ia-V can be prepared by amine substitution. Typically, an amine or its salt (preferably sodium salt) is reacted with a 2-halo-pyridine Ia-IV neat, in an aprotic solvent such as THF, DMF, DMSO, halogenated hydrocarbons or in a protic solvent such as alcohols at temperatures from 60° C. to 160° C. for 1 to 24 hours. In the present invention, the reaction is run with the appropriate amine (50 to 200 equivalents) as the solvent at a temperature from 110° C. to 150° C. for 18 to 24 hours. In cases where the Aa ring is not an optionally substituted 2-halo-pyridine, the coupling can be performed under thermal conditions in the presence of a base such as potassium carbonate, potassium fluoride, hydrides or LDA in a solvent such as DMSO or dioxanes at temperatures from 100° C. to 170° C. for 1 to 48 hours. Alternatively, the coupling can be performed under palladium or copper catalyzed-conditions as reported in J. Organomet. Chem. 1999, 576 (1-2), 125; Angew. Chem., Int. Ed. Engl. 1998, 37, 2046; Org. Lett. 2002, 4(4), 581.

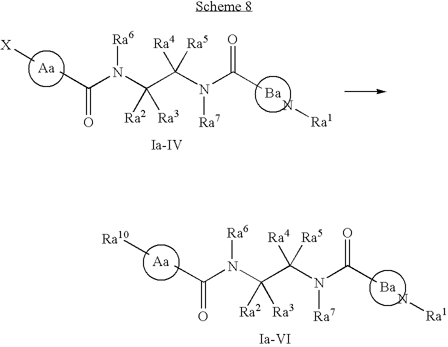

wherein Ra¹⁰ is an optionally substituted hydroxyl group or an optionally substituted mercapto group and other symbols are as defined above.

In the preferred case where Aa is an optionally substituted 2-halo-pyridine ring, compounds of formula Ia-VI can be prepared by halogen displacement. Typically, an alcohol or a thiol is treated with an alkali hydride such as sodium hydride, potassium hydride or a lithium base such as LDA or BuLi to form the corresponding alkoxide or thioalkoxide, which can then react with a 2-halo-pyridine, under conditions similar to those mentioned above, to yield compounds Ia-VI. The 2-halo-pyridine can also be treated with an alcohol or a thiol in the presence of a base such as alkali carbonates in solvents such as DMF or DMSO at temperatures from 100° C. to 170° C. for 10 to 48 hours. In the present invention, the alkoxide or thioalkoxide (1 to 5 equivalents) is formed in the presence of sodium hydride (1 to 5 equivalents) in a solvent such as THF at a temperature from 0° C. to 30° C., and then reacted with a 2-halo-pyridine Ia-IV at temperatures from 60° C. to 80° C. for 3 to 16 hours.

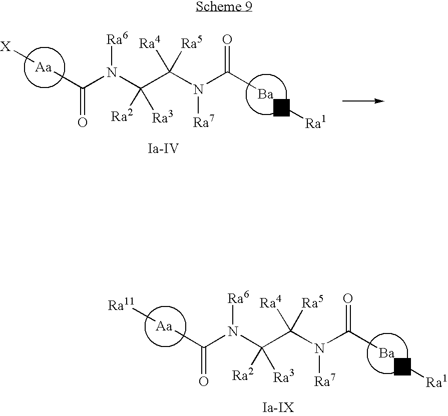

wherein Ra¹¹ is an optionally substituted hydrocarbon group or an optionally substituted heterocyclic group and other symbols are as defined above.

As the “optionally substituted hydrocarbon group” and “optionally substituted heterocyclic group” for Ra¹¹, those exemplarily recited as the “optionally substituted hydrocarbon group” and “optionally substituted heterocyclic group”, which are those exemplarily recited as the “substituent” for Ra¹, Ra², Ra³, Ra⁴, Ra⁵, Ra⁶ or Ra⁷, can be mentioned.

Scheme 9 shows methods for preparing compounds of formula Ia-IX. Compounds Ia-IX can be conveniently prepared from compounds Ia-IV under palladium-catalyzed conditions such as Suzuki (Chem. Rev. 1995, 95, 2457) or Negishi (Negishi, Ei-ichi. Handbook of Organopalladium Chemistry for Organic Synthesis (2002), 1, 767-789; John Wiley & Sons, Inc., Hoboken, N. J). Typically, the coupling is performed between a boronic acid or a zinc halide and compound Ia-IV in the presence of a catalysts such as, but not limited to, Pd(PPh₃)₄, Pd(OAc)₂, PdCl₂(dppf) or PdCl₂(PPh)₂, a base such as alkali carbonates, alkali phosphates (sodium phosphate or potassium phosphate) or potassium fluoride and a ligand (J. Am. Chem. Soc. 1999, 121, 9550-9561) such as phosphines in a solvent such as toluene, THF, alcohols, water or mixtures of the above solvents. In the preferred case where Ra¹¹ is an alkyl group, the reaction is performed using alkyl zinc bromide (2 to 3 equivalents), compound Ia-IV (1 equivalent) and PdCl₂(dppf)-CH₂Cl₂ (0.1 equivalents) in a solvent such as THF at a temperature from 20° C. to 75° C. for 0.5 to 24 hours. In the preferred case where Ra¹¹ is benzyl group, 9-benzyl-9-bora-bicyclo[3.3.1]nonane may be used under similar conditions to those mentioned above. In the preferred case where Ra¹¹ is an aromatic or hetero aromatic group, the reaction is performed in the presence of a boronic acid (1.5-3 equivalents), a palladium catalyst such as Pd(OAc)₂, Pd(PPh₃)₄ or Pd₂(dba)₃ (0.1 to 1 equivalent), a ligand such as 2,8,9-triisobutyl-2,5,8,9-tetraaza-1-phospha-bicyclo[3.3.3]undecane and a base such as alkali carbonates in a solvent such as toluene or THF at a temperature from 45° C. to 120° C., preferably 90° C., for 1 to 16 hours.

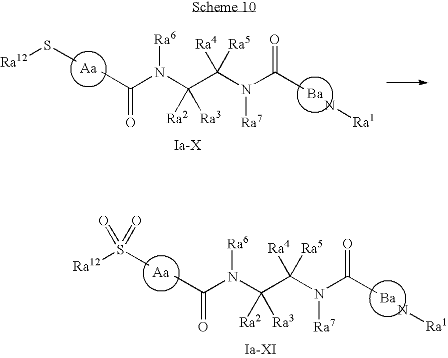

wherein Ra¹² is an optionally substituted hydrocarbon group or an optionally substituted heterocyclic group and other symbols are as defined above.

As the “optionally substituted hydrocarbon group” and “optionally substituted heterocyclic group” for Ra¹², those exemplarily recited as the “optionally substituted hydrocarbon group” and “optionally substituted heterocyclic group”, which are those exemplarily recited as the “substituent” for Ra¹, Ra², Ra³, Ra⁴, Ra⁵, Ra⁶ or Ra⁷, can be mentioned.

Scheme 10 shows methods for preparing compounds of formula Ia-XI. Compounds Ia-XI can be conveniently prepared from sulfides Ia-X, prepared according to similar conditions to those described in Scheme 8, under oxidative conditions. Suitable oxidants include, but are not limited to, KMnO₄, MCPBA, OXONE or hydrogen peroxide. The reaction is typically performed in solvents such as THF, acetone, halogenated hydrocarbons, or a mixture of the mentioned solvents at a temperature from 0° C. to 25° C. for 1 to 24 hours. An acidic co-reagent such as formic acid may be used. In the present invention, a sulfide Ia-X (1 equivalent) is preferably treated with KMnO₄ (1.5 to 4 equivalents) and formic acid (5 to 10 equivalents) in a THF/acetone (1:2) solvent system at 25° C. to 60° C. for 8 to 48 hours.

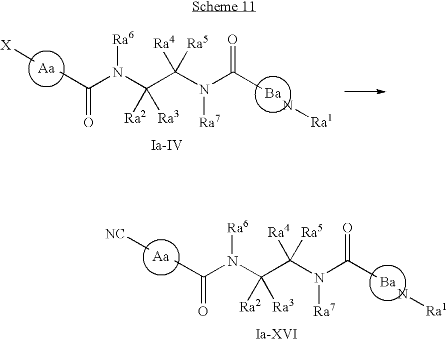

wherein the symbols are as defined above.

Scheme 11 shows methods for preparing compounds of formula Ia-XVI where the Aa ring is substituted with a nitrile group. Compounds Ia-XVI can be prepared by nucleophilic substitution in the presence of nitrile equivalents such as NaCN, KCN or CuCN in solvents such as DMF or DMSO at temperatures from 80° C. to 180° C. for 1 to 48 hours. Compounds Ia-XVI can also be prepared from halides Ia-IV in the presence of Zn(CN)₂ or KCN and a palladium catalyst such as Pd(PPh₃)₄ or Pd(OAc)₂ and a phosphine in solvents such as DMF at 80° C. to 140° C. for 1 to 24 hours (for a review on Pd-catalyzed cyanation of aryl halides see Eur. J. Inorg. Chem. 2003, 19, 3513). Typically, the reaction is performed using CuCN (1 to 2 equivalents) in a solvent such as DMF at a temperature from 130° C. to 160° C. for 16 to 48 hours.

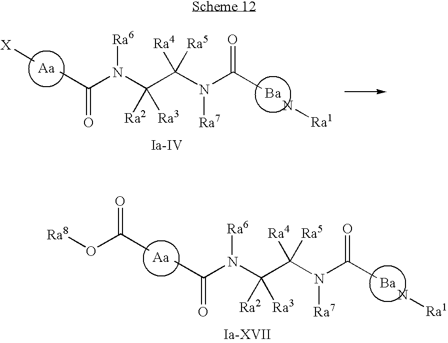

wherein the symbols are as defined above.

Scheme 12 shows methods for preparing compounds of formula Ia-XVII where the Aa ring is substituted with a carboxylic ester group. Compounds Ia-XVII can be prepared by alkoxycarbonylation of halides Ia-IV using a palladium-ligand catalyst such as (R)-(Binap)PdCl₂ or PdCl₂(PPh₃)₂ in the presence of a base such as triethylamine, Hunig's base, alkali carbonates or alkali hydroxides (lithium hydroxide, sodium hydroxide or potassium hydroxide) in solvents such as toluene or alcohols under carbon monoxide atmosphere (J. Organomet. Chem. 2002, 641 (1-2), 30; Synthesis 2002, 15, 2171). In the present invention, halides Ia-IV are preferably treated with (R)-(Binap)PdCl₂ (0.01 to 0.1 equivalents) and triethylamine (1 to 2 equivalents) in alcohol (preferably methanol or ethanol) under carbon monoxide pressure (30-100 psi) at a temperature from 20° C. to 100° C. for 24 to 48 hours.

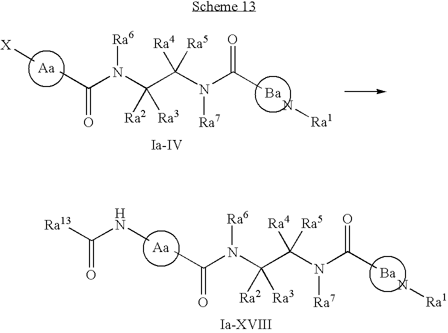

wherein Ra¹³ is an optionally substituted hydrocarbon group or an optionally substituted heterocyclic group and other symbols are as defined above.

As the “optionally substituted hydrocarbon group” and “optionally substituted heterocyclic group” for Ra¹³, those exemplarily recited as the “optionally substituted hydrocarbon group” and “optionally substituted heterocyclic group”, which are those exemplarily recited as the “substituent” for Ra¹, Ra², Ra³, Ra⁴, Ra⁵, Ra⁶ or Ra⁷, can be mentioned.

Scheme 13 shows methods for preparing compounds of formula Ia-XVIII. Compounds Ia-XVIII can be prepared by amidation of halides Ia-IV under copper-mediated or palladium-mediated conditions reported in Schemes 7 and 9 (for specific amidation coupling, see J. Am. Chem. Soc. 2002, 124(25),

7421). In the present invention, halides Ia-IV are treated with an amide Ra¹³CONH₂ (1 to 2 equivalents) in the presence of Pd₂(dba)₃ (0.1 to 1 equivalents), Xantphos (0.1 to 1 equivalent) and an alkali carbonate (1 to 3 equivalents) in a solvent such as dioxane at a temperature from 80° C. to 120° C., preferably 100° C., for 3 to 24 hours.

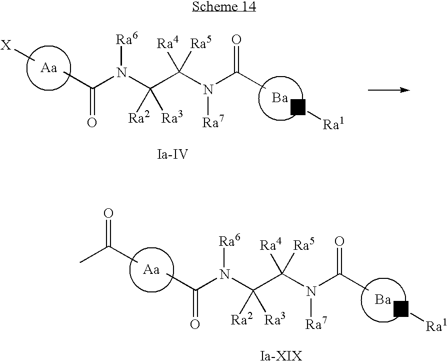

wherein the symbols are as defined above.

Scheme 14 shows methods for preparing compounds of formula Ia-XIX. Compounds Ia-XIX can be prepared under Heck conditions from halides Ia-IV and vinyl alkoxides followed by hydrolysis of the resulting alkyl enol (for a description of the Heck reaction and its applications to organic synthesis see, Negishi, Ei-ichi. Handbook of Organopalladium Chemistry for Organic Synthesis (2002), 1, p 1133-1178; John Wiley & Sons, Inc., Hoboken, N. J; Angew. Chem., Int. Ed. Engl. 2002, 41, 4176; Tetrahedron 2001, 57, 7449). Alternatively, halides Ia-IV can be treated with tributyl(1-ethoxyvinyl)stannane under Stille conditions followed by hydrolysis of the resulting alkyl enol (for a description of the Stille reaction and its applications to organic synthesis see, Aqueous-Phase Organometallic Catalysis (2nd Edition) (2004), 511-523. Publisher: Wiley-VCH Verlag GmbH & Co. KGBA, Weinheim, Germany). Preferably, halides Ia-IV are treated with 1-(vinyloxy)butane (1 equivalent) in the presence of Pd(OAc)₂ (0.1 to 1 equivalent), dppp (0.2 to 2 equivalents) and alkali carbonate (1 to 3 equivalents) in a solvent such as DMF, toluene, water or a mixture of the mentioned solvents at a temperature from 60° C. to 140° C., preferably 80° C., for 1 to 48 hours. The resulting vinyl enol can then be hydrolyzed to the acetyl group by treatment with acid, typically 2N HCl at 20° C. for 1 to 24 hours.

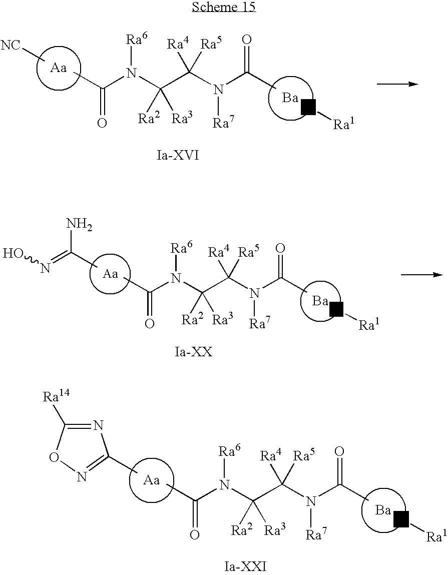

wherein Ra¹⁴ is a hydrogen atom, an optionally substituted hydrocarbon group or an optionally substituted heterocyclic group and other symbols are as defined above.

As the “optionally substituted hydrocarbon group” and “optionally substituted heterocyclic group” for Ra¹⁴, those exemplarily recited as the “optionally substituted hydrocarbon group” and “optionally substituted heterocyclic group”, which are those exemplarily recited as the “substituent” for Ra¹, Ra², Ra³, Ra⁴, Ra⁵, Ra⁶ or Ra⁷, can be mentioned.

Compounds of formula Ia-XX can be prepared by treating a nitrile Ia-XVI with hydroxylamine hydrochloride (1 to 1.5 equivalents) in solvents such as aqueous alcohols (ethanol) at a temperature of DOC to reflux for 1 to 24 hours. In the case where Ra¹⁴ is not a hydrogen atom, Ia-XX or its salts can be treated with an acid (Ra¹⁴CO₂H), an acid anhydride ((Ra¹⁴CO) 20) or an acid chloride (Ra¹⁴COCl) in the presence of a base, CDI, DCC/benzotriazole, DCC or TFFH (Synthesis 2004, (15), 2485-2492) in solvents such as DMF, THF, ACN or halogenated hydrocarbons to yield compounds of formula Ia-XXI. Preferably, Ia-XX is reacted with and acid chloride (Ra¹⁴COCl) (1-1.5 equivalents) in a base such as pyridine at a temperature from 60° C. to 100° C. for 1 to 24 hours. In the case where Ra¹⁴ is a hydrogen atom, Ia-XX is reacted in a trialkyl orthoformate (solvent) in the presence of boron trifluoride etherate (1 equivalent) at a temperature between 60° C. to 140° C. for 1 to 24 hours, to yield compounds Ia-XXI.

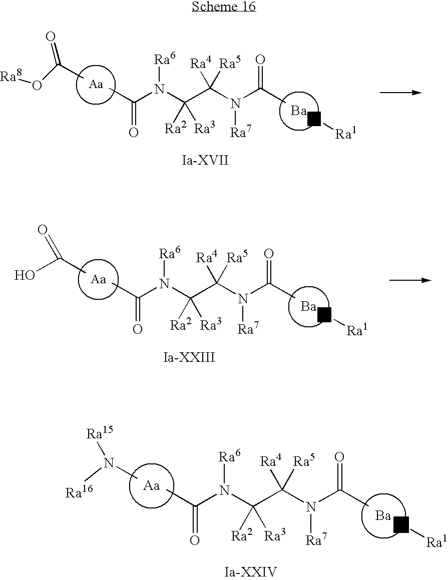

wherein Ra¹⁵ and Ra¹⁶ are each independently a hydrogen atom, an optionally substituted hydrocarbon group, an optionally substituted heterocyclic group or an acyl group and other symbols are as defined above.

As the “optionally substituted hydrocarbon group “optionally substituted heterocyclic group” and “acyl group” for Ra¹⁵ or Ra¹⁶, those exemplarily recited as the “optionally substituted hydrocarbon group”, “optionally substituted heterocyclic group” and “acyl group”, which are those exemplarily recited as the “substituent” for Ra¹, Ra², Ra³, Ra⁴Ra⁵, Ra⁶ or Ra⁷, can be mentioned.

Compounds of formula Ia-XXIII can be prepared from compounds Ia-XVII (preferably Ra⁸ is methyl or ethyl group), using hydrolysis methods similar to those described in Scheme 4. Compounds Ia-XXIII can be converted to the carbamates Ia-XXIV, wherein Ra¹⁵ is a hydrogen atom and Ra¹⁶ is an alkoxycarbonyl group (preferably tert-butoxycarbonyl group) or vice versa, via a Curtius type rearrangement (for a description of the Curtius rearrangement and its applications to organic synthesis, see, Chem. Soc. Rev. 2006, 35(2), 146-56). Preferably, acids Ia-XXIII (1 equivalent), diphenylphosphoryl azide (1 to 1.5 equivalents) and triethylamine (1 to 1.5 equivalents) are reacted in tert-butanol for 1 to 3 days at 80° C. The tert-butoxycarbonyl group is removed using acidic conditions (TFA or 4N HCl in dioxane) at room temperature to provide the unsubstituted amine Ia-XXIV, wherein Ra¹⁵ and Ra¹⁶ are both hydrogen atoms, or its salt. Compounds Ia-XXIV, wherein Ra¹⁵ and Ra¹⁶ are independently a hydrogen atom, an optionally substituted non-aromatic hydrocarbon group or an optionally substituted non-aromatic heterocyclic group (excluding the case where Ra¹⁵ and Ra¹⁶ are both hydrogen atoms), can be prepared by treating the unsubstituted amine Ia-XXIV or its salt with the corresponding halide or the corresponding sulfonate in the presence of an organic base such as pyridine, triethylamine, diisopropylethylamine or an inorganic base such as alkali hydrides or alkali carbonates. Solvents include halogenated hydrochlorides, THF or DMF. The transformation can also be accomplished by treatment with an aldehyde or a ketone in the presence of a reducing agent such as sodium borohydride, sodium cyanoborohydride or sodium triacetoxyborohydride in a solvent such as halogenated hydrocarbons. An acid such as acetic acid may be added to the reaction. The unsubstituted amine Ia-XXIV is reacted with the corresponding aldehyde or ketone (1.1-4 equivalents) and then reduced in the presence of a reducing agent (1.5-6 equivalents) at low pH. Compounds Ia-XXIV, wherein Ra¹⁵ and Ra¹⁶ are independently a hydrogen atom or an acyl group (excluding the case where Ra¹⁵ and Ra¹⁶ are both hydrogen atoms), can be prepared by treating the unsubstituted amine or its salt with the corresponding acid in the presence of a coupling reagent under conditions commonly employed to form amide bonds. In this invention, the coupling reagent of choice is HATU or EDAC.HCl/HOBt.H₂O in a solvent such as halogenated hydrocarbons or DMF at room temperature. The transformation may also be accomplished by treating the unsubstituted amine or its salt with the corresponding acid halide, acid anhydride, sulfonyl halide, isocyanate, carbamic halide, haloformate or dicarbonate in the presence of an organic base such as pyridine, triethylamine, diisopropylethylamine or an inorganic base such as alkali hydrides or alkali carbonates in a solvent such as acetone, THF, halogenated hydrocarbons or DMF at a temperature from 20° C. to 130° C. for 1 to 72 hours. Compounds Ia-XXIV, wherein Ra¹⁵ and Ra¹⁶ are independently a hydrogen atom, an optionally substituted aryl group or an optionally substituted heteroaryl group (excluding the case where Ra¹⁵ or Ra¹⁶ are both hydrogen atoms) can be prepared by reacting an unsubstituted amine Ia-XXIV or its salt with an activated aryl or heteroaryl halide under S_{N}Ar conditions (basic conditions in a polar, protic solvent; suitable bases include potassium hydride, sodium hydride, potassium tert-butoxide, lithium hydroxide or alkali carbonates in solvents such as DMF, DMSO or THF), or an aryl or heteroaryl halide under palladium mediated conditions (conditions for these transformations can be found in Angew. Chem. Int. Ed. 1998, 37, 2046; Organomet. Chem. 1999, 576, 125).

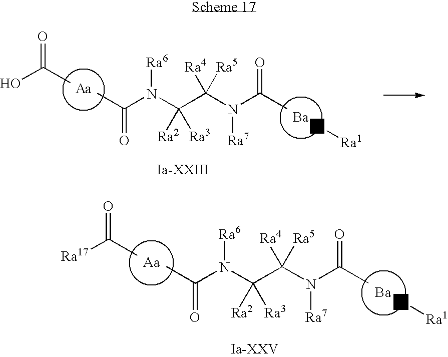

wherein Ra¹⁷ is an optionally substituted amino group and other symbols are as defined above.

As the “optionally substituted amino group” for Ra¹⁷, those exemplarily recited as the “optionally substituted amino group”, which is exemplarily recited as the “substituent” for Ra¹, Ra², Ra³, Ra⁴, Ra⁵, Ra⁶ or Ra⁷, can be mentioned.

Compounds of formula Ia-XXV can be prepared from acids Ia-XXIII (prepared according to the method described in Scheme 16) and an amine under conditions commonly employed for the formation of amide bonds.

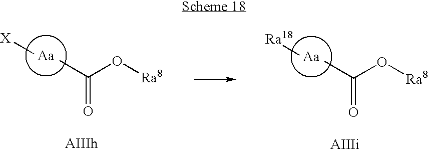

wherein Ra¹⁸ is an optionally substituted hydrocarbon group or an optionally substituted heterocyclic group and other symbols are as defined above.

As the “optionally substituted hydrocarbon group” and “optionally substituted heterocyclic group” for Ra¹⁸, those exemplarily recited as the “optionally substituted hydrocarbon group” and “optionally substituted heterocyclic group”, which are those exemplarily recited as the “substituent” for Ra¹, Ra², Ra³, Ra⁴, Ra⁵, Ra⁶ or Ra⁷, can be mentioned.

Scheme 18 shows a method for preparing intermediates AIIIi which are suitable for use in preparing compounds of formulas Ia and Ia-I as shown in Schemes 3, 4 and 5. Compounds of formula AIIi may be prepared from compounds AIIIh by reaction with alkyl zinc halides using Negishi conditions or with aryl/heteroaryl boronic acids using Suzuki conditions using similar methods to those described in Scheme 9. In the case where the Aa ring has more than one X atom, the bis coupling can be achieved using excess reagent (3 to 5 equivalents). Compounds AIIIi can be converted to the corresponding acids under conditions commonly employed.

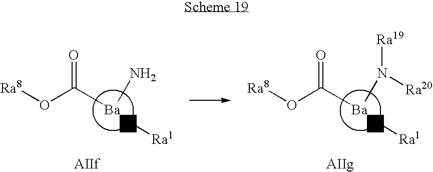

wherein Ra¹⁹ and Ra²⁰ are each independently an optionally substituted hydrocarbon group, an optionally substituted heterocyclic group or acyl group, and other symbols are as defined above.

As the “optionally substituted hydrocarbon group” and “optionally substituted heterocyclic group” for Ra¹⁹ or Ra²⁰, those exemplarily recited as the “optionally substituted hydrocarbon group” and “optionally substituted heterocyclic group”, which are those exemplarily recited as the “substituent” for Ra¹, Ra², Ra³, Ra⁴, Ra⁵, Ra⁶ or Ra⁷, can be mentioned.

Scheme 19 shows a method for preparing intermediates AIIg which are suitable for use in preparing compounds of formulas Ia and Ia-I as shown in Schemes 3, 4 and 5. Compounds of formula AIIg can be prepared from amines of formula AIIf using similar methods to those described in Scheme 16. In the preferred case wherein Ba is an optionally substituted pyrazole ring, compounds AIIf can be prepared according to the procedure described J. Het. Chem. 1967, pp. 325.

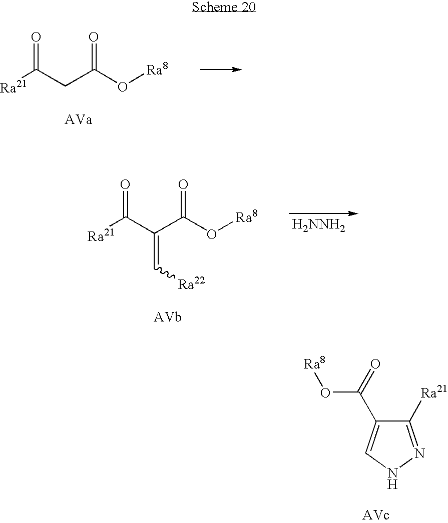

wherein Ra²¹ is an optionally-substituted hydrocarbon group or an optionally substituted heterocyclic group, Ra²² is a C₁-C₂ alkoxy group, NH₂ or NMe₂ and other symbols are as defined above.

As the “optionally substituted hydrocarbon group” and “optionally substituted heterocyclic group” for Ra²¹, those exemplarily recited as the “optionally substituted hydrocarbon group” and “optionally substituted heterocyclic group”, which are those exemplarily recited as the “substituent” for Ra¹, Ra², Ra³, Ra⁴, Ra⁵, Ra⁶ or Ra⁷, can be mentioned.

Scheme 20 shows a method for preparing intermediates AVc which are suitable for use in preparing compounds of formula AIIb as shown in Scheme 2. Compounds of formula AVb may be prepared from β-ketoesters of formula AVa. In the case where Ra²² is a C₁-C₂ alkoxy group, compounds AVb can be prepared by reacting the β-ketoester AVa (1 equivalent) with a trialkyl orthoformate (2 equivalents) and acetic anhydride (5 equivalents) at 50° C. to 100° C. for 4 to 24 hours. In the case where Ra²² is NMe₂, compound AVb can be prepared by reacting the β-ketoester AVa (1 equivalent) in DMF-DMA (2 to 50 equivalents) in the presence of a base such as triethylamine neat or in a solvent such as THF, toluene or halogenated hydrocarbons at a temperature from 0° C. to 100° C. for 1 to 48 hours. In the case where Ra²² is NH₂, compound AVb can be prepared by reacting the β-ketoester AVa with 3-methyl-5-nitropyrimidin-4(3H)-one according to the procedure described in Synlett 2004, 4, 703. From the enamines or enols AVb, pyrazoles AVc are conveniently prepared by reaction with hydrazine, its salts or hydrates in solvents such as alcohols or ethers at temperatures from 60° C. to 100° C. for 2 to 24 hours. Preferably, compound AVb, where Ra²² is a C₁-C₂ alkoxy group, is treated with hydrazine hydrate (1 to 10 equivalents) in ethanol at reflux for 3 to 12 hours.

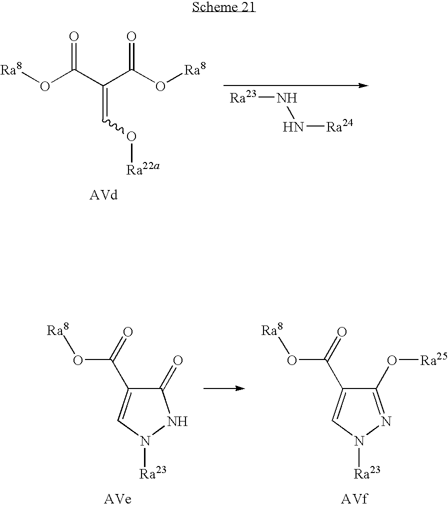

wherein Ra^{22a} is a C₁-C₂ alkyl group, Ra²³ and Ra²⁵ are each independently an optionally substituted hydrocarbon group or an optionally substituted heterocyclic group, provided that R²³ is not optionally substituted quinolyl, Ra²⁴ is a hydrogen atom or acetyl group, and other symbols are as defined above.

As the “optionally substituted hydrocarbon group” and “optionally substituted heterocyclic group” for Ra²³ or Ra²⁵, those exemplarily recited as the “optionally substituted hydrocarbon group” and “optionally substituted heterocyclic group”, which are those exemplarily recited as the “substituent” for Ra¹, Ra², Ra³, Ra⁴, Ra⁵, Ra⁶ or Ra⁷, can be mentioned.

Scheme 21 shows a method for preparing intermediates AVf which are suitable for use in preparing compounds of formulas Ia and Ia-I as shown in Schemes 3, 4 and 5. Compounds of formula AVd may be prepared from alkylmalonates according to a similar method described in Scheme 20. Compound AVd can be treated with substituted hydrazine or substituted acetyl hydrazide in the presence of POCl₃ or a base such as sodium ethoxide following the procedures described in Tetrahedron 1977, 33, 2829; Tetrahedron 1987, 43(3), 607 and WO2001023358, to yield the pyrazolinones AVe. Compound AVe can be converted to compound AVf in the presence of the corresponding activated halides and a base such as alkali hydrides or alkali carbonates in solvents such as acetonitrile or DMF at temperatures from 0° C. to 130° C. for 1 to 24 hours. In the present invention, dialkyl (alkyloxy)malonates AVd are preferably treated with POCl₃ in the presence of an aryl acetylhydrazide to afford the pyrazolinones Ave, which can then be reacted with the corresponding halides (1 to 2 equivalents) in the presence of a base such as potassium carbonate (1 to 3 equivalents) at a temperature from 20° C. to 60° C. for 3 to 12 hours.

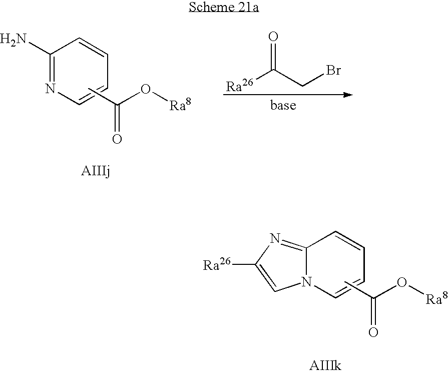

wherein Ra²⁶ is an optionally substituted hydrocarbon group or an optionally substituted heterocyclic (aromatic or non-aromatic) group and other symbols are as defined above.

As the “optionally substituted hydrocarbon group” and “optionally substituted heterocyclic group” for Ra²⁶, those exemplarily recited as the “optionally substituted hydrocarbon group” and “optionally substituted heterocyclic group”, which are those exemplarily recited as the “substituent” for Ra¹, Ra², Ra³, Ra⁴, Ra⁵, Ra⁶ or Ra⁷, can be mentioned.

Scheme 21a shows methods of preparing bicyclic intermediates AIIIk. An aminopyridine AIIIj may be treated with a corresponding optionally substituted α-bromo ketone in the presence of an inorganic base such as sodium bicarbonate in a polar protic solvent such as methanol or ethanol at a temperature of 40° C. to 80° C. for 4 to 24 hours to afford the imidazo[1,2-a]pyridine AIIIk. Compounds AIIIk can be hydrolyzed to the corresponding acids under conditions commonly employed.

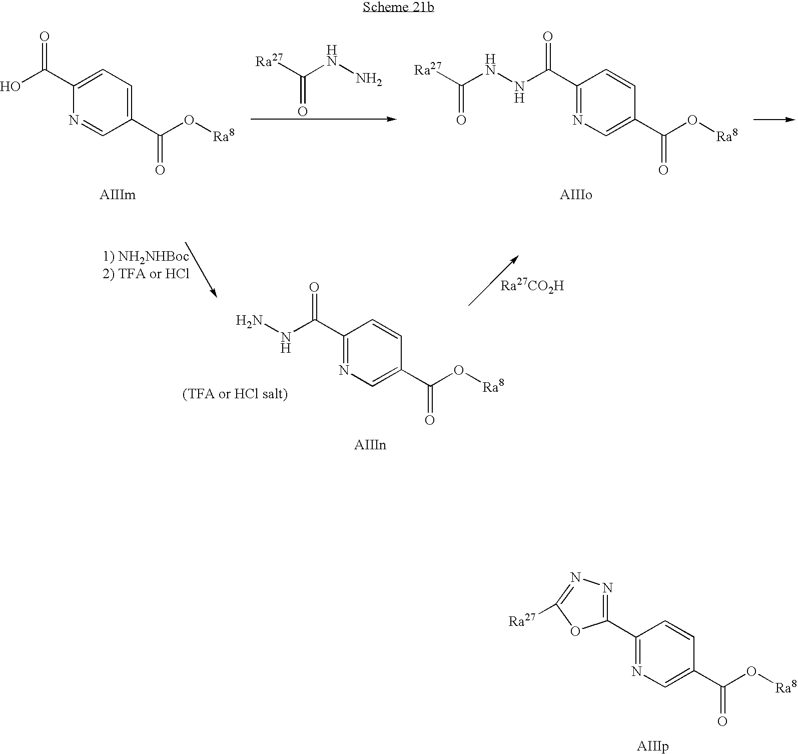

wherein Ra²⁷ is an optionally substituted hydrocarbon group or an optionally substituted heterocyclic (aromatic or non-aromatic) group and other symbols are as defined above.

As the “optionally substituted hydrocarbon group” and “optionally substituted heterocyclic group” for Ra²⁷, those exemplarily recited as the “optionally substituted hydrocarbon group” and “optionally substituted heterocyclic group”, which are those exemplarily recited as the “substituent” for Ra¹, Ra², Ra³, Ra⁴, Ra⁵, Ra⁶ or Ra⁷, can be mentioned.

Scheme 21b shows methods of preparing intermediates AIIIp, in which the pyridine ring is substituted with a 1,3,4-oxadiazolyl group. Compounds AIIIo can be prepared from the acid AIIIm and an acyl hydrazide (Ra²⁷CONHNH₂) in conditions commonly employed to form amide bonds. Compounds AIIIo can be treated with a reagent such as POCl₃, PhPOCl₂, TFAA/Py or TsCl/Py to form the corresponding substituted 1,3,4-oxadiazoles AIIIp. Preferably, compounds AIIIo are treated with POCl₃ (1.1-10 equivalents) in a solvent such as acetonitrile or neat at a temperature of 80° C. for 1 to 24 hours.

Another method consists in treating the acid AIIIm with a suitably protected hydrazine in conditions commonly employed to form amide bonds. In the present invention, the protected hydrazine is preferably Boc-hydrazine. The resulting protected hydrazide may be hydrolyzed in acidic conditions such as TFA or HCl in dioxane (2-10 equivalents) at temperatures from 20° C. to 90° C. for 1 to 24 hours to yield the hydrazide salts AIIIn. Compounds AIIIn can be reacted with a corresponding acid (Ra²⁷COOH) under conditions commonly used to form amide bonds to give compounds AIIIo, which can be converted to compounds AIIIp under the conditions mentioned above. Compounds AIIIp may be hydrolyzed to the corresponding acids under conditions commonly employed.

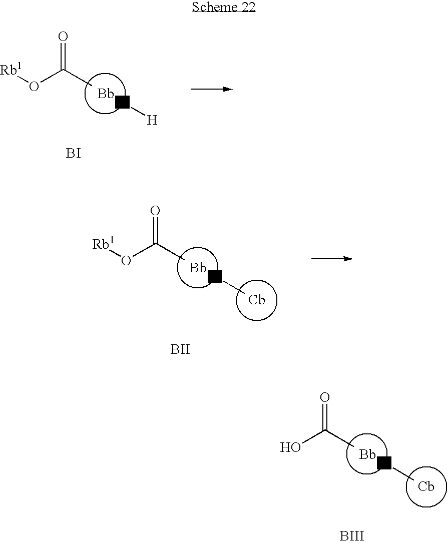

wherein Rb¹ is a C₁-C₄ alkyl or benzyl group and other symbols are as defined above.

Intermediate esters BII and acids BIII which are suitable for use in preparing compounds of formula Ib can be prepared according to the following references: Tetrahedron Lett. 1998, 39, 2941-2944; Eur. J. Org. Chem. 2004, 695-709; J. Am. Chem. Soc 2001, 123, 7727-7729; J. Am. Chem. Soc. 2002, 124, 11684-11688; J. Org. Chem. 2004, 69, 5578. Typically, the N-heteroarylation of the Bb ring is performed with a heteroaryl halide (preferably iodide) in the presence of copper catalyst such as copper iodide or copper oxide in the presence of a ligand such as substituted ethylene diamines, salicylaldoximes or other ligands reported in Eur. J. Org. Chem. 2004, 695-709. The reaction requires a base such as potassium phosphate or cesium carbonate and is performed in a degassed solvent such as acetonitrile, toluene or DMF at a temperature of 20° C. to 150° C. for 0.5 to 48 hours under inert atmosphere. Preferably, the N-heteroarylation is conducted according to the method described in J. Org. Chem. 2004, 69, 5578, in toluene with 1 equivalent of BI, 1.1-10 equivalents of heteroaryl halide, 2 equivalents of diamine ligand, 2-3 equivalents of base and 0.05 equivalents of copper(I) iodide or according to the method described in Eur. J. Org. Chem. 2004, 695-709, in DMF with 1 equivalent of BI, 1.5-10 equivalents of heteroaryl halide, 0.2-0.4 equivalents of oxime ligand, 2-3 equivalents of base and 0.05 equivalents of copper(II) oxide.

Acids BIII can be prepared from the corresponding esters BII by using a base such as lithium hydroxide, sodium hydroxide, alkali carbonates (sodium carbonate, potassium carbonate or cesium carbonate) in a polar protic solvent such as methanol, ethanol, water or in mixtures of solvents including alcohols and water, or aprotic solvents. Typically, the hydrolysis is performed in an alcohol (methanol or ethanol) or in a 1:1 mixture of alcohol/THF, with water in the presence of sodium hydroxide (1-10 equivalents) at a temperature ranging from 20° C. to 100° C. for 0.5 to 24 hours. Acids BIII can also be prepared from the corresponding esters BII by acid hydrolysis using an acid such as TFA, HCl, H₂SO₄, AcOH or in a mixture of these acids in neat or aqueous condition at a temperature ranging from 20° C. to 100° C. for 0.5 to 24 hours. In the case where Rb¹ is benzyl group, acids BIII can be prepared from the corresponding esters BII by hydrogenolysis using catalysts such as palladium on carbon or palladium hydroxide in a protic solvent such as EtOH or aprotic solvent such as EtOAc under hydrogen atmosphere at a pressure of 15 to 150 psi, at a temperature of 20° C. to 100° C. for 1 to 48 hours. Additional conditions for the hydrolysis of ester groups can be found in T. W. Green, Protective Groups in Organic Synthesis, John Wiley and Sons, Inc., 1981.

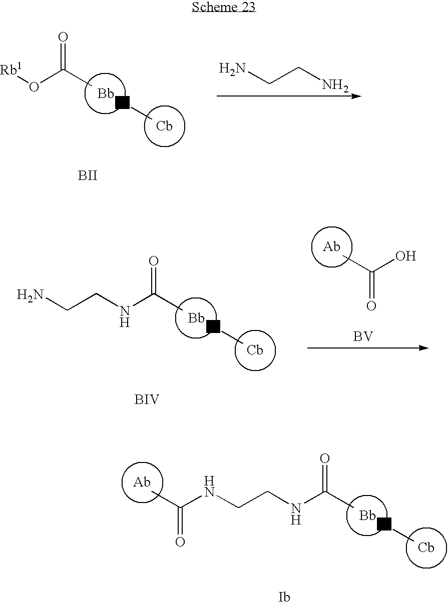

wherein the symbols are as defined above.

Compounds Ib can be prepared according to the sequence described in Scheme 23. Esters BII, where Rb¹ is preferably methyl or ethyl group, can be treated with ethylenediamine at refluxing temperature to produce amines BIV. Compounds Ib can be conveniently prepared from an amine BIV or its salt and an acid BV in the presence of various condensing reagents. Known condensing reagents that effect amide bond formation include, but are not limited to, N,N-carbonyldiimidazole, halopyridine salts, 2,4,6-trichlorobenzoyl chloride, HATU, BOP-Cl or EDAC.HCl/HOBt.H₂O. In the present invention, the preferred reagent is EDAC.HCl/HOBt.H₂O. The reaction can be conducted in a variety of aprotic solvents such as halogenated hydrocarbons (DCM or CHCl₃), acetonitrile or dimethylformamide, or a mixture of these solvents, at a temperature from 0° C. to 130° C., preferably 20° C. to 70° C., for a time ranging from 0.5 to 48 hours. A base such as triethylamine or diisopropylethylamine may be used especially if the reacting amine is in a salt form. While the amount of reagent varies depending on the condensing reagent used, the following stoichiometry is used preferably with EDAC.HCl/HOBt.H₂O: amine or its salt (1 equivalent), acid (1 equivalent), EDAC.HCl (1 to 2 equivalents), HOBt.H₂O (1 to 2 equivalents) and base (1 to 3 equivalents).

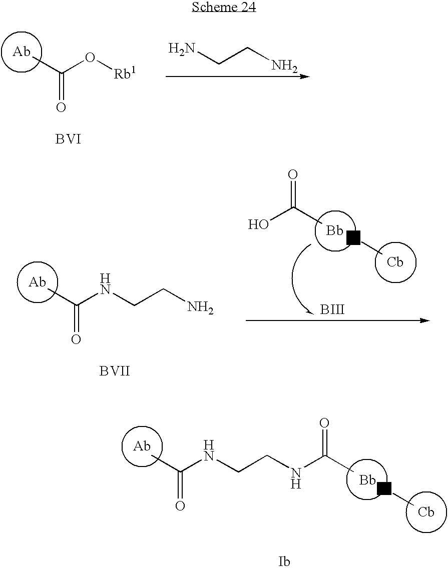

wherein the symbols are as defined above.

Alternatively, compounds Ib can be prepared following the sequence described in Scheme 24. Esters BVI, where Rb¹ is preferably methyl or ethyl group, can be treated with ethylenediamine at refluxing temperature to produce amines BVII. Compounds Ib can be prepared from the amine BVII or its salt and acid BIII in the conditions commonly employed to form amide bonds such as those mentioned in Scheme 23.

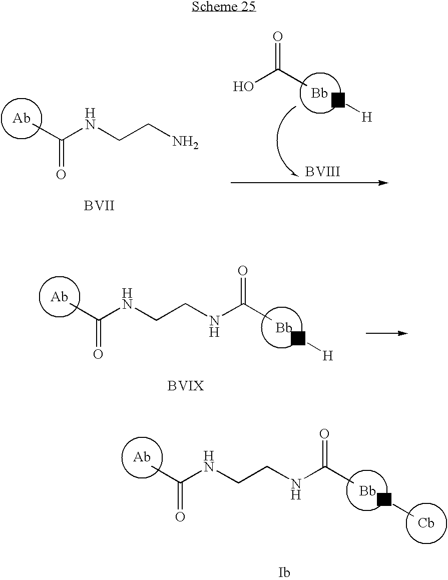

wherein the symbols are as defined above.

Alternatively, compounds Ib can be prepared according to Scheme 25. The amine BVII can be coupled to the acid BVIII under conditions commonly employed to form amide bonds. The amine BVIX can be further transformed to compound Ib under conditions similar to those described in Scheme 22.

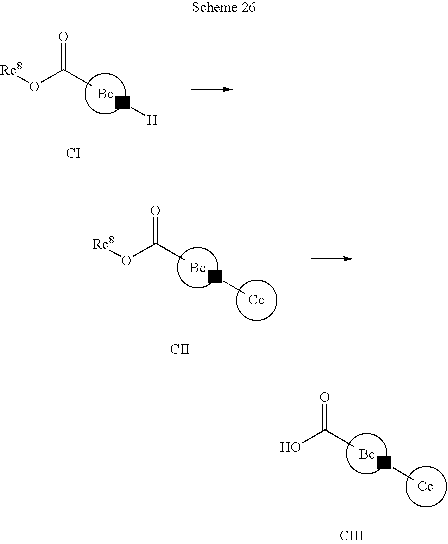

wherein Rc⁸ is a C₁-C₄ alkyl or benzyl group and other symbols are as defined above.

Intermediate esters CII and acids CIII which are suitable for use in preparing compounds of formula Ic can be prepared according to the following references: Tetrahedron Lett. 1998, 39, 2941-2944; Eur. J. Org. Chem. 2004, 695-709; J. Am. Chem. Soc 2001, 123, 7727-7729; J. Am. Chem. Soc. 2002, 124, 11684-11688; J. Org. Chem. 2004, 69, 5578. Typically, the N-arylation or N-heteroarylation of the Bc ring is performed with a aryl or heteroaryl halide (preferably iodide) in the presence of copper catalyst such as copper iodide or copper oxide in the presence of a ligand such as substituted ethylene diamines, salicylaldoximes or other ligands reported in Eur. J. Org. Chem. 2004, 695-709. The reaction requires a base such as potassium phosphate or cesium carbonate and is performed in a degassed solvent such as acetonitrile, toluene or DMF at a temperature of 20° C. to 150° C. for 0.5 to 48 hours under inert atmosphere. Preferably, the N-arylation or N-heteroarylation is conducted according to the method described in J. Org. Chem. 2004, 69, 5578, in toluene with 1 equivalent of CI, 1.1-10 equivalents of aryl or heteroaryl halide, 2 equivalents of 1 diamine ligand, 2-3 equivalents of base and 0.05 equivalents of copper(I) iodide or according to the method described in Eur. J. Org. Chem. 2004, 695-709, in DMF with 1 equivalent of CI, 1.5-10 equivalents of aryl or heteroaryl halide, 0.2-0.4 equivalents of oxime ligand, 2-3 equivalents of base and 0.05 equivalents of copper(II) oxide.

Acids CIII can be prepared from the corresponding esters CII by using a base such as lithium hydroxide, sodium hydroxide, alkali carbonates (sodium carbonate, potassium carbonate or cesium carbonate) in a polar protic solvent such as methanol, ethanol, water or in mixtures of solvents including alcohols and water, or aprotic solvents. Typically, the hydrolysis is performed in an alcohol (methanol or ethanol) or in a 1:1 mixture of alcohol/THF, with water in the presence of sodium hydroxide (1-10 equivalents) at a temperature ranging from 20° C. to 100° C. for 0.5 to 24 hours. Acids CIII can also be prepared from the corresponding esters CII by acid hydrolysis using an acid such as TFA, HCl, H₂SO₄, AcOH or in a mixture of these acids in neat or aqueous condition at a temperature ranging from 20° C. to 100° C. for 0.5 to 24 hours. In the case wherein Rb¹ is benzyl group, acids CIII can be prepared from the corresponding esters CII by hydrogenolysis using catalysts such as palladium on carbon or palladium hydroxide in a protic solvent such as EtOH or aprotic solvent such as EtOAc under hydrogen atmosphere at a pressure of 15 to 150 psi, at a temperature of 20° C. to 100° C. for 1 to 48 hours. Additional conditions for the hydrolysis of ester groups can be found in T. W. Green, Protective Groups in Organic Synthesis, John Wiley and Sons, Inc., 1981.

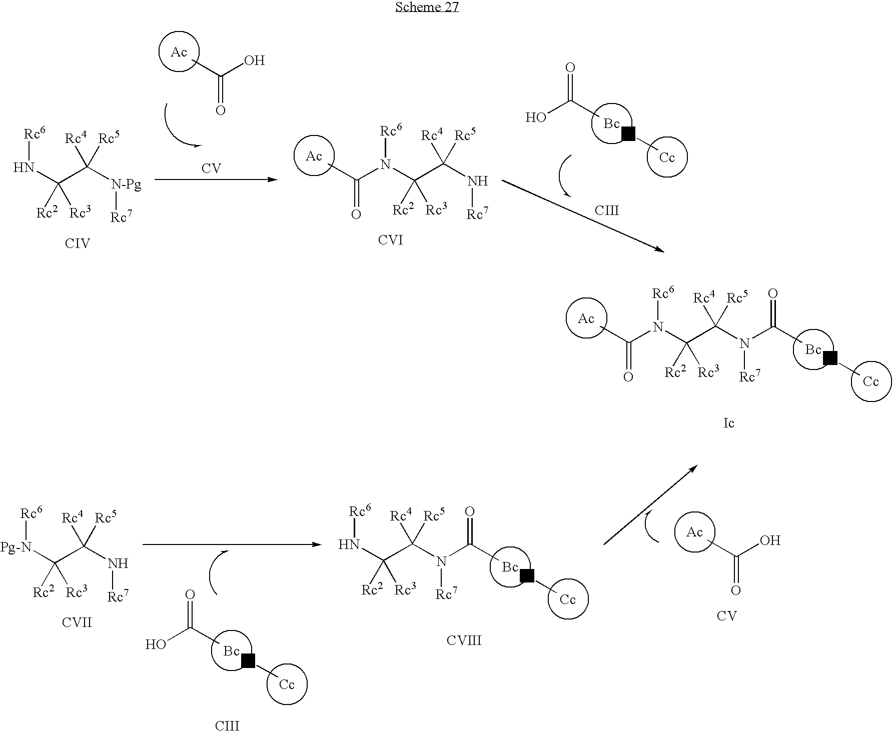

wherein the symbols are as defined above.

Compounds Ic can be prepared according to Scheme 27.

Compounds CVI can be the result of an amide coupling between a suitably protected amine CIV (Pg is preferably Boc or Cbz group) and an acid CV in the presence of various condensing reagents followed by deprotection of the amino group. Known condensing reagents that effect amide bond formations include, but are not limited to, N,N-carbonyldiimidazole, halopyridine salts, 2,4,6-trichlorobenzoyl chloride, HATU, BOP-Cl or EDAC-HCl/HOBt.H₂O. In the present invention, the preferred reagent is EDAC-HCl/HOBt.H₂O. The reaction can be conducted in a variety of aprotic solvents such as halogenated hydrocarbons (DCM or CHCl₃), acetonitrile or dimethylformamide, or a mixture of these solvents, at a temperature from 0° C. to 130° C., preferably 20° C. to 70° C., for a time ranging from 0.5 to 48 hours. A base such as triethylamine or diisopropylethylamine may be used especially if the reacting amine is in a salt form. While the amount of reagent varies depending on the condensing reagent used, the following stoichiometry is used preferably with EDAC.HCl/HOBt.H₂O: amine or its salt (1 equivalent), acid (1 equivalent), EDAC-HCl (1 to 2 equivalents), HOBt.H₂O (1 to 2 equivalents) and base (1 to 3 equivalents).

In the preferred case wherein Pg is Boc group, the deprotection is conveniently performed in the presence of acids such as TFA or HCl, neat or in a solvent such as ethyl ether or dioxane at a temperature from 0° C. to 100° C. for 5 minutes to 24 hours. In the present invention, the preferred deprotection method for Boc-protected amines consists in treating the protected amine in TFA or in 4N HCl in dioxanes at 20° C. for 10 minutes to 24 hours. Additional conditions for the deprotection of amines can be found in T. W. Green, Protective Groups in Organic Synthesis, John Wiley and Sons, Inc., 1981.

Compounds CVI can be further coupled to an acid CIII in conditions commonly employed to form amide bonds to afford compounds Ic.

Alternatively, compounds CVIII can be the result of the coupling between a suitably protected amine CVII (Pg is preferably Boc or Cbz group) and an acid CIII under conditions commonly employed to form amide bonds followed by deprotection of the amino group. Compounds Ic can be produced by further coupling the amine CVIII with an acid CV under conditions commonly employed to form amide bonds.

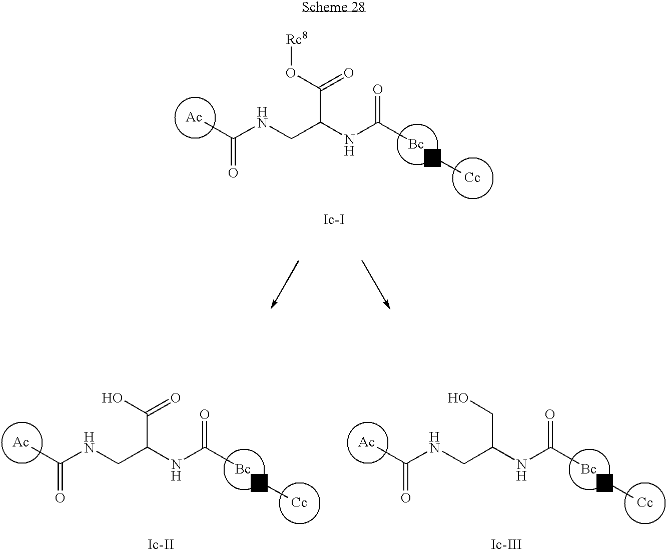

wherein the symbols are as defined above.

Compounds of formula Ic-I can be prepared from a Pg protected diamino ester following the method described in Scheme 27. The esters Ic-I may be hydrolyzed to the acids Ic-II using conditions commonly employed for the hydrolysis of ester. Esters Ic-I can also be reduced to the corresponding alcohols Ic-III in the presence of a reducing agent such as sodium borohydride. In the present invention, Ic-I is preferably reduced in the presence of the couple sodium borohydride/lithium chloride (1 to 3 equivalents) in a solvent system such as THF/ethanol at a temperature from 0° C. to 80° C. for 1 to 24 hours.

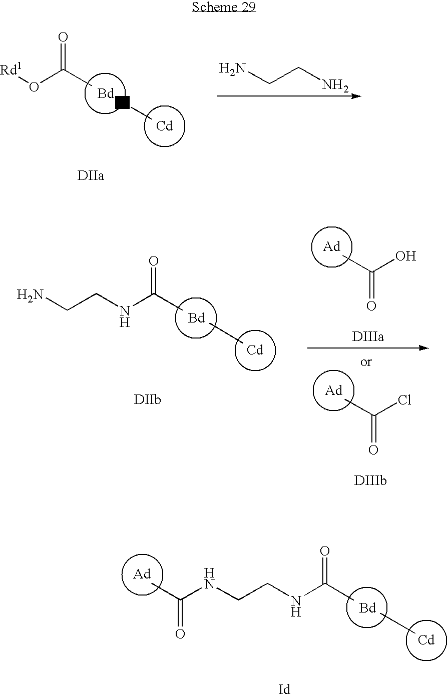

wherein Rd¹ is a C₁-C₄ alkyl or benzyl group and other symbols are as defined above.

Compounds Id can be prepared according to the sequence shown in Scheme 29. Esters DIIa, where Rd¹ is preferably methyl or ethyl group, can be treated with ethylenediamine at refluxing temperature to produce amines DIIb. Compounds of formula Id can then be prepared from acids DIIIa and amines DIIb or their salts by reacting both intermediates in the presence of various condensing reagents. Known condensing reagents that effect amide bond formation include N,N-carbonyldiimidazole, halopyridine salts, 2,4,6-trichlorobenzoyl chloride, HATU, BOP-Cl, EDAC.HCl/HOBt.H₂O or TsCl/N-methyl imidazole. In the present invention, the preferred reagent is either HATU or EDAC.HCl/HOBt.H₂O. The reaction can be conducted in aprotic solvents such as tetrahydrofuran, halogenated hydrocarbons (DCM or CHCl₃), acetonitrile, dimethylformamide, or a mixture of these solvents, at a temperature from 0° C. to 130° C., preferably 20° C. to 70° C., for a time ranging from 1 to 48 hours, preferably 10 to 20 hours. A base such as triethylamine or diisopropylethylamine may be used especially if the reacting amine is in a salt form. While the amount of reagent varies depending on the coupling reagent used, the following amounts are used preferably with HATU or EDAC.HCl/HOBt.H₂O: amine or its salt (1 equivalent), acid (1 equivalent), HATU or EDAC.HCl/HOBt.H₂O (1 to 2 equivalents), base (1 to 3 equivalents if salt form of amine is used). Compounds Id can also be prepared from acid chlorides DIIIb and amines DIIb in the presence of a base such as triethylamine, diisopropylethylamine or pyridine in an aprotic solvent such as THF, benzene, halogenated hydrocarbons at temperatures from 20° C. to 90° C. for 0.5 to 24 hours.

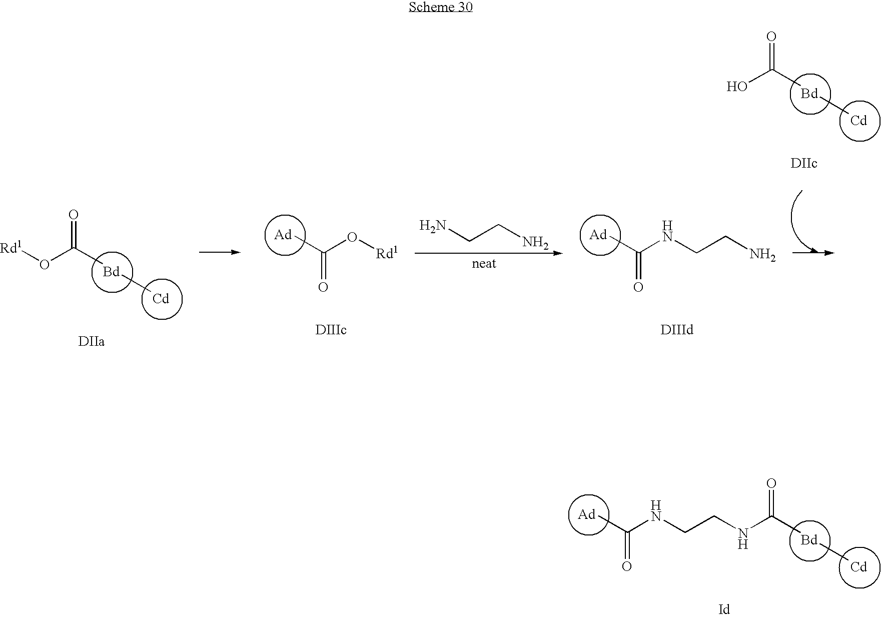

wherein the symbols are as defined above.

Alternatively, compounds Id can be prepared following the sequence described in Scheme 30. Esters DIIIc, where Rd¹ is preferably methyl or ethyl group, can be treated with ethylenediamine at refluxing temperature to produce amines DIIId. Compounds Id can be prepared under the conditions mentioned in Scheme 29 using an amine DIIId or its salt form, and an acid DIIc. Acids DIIc can be prepared from the corresponding esters DIIa by using a base such as lithium hydroxide, sodium hydroxide, alkali carbonates (sodium carbonate, potassium carbonate or cesium carbonate) in a polar protic solvent such as methanol, ethanol, water or in mixtures of solvents including the mentioned polar protic solvent or other aproptic solvents. Typically, the hydrolysis is performed in an alcohol (methanol or ethanol) or in a 1:1 mixture of alcohol/THF, with water in the presence of sodium hydroxide (1-10 equivalents) at a temperature ranging from 20° C. to 100° C. for 4 to 24 hours. Acids DIIc can also be prepared from the corresponding esters DIIa by acid hydrolysis using an acid such as TFA, HCl, H₂SO₄, AcOH or in a mixture of these acids in neat or aqueous condition at a temperature ranging from 20° C. to 100° C. for 0.5 to 24 hours. In the case where Rd¹ is benzyl group, acids DIIc can be prepared from DIIa by hydrogenolysis using catalysts such as palladium on carbon or palladium hydroxide in a protic solvent such as EtOH or aprotic solvent such as EtOAc under hydrogen atmosphere at a pressure of 15 to 150 psi, at a temperature of 20° C. to 100° C. for 1 to 48 hours. Additional conditions for the hydrolysis of ester groups can be found in T. W. Green, Protective Groups in Organic Synthesis, John Wiley and Sons, Inc., 1981.

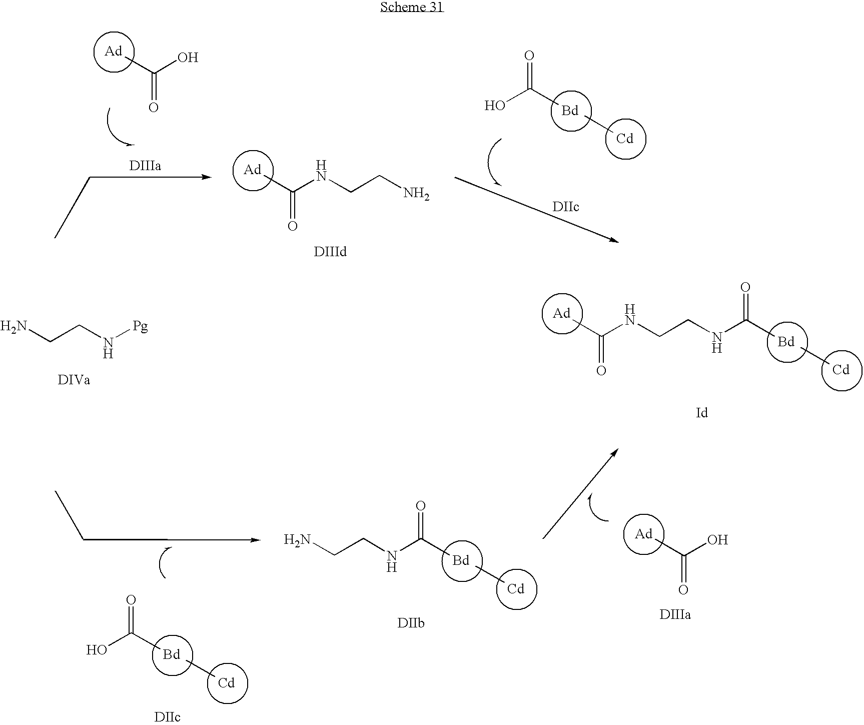

wherein the symbols are as defined above.

Compounds of formula Id can be prepared according to Scheme 31. Compounds DIIId can be the result of an amide coupling between a suitably protected ethylenediamine DIVa, where Pg is preferably Boc or Cbz group, and an acid DIIIa in conditions commonly employed to form amide bonds, followed by the deprotection of the amino group. In the preferred case wherein Pg is Boc group, the deprotection is conveniently performed in the presence of acids such as TFA or HCl, neat or in a solvent such as ethyl ether or dioxane at a temperature from 0° C. to 100° C. for 5 minutes to 24 hours. Additional conditions for the deprotection of amines can be found in T. W. Green, Protective Groups in Organic Synthesis, John Wiley and Sons, Inc., 1981. In the present invention, the preferred deprotection method for Boc-protected amines consists in treating the protected amine in 4N HCl in dioxanes (1-10 equivalents) at 20° C. for 10 minutes to 3 hours. Compounds DIIId or their salts can be further coupled to acids DIIc in conditions commonly employed to form amide bonds to produce compounds Id.

Alternatively, compounds DIIb can be the result of the coupling between a suitably protected ethylenediamine DIVa, where Pg is preferably Boc or Cbz group, and an acid DIIc under conditions commonly employed to form amide bonds, followed by deprotection of the amino group. Compounds Id can be produced by further coupling the amine DIIb or its salt with an acid DIIIa under conditions commonly employed to form amide bonds.

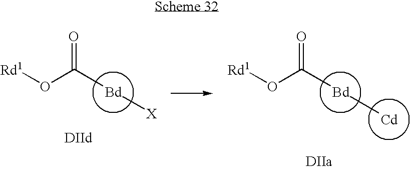

wherein X is a halogen atom and other symbols are as defined above.

Scheme 32 shows a method for preparing intermediates DIIa which are suitable for use in preparing compounds of formula Id as shown in Schemes 29, 30 and 31. Compounds DIIa can be conveniently prepared from the halogen substituted DIId under palladium-catalyzed conditions such as Suzuki (Chem. Rev. 1995, 95, 2457), Negishi (Negishi, Ei-ichi. Handbook of Organopalladium Chemistry for Organic Synthesis (2002), 1, 767-789; John Wiley & Sons, Inc., Hoboken, N. J) or Stille (Aqueous-Phase Organometallic Catalysis (2nd Edition) (2004), 511-523. Publisher: Wiley-VCH Verlag GmbH & Co. KGAA, Weinheim, Germany). Typically, the coupling is performed between a boronic acid, a zinc halide or a trialkylstanane and the halogen substituted DIId in the presence of a catalysts such as, but not limited to, Pd(PPh₃)₄, Pd(OAc)₂, PdCl₂(dppf) or PdCl₂(PPh)₂, a base such as alkali carbonates, alkali phosphates or potassium fluoride and a ligand (J. Am. Chem. Soc. 1999, 121, 9550-9561) such as phosphines in a solvent such as toluene, THF, alcohols, water or mixtures of the above solvents.

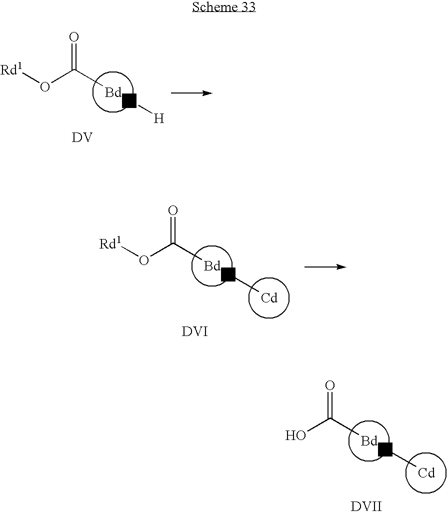

wherein the symbols are as defined above.

Intermediate esters DVI and acids DVII which are suitable for use in preparing compounds of formula Id as shown in Schemes 30 and 31, can be prepared according to the following references: Tetrahedron Lett. 1998, 39, 2941-2944; Eur. J. Org. Chem. 2004, 695-709; J. Am. Chem. Soc 2001, 123, 7727-7729; J. Am. Chem. Soc. 2002, 124, 11684-11688; J. Org. Chem. 2004, 69, 5578. Typically, the N-arylation or N-heteroarylation of the Bd ring is performed with a aryl or heteroaryl halide (preferably iodide) in the presence of copper catalyst such as copper iodide or copper oxide in the presence of a ligand such as substituted ethylene diamines, salicylaldoximes or other ligands reported in Eur. J. Org. Chem. 2004, 695-709. The reaction requires a base such as potassium phosphate or cesium carbonate and is performed in a degassed solvent such as acetonitrile, toluene or DMF at a temperature of 20° C. to 150° C. for 0.5 to 48 hours under inert atmosphere. Preferably, the N-arylation or N-heteroarylation is conducted according to the method described in J. Org. Chem. 2004, 69, 5578, in toluene with 1 equivalent of DV, 1.1-10 equivalents of aryl or heteroaryl halide, 2 equivalents of diamine ligand, 2-3 equivalents of base and 0.05 equivalents of copper(I) iodide or according to the method described in Eur. J. Org. Chem. 2004, 695-709, in DMF with 1 equivalent of DV, 1.5-10 equivalents of aryl or heteroaryl halide, 0.2-0.4 equivalents of oxime ligand, 2-3 equivalents of base and 0.05 equivalents of copper(II) oxide.

Acids DVII can be prepared from the corresponding esters DVI by using similar methods to those described in Scheme 30.

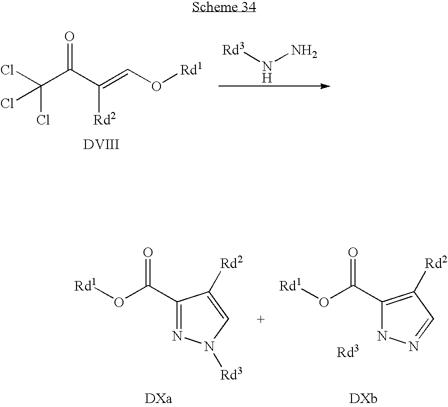

wherein Rd² is an optionally substituted hydrocarbon group, Rd³ is an optionally substituted aromatic hydrocarbon group and other symbols are as defined above.

As the “optionally substituted hydrocarbon group” for Rd², those exemplarily recited as the “optionally substituted hydrocarbon group”, which is exemplarily recited as the “substituent” for Ra¹, Ra², Ra³, Ra⁴, Ra⁵, Ra⁶ or Ra⁷, can be mentioned.

As the “optionally substituted aromatic hydrocarbon group” for Rd³, groups corresponding to those exemplarily recited as the “optionally substituted aromatic hydrocarbon”, which is exemplarily recited as the “optionally substituted aromatic ring” for ring Cd, can be mentioned.

Scheme 34 shows a method for preparing intermediates DXa and DXb which are suitable for use in preparing compounds of formula Id as shown in Schemes 29, 30 and 31. Compounds DXa and DXb can be conveniently prepared from enol ethers DVIII (Chem. Ber. 1982, 115(8), 2766) by reaction with a Rd³-substituted hydrazine in solvents such as alcohols at temperatures from 50° C. to 100° C. for 2 to 24 hours.

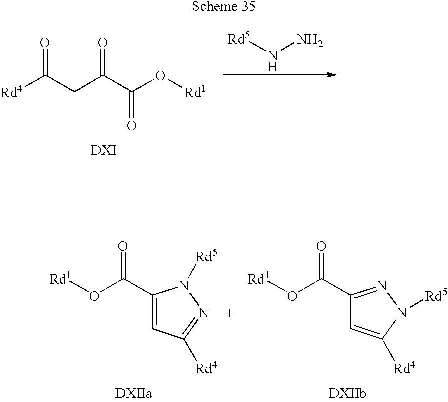

wherein at least one of Rd⁴ and Rd⁵ is an optionally substituted aromatic hydrocarbon group, the other is an optionally substituted hydrocarbon group or an optionally substituted non-aromatic heterocyclic group, and other symbols are as defined above.

As the “optionally substituted aromatic hydrocarbon group” for Rd⁴ or Rd⁵, groups corresponding to those exemplarily recited as the “optionally substituted aromatic hydrocarbon”, which is exemplarily recited as the “optionally substituted aromatic ring” for ring Cd, can be mentioned.

As the “optionally substituted hydrocarbon group” and “optionally substituted non-aromatic heterocyclic group” for Rd⁴ or Rd⁵, those exemplarily recited as the “optionally substituted hydrocarbon group” and “optionally substituted non-aromatic heterocyclic group”, which are those exemplarily recited as the “substituent” for Ra¹, Ra², Ra³, Ra⁴, Ra⁵, Ra⁶ or Ra⁷, can be mentioned.

Scheme 35 shows a method for preparing intermediates DXIIa and DXIIb which are suitable for use in preparing compounds of formula Id as shown in Schemes 29, 30 and 31. Compounds DXIIa and DXIIb can be conveniently prepared from diketo esters DXI and Rd⁵-substituted hydrazines in solvents such as alcohols or mixtures of alcohols and water at temperatures from 50° C. to 100° C. for 2 to 24 hours. Typically, the diketo ester DXI is treated with an aryl hydrazine (1 to 3 equivalents) in ethanol at reflux temperature for 2 to 8 hours to yield a mixture of isomers DXIIa and DXIIb which can be separated by chromatography.

The conditions (solvent, reaction temperature, reaction time, chemical equivalent ratio) for each reaction in each of the above-mentioned production methods can be appropriately determined depending on the compound to be produced, the kind of reaction and the like.

In the thus-obtained compound of the present invention, the functional group in a molecule can also be converted to an objective functional group by combining chemical reactions known per se. As such chemical reactions, oxidation reaction, reduction reaction, alkylation reaction, hydrolysis, amination reaction, esterification reaction, aryl coupling reaction, deprotection and the like can be mentioned.

In the above-mentioned production method, when the starting compound has an amino group, a carboxyl group, a hydroxy group or a carbonyl group as a substituent, a protecting group generally used in peptide chemistry and the like may be introduced into these groups. By eliminating the protecting group as necessary after the reaction, the objective compound can be obtained.

As the amino-protecting group, for example, formyl group, C₁₋₆ alkyl-carbonyl group, C₁₋₆ alkoxy-carbonyl group, benzoyl group, C₇₋₁₀ aralkyl-carbonyl group (e.g., benzylcarbonyl), C₇₋₁₄ aralkyloxy-carbonyl group (e.g., benzyloxycarbonyl, 9-fluorenylmethoxycarbonyl), trityl group, phthaloyl group, N,N-dimethylaminomethylene group, substituted silyl group (e.g., trimethylsilyl, triethylsilyl, dimethylphenylsilyl, tert-butyldimethylsilyl, tert-butyldiethylsilyl), C₂₋₆ alkenyl group (e.g., 1-allyl) and the like can be mentioned. These groups are optionally substituted by 1 to 3 substituents selected from halogen atom, C₁₋₆ alkoxy group and nitro group.

As the carboxyl-protecting group, for example, C₁₋₆ alkyl group, C₇₋₁₁ aralkyl group (e.g., benzyl), phenyl group, trityl group, substituted silyl group (e.g., trimethylsilyl, triethylsilyl, dimethylphenylsilyl, tert-butyldimethylsilyl, tert-butyldiethylsilyl), C₂₋₆ alkenyl group (e.g., 1-allyl) and the like can be mentioned. These groups are optionally substituted by 1 to 3 substituents selected from halogen atom, C₁₋₆ alkoxy group and nitro group.

As the hydroxy-protecting group, for example, C₁₋₆ alkyl group, phenyl group, trityl group, C₇₋₁₀ aralkyl group (e.g., benzyl), formyl group, C₁₋₆ alkyl-carbonyl group, benzoyl group, C₇₋₁₀ aralkyl-carbonyl group (e.g., benzylcarbonyl), 2-tetrahydropyranyl group, 2-tetrahydrofuranyl group, substituted silyl group (e.g., trimethylsilyl, triethylsilyl, dimethylphenylsilyl, tert-butyldimethylsilyl, tert-butyldiethylsilyl), C₂₋₆ alkenyl group (e.g., 1-allyl) and the like can be mentioned. These groups are optionally substituted by 1 to 3 substituents selected from halogen atom, C₁₋₆ alkyl group, C₁₋₆ alkoxy group and nitro group.

As the carbonyl-protecting group, for example, cyclic acetal (e.g., 1,3-dioxane), non-cyclic acetal (e.g., di-C₁₋₆ alkylacetal) and the like can be mentioned.

For elimination of the above-mentioned protecting group, a method known per se, for example, a method described in Protective Groups in Organic Synthesis, John Wiley and Sons (1980) and the like can be mentioned. For example, employed is a method using acid, base, UV light, hydrazine, phenyl hydrazine, sodium N-methyldithiocarbamate, tetrabutylammonium fluoride, palladium acetate, trialkylsilyl halide (e.g., trimethylsilyl iodide, trimethylsilyl bromide and the like) and the like, reduction and the like.

In the above-mentioned production methods, the starting compound may be in the form of a salt. As such salt, those similar to the salts of the aforementioned compound of the present invention can be mentioned.

When the compound of the present invention contains an optical isomer, a stereoisomer, a positional isomer or a rotational isomer, these can be obtained as a single product according to a synthetic method and separation method known per se.

The compound of the present invention may be in the form of a crystal.

The crystal of the compound of the present invention can be produced by crystallization of the compound of the present invention-according to a crystallization method known per se.

The crystal the compound of the present invention is superior in physicochemical properties (e.g., melting point, solubility, stability and the like) and biological properties (e.g., pharmacokinetics (absorption, distribution, metabolism, excretion), efficacy expression and the like), and is extremely useful as a pharmaceutical agent.

### EXAMPLES

The present invention is explained in more detail by referring to the following Examples, Formulation Examples and Experimental Example, which are not to be construed as limitative.

In the examples described below, unless otherwise indicated all temperatures are set forth in degrees Celsius and ambient temperature, or room temperature, is typically 18° C. to 25° C.

Starting materials, the preparation of which are not described, are commercially available or can be readily prepared by known techniques from commercially available starting materials.

Reagents were purchased from commercial suppliers such as Aldrich Chemical Company, Lancaster, Acros international, TCI or Maybridge, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe.

Yields are given for illustration only and are not necessarily the maximum attainable.

The intermediates and final products described herein may be isolated and purified by the conventional techniques known to artisans of organic chemistry. These techniques include, but are not limited to, concentration, concentration under reduced pressure, extraction with solvents, crystallization, recrystallization, transfer dissolution and chromatography. Chromatography was performed using glass column and silica gel 60 (230-400 mesh ASTM from EMD) or using medium pressure liquid chromatography (MPLC) Biotage systems (Flash+™ or Horizon™ HPFC™, manufacturer: Dyax Corporation) using normal phase silica Flash+™ cartridges or reversed phase C18 Flash+™ cartridges. Reversed phase high pressure liquid chromatography (HPLC) was performed on a Parallex Flex™ Biotage system using an Xterra® prep RP18 OBD 10 μM 19×250 mm column from Waters. ¹H NMR spectra were recorded on a Varian instrument operating at 400 MHz. ¹H-NMR spectra were obtained as CDCl₃ or DMSO-d₆ solutions (reported in ppm), using chloroform (7.25 ppm) or tetramethylsilane (0.00 ppm) as the reference standards. When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz). LCMS were recorded on a Finnigan LCQduo from Thermoquest equipped with an Agilent Zorbax C18 rapid resolution 4.6×50 mm, 3.5 μm 80 Å column with APCI ionization or on a Surveyor MSQ from ThermoFinnigan direct injection with ESI ionization.

### Example A2

### 6-Methoxy-N-(2-(1-phenyl-3-(trifluoromethyl)-1H-pyrazole-4-carboxamido)ethyl)nicotinamide

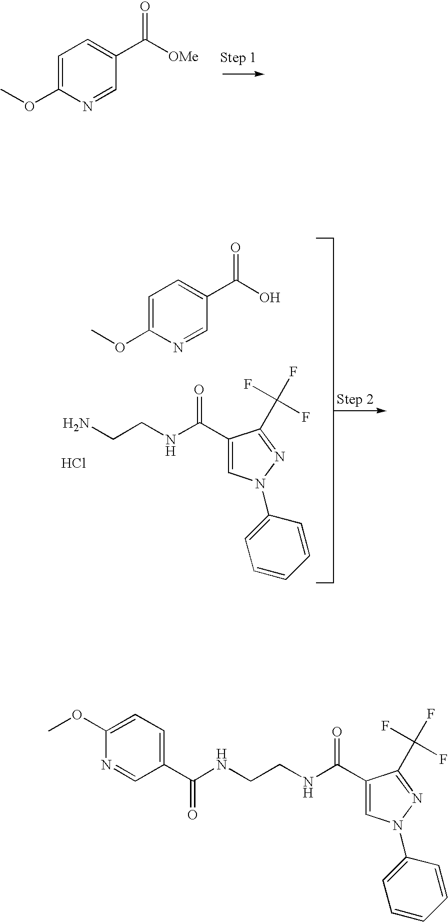

### Step 1

NaOH (0.36 g, 9.0 mmol) was dissolved in MeOH (5 mL) and water (5 mL). Methyl 6-methoxynicotinate (1.00 g, 5.98 mmol) was added and the mixture was stirred at 70° C. for 90 minutes. The solution was cooled and diluted with 1N NaOH (25 mL). The solution was extracted with EtOAc and the separated aqueous layer was acidified to pH 1. The aqueous layer was extracted with EtOAc (3 times) and the combined organic layers were dried and concentrated to yield 6-methoxynicotinic acid as a white solid (0.84 g, 92%): m/z (APCI pos) 154.1 (100%) (M+H).

Compounds of the following structures were prepared from the corresponding esters using a similar method to that described above.

| Structure | Name | Physical Data |
|---|---|---|
| 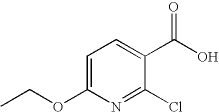 | 2-chloro-6- ethoxynicotinic acid | ¹H NMR (400 MHz, DMSO- d₆) δ 1.33 (t, J =7.2 Hz, 3H), 4.34 (q, J = 7.6, 14.4 Hz, 2H), 6.88 (d, J = 8.8 Hz, 1H), 8.17 (d, J = 8.4 Hz, 1H), 13.37 (br, 1H); .m/z (APCI pos) 201.9 (20%) (M + H) |
| 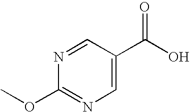 | 2-methoxy- pyrimidine-5- carboxylic acid | m/z (APCI pos) 110.9 (85%) [M− (CO₂H) +H] |
| 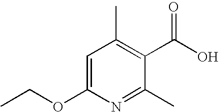 | 6-ethoxy-2,4- dimethyl- nicotinic acid | m/z (APCI pos) 196.1 (100%) (M + H) |
| 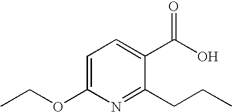 | 6-ethoxy-2- propylnicotinic acid | m/z (APCI pos) 210.1 (100%) (M + H) |
| 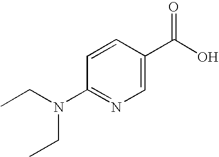 | 6- (diethylamino) nicotinic acid | m/z (APCI pos) 195.2 (100%) (M + H). |
| 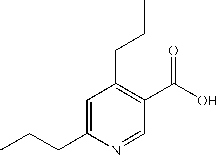 | 4,6-di- propylnicotinic acid | m/z (APCI pos) 208.2 (100%) (M + H). |

### Step 2

N-(2-Aminoethyl)-1-phenyl-3-(trifluoromethyl)-1H-pyrazole-4-carboxamide hydrochloride (0.20 g, 0.60 mmol), 6-methoxynicotinic acid (0.091 g, 0.60 mmol), and HATU (0.27 g, 0.72 mmol) were suspended in THF (10 mL). DIPEA (0.34 ml, 2.0 mmol) was added last. The mixture was stirred at room temperature overnight. The solution was poured into water (75 mL) and the solid was collected by filtration, dried and recrystallized in hexanes/EtOAc to provide 6-methoxy-N-(2-(1-phenyl-3-(trifluoromethyl)-1H-pyrazole-4-carboxamido)ethyl)nicotinamide (0.155 g, 60%): ¹H NMR (400 MHz, CDCl₃) δ 3.65-3.75 (m, 4H), 3.98 (s, 3H), 6.77 (d, J=8.8 Hz, 1H), 6.81 (br, 1H), 7.12 (br, 1H), 7.40-7.42 (m, 1H), 7.49-7.53 (m, 2H), 7.68-7.71 (m, 2H), 8.00 (dd, J=2.4, 8.4 Hz, 1H), 8.44 (s, 1H), 8.65 (d, J=2.0 Hz, 1H): m/z (APCI pos) 434.1 (100%) (M+H).

Compounds of the following structures were prepared from N-(2-aminoethyl)-1-phenyl-3-(trifluoromethyl)-1H-pyrazole-4-carboxamide hydrochloride and the corresponding acids, using a similar method to that described above. In some cases, compounds were purified by silica gel column chromatography or preparative HPLC. In some cases, compounds were converted to an acid form by a method commonly employed.

| Ex # | Structure | Name | Physical Data |
|---|---|---|---|
| A1 | 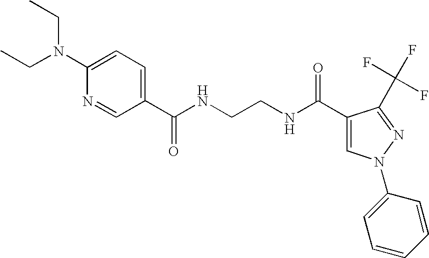 | 6-(diethylamino)-N-(2-(1- phenyl-3-(trifluoro-methyl)- 1H-pyrazole-4-carboxamido) ethyl)nicotinamide | ¹H NMR (400 MHz, DMSO- d₆) δ 1.18 (t, J = 6.8 Hz, 6H), 3.54 (q, J = 7.2, 14.0 Hz, 4H), 3.66 (t, J = 2.4 Hz, 4H), 6.43 (d, J = 9.2 Hz, 1H), 6.84 (br, 1H), 7.20 (br, 1H), 7.38-7.42 (m, 1H), 7.49 (t, J = 8.0 Hz, 2H), 7.69 (d, J = 7.6 Hz, 2H), 7.83 (dd, J = 2.0, 8.8 Hz, 1H), 8.46 (s, 1H), 8.62 (d, J = 2.4 Hz, 1H); m/z (APCI pos) 475.2 (100%) (M + H). |
| A3 | 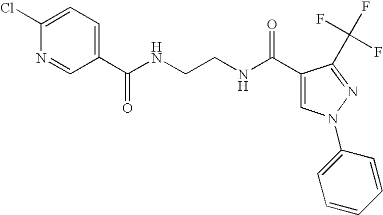 | 6-chloro-N-(2-(1- phenyl-3-(trifluoro- methyl)-1H-pyrazole- 4-carboxamido)ethyl) nicotinamide | ¹H NMR (400 MHz, DMSO-d₆) δ 3.43-3.45 (m, 4H), 7.48 (t, J = 7.6 Hz, 1H), 7.59-7.67 (m, 3H), 7.79-7.82 (m, 2H), 8.23 (dd, J = 2.8, 8.4 Hz, 1H), 8.53 (br, 1H), 8.84 (d, J = 2.4 Hz, 1H), 8.89 (br, 1H), 9.07 (s, 1H); m/z (APCI pos) 438 (5%) (M + H). |
| A4 | 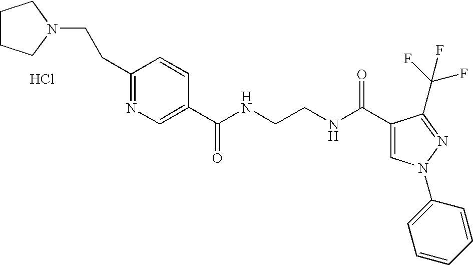 | N-(2-(1-phenyl-3- (trifluoromethyl)-1H- pyrazole-4- carboximido)ethyl)-6- (2-(pyrrolidin-1-yl) ethyl)nicotinamide hydrochloride | ¹H NMR (400 MHz, DMSO-d₆) δ 1.90 (br, 2H), 1.98 (br, 2H), 2.51 (br, 2H), 3.05 (br, 2H), 3.45 (br, 4H), 3.55 (br, 4H), 7.50 (t, J = 7.4 Hz, 2H), 7.59-7.61 (m, 2H), 7.83 (d, J = 7.4 Hz, 2H), 8.28 (d, J = 6.8 Hz, 1H), 8.68 (s, 1H), 8.98 (s, 1H), 9.03 (s, 1H), 9.31 (s, 1H), 10.59 (br, 1H); m/z (APCI pos) 501 (M + H). |
| A6 | 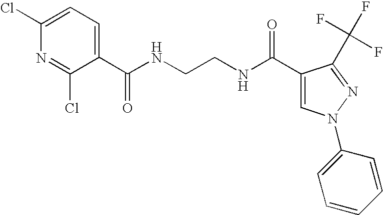 | 2,6-dichloro-N-(2-(1- phenyl-3-(trifluoro- methyl)-1H-pyrazole- 4-carboxamido)ethyl) nicotinamide | m/z (APCI pos) 472.0 (35%) (M + H). |
| A7 | 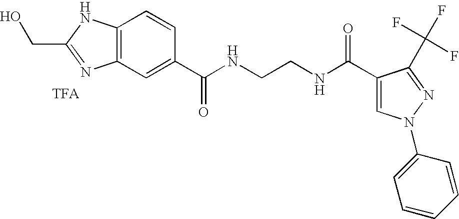 | 2-(hydroxmethyl)-N- (2-(1-phenyl-3- (trifluoromethyl)-1H- pyrazole-4- carboxamido)ethyl)- 1H-benzo[d]imidazole- 5-carboximide tri- fluoroacetate | ¹H NMR (400 MHz, DMSO-d₆) δ 3.46 (s, 4H), 4.95 (s, 2H), 7.48 (t, J = 7.4 Hz, 1H), 7.61 (t, J = 8.0 Hz, 2H), 7.75 (d, J = 8.4 Hz, 1H), 7.81 (d, J = 7.6 Hz, 2H), 7.95 (d, J = 8.2 Hz, 1H), 8.18 (s, 1H), 8.52 (s, 1H), 8.79 (s, 1H), 9.08 (s, 1H); m/z (APCI pos) 473 (M + H). |
| A10 | 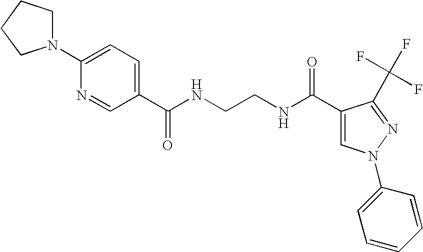 | N-(2-(1-phenyl-3- (trifluoromethyl)-1H- pyrazole-4- carboxamido)ethyl)-6- (pyrrolidin-1-yl) nicotinamide | ¹H NMR (400 MHz, CDCl₃) δ 1.86-2.08 (m, 4H), 3.44-3.54 (m, 4H), 3.64-3.72 (m, 4H), 6.32 (d, J = 8.8 Hz, 1H), 6.88 (br, 1H), 7.16 (br, 1H), 7.37-7.44 (m, 1H), 7.49 (t, J = 7.2 Hz, 2H), 7.69 (d, J = 7.2 Hz, 2H), 7.85 (dd, J = 1.6, 8.8 Hz, 1H), 8.44 (s, 1H), 8.64 (s, 1H); m/z (APCI pos) 473.2, (100%) (M + H). |
| A12 | 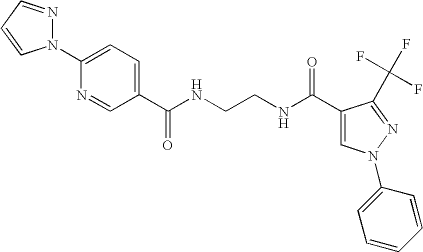 | N-(2-(1-phenyl-3- (trifluoromethyl)-1H- pyrazole-4- carboxamido)ethyl)-6- (1H-pyrazol-1-yl) nicotinamide | ¹H NMR (400 MHz, DMSO-d₆) δ 3.41-3.52 (m, 4H), 6.62 (br, 1H), 7.44-7.52 (m, 1H), 7.61 (t, J = 7.6 Hz, 2H), 7.81 (d, J = 7.6 Hz, 2H). 7.88 (br, 1H), 8.00 (d, J = 8.4 Hz, 1H), 8.40 (d, J = 7.6 Hz, 1H), 8.51 (br, 1H), 8.68 (br, 1H), 8.83 (br, 1H) 8.91 (br, 1H), 9.07 (br, 1H); m/z (APCI pos) 470.0 (30%) (M + H). |
| A13 | 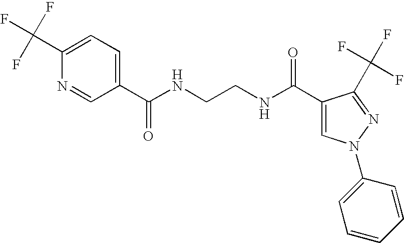 | N-(2-(1-phenyl-3-(trifluoro- methyl)-1H-pyrazole-4- carboxamido)ethyl)-6- (trifluoromethyl)nicotinamide | ¹H NMR (400 MHz, CDCl₃) δ 3.68-3.80 (m, 4H), 6.68 (br, 1H), 7.43 (t, J = 7.6 Hz, 1H), 7.52 (t, J = 7.6 Hz, 2H), 7.71 (d, 4 = 8.0 Hz, 2H), 7.78 (d, J = 8.4 Hz, 1H), 7.88 (br, 1H), 8.35 (dd, J = 1.6, 8.4 Hz, 1H), 8.48 (s, 1H), 8.17 (s, 1H); m/z (APCI pos) 472.1 (100%) (M + H). |
| A15 | 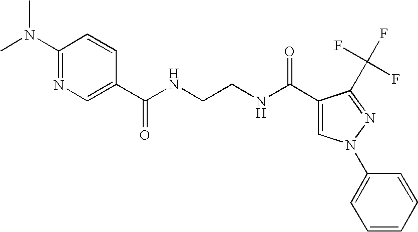 | 6-(dimethylamino)-N-(2-(1- phenyl-3-(trifluoromethyl)- 1H-pyrazole-4-carboxamido) ethyl)nicotinamide | ¹H NMR (400 MHz, DMSO- d₆) δ 3.07 (s, 6H), 3.39-3.40 (m, 4H), 6.64 (d, J = 8.8 Hz, 1H), 7.48 (t, J = 7.4 Hz, 1H), 7.61 (t, J = 7.9 Hz, 2H), 7.81 (d, J = 7.6 Hz, 2H), 7.91-7.94 (m, 1H), 8.33 (br, 1H), 8.48 (br, 1H), 8.59 (s, 1H), 9.06 (s, 1H); m/z (APCI pos) 447 (M + H). |
| A16 | 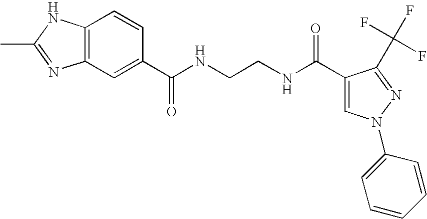 | 2-methyl-N-(2-(1-phenyl-3- (trifluoromethyl)-1H- pyrazole-4-carboxamido) ethyl)-1H-benzo[d]imidazole- 5-carboxamide | ¹H NMR (400 MHz, CD₃OD) δ 2.58 (s, 3H), 3.59-3.65 (m, 4H), 7.42-7.46 (m, 1H), 7.50- 7.58 (m, 3H), 7.70-7.72 (m, 1H), 7.78 (s, 1H), 7.80 (s, 1H), 8.02 (s, 1H), 8.72 (s, 1H); m/z (APCI pos) 457 (M + H). |
| A17 | 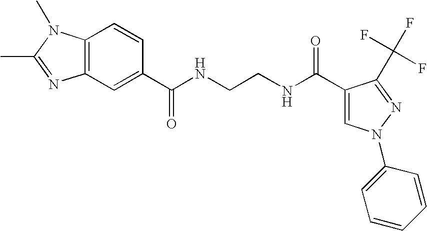 | 1,2-dimethyl-N-(2-(1-phenyl- 3-(trifluoromethyl)-1H- pyrazole-4-carboxamido) ethyl)-1H-benzo[d]imidazole- 5-carboxamide | ¹H NMR (400 MHz, DMSO- d₆) δ 2.54 (s, 3H), 3.44 (br, 4H), 3.75 (s, 3H), 7.48 (t, J = 7.3 Hz, 1H), 7.53 (d, J = 8.4 Hz, 1H), 7.61 (t, J = 8.0 Hz, 2H), 7.76 (d, J = 8.4 Hz, 1H), 7.81 (d, J = 7.6 Hz, 2H), 8.09 (s, 1H), 8.51 (br, 1H), 8.54 (br, 1H), 9.07 (s, 1H); m/z (APCI pos) 471 (M + H). |
| A18 | 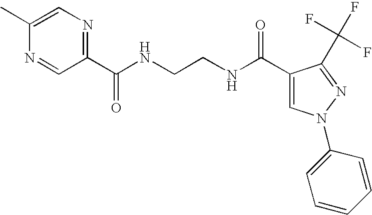 | 5-methyl-N-(2-(1-phenyl-3- (trifluoromethyl)-1H- pyrazole-4-carboxamido) ethyl)pyrazine-2-carboxamide | ¹H NMR (400 MHz, DMSO- d₆) δ 2.65 (s, 3H), 3.70-3.76 (m, 4H), 6.82 (br, 1H), 7.41 (t, J = 7.6 Hz, 1H), 7.51 (t, J = 8.4 Hz, 2H), 7.71 (d, J = 7.6 Hz, 2H), 8.20 (br, 1H), 8.41 (d, J = 4.8 Hz, 2H), 9.26 (s, 1H); m/z (APCI pos) 419.1 (100%) (M + H). |
| A19 | 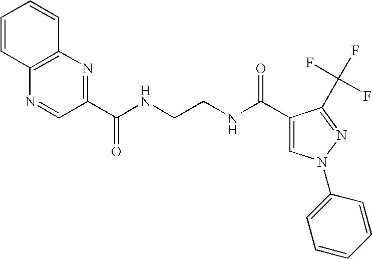 | N-(2-(1-phenyl-3-(trifluoro- methyl)-1H-pyrazole-4- carboxamido)ethyl) quinoxaline-2-carboxamide | ¹H NMR (400 MHz, CDCl₃) δ 3.76-3.84 (m, 4H), 6.80 (br, 1H), 7.42 (t, J = 7.6 Hz, 1H), 7.51 (t, J = 7.6 Hz, 2H), 7.70 (d, J = 7.6 Hz, 2H), 7.84-7.91 (m, 2H), 8.12-8.21 (m, 2H), 8.40-8.44 (m, 2H), 9.67 (s, 1H); m/z (APCI pos) 455.2, (100%) (M + H). |
| A20 | 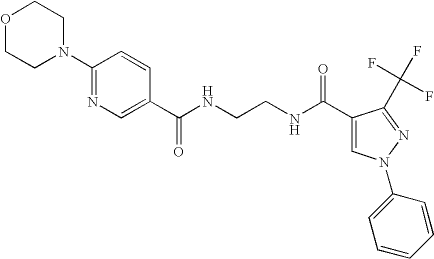 | 6-morpholine-N-(2-(1-phenyl- 3-(trifluoromethyl)-1H- pyrazole-4-carboxamido) ethyl)nicotinamide | ¹H NMR (400 MHz, DMSO- d₆) δ 3.40 (s, 4H), 3.55 (s, 4H), 3.69 (s, 4H), 6.86 (d, J = 8.7 Hz, 1H), 7.48 (t, J = 6.9 Hz, 1H), 7.61 (t, J = 7.3 Hz, 2H), 7.81 (d, J = 7.6 Hz, 2H), 7.98 (d, J = 8.2 Hz, 1H), 8.41 (s, 1H), 8.48 (s, 1H), 8.63 (s, 1H), 9.06 (s, 1H); m/z (APCI pos) 489 (M + H). |
| A21 | 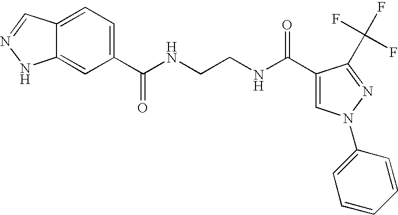 | N-(2-(1-phenyl-3-(trifluoro- methyl)-1H-pyrazole-4- carboxamido)ethyl)-1H- indazole-6-carboxamide | ¹H NMR (400 MHz, DMSO- d₆) δ 3.45 (s, 4H), 7.47 (t, J = 7.2 Hz, 1H), 7.59 (q, J = 7.2, 16.0 Hz, 3H), 7.81 (d, J = 8.0 Hz, 2H), 7.86 (dd, J = 1.6, 8.8 Hz, 1H), 8.19 (s, 1H), 8.34 (s, 1H), 8.51 (br, 1H), 8.59 (br, 1H), 9.07 (s, 1H); m/z (APCI pos) 443.0 (100%) (M + H). |
| A23 | 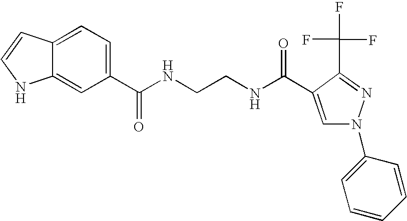 | N-(2-(1-phenyl-3-(trifluoro- methyl)-1H-pyrazole-4- carboxamido)ethyl)-1H- indole-6-carboxamide | ¹H NMR (400 MHz, CDCl₃) δ 3.43 (br, 4H), 6.51 (s, 1H), 7.40-7.49 (m, 3H), 7.58-7.64 (m, 3H), 7.81 (d, J = 7.2 Hz, 2H), 8.13 (s, 1H), 8.42 (br, 1H), 8.50 (br, 1H), 9.07 (s, 1H), 11.30 (s, 1H); m/z (APCI pos) 442.0 (100%) (M + H). |
| A24 | 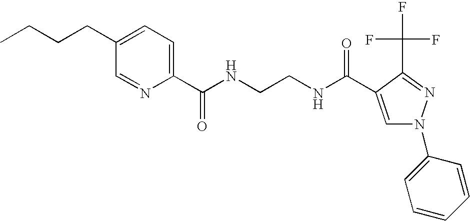 | 5-butyl-N-(2-(1-phenyl-3- (trifluoromethyl)-1H- pyrazole-4-carboxamido) ethyl)picolinamide | ¹H NMR (400 MHz, CDCl₃) δ 0.93 (t, J = 7.6 Hz, 3H), 1.31-1.41 (m, 2H), 1.58-1.64 (m, 2H), 2.67 (t, J = 7.6 Hz, 2H), 3.68-3.74 (m, 4H), 7.06 (br, 1H), 7.41 (t, J = 7.6 Hz, 1H), 7.51 (t, J = 7.2 Hz, 2H), 7.64 (dd, J = 2.4, 8.0 Hz, 1H), 7.71 (d, J = 7.2 Hz, 2H), 8.07 (d, J = 8.0 Hz, 1H), 8.37-8.41 (m, 3H); m/z (APCI pos) 460.2 (100%) (M + H). |
| A25 | 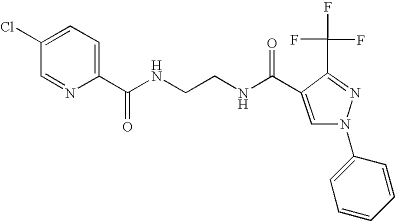 | 5-chloro-N-(2-(1-phenyl-3- (trifluoromethyl)-1H- pyrazole-4-carboxamido) ethyl)picolinamide | ¹H NMR (400 MHz, CDCl₃) δ 3.71 (br, 4H), 6.83 (br, 1H), 7.41 (t, J = 7.2 Hz, 1H), 7.51 (t, J = 8.4 Hz, 2H), 7.68-7.71 (m, 2H), 7.82 (dd, J = 2.4, 8.4 Hz, 1H), 8.13 (d, J = 8.4 Hz, 1H), 8.31 (br, 1H), 8.42 (s, 1H), 8.52 (d, J = 1.6 Hz, 1H); m/z (APCI pos) 438.0 (100%) (M + H). |
| A39 | 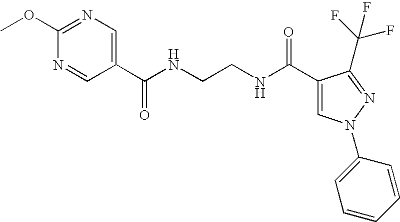 | 2-methoxy-N-(2-(1-phenyl-3- (trifluoromethyl)-1H- pyrazole-4-carboxamido) ethyl)pyrimidine-5- carboxamide | ¹H NMR (400 MHz, DMSO- d₆) δ 3.42-3.43 (m, 4H), 3.98 (s, 3H), 7.48 (t, J = 7.2 Hz, 1H), 7.61 (t, J = 7.6 Hz, 2H), 7.80 (d, J = 7.6 Hz, 2H), 8.50 (br, 1H), 8.77 (br, 1H), 8.99 (s, 2H), 9.05 (s, 1H); m/z (APCI pos) 435.1 (M + H). |
| A51 | 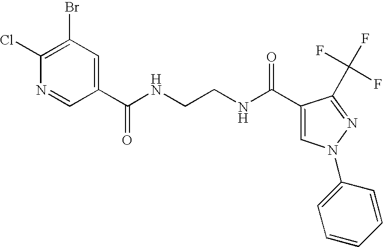 | 5-bromo-6-chloro-N-(2-(1- phenyl-3-(trifluoromethyl)- 1H-pyrazole-4-carboxamido) ethyl)nicotinamide | ¹H NMR (400 MHz, CDCl₃) δ 3.68-3.76 (m, 4H), 6.64 (br, 1H), 7.43 (t, J = 7.6 Hz, 1H), 7.50-7.54 (m, 2H), 7.70-7.72 (m, 2H), 7.74 (br, 1H), 8.42 (d, J = 2.4 Hz, 1H), 8.47 (d, J = 1.2 Hz, 1H), 8.76 (d, J = 2.0 Hz, 1H); m/z (APCI pos) 516 (M + H). |
| A65 | 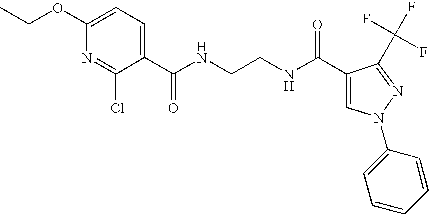 | 2-chloro-6-ethoxy-N-(2-(1-phenyl-3-(trifluoromethyl)-1H-pyrazole-4-carboxamido)ethyl)nicotinamide | ¹H NMR (400 MHz, CDCl₃) δ 1.39 (t, J = 6.8 Hz, 3H), 3.71- 3.73 (m, 4H), 4.39 (q, J = 7.2, 14.0 Hz, 2H), 6.70 (d, J = 8.8 Hz, 1H), 6.76 (br, 1H), 7.42 (t, J = 7.2 Hz, 1H), 7.49-7.53 (m, 2H), 7.70 (d, J = 8.0 Hz, 2H), 8.04 (d, J = 8.4 Hz, 1H), 8.42 (s, 1H); m/z (APCI pos) 482.1 (M + H). |
| A67 | 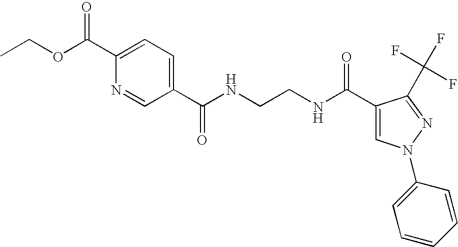 | ethyl 5-((2-(1-phenyl-3- (trifluoromethyl)-1H- pyrazole-4-carboxamido) ethyl)carbamoyl)picolinate | ¹H NMR (400 MHz, DMSO- d₆) δ 1.35 (t, J = 7.1 Hz, 3H), 3.46 (br, 4H), 4.35-4.40 (m, 2H), 7.48 (t, J = 7.4 Hz, 1H), 7.61 (t, J = 7.9 Hz, 2H), 7.81 (d, J = 7.6 Hz, 2H), 8.15 (d, J = 8.2 Hz, 1H), 8.36-8.38 (m, 1H), 8.52 (br, 1H), 8.98 (br, 1H), 9.07 (s, 1H), 9.11 (d, J = 1.6 Hz, 1H); m/z (APCI pos) 476 (M + H). |
| A69 | 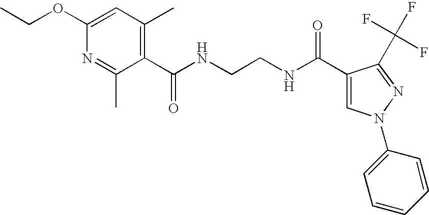 | 6-ethoxy-2,4-dimethyl-N-(2- (1-phenyl-3-(trifluoromethyl)- 1H-pyrazole-4-carboxamido) ethyl)nicotinamide | ¹H NMR (400 MHz, CDCl₃) δ 1.35 (t, J = 6.8 Hz, 3H), 2.22 (s, 3H), 2.39 (s, 3H), 3.69-3.71 (m, 4H), 4.30 (q, J = 7.2, 14.0 Hz, 2H), 6.35 (s, 2H), 6.94 (br, 1H), 7.39-7.42 (m, 1H), 7.48-7.52 (m, 2H), 7.69 (d, J = 7.6 Hz, 2H), 8.42 (s, 1H); m/z (APCI pos) 476.1 (M + H). |
| A75 | 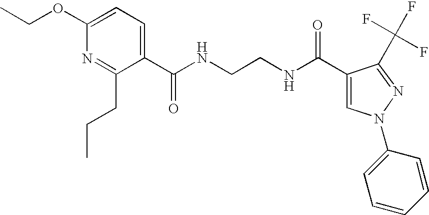 | 6-ethoxy-N-(2-(1-phenyl-3- (trifluoromethyl)-1H- pyrazole-4-carboxamido) ethyl)-2-propylnicotinamide | ¹H NMR (400 MHz, CDCl₃) δ 0.82 (t, J = 7.6 Hz, 3H), 1.30 (t, J = 6.8 Hz, 3H), 1.60- 1.66 (m, 2H), 2.74 (t, J = 8.0 Hz, 2H), 3.37-3.41 (m, 4H), 4.31 (q, J = 7.2, 14.0 Hz, 2H), 6.60 (d, J = 8.4 Hz, 1H), 7.47 (t, J = 7.6 Hz, 1H), 7.61 (t, J = 8.4 Hz, 2H), 7.66 (d, J = 7.6 Hz, 1H), 7.80 (d, J = 7.6 Hz, 2H), 8.35 (br, 1H), 8.43 (br, 1H), 9.07 (s, 1H); m/z (APCI pos) 490.2 (M + H). |
| A76 | 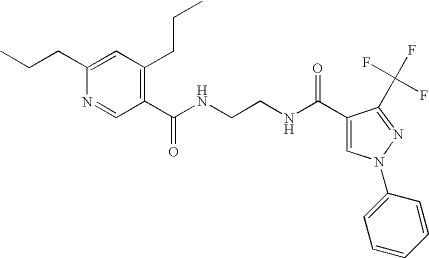 | N-(2-(1-phenyl-3-(trifluoro- methyl)-1H-pyrazole-4- carboxamido)ethyl)-4,6-di- propylnicotinamide | ¹H NMR (400 MHz, DMSO- d₆) δ 0.80 (t, J = 7.6 Hz, 3H), 0.88 (t, J = 7.6 Hz, 3H), 1.49- 1.52 (m, 2H), 1.63-1.69 (m, 2H), 2.66 (q, J = 8.4, 15.2 Hz, 4H), 3.40-3.41 (m, 4H), 0.47 (s, 1H), 7.48 (t, J = 7.2 Hz, 1H), 7.61 (t, J = 8.4 Hz, 2H), 7.81 (d, J = 8.0 Hz, 2H), 8.42 (s, 1H), 8.43 (br, 1H), 8.52 (br, 1H), 9.07 (s, 1H); m/z (APCI pos) 488.2 (M + H). |

### Example A5

### 6-(3-(Dimethylamino)propylamino)-N-(2-(1-phenyl-3-(trifluoromethyl)-1H-pyrazole-4-carboxamido)ethyl)nicotinamide

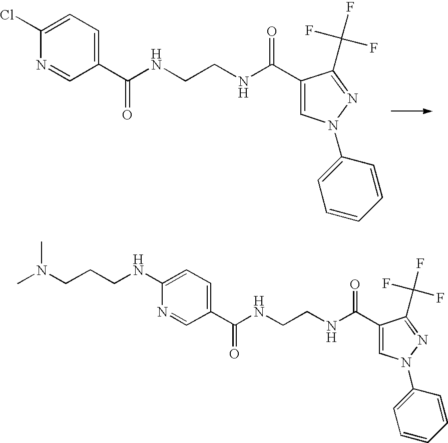

6-Chloro-N-(2-(1-phenyl-3-(trifluoromethyl)-1H-pyrazole-4-carboxamido)ethyl)nicotinamide (0.10 g, 0.23 mmol) was suspended in N1,N1-dimethylpropane-1,3-diamine (4.67 g, 45.7 mmol). The solution was stirred at 100° C. for 6 hours. The solution was cooled and concentrated. Reverse phase HPLC purification afforded 6-(3-(dimethylamino)propylamino)-N-(2-(1-phenyl-3-(trifluoromethyl)-1H-pyrazole-4-carboxamido)ethyl)nicotinamide (0.115 g, 27%): ¹H NMR (400 MHz, DMSO-d₆) δ 1.80 (t, J=6.8 Hz, 2H), 2.33 (s, 6H), 2.51 (t, J=6.4 Hz, 2H), 3.38 (br, 2H), 3.64 (br, 4H), 6.07 (br, 1H), 6.33 (d, J=8.4 Hz, 1H), 7.37-7.39 (m, 2H), 7.47 (t, J=7.6 Hz, 2H), 7.55 (br, 1H), 7.68 (d, J=7.6 Hz, 2H), 7.83 (d, J=8.4 Hz, 1H), 8.60 (s, 1H), 8.62 (s, 1H); m/z (APCI pos) 504.1 (35%) (M+H).

Compounds of the following structures were prepared from 6-chloro-N-(2-(1-phenyl-3-(trifluoromethyl)-1H-pyrazole-4-carboxamido)ethyl)nicotinamide and the corresponding amines, using a similar method to that described above.

| Ex # | Structure | Name | Physical Data |
|---|---|---|---|
| A8 | 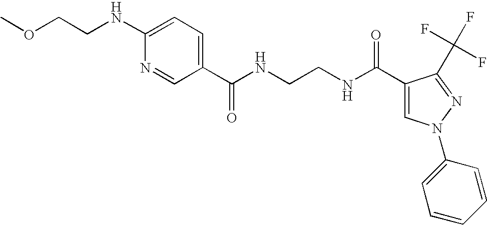 | 6-(2-methoxy- ethylamino)-N- (2-(1-phenyl-3- (trifluoromethyl)- 1H-pyrazole-4- carboxamido)ethyl) nicotinamide | ¹H NMR (400 MHz, CDCl₃) δ 3.38 (s, 3H), 3.52- 3.60 (m, 4H), 3.64- 3.72 (m, 4H), 5.15 (br, 1H), 6.39 (d, J = 9.2 Hz, 1H), 6.87 (br, 1H), 7.00 (br, 1H), 7.41 (t, J = 7.6 Hz, 1H), 7.51 (t, J − 7.6 Hz, 2H), 7.70 (d, J = 8.0 Hz, 2H), 7.83 (dd, J = 2.4, 8.8 Hz, 1H), 8.44 (s, 1H), 8.57 (d, J = 2.0 Hz, 1H); m/z (APCI pos) 477.2 (100%) (M + H). |
| A9 | 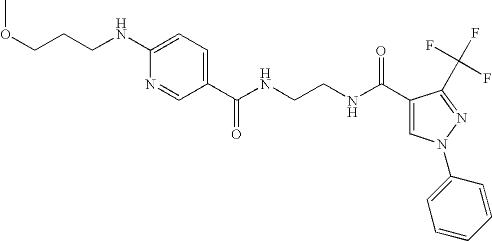 | 6-(3-methoxy- propylamino)-N- (2-(1-phenyl-3- (trifluoromethyl)- 1H-pyrazole-4- carboxamido)ethyl) nicotinamide | ¹H NMR (400 MHz, CDCl₃) δ 1.86-1.92 (m, 2H), 3.35 (s, 3H), 3.48- 3.52 (m, 4H), 3.67 (t, J = 2.4 Hz, 4H), 5.27 (t, J = 5.6 Hz, 1H), 6.37 (d, J = 1H), 6.85 (br, 1H), 7.02 (br, 1H), 7.39-7.43 (m, 1H), 7.49-7.53 (m, 2H), 7.70 (d, J = 7.6 Hz, 2H), 7.84 (dd, J = 3.2, 9.2 Hz, 1H), 8.43 (s, 1H), 8.56 (d, J = 2.4 Hz, 1H); m/z (APCI pos) 491.2 (100%) (M + H). |
| A11 | 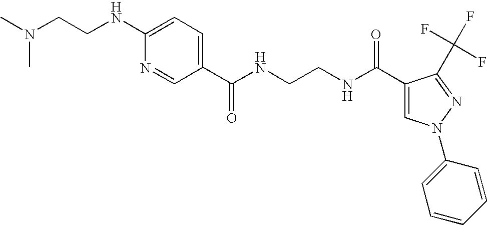 | 6-(2-(dimethyl- amino)ethylamino)- N-(2-(1- phenyl-3- (trifluoromethyl)- 1H-pyrazole-4- carboxamido)ethyl) nicotinamide | ¹H NMR (400 MHz, CDCl₃) δ 2.27 (s, 6H), 2.55 (t, J = 5.6 Hz, 2H), 3.33-3.41 (m, 2H), 3.58-3.68 (m, 4H), 5.57 (br, 1H), 6.33 (d, J = 8.4 Hz, 1H), 7.32- 7.40 (m, 2H), 7.46 (t, J = 7.2 Hz, 1H), 7.57 (br, 1H), 7.66 (d, J = 7.6 Hz, 2H), 7.81 (d, J = 7.6 Hz, 1H), 8.55 (s, 1H), 8.59 (s, 1H); m/z (APCI pos) 490.2 (100%) (M + H). |
| A14 | 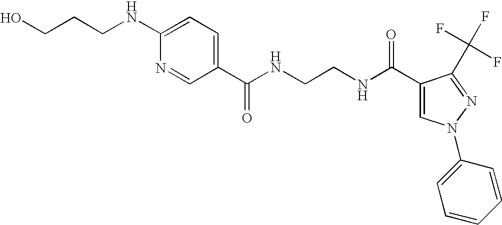 | 6-(3-hydroxy- propylamino)-N- (2-(1-phenyl-3- (trifluoromethyl)- 1H-pyrazole-4- carboxamido)ethyl) nicotinamide | ¹H NMR (400 MHz, DMSO- d₆) δ 1.65-1.70 (m, 2H), 3.28-3.25 (m, 2H), 3.38 (br, 4H), 3.44-3.50 (m, 2H), 4.49 (t, J = 4.8 Hz, 1H), 6.44 (d, J = 8.8 Hz, 1H), 7.02 (t, J = 5.2 Hz, 1H), 7.48 (t, J = 7.2 Hz, 1H), 7.61 (t, J = 8.4 Hz, 2H), 7.76- 7.84 (m, 3H), 8.25 (br, 1H), 8.47 (br, 1H), 8.49 (d, J = 2.0 H, 1H), 9.06 (s, 1H); m/z (APCI pos) 477.1 (100%) (M + H). |

### Example A30

### 6-(2-Ethoxyethoxy)-N-(2-(1-phenyl-3-(trifluoromethyl)-1H-pyrazole-4-carboxamido)ethyl)nicotinamide

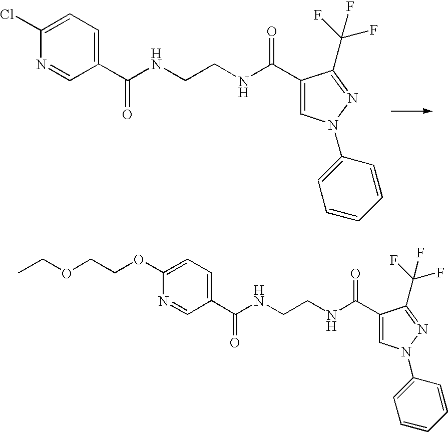

2-Ethoxyethanol (0.285 ml, 2.85 mmol) in THF (10 mL) was charged with NaH (0.058 g, 2.3 mmol) and the mixture was stirred for 30 minutes at room temperature. 6-Chloro-N-(2-(1-phenyl-3-(trifluoromethyl)-1H-pyrazole-4-carboxamido)ethyl)nicotinamide (0.250 g, 0.571 mmol) was added and the mixture was stirred at 75° C. for 4 hours. The solution was then cooled and concentrated. The residue was diluted with water and extracted with DCM. The organic layer was then dried and concentrated. Recrystallization from DCM gave 6-(2-ethoxyethoxy)-N-(2-(1-phenyl-3-(trifluoromethyl)-1H-pyrazole-4-carboxamido)ethyl)nicotinamide (0.149 g, 53%) as a light yellow solid: ¹H NMR (400 MHz, DMSO-d₆) δ 1.11 (t, J=7.0 Hz, 3H), 3.42 (br, 4H), 3.46-3.51 (m, 2H), 3.70 (t, J=4.6 Hz, 2H), 4.42 (t, J=4.7 Hz, 2H), 6.90 (d, J=8.6 Hz, 1H), 7.48 (t, J=7.2 Hz, 1H), 7.61 (t, J=7.9 Hz, 2H), 7.81 (d, J=7.6 Hz, 2H), 8.10-8.13 (m, 1H), 8.49 (br, 1H), 8.60 (br, 1H), 8.64 (s, 1H), 9.06 (s, 1H); m/z (APCI pos) 492 (M+H).

Compounds of the following structures were prepared from 6-chloro-N-(2-(1-phenyl-3-(trifluoromethyl)-1H-pyrazole-4-carboxamido)ethyl)nicotinamide and the corresponding alcohols or thiol, using a similar-method to that described above. In some cases, compounds were purified by silica gel column chromatography or preparative HPLC. In some cases, compounds were converted to an acid form by a method commonly employed.

| Ex # | Structure | Name | Physical Data |
|---|---|---|---|
| A22 | 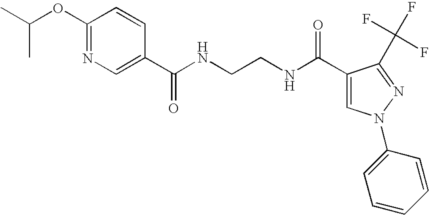 | 6-isopropoxy- N-(2-(1- phenyl-3- (trifluoromethyl)- 1H-pyrazole-4- carboxamido)ethyl) nicotinamide | ¹H NMR (400 MHz, DMSO- d₆) δ 1.30 (d, J = 6.0 Hz, 6H), 3.41-3.42 (m, 4H), 5.25-5.35 (m, 1H), 6.80 (d, J = 8.6 Hz, 1H), 7.48 (t, J = 7.3 Hz, 1H), 7.61 (t, J = 8.0 Hz, 2H), 7.81 (d, J = 7.4 Hz, 2H), 8.07-8.10 (m, 1H), 8.48 (br, 1H), 8.57 (br, 1H), 8.63 (d, J = 2.1 Hz, 1H), 9.06 (s, 1H); m/z (APCI pos) 462 (M + H). |
| A26 | 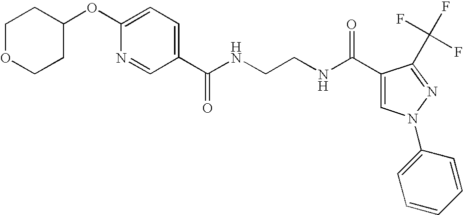 | N-(2-(1- phenyl-3- (trifluoromethyl)- 1H-pyrazole-4- carboxamido)ethyl)- 6-(tetrahydro- 2H-pyran-4-yloxy) nicotinamide | ¹H NMR (400 MHz, CDCl₃) δ 1.78-1.83 (m, 2H), 2.04-2.09 (m, 2H), 3.58-3.64 (m, 2H), 3.70 (s, 4H), 3.95- 4.00 (m, 2H), 5.27- 5.32 (m, 1H), 6.75 (d, J = 8.8 Hz, 1H), 6.84 (br, 1H), 7.14 (br, 1H), 7.42 (t, J = 6.4 Hz, 1H), 7.48-7.53 (m, 2H), 7.70 (d, J = 8.0 Hz, 2H), 8.00 (dd, J = 2.8, 9.2 Hz, 1H), 8.44 (s, 1H) 8.61 (d, J = 2.0 Hz, 1H); m/z (APCI pos) 504.0 (M + H). |
| A27 | 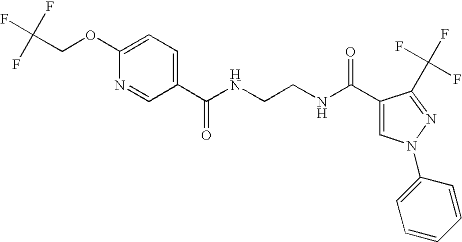 | N-(2-(1- phenyl-3- (trifluoromethyl)- 1H-pyrazole-4- carboxamido)ethyl)- 6-(2,2,2- trifluoroethoxy) nicotinamide | ¹H NMR (400 MHz, CDCl₃) δ 3.68-3.76 (m, 4H), 4.80 (q, J = 8.4, 17.2 Hz, 2H), 6.76 (br, 1H), 6.91 (d, J = 8.4 Hz, 1H), 7.30 (br, 1H), 7.42 (t, J = 7.6 Hz, 1H), 7.52 (t, J = 7.2 Hz, 2H), 7.71 (d, J = 7.2 Hz, 2H), 8.09 (dd, J = 2.8, 8.8 Hz, 1H), 8.45 (s, 1H), 8.63 (d, J = 2.4 Hz, 1H); m/z (APCI pos) 502.1 (M + H). |
| A28 | 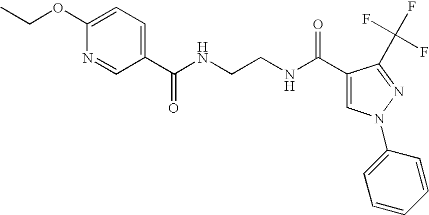 | 6-ethoxy-N-(2- (1-phenyl-3- (trifluoromethyl)- 1H-pyrazole-4- carboxamido)ethyl) nicotinamide | ¹H NMR (400 MHz, DMSO- d₆) δ 1.32 (t, J = 7.0 Hz, 3H), 3.42 (br, 4H), 4.33-4.38 (m, 2H), 6.85 (d, J = 8.8 Hz, 1H), 7.48 (t, J = 7.3 Hz, 1H), 7.61 (t, J = 7.9 Hz, 2H), 7.81 (d, J = 7.8 Hz, 2H), 8.09-8.12 (m, 1H), 8.48 (br, 1H), 8.59 (br, 1H), 8.64 (s, 1H), 9.06 (s, 1H); m/z (APCI pos) 448 (M + H). |
| A29 | 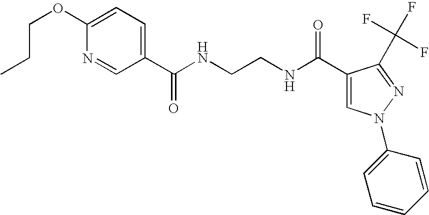 | N-(2-(1- phenyl-3- (trifluoromethyl)- 1H-pyrazole-4- carboxamido) ethyl)-6-propoxy- nicotinamide | ¹H NMR (400 MHz, DMSO- d₆) δ 0.96 (t, J = 7.4 Hz, 3H), 1.69-1.78 (m, 2H), 3.42 (br, 4H), 4.26 (t, J = 6.7 Hz, 2H), 6.87 (d, J = 8.6 Hz, 1H), 7.48 (t, J = 7.3 Hz, 1H), 7.61 (t, J = 7.9 Hz, 2H), 7.81 (d, J = 7.6 Hz, 2H), 8.09-8.12 (m, 1H), 8.49 (br, 1H), 8.59 (br, 1H), 8.65 (s, 1H), 9.06 (s, 1H); m/z (APCI pos) 462 (M + H). |
| A31 | 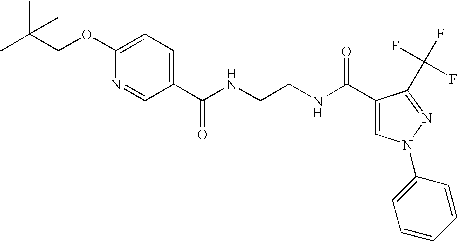 | 6-(neopentyl- oxy)-N-(2-(1- phenyl-3-(trifluoromethyl)- 1H-pyrazole-4- carboxamido)ethyl) nicotinamide | ¹H NMR (400 MHz, DMSO- d₆) δ 0.99 (s, 9H), 3.42 (br, 4H), 4.00 (s, 2H), 6.89 (d, J = 8.8 Hz, 1H), 7.48 (t, J = 7.3 Hz, 1H), 7.61 (t, J = 7.9 Hz, 2H), 7.81 (d, J = 7.8 Hz, 2H), 8.10-8.13 (m, 1H), 8.49 (br, 1H), 8.59 (br, 1H), 8.63 (s, 1H), 9.06 (s, 1H); m/z (APCI pos) 490 (M + H). |
| A32 | 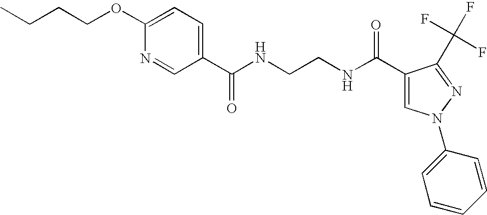 | 6-butoxy-N- (2-(1-phenyl-3- (trifluoromethyl)- 1H-pyrazole-4- carboxamido)ethyl) nicotinamide | ¹H NMR (400 MHz, DMSO- d₆) δ 0.92 (t, J = 7.4 Hz, 3H), 1.37-1.46 (m, 2H), 1.66-1.73 (m, 2H), 3.42 (br, 4H), 4.30 (t, J = 6.6 Hz, 2H), 6.86 (d, J = 8.6 Hz, 1H), 7.48 (t, J = 7.4 Hz, 1H), 7.61 (t, J = 7.9 Hz, 2H), 7.81 (d, J = 7.6 Hz, 2H), 8.09-8.12 (m, 1H), 8.48 (br, 1H), 8.59 (br, 1H), 8.64 (s, 1H), 9.06 (s, 1H); m/z (APCI pos) 476 (M + H). |
| A33 | 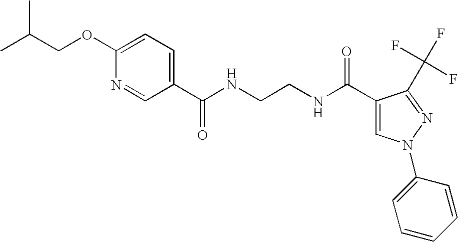 | 6-isobutoxy- N-(2-(1- phenyl-3- (trifluoromethyl)- 1H-pyrazole-4- carboxamido)ethyl) nicotinamide | ¹H NMR (400 MHz, DMSO- d₆) δ 0.96 (d, J = 6.6 Hz, 6H), 2.00-2.07 (m, 1H), 3.42 (br, 4H), 4.08 (t, J = 6.6 Hz, 2H), 6.88 (d, J = 8.6 Hz, 1H), 7.48 (t, J = 7.4 Hz, 1H), 7.61 (t, J = 7.9 Hz, 2H), 7.81 (d, J = 7.6 Hz, 2H), 8.10-8.12 (m, 1H), 8.49 (br, 1H), 8.59 (br, 1H), 8.64 (s, 1H), 9.06 (s, 1H); m/z (APCI pos) 476 (M + H). |
| A34 | 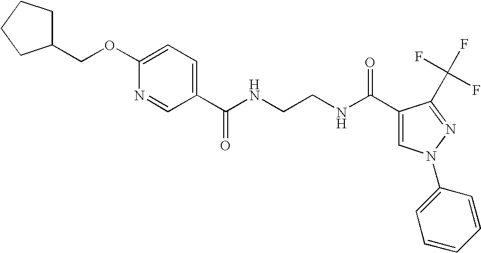 | 6-(cyclopentyl- methoxy)-N-(2- (1-phenyl-3- (trifluoromethyl)- 1H-pyrazole-4- carboxamido)ethyl) nicotinamide | ¹H NMR (400 MHz, DMSO- d₆) δ 1.29-1.34 (m, 2H), 1.51-1.63 (m, 4H), 1.74-1.76 (m, 2H), 2.28-2.35 (m, 1H), 3.42 (br, 4H), 4.18 (d, J = 7.0 Hz, 2H), 6.86 (d, J = 8.6 Hz, 1H), 7.48 (t, J = 7.4 Hz, 1H), 7.61 (t, J = 7.9 Hz, 2H), 7.81 (d, J = 7.8 Hz, 2H), 8.09- 8.12 (m, 1H), 8.48 (br, 1H), 8.59 (br, 1H), 8.63 (s, 1H), 9.06 (s, 1H); m/z (APCI pos) 502 (M + H). |
| A35 | 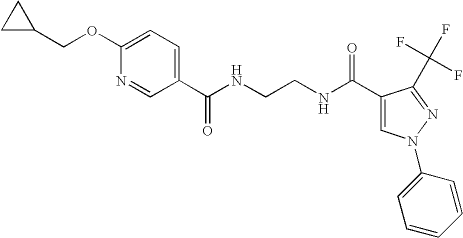 | 6-(cyclopropyl- methoxy)-N-(2- (1-phenyl-3- (trifluoromethyl)- 1H-pyrazole-4- carboxamido)ethyl) nicotinamide | ¹H NMR (400 MHz, DMSO- d₆) δ 0.31-0.35 (m, 2H), 0.53-0.57 (m, 2H), 1.21-1.26 (m, 1H), 3.42 (br, 4H), 4.14 (d, J = 7.2 Hz, 2H), 6.88 (d, J = 8.6 Hz, 1H), 7.48 (t, J = 7.3 Hz, 1H), 7.61 (t, J = 7.9 Hz, 2H), 7.81 (d, J = 7.6 Hz, 2H), 8.09-8.12 (m, 1H), 8.49 (br, 1H), 8.59 (br, 1H), 8.63 (s, 1H), 9.06 (s, 1H); m/z (APCI pos) 474 (M + H). |
| A36 | 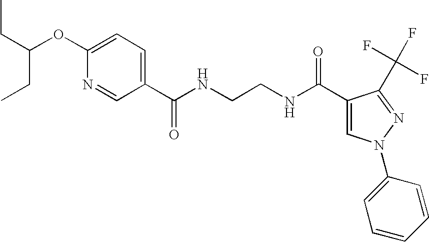 | 6-(pentan-3- yloxy)-N-(2- (1-phenyl-3- (trifluoromethyl)- 1H-pyrazole-4- carboxamido)ethyl) nicotinamide | ¹H NMR (400 MHz, DMSO- d₆) δ 0.87 (t, J = 7.4 Hz, 6H), 1.58-1.70 (m, 4H), 3.41 (br, 4H), 5.08-5.12 (m, 1H), 6.83 (d, J = 8.6 Hz, 1H), 7.48 (t, J = 7.4 Hz, 1H), 7.61 (t, J = 7.9 Hz, 2H), 7.81 (d, J = 7.8 Hz, 2H), 8.07- 8.10 (m, 1H), 8.47 (br, 1H), 8.55 (br, 1H), 8.62 (s, 1H), 9.06 (s, 1H); m/z (APCI pos) 490 (M + H). |
| A37 | 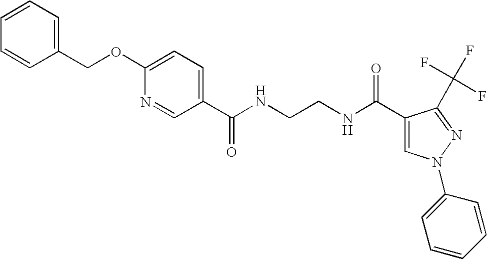 | 6-(benzyloxy)- N-(2-(1- phenyl-3- (trifluoromethyl)- 1H-pyrazole-4- carboxamido)ethyl) nicotinamide | ¹H NMR (400 MHz, DMSO- d₆) δ 3.42 (d, J = 2.6 Hz, 4H), 5.41 (s, 2H), 6.95 (d, J = 8.8 Hz, 1H), 7.31-7.40 (m, 3H), 7.44-7.50 (m, 3H), 7.61 (t, J = 8.0 Hz, 2H), 7.81 (d, J = 7.6 Hz, 2H), 8.13- 8.15 (m, 1H), 8.49 (br, 1H), 8.61 (br, 1H), 8.67 (d, J = 2.2 Hz, 1H), 9.06 (s, 1H); m/z (APCI pos) 510 (M + H). |
| A38 | 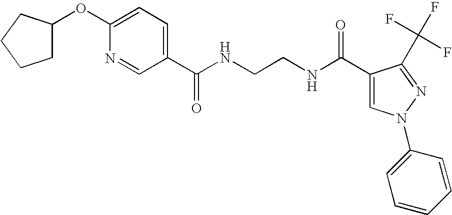 | 6-(cyclopentyl- oxy)-N-(2-(1- phenyl-3- (trifluoromethyl)- 1H-pyrazole-4- carboxamido)ethyl) nicotinamide | ¹H NMR (400 MHz, DMSO- d₆) δ 1.59-1.68 (m, 2H), 1.68-1.71 (m, 4H), 1.92-1.96 (m, 2H), 3.41-3.42 (m, 4H), 5.39-5.42 (m, 1H), 6.81 (d, J = 8.8 Hz, 1H), 7.48 (t, J = 7.4 Hz, 1H), 7.61 (t, J = 7.9 Hz, 2H), 7.81 (d, J = 7.7 Hz, 2H), 8.07-8.09 (m, 1H), 8.48 (br, 1H), 8.57 (br, 1H), 8.64 (d, J = 2.4 Hz, 1H), 9.06 (s, 1H); m/z (APCI pos) 488 (M + H). |
| A40 | 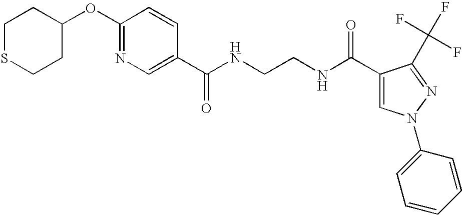 | N-(2-(1- phenyl-3- (trifluoromethyl)- 1H-pyrazole-4- carboxamido)ethyl)- 6-(tetrahydro- 2H-thiopyran- 4-yloxy) nicotinamide | ¹H NMR (400 MHz, CDCl₃) δ 1.95-2.07 (2H, m), 2.18-2.28 (2H, m), 2.61-2.71 (2H, m), 2.82-2.91 (2H, m), 3.66-3.76 (4H, m), 5.21 (1H, m), 6.75 (1H, dd, J = 0.4, 8.8 Hz), 6.77 (1H, br), 7.09 (1H, br), 7.43 (1H, t, J = 7.6 Hz), 7.52 (2H, t, J = 8.4 Hz), 7.70 (2H, d, J = 8.0 Hz), 8.00 (1H, dd, J = 2.4, 8.8 Hz), 8.44 (1H, d, J = 0.8 Hz), 8.60 (1H, d, J = 2.4 Hz); m/z (APCI pos) 520.0 (30%) (M + H). |
| A42 | 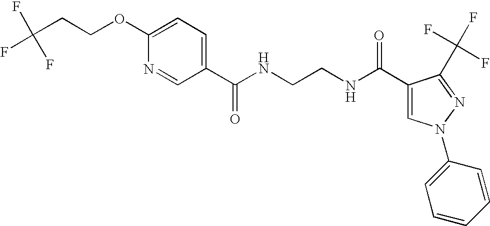 | N-(2-(1- phenyl-3- (trifluoromethyl)- 1H-pyrazole-4- carboxamido)ethyl)- 6-(3,3,3- trifluoropropoxy) nicotinamide | ¹H NMR (400 MHz, DMSO- d₆) δ 2.75-2.86 (m, 2H), 3.43 (d, J = 2.5 Hz, 4H), 4.54 (t, J = 6.0 Hz, 2H), 6.91 (d, J = 8.6 Hz, 1H), 7.48 (t, J = 7.4 Hz, 1H), 7.61 (t, J = 7.9 Hz, 2H), 7.81 (d, J = 7.6 Hz, 2H), 8.13-8.15 (m, 1H), 8.48 (br, 1H), 8.63 (br, 1H), 8.66 (d, J = 2.3 Hz, 1H), 9.06 (s, 1H); m/z (APCI pos) 515 (M + H). |
| A45 | 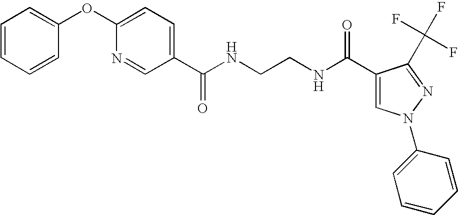 | 6-phenoxy-N- (2-(1-phenyl-3- (trifluoromethyl)- 1H-pyrazole-4- carboxamido)ethyl) nicotinamide | ¹H NMR (400 MHz, CDCl₃) δ 3.67-3.72 (m, 4H), 6.81 (br, 1H), 6.94 (d, J = 8.4 Hz, 1H), 7.14 (d, J = 8.0 Hz, 2H), 7.22-7.25 (m, 1H), 7.33 (br, 1H), 7.41 (t, J = 8.4 Hz, 3H), 7.50 (t, J = 7.6 Hz, 2H), 7.70 (d, J = 7.6 Hz, 2H), 8.14 (dd, J = 8.8, 2.4 Hz, 1H), 8.45 (s, 1H), 8.64 (d, J = 2.0 Hz, 1H); m/z (APCI pos) 496.1 (M + H). |
| A46 | 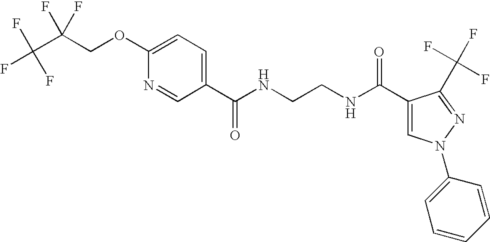 | 6-(2,2,3,3,3- pentafluoro- propoxy)-N-(2- (1-phenyl-3- (trifluoromethyl)- 1H-pyrazole-4- carboxamido)ethyl) nicotinamide | ¹H NMR (400 MHz, CDCl₃) δ 3.69-3.72 (m, 4H), 4.88 (t, J = 13.2 Hz, 2H), 6.83 (br, 1H), 6.90 (d, J = 8.8 Hz, 1H), 7.37 (br, 1H), 7.41 (t, J = 4.8 Hz, 1H), 7.44-7.53 (m, 2H), 7.69 (d, J = 7.2 Hz, 2H), 8.09 (dd, J = 8.8, 2.4 Hz, 1H), 8.45 (s, 1H), 8.64 (d, J = 2.8 Hz, 1H); m/z (APCI pos) 552.0 (M + H). |
| A48 | 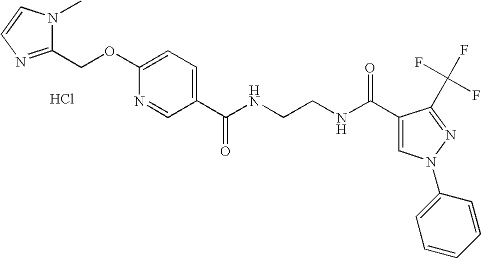 | 6-((1-methyl- 1H-imidazol- 2-yl)methoxy)- N-(2-(1- phenyl-3- (trifluoromethyl)- 1H-pyrazole-4- carboxamido)ethyl) nicotinamide hydrochloride | ¹H NMR (400 MHz, DMSO- d₆) δ 3.41-3.45 (m, 4H), 3.92 (s, 3H), 5.69 (s, 2H), 7.05 (d, J = 8.8 Hz, 1H), 7.48 (t, J = 7.4 Hz, 1H), 2H), 7.67 (d, J = 1.8 Hz, 1H), 7.73 (d, J = 1.8 Hz, 1H), 7.83 (d, J = 7.6 Hz, 2H), 8.26- 8.29 (m, 1H), 8.68 (m, 1H), 8.73 (d, J = 2.1 Hz, 1H), 8.87 (m, 1H), 9.33 (s, 1H); m/z (APCI pos) 514 (M + H). |
| A84 | 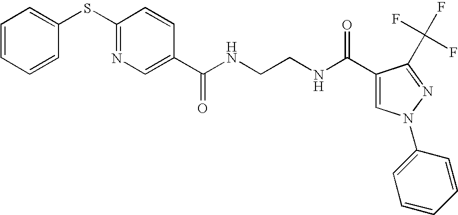 | N-(2-(1- phenyl-3- (trifluoromethyl)- 1H-pyrazole-4- carboxamido)ethyl)- 6-(phenylthio) nicotinamide | ¹H NMR (400 MHz, CDCl₃) δ 3.64-3.76 (4H, m), 6.74 (1H, br), 6.93 (1H, dd, J = 0.8, 8.4 Hz), 7.34 (1H, m), 7.39-7.55 (6H, m), 7.57-7.64 (2H, m), 7.68-7.73 (2H, m), 7.88 (1H, dd, J = 2.4, 8.4 Hz), 8.44 (1H, d, J = 0.8 Hz), 8.84 (1H, d, J = 2.4 Hz); m/z (APCI pos) 512.1 (100%) (M + H). |

### Example A41

### 6-((1,1-Dioxidotetrahydro-2H-thiopyran-4-yl)oxy)-N-(2-(((1-phenyl-3-(trifluoromethyl)-1H-pyrazol-4-yl)carbonyl)amino)ethyl)nicotinamide

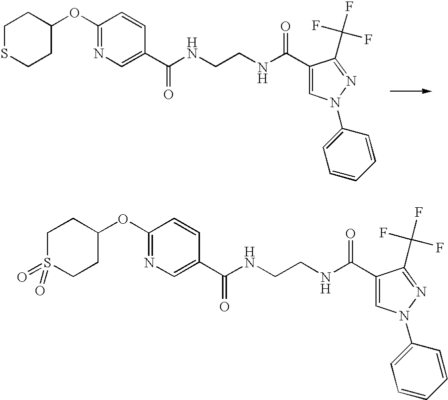

To a mixture of N-(2-(1-phenyl-3-(trifluoromethyl)-1H-pyrazole-4-carboxamido)ethyl)-6-(tetrahydro-2H-thiopyran-4-yloxy)nicotinamide (0.20 g, 0.385 mmol), formic acid (0.15 mL, 3.85 mmol), THF (4 mL) and acetone (8 mL) was added potassium permanganate (0.15 g, 0.96 mmol) in portions at 0° C. and the mixture was stirred at room temperature for 12 hours. The mixture was filtered and the filtrate was concentrated in vacuo. The residue was recrystallized (EtOAc/hexanes) to afford 6-((1,1-dioxidotetrahydro-2H-thiopyran-4-yl)oxy)-N-(2-(((1-phenyl-3-(trifluoromethyl)-1H-pyrazol-4-yl)carbonyl)amino)ethyl)nicotinamide (0.165 g, 78%) as colorless crystals: ¹H NMR (400 MHz, CDCl₃) δ 2.36-2.57 (4H, m), 2.94-3.04 (2H, m), 3.28-3.41 (2H, m), 3.64-3.78 (4H, m), 5.48 (1H, m), 6.75 (1H, br), 6.80 (1H, d, J=8.8 Hz), 7.43 (1H, t, J=7.6 Hz), 7.52 (2H, t, J=7.6 Hz), 7.70 (2H, d, J=7.6 Hz), 8.06 (1H, dd, J=2.4, 8.8 Hz), 8.44 (1H, s), 8.62 (1H, d, J=2.4H) m/z (APCI pos) 552.0 (100%) (M+H).

### Example A43

### 6-(2-(Ethylsulfonyl)ethoxy)-N-(2-(1-phenyl-3-(trifluoromethyl)-1H-pyrazole-4-carboxamido)ethyl)nicotinamide

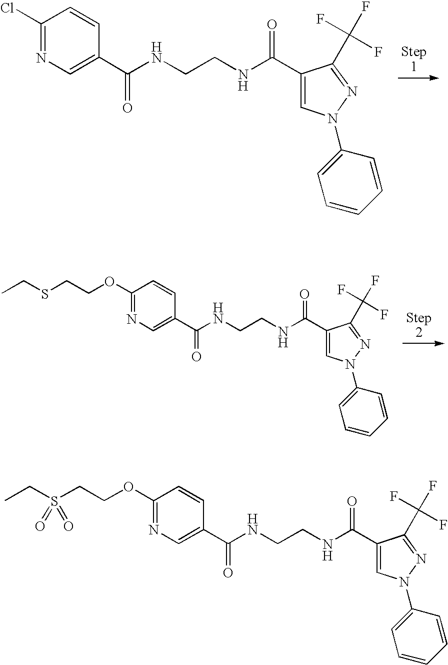

### Step 1

To a solution of 2-(ethylthio)ethanol (0.61 mL, 5.71 mmol) in THF (5 mL) was added NaH (0.14 g, 5.7 mmol) at 0° C. and the mixture was stirred for 30 min. 6-Chloro-N-(2-(1-phenyl-3-(trifluoromethyl)-1H-pyrazole-4-carboxamido)ethyl)nicotinamide (0.50 g, 1.1 mmol) was added to the mixture and the mixture was stirred for 18 h at 90° C. in a sealed tube. After being cooled, the mixture was poured into water, and extracted with EtOAc. The EtOAc extract was dried and concentrated in vacuo. The residue was purified by silica gel chromatography (hexanes/EtOAc=1/4 to EtOAc/MeOH=50/1) to give 6-(2-(ethylthio)ethoxy)-N-(2-(1-phenyl-3-(trifluoromethyl)-1H-pyrazole-4-carboxamido)ethyl)nicotinamide (0.48 g, 83%) as colorless crystals.

### Step 2

To a mixture of 6-(2-(ethylthio)ethoxy)-N-(2-(1-phenyl-3-(trifluoromethyl)-1H-pyrazole-4-carboxamido)ethyl)nicotinamide (0.36 g, 0.71 mmol), formic acid (0.27 mL, 7.09 mmol), THF (5 mL) and acetone (10 mL) was added potassium permanganate (0.28 g, 1.77 mmol) in portions at 0° C. and the mixture was stirred for 12 hours at room temperature. The mixture was filtered and the filtrate was concentrated in vacuo. The residue was partitioned between saturated aqueous sodium bicarbonate and EtOAc. The EtOAc layer was washed with brine, dried, and concentrated. Flash chromatography on silica gel (EtOAc to EtOAc/MeOH=10/1) gave 6-(2-(ethylsulfonyl)ethoxy)-N-(2-(1-phenyl-3-(trifluoromethyl)-1H-pyrazole-4-carboxamido)ethyl)nicotinamide (0.30 g, 78% yield) as colorless crystals: ¹H NMR (400 MHz, CDCl₃) δ 1.41 (3H, t, J=7.6 Hz), 3.11 (2H, q, J=7.6 Hz), 3.45 (2H, t, J=5.6 Hz), 3.66-3.78 (4H, m), 4.83 (2H, t, J=5.6 Hz), 6.74 (1H, br), 6.79 (1H, d, J=8.8 Hz), 7.28 (1H, br), 7.43 (1H, t, J=7.6 Hz), 7.52 (2H, t, J=7.6 Hz), 7.71 (2H, d, J=7.6 Hz), 8.07 (1H, dd, J=2.4, 8.8 Hz), 8.45 (1H, d, J=0.8 Hz), 8.64 (1H, d, J=2.4 Hz); m/z (APCI pos) 540.1 (100%) (M+H).

### Example A44

### 6-Chloro-N-(2-(1-phenyl-3-(trifluoromethyl)-1H-pyrazole-4-carboxamido)ethyl)-2-(2,2,2-trifluoroethoxy)nicotinamide

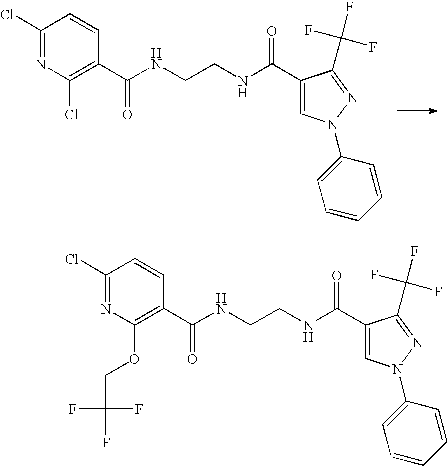

NaH (0.085 g, 3.4 mmol) was added to THF (35 mL). 2,2,2-Trifluoroethanol (0.244 ml, 3.39 mmol) was added and the mixture was stirred at room temperature for 30 minutes. The solution was then cooled to 0° C., 2,6-dichloro-N-(2-(1-phenyl-3-(trifluoromethyl)-1H-pyrazole-4-carboxamido)ethyl)nicotinamide (0.320 g, 0.678 mmol) in DMF (2 mL) was added and the mixture was stirred at 0° C. for 2 hours. The solution was then cooled and diluted with water. The mixture was extracted with EtOAc, dried, and concentrated. Flash chromatography and recrystallization with hexanes/EtOAc gave 6-chloro-N-(2-(1-phenyl-3-(trifluoromethyl)-1H-pyrazole-4-carboxamido)ethyl)-2-(2,2,2-trifluoroethoxy)nicotinamide (0.250 g, 69%) as a white solid: ¹H NMR (400 MHz, CDCl₃) δ 3.69-3.73 (m, 4H), 4.95 (q, J=8.0, 16.0 Hz, 2H), 6.73 (br, 1H), 7.18 (d, J=8.0 Hz, 1H), 7.43 (t, J=6.8 Hz, 1H), 7.51 (t, J=7.6 Hz, 2H), 7.70 (d, J=7.2 Hz, 2H), 7.80 (br, 1H), 8.40 (s, 1H), 8.49 (d, J=8.4 Hz, 1H); m/z (APCI pos) 536.0 (M+H).

### Example A47

### N-(2-(1-Phenyl-3-(trifluoromethyl)-1H-pyrazole-4-carboxamido)ethyl)-6-propylnicotinamide

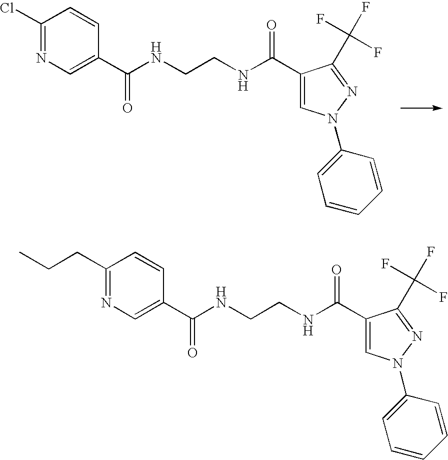

6-Chloro-N-(2-(1-phenyl-3-(trifluoromethyl)-1H-pyrazole-4-carboxamido)ethyl)nicotinamide (0.100 g, 0.228 mmol) and PdCl₂(dppf) dichloromethane adduct (0.009 g, 0.011 mmol) were added together in degassed THF (5 mL) under argon. Propylzinc(II) bromide (1.14 ml, 0.571 mmol, 0.5 M in THF) was added and the mixture was stirred under argon at 55° C. for 1 hr. The solution was cooled and filtered through a silica plug (rinsing with EtOAc) and concentrated. Flash chromatography and recrystallization (EtOAc/hexanes) afforded N-(2-(1-phenyl-3-(trifluoromethyl)-1H-pyrazole-4-carboxamido)ethyl)-6-propylnicotinamide (0.020 g, 20%) as a white solid: ¹H NMR (400 MHz, CDCl₃) δ 0.96 (t, J=7.6 Hz, 3H), 1.76 (q, J=14.8, 7.6 Hz, 2H), 2.82 (t, J=7.6 Hz, 2H), 3.68-3.77 (m, 4H), 6.75 (br, 1H), 7.22 (d, J=8.4 Hz, 1H), 7.26 (br, 1H), 7.42 (t, J=7.2 Hz, 1H), 7.52 (t, J=8.0 Hz, 2H), 7.70 (d, J=6.8 Hz, 2H), 8.03 (dd, J=8.4, 2.4 Hz, 1H), 8.45 (s, 1H), 8.95 (d, J=2.4 Hz, 1H); m/z (APCI pos) 446.2 (M+H).

The compound of the following structure was prepared from 6-chloro-N-(2-(1-phenyl-3-(trifluoromethyl)-1H-pyrazole-4-carboxamido)ethyl)nicotinamide and 2-thiazolylzinc bromide, using a similar method to that described above.

| Ex # | Structure | Name | Physical Data |
|---|---|---|---|
| A93 | 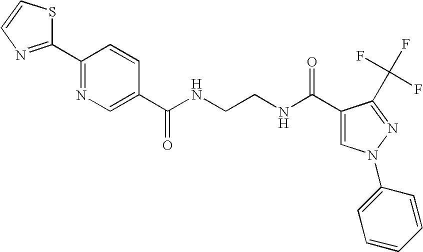 | N-(2-(1- phenyl-3- (trifluoromethyl)- 1H-pyrazole-4- carboxamido)ethyl)- 6-(thiazol-2-yl) nicotinamide | ¹H NMR (400 MHz, DMSO- d₆) δ 3.46 (s, 4H), 7.48 (t, J = 7.3 Hz, 1H), 7.61 (t, J = 7.9 Hz, 2H), 7.81 (d, J = 7.6 Hz, 2H), 7.95 (d, J = 3.1 Hz, 1H), 8.05 (d, J = 3.1 Hz, 1H), 8.22 (d, J = 8.2 Hz, 1H), 8.35-8.37 (m, 1H), 8.52 (s, 1H), 8.89 (s, 1H), 9.04 (s, 1H), 9.07 (s, 1H); m/z (APCI pos) MS Calcd.: 486; Found: 487 (M + H). |

### Example A49

### N-(2-(1-(Pyrimidin-2-yl)-3-(trifluoromethyl)-1H-pyrazole-4-carboxamido)ethyl)-6-(2,2,2-trifluoroethoxy)nicotinamide

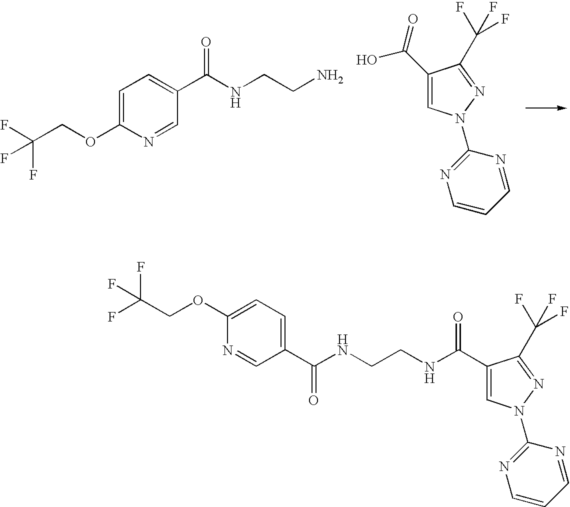

To a round bottom flask were added EDAC.HCl (218 mg, 1.14 mmol), HOBt.H₂O (175 mg, 1.14 mmol), and 1-(pyrimidin-2-yl)-3-(trifluoromethyl)-1H-pyrazole-4-carboxylic acid (294 mg, 1.14 mmol). These components were dissolved in DMF (3 ml) and the mixture was stirred for 5 minutes, then N-(2-aminoethyl)-6-(2,2,2-trifluoroethoxy)nicotinamide (250 mg, 0.95 mmol) was added as a solution in DMF (1 ml). The mixture was stirred for 16 h, diluted with water, and extracted with ethyl acetate several times. The combined organic layers were dried with sodium sulfate and concentrated in vacuo to give the crude product, which was purified by silica gel chromatography (5% MeOH/DCM) to give N-(2-(1-(pyrimidin-2-yl)-3-(trifluoromethyl)-1H-pyrazole-4-carboxamido)ethyl)-6-(2,2,2-trifluoroethoxy)nicotinamide (0.178 g, 37%) as a solid: ¹H NMR (400 MHz, DMSO-d₆) δ 3.42 (m, 4H), 5.06 (q, 2H, J=9.0 Hz), 7.08 (d, 1H, J=8.6 Hz), 7.66 (t, 1H, J=4.7 Hz), 8.21 (m, 1H), 8.69 (m, 3H), 8.99 (d, 2H, J=4.7 Hz), 9.41 (s, 1H); m/z (APCI pos) 504.0 (100%) (M+H).

Compounds of the following structures were made by reacting N-(2-aminoethyl)-6-(2,2,2-trifluoroethoxy)nicotinamide and the corresponding acids, using the procedure outlined above.

| Ex # | Structure | Name | Physical Data |
|---|---|---|---|
| A50 | 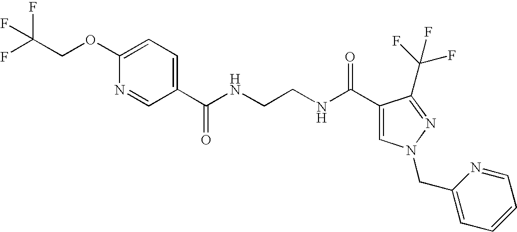 | N-(2-(1- (pyridin-2- ylmethyl)-3- (trifluoromethyl)- 1H-pyrazole-4- carboxamido)ethyl)- 6-(2,2,2- trifluoroethoxy) nicotinamide | Solid (47%); ¹H NMR (400 MHz, DMSO-d₆) δ 3.37 (m, 4H), 5.06 (q, 2H, J = 9.0 Hz), 5.56 (s, 2H), 7.07 (d, 1H, J = 8.2 Hz), 7.32 (m, 1H), 7.37 (m, 1H), 7.84 (m, 1H), 8.19 (m, 1H), 8.43 (m, 2H), 8.55 (m, 1H), 8.66 (m, 2H); m/z (APCI pos) 517.1 (100%) (M + H). |
| A80 | 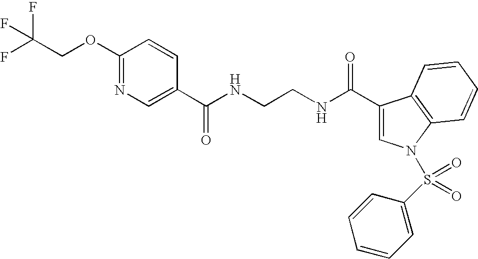 | 1-(phenyl- sulfonyl)-N-(2-(6- (2,2,2- trifluoroethoxy) nicotinamido)ethyl)- 1H-indole-3- carboxamide | Solid (41%); ¹H NMR (400 MHz, DMSO-d₆) δ 3.45 (s, 4H), 5.07 (q, 2H, J = 8.98 Hz), 7.08 (d, 1H, J = 8.59 Hz), 7.32 (m, 1H), 7.39 (m, 1H), 7.63 (m, 2H), 7.74 (m, 1H), 7.93 (d, 1H, J = 8.20 Hz), 8.02 (m, 2H), 8.13-8.23 (m, 2H), 8.51 (s, 1H), 8.58 (m, 1H), 8.69 (m, 2H); m/z (APCI pos) 547.0 (100%) (M + H). |

### Example A52

### 5-Bromo-N-(2-(1-phenyl-3-(trifluoromethyl)-1H-pyrazole-4-carboxamido) ethyl)-6-(2,2,2-trifluoroethoxy)nicotinamide

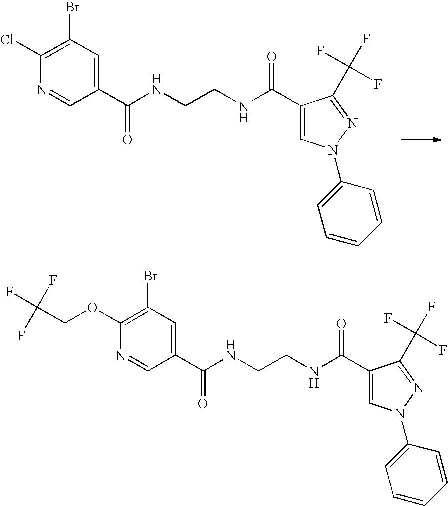

2,2,2-Trifluoroethanol (0.835 ml, 11.6 mmol) was diluted in THF (35 mL). NaH (0.293 g, 11.6 mmol) was added and the mixture was stirred at room temperature for 30 minutes. The solution was cooled to 0° C. and 5-bromo-6-chloro-N-(2-(1-phenyl-3-(trifluoromethyl)-1H-pyrazole-4-carboxamido)ethyl)nicotinamide (1.20 g, 2.32 mmol) was added. The mixture was stirred at 40° C. overnight. The solution was cooled, quenched with water, extracted from EtOAc, dried, and concentrated. The material was recrystallized from hexanes/EtOAc to give 5-bromo-N-(2-(1-phenyl-3-(trifluoromethyl)-1H-pyrazole-4-carboxamido) ethyl)-6-(2,2,2-trifluoroethoxy)nicotinamide (0.95 g, 70%) as a white solid: ¹H NMR (400 MHz, CDCl₃) δ 3.66-3.75 (m, 4H), 4.84 (q, J=8.0, 16.8 Hz, 2H), 6.76 (br, 1H), 7.42 (t, J=7.2 Hz, 1H), 7.49-7.53 (m, 3H), 7.70 (d, J=7.2 Hz, 2H), 8.35 (d, J=2.4 Hz, 1H), 8.45 (s, 1H), 8.54 (d, J=2.0 Hz, 1H); m/z (APCI pos) 580 (M+H).

### Example A53

### 1-Phenyl-N-(2-(6-(2,2,2-trifluoroethoxy)nicotinamido) ethyl)-1H-indole-3-carboxamide

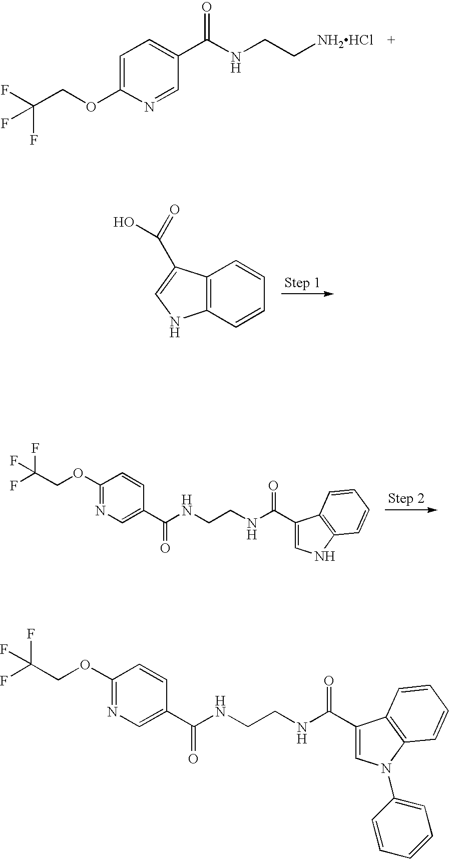

### Step 1

To a solution of 1H-indole-3-carboxylic acid (0.269 g, 1.67 mmol), EDAC.HCl (0.352 g, 1.84 mmol), and HOBt.H₂O (0.281 g, 1.84 mmol) in DMF (5 ml) was added N-(2-aminoethyl)-6-(2,2,2-trifluoroethoxy)nicotinamide hydrochloride (0.500 g, 1.67 mmol) followed by DIPEA (0.87 ml, 5.0 mmol). The solution was stirred for 16 h at room temperature, diluted with EtOAc (100 ml) and washed with water (3×), saturated aqueous sodium bicarbonate, and brine. The aqueous layer was back extracted and the combined organic layers were dried (sodium sulfate), filtered through a short pad of silica which was rinsed with 5% MeOH/DCM, and concentrated in vacuo to give N-(2-(6-(2,2,2-trifluoroethoxy)nicotinamido)ethyl)-1H-indole-3-carboxamide (0.450 g, 66%) as a solid: ¹H NMR (CDCl₃) δ 3.63 (s, 4H), 4.92 (q, 2H, J=8.59 Hz), 6.95 (m, 1H), 7.16 (m, 2H), 7.41 (m, 1H), 8.07 (m, 1H), 8.16 (m, 1H), 8.66 (m, 1H); m/z (APCI pos) 407.1 (100%) (M+H).

Compounds of the following structures were prepared by reacting N-(2-aminoethyl)-6-(2,2,2-trifluoroethoxy)nicotinamide hydrochloride and the corresponding acids using the procedure outlined above.

| Ex # | Structure | Name | Physical Data |
|---|---|---|---|
| A71 | 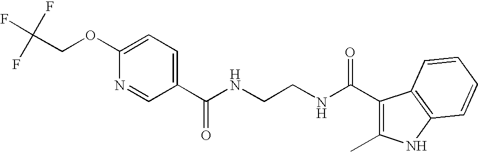 | 2-methyl-N- (2-(6-(2,2,2- trifluoroethoxy) nicotinamide) ethyl)-1H-indole- 3-carboxamide | Pink solid (27%); ¹H NMR (400 MHz, DMSO-d₆) δ 2.56 (s, 3H), 3.49 (m, 4H), 5.06 (q, 2H, J = 9.0 Hz), 6.98-7.10 (m, 3H), 7.30 (m, 1H), 7.51 (m, 1H), 7.77 (m, 1H), 8.22 (dd, 1H, J = 8.7, 2.2 Hz), 8.68-8.72 (m, 2H), 11.40 (s, 1H); m/z (APCI pos) 421.1 (40%) (M + H), 158.1 (100%). |
| A100 | 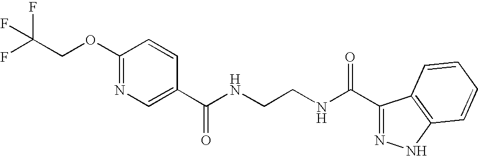 | N-(2-(6-(2,2,2- trifluoroethoxy) nicotinamido)ethyl- 1H-indazole-3- carboxamide | Solid (66%); ¹H NMR (CDCl₃) δ 3.66 (m, 4H), 4.92 (q, 2H, J = 8.59 Hz), 6.95 (m, 1H), 7.24 (m, 1H), 7.41 (m, 1H), 7.56 (m, 1H), 8.17 (m, 2H), 8.65 (m, 1H); m/z (APCI pos) 408.1 (100%) (M + H) |
| A101 | 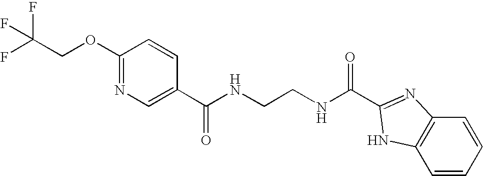 | N-(2-(6-(2,2,2- trifluoroethoxy) nicotinamido)ethyl)- 1H-benzo[d] imidazole-2- carboxamide | Solid (56%); ¹H NMR (CDCl₃) δ 3.50 (m, 4H), 5.06 (q, 2H, J = 8.59 Hz), 7.07 (m, 1H), 8.20 (m, 1H), 8.69 (m, 2H), 9.10 (m, 1H); m/z (APCI pos) 408.1 (100%) (M + H). |

### Step 2

Following the procedure of Buchwald et al. (J. Org. Chem. 2004, 69, 5578), to a 250 mL sealed tube under a sweep of nitrogen were added N-(2-(6-(2,2,2-trifluoroethoxy)nicotinamido)ethyl)-1H-indole-3-carboxamide g, 1.11 mmol), potassium carbonate (0.321 g, 2.33 mmol), 1-iodobenzene (0.148 ml, 1.33 mmol), copper(I) iodide (0.0105 g, 0.06 mmol) and (1R,2R)—N1,N2-dimethylcyclohexane-1,2-diamine (0.0315 g, 0.22 mmol). These components were dissolved in degassed (Argon) dioxane (8 ml). The tube was sealed and the mixture was heated at 110° C. for 20 h. The mixture was dissolved in EtOAc and filtered through a pad of silica, and the filtrate was concentrated in vacuo. The residue was purified by silica gel chromatography (5% MeOH/DCM) to give 1-phenyl-N-(2-(6-(2,2,2-trifluoroethoxy)nicotinamido)ethyl)-1H-indole-3-carboxamide (0.284 g, 53% yield) as a solid: ¹H NMR (CDCl₃) δ 3.47 (s, 4H), 5.05 (q, 2H, J=9.0 Hz), 7.07 (d, 1H, J=9.0 Hz), 7.25 (m, 2H), 7.49 (m, 1H), 7.54 (m, 1H), 7.63 (m, 4H), 8.21-8.27 (m, 3H), 8.29 (s, 1H), 8.69 (m, 1H), 8.72 (m, 1H); m/z (APCI pos) 483.1 (100%) (M+H).

Compounds of the following structures were prepared using a similar method to that described above.

| Ex # | Structure | Name | Physical Data |
|---|---|---|---|
| A54 | 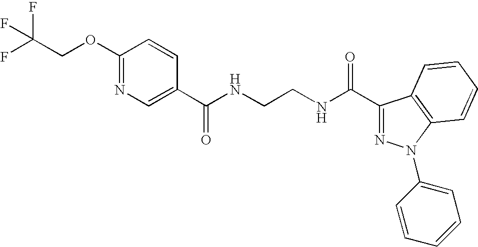 | 1-phenyl-N- (2-(6-(2,2,2- trifluoroethoxy) nicotinamido)ethyl)- 1H-indazole-3- carboxamide | Solid (62%); ¹H NMR (CDCl₃) δ 3.53 (m, 4H), 5.05 (q, 2H, J = 9.0 Hz), 7.06 (m, 1H), 7.39 (m, 1H), 7.48-7.57 (m, 2H), 7.65 (m, 2H), 7.86 (m, 3H), 8.21 (m, 1H), 8.30 (m, 1H), 8.68 (m, 1H), 8.72 (m, 2H); m/z (APCI pos) 484.0 (100%) (M + H). |
| A61 | 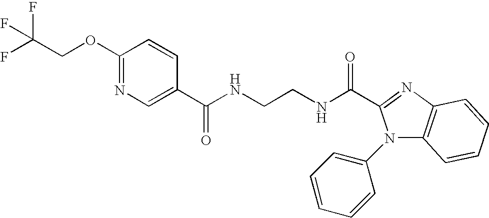 | 1-phenyl-N- (2-(6-(2,2,2- trifluoroethoxy) nicotinamido)ethyl)- 1H-benzo[d] imidazole-2- carboxamide | Solid (6%); ¹H NMR (400 MHz, ACN-d₃) δ 3.60 (m, 4H), 4.85 (q, 2H), 6.84 (m, 1H), 7.15- 7.61 (m, 11H), 8.06 (d, 1H, J = 7.4 Hz), 8.55 (s, 1H); m/z (APCI pos) 484.0 (100%) (M + H). |
| A79 | 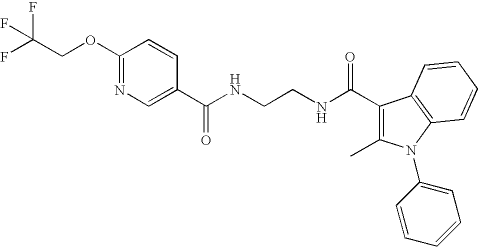 | 2-methyl-1- phenyl-N-(2- (6-(2,2,2- trifluoroethoxy) nicotinamido)ethyl)- 1H-indole-3- carboxamide | Solid (8%); ¹H NMR (400 MHz, DMSO-d₆) δ 2.41 (s, 3H), 3.52 (s, 4H), 5.06 (q, 2H, J = 9.0 Hz), 6.97 (m, 1H), 7.07-7.14 (m, 3H), 7.42 (d, 2H), 7.57 (m, 1H), 7.64 (m, 2H), 7.83 (m, 2H), 8.22 (m, 1H), 8.70 (m, 2H); m/z (APCI pos) 497.1 (30%) (M + H). |

### Example A60

### tert-Butyl 1-phenyl-4-(2-(6-(2,2,2-trifluoroethoxy)nicotinamido)ethylcarbamoyl)-1H-pyrazol-3-ylcarbamate

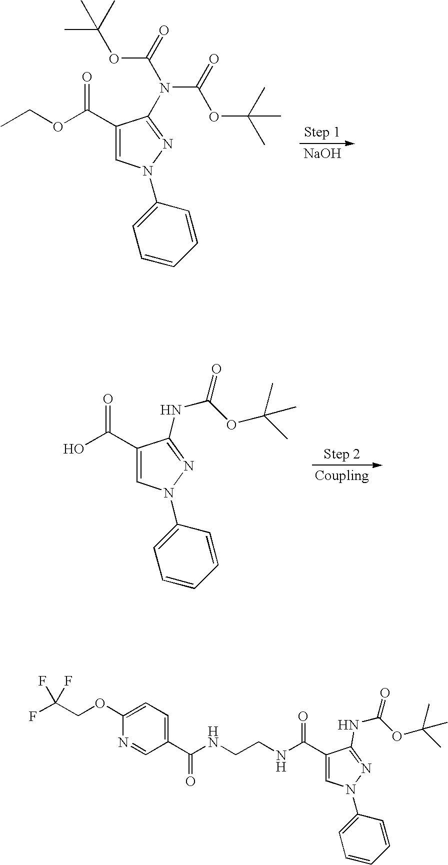

### Step 1

To a mixture of ethyl 3-(bis(tert-butoxycarbonyl)amino)-1-phenyl-1H-pyrazole-4-carboxylate (2.11 g, 4.89 mmol) in EtOH (20 mL) was added 2 N NaOH (12.2 ml, 24.5 mmol) and the mixture was stirred at 70° C. for 3 hrs. The mixture was cooled, concentrated off the EtOH, water was added and the aqueous layer was washed with EtOAc. The aqueous layer was then acidified to pH 2 using 1 N HCl. The resulting solids were filtered, washed with water and dried under high vacuum to give 3-(tert-butoxycarbonylamino)-1-phenyl-1H-pyrazole-4-carboxylic acid (1.24 g, 83%): ¹H NMR (400 MHz, DMSO-d₆) δ 1.55 (s, 9H), 7.34 (t, J=7.4 Hz, 1H), 7.51 (t, J=8.0 Hz, 2H), 7.87 (d, J=7.6 Hz, 2H), 8.79 (s, 1H), 8.91 (s, 1H), 12.81 (s, 1H): m/z (APCI pos) 304 (40%) (M+H).

Compounds of the following structures were prepared from the corresponding esters using a similar method to that described above.

| Structure | Name | Physical Data |
|---|---|---|
| 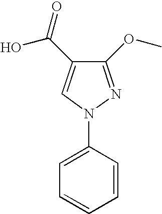 | 3-methoxy-1- phenyl-1H- pyrazole-4- carboxylic acid | ¹H NMR (400 MHz, CDCl₃) δ 4.13 (s, 3H), 7.32 (t, J = 7.4 Hz, 1H), 7.47 (t, J = 8.0 Hz, 2H), 7.67 (d, J = 7.6 Hz, 2H), 8.32 (s, 1H); m/z (APCI neg) 217 (100%) [M − H]. |
| 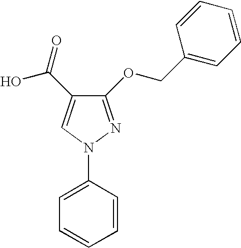 | 3-(benzyloxy)- 1-phenyl-1H- pyrazole-4- carboxylic acid | m/z (APCI neg) 293 (100%) [M − H]. |
| 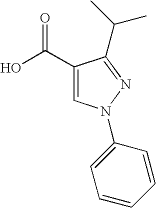 | 3-isopropyl-1- phenyl-1H- pyrazole-4- carboxylic acid | m/z (APCI neg) 229 (100%) [M − H]. |
| 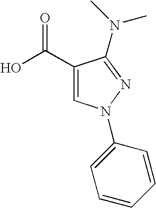 | 3-(dimethyl- amino)-1-phenyl- 1H-pyrazole- 4-carboxylic acid | m/z (APCI neg) 230 (100%) [M − H]. |

### Step 2

A mixture of N-(2-aminoethyl)-6-(2,2,2-trifluoroethoxy)nicotinamide hydrochloride (0.741 g, 2.47 mmol), 3-(tert-butoxycarbonylamino)-1-phenyl-1H-pyrazole-4-carboxylic acid (0.750 g, 2.47 mmol), HATU (1.13 g, 2.97 mmol), and DIPEA (2.15 ml, 12.4 mmol) in THF (20 mL) was stirred at room temperature for 3 hrs. The mixture was quenched with water (200 mL) and extracted with DCM, dried over Na₂SO₄ and concentrated to a residue that was purified by silica gel chromatography eluting with 75% EtOAc/hexanes. This material was dissolved in EtOAc and the solution was washed with water, dried and concentrated to a residue that was recrystallized from EtOAc/hexanes to give tert-butyl 1-phenyl-4-(2-(6-(2,2,2-trifluoroethoxy)nicotinamido)ethylcarbamoyl)-1H-pyrazol-3-ylcarbamate (0.968 g, 71%) as a white solid: ¹H NMR (400 MHz, DMSO-d₆) δ 1.46 (s, 9H), 3.42 (br, 4H), 5.03-5.10 (m, 2H), 7.08 (d, J=8.6 Hz, 1H), 7.35 (t, J=7.4 Hz, 1H), 7.54 (t, J=8.0 Hz, 2H), 7.71 (d, J=7.6 Hz, 2H), 8.20-8.23 (m, 1H), 8.40 (br, 1H), 8.68-8.70 (m, 2H), 8.86 (s, 1H), 9.30 (s, 1H); m/z (APCI pos) 549 (M+H).

Compounds of the following structures were prepared by reacting N-(2-aminoethyl)-6-(2,2,2-trifluoroethoxy)nicotinamide hydrochloride and the corresponding acids, using the procedure outlined above.

| Ex # | Structure | Name | Physical Data |
|---|---|---|---|
| A55 | 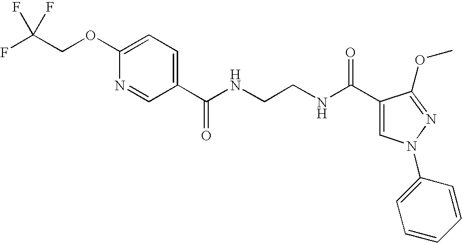 | N-(2-(3- methoxy-1- phenyl-1H- pyrazole-4- carboxamido) ethyl)-6-(2,2,2- trifluoroethoxy) nicotinamide | ¹H NMR (400 MHz, DMSO- d₆) δ 3.44 (br, 4H), 4.00 (s, 3H), 5.03-5.10 (m, 2H), 7.09 (d, J = 8.6 Hz, 1H), 7.29 (t, J = 7.4 Hz, 1H), 7.46-7.51 (m, 3H), 7.82 (d, J = 7.6 Hz, 2H), 8.20-8.23 (m, 1H), 8.68-8.70 (m, 2H), 8.78 (s, 1H); m/z (APCI pos) 464 (M + H). |
| A56 | 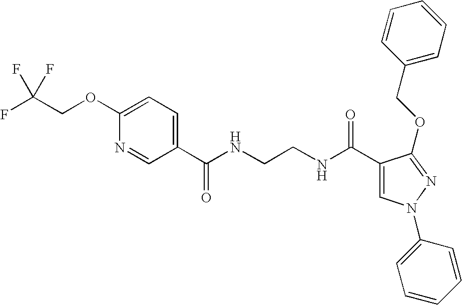 | N-(2-(3- (benzyloxy)- 1-phenyl-1H- pyrazole-4- carboxamido) ethyl)-6-(2,2,2- trifluoroethoxy) nicotinamide | ¹H NMR (400 MHz, DMSO- d₆) δ 3.32 (br, 4H), 5.03-5.10 (m, 2H), 5.42 (s, 2H), 7.06 (d, J = 8.6 Hz, 1H), 7.31 (m, 4H), 7.47-7.50 (m, 5H), 7.81 (d, J = 8.0 Hz, 2H), 8.20 (d, J = 8.3 Hz, 1H), 8.68 (s, 1H), 8.71 (s, 1H), 8.80 (s, 1H); m/z (APCI pos) 540 (M + H). |
| A57 | 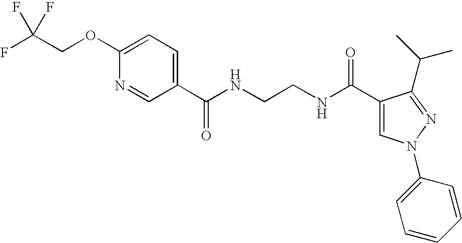 | N-(2-(3- isopropyl-1- phenyl-1H- pyrazole-4- carboxamido) ethyl)-6-(2,2,2- trifluoroethoxy) nicotinamide | ¹H NMR (400 MHz, DMSO- d₆) δ 1.24 (d, J = 6.8 Hz, 6H), 3.41 (br, 4H), 3.53-3.60 (m, 1H), 5.03-5.09 (m, 2H), 7.07 (d, J = 8.6 Hz, 1H), 7.33 (t, 7.4 Hz, 1H), 7.53 (t, J = 7.9 Hz, 2H), 7.73 (d, J = 7.6 Hz, 2H), 8.15 (br, 1H), 8.20-8.23 (m, 1H), 8.68 (d, J = 1.7 Hz, 2H), 8.80 (s, 1H); m/z (APCI pos) 476 (M + H). |
| A58 | 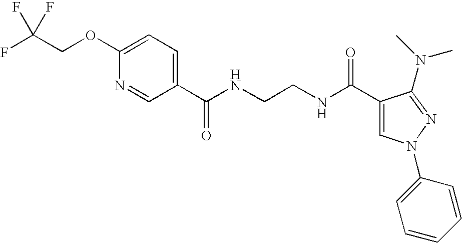 | N-(2-(3- (dimethylamino)- 1-phenyl-1H- pyrazole-4- carboxamido) ethyl)-6-(2,2,2- trifluoroethoxy) nicotinamide | ¹H NMR (400 MHz, DMSO- d₆) δ 2.81 (s, 6H), 3.42 (br, 4H), 5.03- 5.09 (m, 2H), 7.08 (d, J = 8.6 Hz, 1H), 7.26 (t, J = 7.4 Hz, 1H), 7.48 (t, J = 8.0 Hz, 2H), 7.72 (d, J = 7.6 Hz, 2H), 8.08 (br, 1H), 8.2-8.23 (m, 1H), 8.66- 8.68 (m, 3H); m/z (APCI pos) 477 (M + H). |
| A59 | 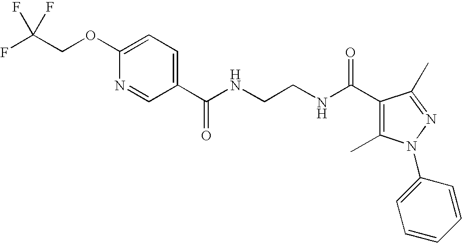 | N-(2-(3,5- dimethyl-1- phenyl-1H- pyrazole-4- carboxamido) ethyl)-6-(2,2,2- trifluoroethoxy) nicotinamide | ¹H NMR (400 MHz, DMSO- d₆) δ 2.30 (s, 3H), 2.36 (s, 3H), 3.44 (br, 4H), 5.03-5.09 (m, 2H), 7.08 (d, J = 8.6 Hz, 1H), 7.42-7.46 (m, 3H), 7.51-7.55 (m, 2H), 7.76 (br, 1H), 8.20- 8.23 (m, 1H), 8.68 (br, 2H); m/z (APCI pos) 462 (M + H). |

### Example A62

### N-(2-(1-Phenyl-3-(trifluoromethyl)-1H-pyrazole-4-carboxamido)ethyl)-5-propyl-6-(2,2,2-trifluoroethoxy)nicotinamide

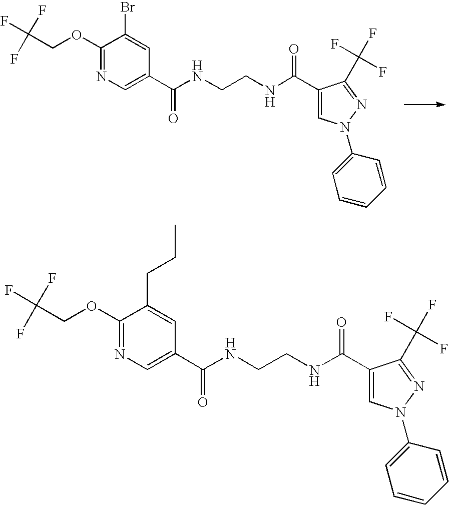

5-Bromo-N-(2-(1-phenyl-3-(trifluoromethyl)-1H-pyrazole-4-carboxamido)ethyl)-6-(2,2,2-trifluoroethoxy)nicotinamide (0.200 g, 0.345 mmol) and PdCl₂(dppf) dichloromethane adduct (0.014 g, 0.017 mmol) were added together in degassed THF (2 mL) under an argon atmosphere. Propylzinc(II) bromide (1.72 ml, 0.862 mmol, 0.5 M in THF) was then added. The mixture was stirred under argon at 55° C. for 1 hr. The solution was cooled and filtered through a silica plug (rinsing with EtOAc) and concentrated. Flash chromatography on silica gel gave N-(2-(1-phenyl-3-(trifluoromethyl)-1H-pyrazole-4-carboxamido)ethyl)-5-propyl-6-(2,2,2-trifluoroethoxy)nicotinamide (0.063 g, 34%): ¹H NMR (400 MHz, CDCl₃) δ 0.94 (t, J=7.6 Hz, 3H), 1.61-1.66 (m, 2H), 2.60 (t, J=7.2 Hz, 2H), 3.71 (br, 4H), 4.80 (q, J=8.4, 16.8 Hz, 2H), 6.81 (br, 1H), 7.21 (br, 1H), 7.42 (t, J=7.2 Hz, 1H), 7.51 (t, J=7.6 Hz, 2H), 7.69 (d, J=7.6 Hz, 2H), 7.90 (s, 1H), 8.44 (s, 2H); m/z (APCI pos) 544.1 (M+H).

The compound of the following structure was prepared from 5-bromo-N-(2-(1-phenyl-3-(trifluoromethyl)-1H-pyrazole-4-carboxamido)ethyl)-6-(2,2,2-trifluoroethoxy)nicotinamide and phenethylzinc bromide, using a similar method to that described above.

| Ex # | Structure | Name | Physical Data |
|---|---|---|---|
| A64 | 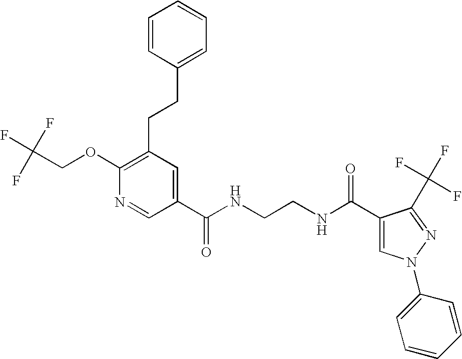 | 5-phenethyl- N-(2-(1- phenyl-3- (trifluoromethyl)- 1H-pyrazole-4- carboxamido)ethyl)- 6-(2,2,2- trifluoroethoxy) nicotinamide | ¹H NMR (400 MHz, CDCl₃) δ 2.90-2.92 (m, 4H), 3.69-3.73 (m, 4H), 4.82 (q, J = 8.0, 16.4 Hz, 2H), 6.73 (br, 1H), 7.10 (br, 1H), 7.15- 7.18 (m, 3H), 7.25- 7.27 (m, 2H), 7.39- 7.43 (m, 1H), 7.50 (t, J = 6.8 Hz, 2H), 7.68 (d, J = 8.4 Hz, 2H), 7.84 (d, J = 2.4 Hz, 1H), 8.42 (s, 1H), 8.46 (d, J = 2.4 Hz, 1H); m/z (APCI pos) 606.1 (M + H). |

### Example A63

### 5-Phenyl-N-(2-(1-phenyl-3-(trifluoromethyl)-1H-pyrazole-4-carboxamido)ethyl)-6-(2,2,2-trifluoroethoxy)nicotinamide

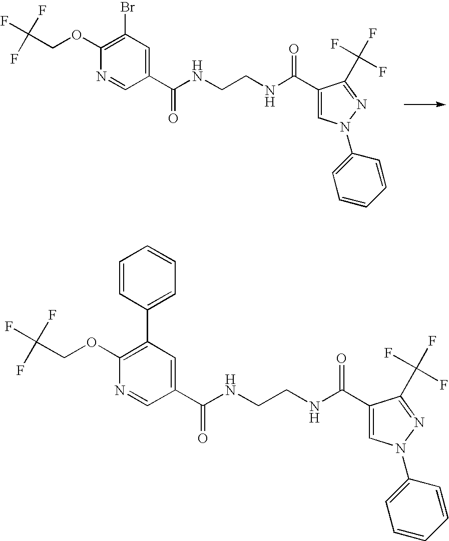

5-Bromo-N-(2-(1-phenyl-3-(trifluoromethyl)-1H-pyrazole-4-carboxamido)ethyl)-6-(2,2,2-trifluoroethoxy)nicotinamide (0.100 g, 0.172 mmol), phenylboronic acid (0.031 g, 0.26 mmol), Pd(OAc)₂ (0.004 g, 0.017 mmol), and cesium carbonate (0.112 g, 0.345 mmol) were added together under nitrogen. 2,8,9-Triisobutyl-2,5,8,9-tetraaza-1-phosphabicyclo[3.3.3]undecane (0.012 g, 0.035 mmol) was then added. The mixture was diluted in toluene (3 mL) and stirred at 80° C. for 3 hours. The solution was filtered and concentrated. Flash chromatography on silica gel (70%-100% EtOAc/hexanes) gave 5-phenyl-N-(2-(1-phenyl-3-(trifluoromethyl)-1H-pyrazole-4-carboxamido)ethyl)-6-(2,2,2-trifluoroethoxy)nicotinamide (0.063 g, 63%) as a white solid: ¹H NMR (400 MHz, CDCl₃) δ 3.72-3.74 (m, 4H), 4.87 (q, J=84, 17.2 Hz, 2H), 6.75 (br, 1H), 7.34 (br, 1H), 7.38-7.52 (m, 6H), 7.58-7.60 (m, 2H), 7.65-7.67 (m, 2H), 8.16 (d, J=2.4 Hz, 1H), 8.40 (s, 1H), 8.59 (d, J=2.4 Hz, 1H); m/z (APCI pos) 578.1 (M+H).

### Example A66

### N-(2-(3-Amino-1-phenyl-1H-pyrazole-4-carboxamido)ethyl)-6-(2,2,2-trifluoroethoxy)nicotinamide

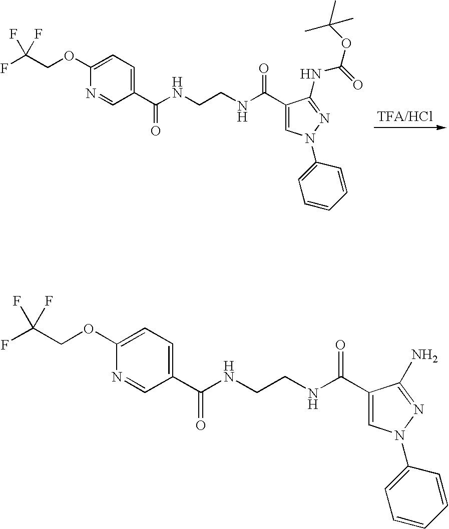

To a mixture of tert-butyl 1-phenyl-4-(2-(6-(2,2,2-trifluoroethoxy)nicotinamido)ethylcarbamoyl)-1H-pyrazol-3-ylcarbamate (0.200 g, 0.365 mmol) in DCM (20 mL) was added TFA (0.140 ml, 1.82 mmol) and the mixture was stirred for 40 minutes at room temperature. TFA (5 mL) was added and the mixture was stirred for 5 hours. The mixture was concentrated to a residue and the residue dissolved in DCM. The solution was washed with saturated aqueous sodium carbonate and water, dried over Na₂SO₄ and concentrated to a residue that was recrystallized from EtOAc/hexanes to give N-(2-(3-amino-1-phenyl-1H-pyrazole-4-carboxamido)ethyl)-6-(2,2,2-trifluoroethoxy)nicotinamide (0.115 g, 70%): ¹H NMR (400 MHz, DMSO-d₆) δ 3.4 (br, 4H), 5.03-5.10 (m, 2H), 5.72 (s, 2H), 7.08 (d, J=8.7 Hz, 1H), 7.24 (t, J=7.4 Hz, 1H), 7.48 (t, J=7.9 Hz, 2H), 7.60 (d, J=7.8 Hz, 2H), 8.10 (br, 1H), 8.20-8.23 (m, 1H), 8.68-8.71 (m, 3H): m/z (APCI pos) 449 (M+H).

### Example A68

### 1-Methyl-N-(2-(6-(2,2,2-trifluoroethoxy)nicotinamido)ethyl)-1H-indole-3-carboxamide

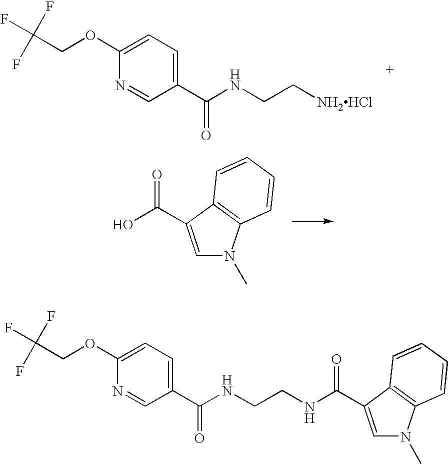

To N-(2-aminoethyl)-6-(2,2,2-trifluoroethoxy)nicotinamide hydrochloride (342 mg, 1.14 mmol) in DCM (100 mL) and DMF (10 mL) were successively added 1-methyl-1H-indole-3-carboxylic acid (200 mg, 1.14 mmol), EDAC.HCl (285 mg, 1.48 mmol), HOBt.H₂O (216 mg, 1.60 mmol) and triethylamine (231 mg, 2.28 mmol). The mixture was stirred at room temperature for 18 hours and DCM (300 mL) was added. The organic layer was successively washed with water (3×200 mL), 1N HCl (100 mL), 10% potassium carbonate aqueous solution (100 mL) and brine (2×200 mL). The organic layer was dried over MgSO₄ then concentrated to yield an orange solid. The crude solid was purified by silica gel chromatography (DCM/MeOH 95/5) to yield 1-methyl-N-(2-(6-(2,2,2-trifluoroethoxy)nicotinamido)ethyl)-1H-indole-3-carboxamide as a white solid (120 mg, 25%): ¹H NMR (400 MHz, DMSO-d₆) δ 3.42-3.46 (m, 4H), 3.82 (s, 3H), 5.06 (q, 2H, J=9.0 Hz), 7.08 (m, 1H), 7.14 (m, 1H), 7.22 (m, 1H), 7.48 (m, 1H), 7.94 (s, 1H), 8.00-8.06 (m, 1H), 8.14 (m, 1H), 8.22 (m, 1H), 8.68-8.73 (m, 2H); m/z (APCI pos) 421.0 (100%) (M+H).

The compound of the following structure was prepared from N-(2-aminoethyl)-6-(2,2,2-trifluoroethoxy)nicotinamide hydrochloride and the corresponding acid, using a similar method to that described above.

| Ex # | Structure | Name | Physical Data |
|---|---|---|---|
| A72 | 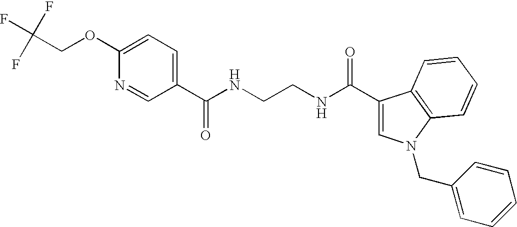 | 1-benzyl- N-(2-(6- (2,2,2- trifluoroeth- oxy)nico tinamido)eth- yl)-1H- indole-3- carboxamide | White solid (60%); ¹H NMR (400 MHz, DMSO-d₆) δ 3.42- 3.46(m, 4 H), 5.05(q, 2 H), J = 9.0 Hz), 5.46(s, 2 H), 7.05-7.35(m, 8 H), 7.52 (m, 1 H), 8.07-8.13(m, 2 H), 8.15(m, 1 H), 8.22 (dd, 1 H, J = 2.4, 8.6 Hz), 8.68-8.73(m, 2 H); m/z (APCI pos) 497.1 (80%) (M + H). |

### Example A70

### 5-Benzyl-N-(2-(1-phenyl-3-(trifluoromethyl)-1H-pyrazole-4-carboxamido)ethyl)-6-(2,2,2-trifluoroethoxy)nicotinamide

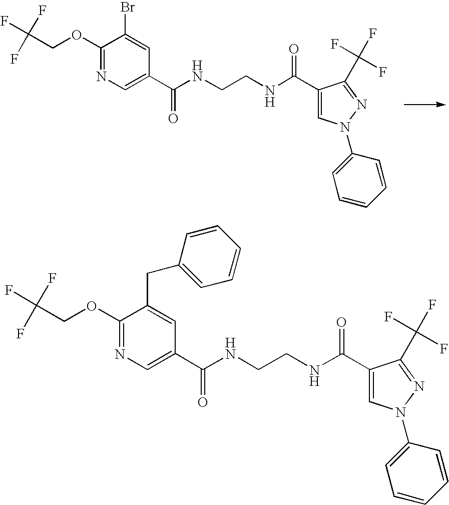

9-Benzyl-9-bora-bicyclo[3.3.1]nonane (0.86 ml, 1.72 mmol, 0.5 M in THF) was added to DMF (5 mL) under an argon atmosphere. 5-Bromo-N-(2-(1-phenyl-3-(trifluoromethyl)-1H-pyrazole-4-carboxamido)ethyl)-6-(2,2,2-trifluoroethoxy)nicotinamide (0.200 g, 0.345 mmol) and PdCl₂(dppf) dichloromethane adduct (0.014 g, 0.017 mmol) were then added. The mixture was heated at 60° C. for 90 minutes under argon. The solution was cooled and poured onto water and extracted with EtOAc. The organic layer was dried and concentrated. Reverse phase HPLC purification and recrystallization from EtOAc gave 5-benzyl-N-(2-(1-phenyl-3-(trifluoromethyl)-1H-pyrazole-4-carboxamido)ethyl)-6-(2,2,2-trifluoroethoxy)nicotinamide (0.040 g, 20%): ¹H NMR (400 MHz, DMSO-d₆) δ 3.40-3.42 (m, 4H), 3.93 (s, 2H), 5.03 (q, J=8.8, 17.6 Hz, 2H), 7.16-7.28 (m, 5H), 7.47 (t, J=7.6 Hz, 1H), 7.60 (t, J=8.4 Hz, 2H), 7.80 (d, J=7.6 Hz, 2H), 8.10 (d, J=2.4 Hz, 1H), 8.48 (br, 1H), 8.54 (d, J=2.4 Hz, 1H), 8.67 (br, 1H), 9.05 (s, 1H); m/z (APCI pos) 592.1 (M+H).

### Example A73

### N-(2-(1-Phenyl-3-(trifluoromethyl)-1H-pyrazole-4-carboxamido)ethyl)-6-o-tolylnicotinamide

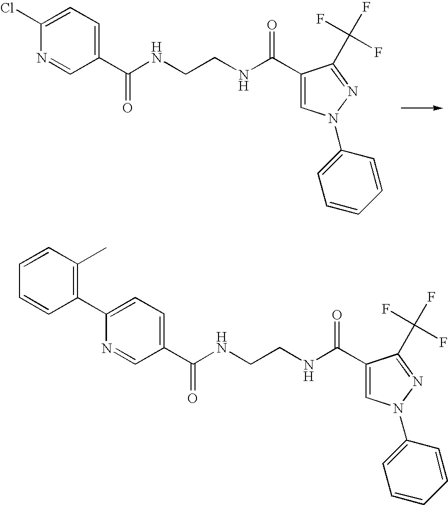

6-Chloro-N-(2-(1-phenyl-3-(trifluoromethyl)-1H-pyrazole-4-carboxamido)ethyl)nicotinamide (0.200 g, 0.457 mmol), o-tolylboronic acid (0.087 g, 0.640 mmol), PdCl₂(PPh₃)₂ (0.096 g, 0.137 mmol), and aqueous K₃PO₄ (0.685 ml, 1.37 mmol, 2 M) were suspended in toluene (2 mL). The mixture was stirred at 90° C. overnight. The material was filtered and concentrated. Reverse phase HPLC purification followed by recrystallized in EtOAc/hexanes gave N-(2-(1-phenyl-3-(trifluoromethyl)-1H-pyrazole-4-carboxamido)ethyl)-6-o-tolylnicotinamide (0.020 g, 9%) as a white solid: ¹H NMR (400 MHz, CDCl₃) δ 2.33 (s, 3H), 3.47 (br, 4H), 7.27-7.52 (m, 8H), 7.81 (d, J=8.0 Hz, 2H), 8.08-8.30 (m, 1H), 8.52 (br, 1H), 8.82 (br, 1H), 9.07-9.10 (m, 2H); m/z (APCI pos) 494.2 (M+H).

### Example A74

### 6-Ethoxy-2-phenyl-N-(2-(1-phenyl-3-(trifluoromethyl)-1H-pyrazole-4-carboxamido)ethyl)nicotinamide

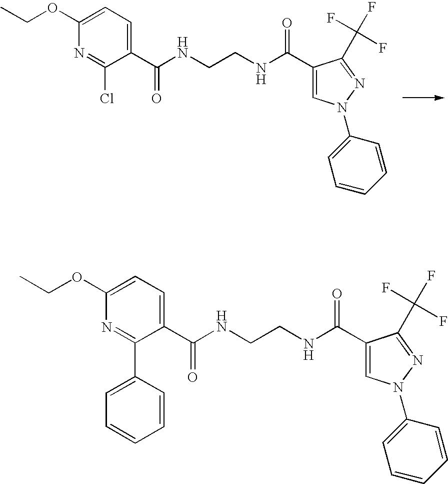

2-Chloro-6-ethoxy-N-(2-(1-phenyl-3-(trifluoromethyl)-1H-pyrazole-4-carboxamido)ethyl)nicotinamide (0.200 g, 0.415 mmol), phenylboronic acid (0.075 g, 0.62 mmol), Pd(OAc)₂ (0.009 g, 0.042 mmol), and cesium carbonate (0.270 g, 0.830 mmol) were added together under nitrogen. 2,8,9-Triisobutyl-2,5,8,9-tetraaza-1-phosphabicyclo[3.3.3]undecane (0.028 g, 0.083 mmol) was then added. The mixture was diluted in toluene (3 mL) and stirred at 80° C. for 4 hours. The solution was cooled and filtered. Reverse phase HPLC purification and recrystallization from EtOAc/hexanes gave 6-ethoxy-2-phenyl-N-(2-(1-phenyl-3-(trifluoromethyl)-1H-pyrazole-4-carboxamido)ethyl)nicotinamide (0.025 g, 12%): ¹H NMR (400 MHz, CDCl₃) δ 1.34 (t, J=6.8 Hz, 3H), 6.26 (br, 4H), 4.40 (q, J=7.2, 14.0 Hz, 2H), 6.79 (d, J=8.4 Hz, 1H), 7.34-7.38 (m, 3H), 7.48 (t, J=7.2 Hz, 1H), 7.58-7.65 (m, 4H), 7.76-7.81 (m, 3H), 8.33-8.36 (m, 2H), 8.99 (s, 1H); m/z (APCI pos) 524.1 (M+H).

### Example A77

### 5-Acetamido-N-(2-(1-phenyl-3-(trifluoromethyl)-1H-pyrazole-4-carboxamido)ethyl)-6-(2,2,2-trifluoroethoxy)nicotinamide

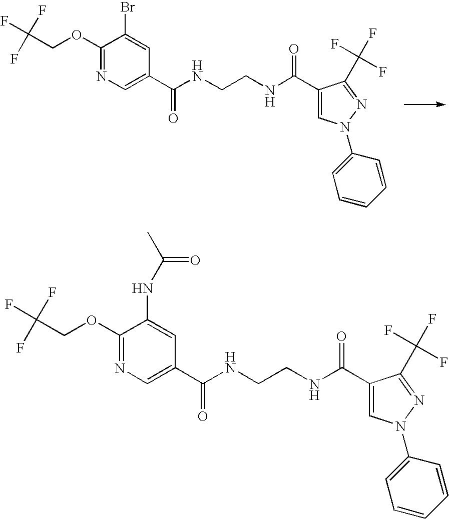

Xantphos (0.012 g, 0.021 mmol), Pd₂(dba)₃ (0.006 g, 0.007 mmol), 5-bromo-N-(2-(1-phenyl-3-(trifluoromethyl)-1H-pyrazole-4-carboxamido)ethyl)-6-(2,2,2-trifluoroethoxy)nicotinamide (0.10 g, 0.17 mmol), cesium carbonate (0.079 g, 0.241 mmol), and acetamide (0.015 g, 0.26 mmol) were added and the reaction tube was filled with argon. Dioxane (2 mL) was added via syringe and the vial was capped with a teflon sealed cap and the reaction mixture was stirred at 100° C. overnight. The solution was cooled, filtered and concentrated. Flash chromatography on silica gel (2% MeOH/EtOAc) followed by recrystallization in EtOAc/hexanes gave 5-acetamido-N-(2-(1-phenyl-3-(trifluoromethyl)-1H-pyrazole-4-carboxamido)ethyl)-6-(2,2,2-trifluoroethoxy)nicotinamide (0.047 g, 49%) as a white solid: ¹H NMR (400 MHz, DMSO-d₆) δ 2.12 (s, 3H), 3.41 (br, 4H), 5.12 (q, J=8.8 Hz, 2H), 7.47 (t, J=7.6 Hz, 1H), 7.61 (t, J=7.6 Hz, 2H), 7.81 (d, J=7.6 Hz, 2H), 8.40 (d, J=2.0 Hz, 1H), 8.50 (br, 1H), 8.68 (br, 2H), 9.05 (s, 1H), 9.54 (br, 1H); m/z (APCI pos) 559.0 (M+H).

### Example A78

Methyl 5-((2-(1-phenyl-3-(trifluoromethyl)-1H-pyrazole-4-carboxamido) ethyl)carbamoyl)-2-(2,2,2-trifluoroethoxy)nicotinate

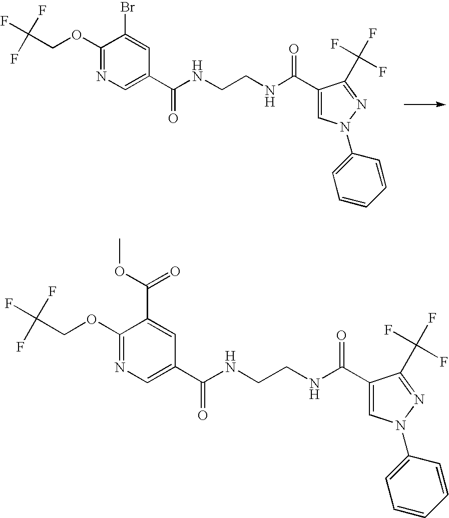

5-Bromo-N-(2-(1-phenyl-3-(trifluoromethyl)-1H-pyrazole-4-carboxamido)ethyl)-6-(2,2,2-trifluoroethoxy)nicotinamide (0.200 g, 0.345 mmol) and triethylamine (0.0625 ml, 0.448 mmol) were dissolved in MeOH (2 mL). The solution was degassed with nitrogen for 10 minutes. (R)-(BINAP)PdCl₂ (0.0138 g, 0.0172 mmol) was added and the solution was purged with nitrogen followed by CO gas (3 times). The vessel was pressurized to 70 psi and heated at 75° C. for 24 hours. The solution was cooled, filtered and concentrated. Flash chromatography on silica gel (EtOAc) followed by recrystallization gave methyl 5-((2-(1-phenyl-3-(trifluoromethyl)-1H-pyrazole-4-carboxamido)ethyl)carbamoyl)-2-(2,2,2-trifluoroethoxy)nicotinate (0.025 g, 13%) as a white solid: ¹H NMR (400 MHz, DMSO-d₆) δ 3.44 (br, 4H), 3.85 (s, 3H), 5.14 (q, J=8.8 Hz, 2H), 7.48 (t, J=7.6 Hz, 1H), 7.61 (t, J=8.4 Hz, 2H), 7.80 (d, J=7.6 Hz, 2H), 8.48 (br, 1H), 8.66 (d, J=2.4 Hz, 1H), 8.85 (d, J=2.4 Hz, 1H), 8.87 (br, 1H), 9.05 (s, 1H); m/z (APCI pos) 560.0 (M+H).

### Example A81

### 5-((2-(1-Phenyl-3-(trifluoromethyl)-1H-pyrazole-4-carboxamido)ethyl)carbamoyl)picolinic acid

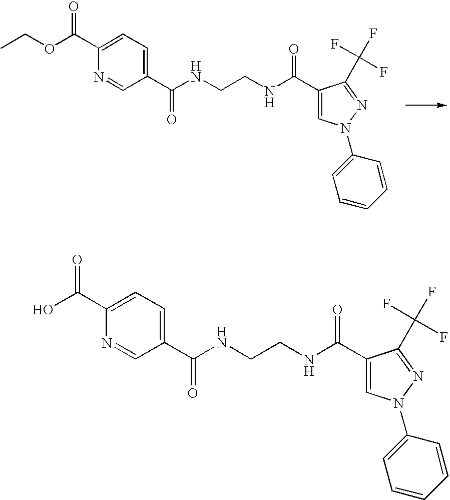

Ethyl 5-((2-(1-phenyl-3-(trifluoromethyl)-1H-pyrazole-4-carboxamido)ethyl)carbamoyl)picolinate (0.350 g, 0.736 mmol) and lithium hydroxide monohydrate (0.0927 g, 2.21 mmol) were dissolved in EtOH (5 mL) and water (5 mL), and the mixture was stirred at 50° C. for 90 minutes. The solution was cooled and concentrated. The residue was diluted with water and the mixture was extracted with EtOAc. The aqueous layer was then acidified with 6N HCl to pH<3. The aqueous solution was then extracted with DCM and the combined organic layers were dried and concentrated to give 5-((2-(1-phenyl-3-(trifluoromethyl)-1H-pyrazole-4-carboxamido)ethyl)carbamoyl)picolinic acid (0.257 g, 78%): ¹H NMR (400 MHz, DMSO-d₆) δ 3.46 (s, 4H), 7.48 (t, J=7.4 Hz, 1H), 7.61 (t, J=8.0 Hz, 2H), 7.81 (d, J=7.6 Hz, 2H), 8.12 (d, J=8.0 Hz, 1H), 8.33-8.35 (m, 1H), 8.53 (s, 1H), 8.96 (s, 1H), 9.07-9.09 (m, 2H); m/z (APCI pos) 448 (M+H).

### Example A82

### 1-Benzoyl-N-(2-(6-(2,2,2-trifluoroethoxy)nicotinamido)ethyl)-1H-indole-3-carboxamide

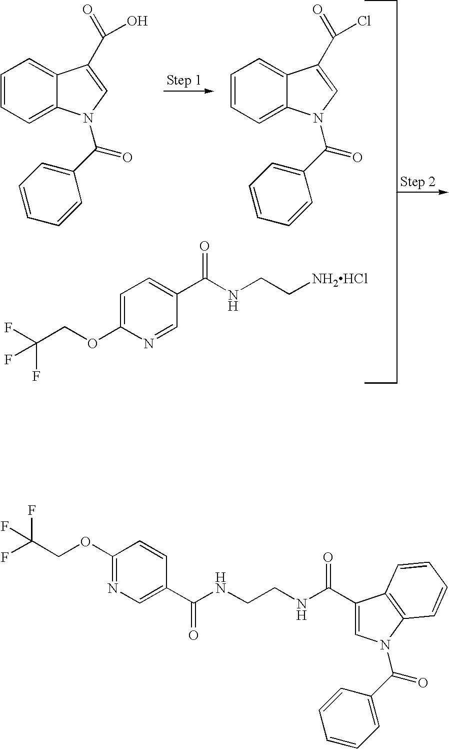

### Step 1

To a solution of 1-benzoyl-1H-indole-3-carboxylic acid (0.200 g, 0.75 mmol) in DCM and 1 drop of DMF was added oxalyl chloride (0.132 ml, 1.51 mmol) and the mixture was stirred for 1 hour. The solvent was removed to yield the corresponding acid chloride, which was used without further purification.

### Step 2

To N-(2-aminoethyl)-6-(2,2,2-trifluoroethoxy)nicotinamide hydrochloride (0.226 g, 0.75 mmol) and triethylamine (0.105 ml, 0.75 mmol) in DMF (15 ml) was added 1-benzoyl-1H-indole-3-carbonyl chloride (0.214 g, 0.75 mmol). The mixture was stirred overnight at room temperature and concentrated in vacuo, and the residue was dissolved in EtOAc. The mixture was then washed with water (2×), saturated aqueous sodium bicarbonate (2×), and brine. The combined organic layers were dried (sodium sulfate), filtered, and concentrated in vacuo to give a residue which was purified by silica gel chromatography (3-5% MeOH/DCM) and triturated with ether/methanol to give 1-benzoyl-N-(2-(6-(2,2,2-trifluoroethoxy)nicotinamido)ethyl)-1H-indole-3-carboxamide (0.022 g, 6%) as a solid: ¹H NMR (400 MHz, DMSO-d₆) δ 3.40 (m, 4H), 5.06 (q, 2H, J=8.98 Hz), 7.06 (m, 1H), 7.41 (m, 2H), 7.64 (m, 2H), 7.74 (m, 1H), 7.82 (m, 2H), 8.15 (s, 1H), 8.17 (m, 1H), 8.27 (m, 2H), 8.51 (m, 1H), 8.65 (m, 1H), 8.69 (m, 1H); m/z (APCI pos) 511.1 (100%) (M+H).

### Example A83

### 6-(Ethylthio)-N-(2-(1-phenyl-3-(trifluoromethyl)-1H-pyrazole-4-carboxamido)ethyl)nicotinamide

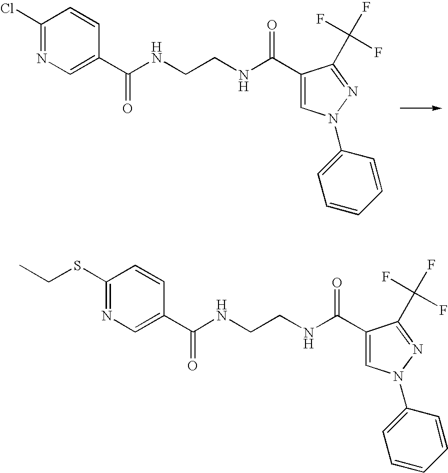

A mixture of 6-chloro-N-(2-(1-phenyl-3-(trifluoromethyl)-1H-pyrazole-4-carboxamido)ethyl)nicotinamide (0.234 g, 0.53 mmol), sodium thioethoxide (0.17 g, 1.6 mmol) and THF (2 mL) was stirred at 80° C. for 3 h. After cooling to room temperature, the mixture was diluted with EtOAc, washed with water and brine, dried and concentrated in vacuo. The residue was purified by silica gel column chromatography (EtOAc to EtOAc/MeOH=10/1) to give 6-(ethylthio)-N-(2-(1-phenyl-3-(trifluoromethyl)-1H-pyrazole-4-carboxamido)ethyl)nicotinamide (0.23 g, 94%) as colorless crystals: ¹H NMR (400 MHz, CDCl₃) δ 1.38 (3H, t, J=7.6 Hz), 3.20 (2H, q, J=7.6 Hz), 3.66-3.76 (4H, m), 6.77 (1H, br), 7.20 (1H, d, J=8.4 Hz), 7.24 (1H, m), 7.42 (1H, t, J=7.6 Hz), 7.52 (2H, t, J=7.6 Hz), 7.70 (2H, d, J=7.6 Hz), 7.89 (1H, dd, J=2.4, 8.4 Hz), 8.44 (1H, d, J=0.4 Hz), 8.86 (1H, d, J=2.4 Hz); m/z (APCI pos) 464.1 (100%) (M+H).

### Example A85

### 6-(Ethylsulfonyl)-N-(2-(1-phenyl-3-(trifluoromethyl)-1H-pyrazole-4-carboxamido)ethyl)nicotinamide

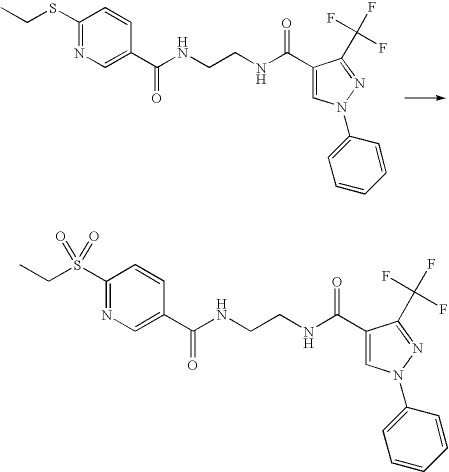

To a mixture of 6-(ethylthio)-N-(2-(1-phenyl-3-(trifluoromethyl)-1H-pyrazole-4-carboxamido)ethyl)nicotinamide (0.21 g, 0.45 mmol), formic acid (0.17 mL, 4.53 mmol), acetone (4 mL) and THF (2 mL) was added potassium permanganate (0.18 g, 1.13 mmol) at 0° C. and the mixture was stirred for 16 h. The same amounts of reagents (formic acid and potassium permanganate) were added to the mixture at 0° C. and the mixture was stirred for 2 h at room temperature. This handling was repeated twice. The insoluble material was removed by filtration and the filtrate was concentrated in vacuo. The residue was diluted with EtOAc, washed with saturated aqueous sodium bicarbonate and brine, dried and concentrated in vacuo. The residue was purified by silica gel column chromatograpy (EtOAc to EtOAc/MeOH=10/1) to give 6-(ethylsulfonyl)-N-(2-(1-phenyl-3-(trifluoromethyl)-1H-pyrazole-4-carboxamido)ethyl)nicotinamide (0.12 g, 55%) as colorless crystals: ¹H NMR (400 MHz, CDCl₃) δ 1.31 (3H, t, J=7.6 Hz), 3.45 (2H, q, J=7.6 Hz), 3.68-3.82 (4H, m), 6.68 (1H, br), 7.44 (1H, t, J=7.6 Hz), 7.53 (2H, t, J=7.6 Hz), 7.72 (2H, d, J=8.4 Hz), 7.98 (1H, br), 8.17 (1H, d, J=8.0 Hz), 8.41 (1H, dd, J=2.0, 8.0 Hz), 8.48 (1H, s), 9.17 (1H, d, J=2.0 Hz); m/z (APCI pos) 496.0 (100%) (M+H).

### Example A86

### N-(2-(1-Phenyl-3-(trifluoromethyl)-1H-pyrazole-4-carboxamido)ethyl)-6-(phenylsulfonyl)nicotinamide

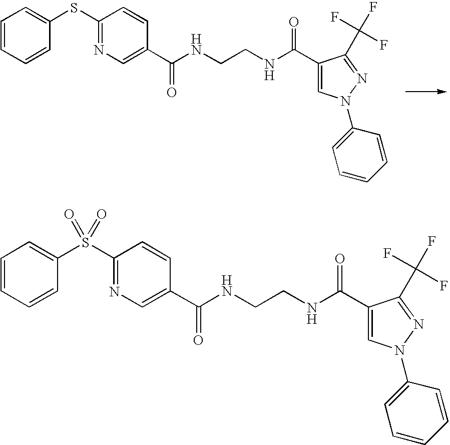

To a solution of N-(2-(1-phenyl-3-(trifluoromethyl)-1H-pyrazole-4-carboxamido)ethyl)-6-(phenylthio)nicotinamide (0.11 g, 0.21 mmol) in DCM (4 mL) was added 3-chloroperbenzoic acid (0.13 g, 70-75%, 0.51 mmol) at 0° C. and the mixture was stirred for 12 h at room temperature. The mixture was washed with saturated aqueous sodium carbonate and brine, dried and concentrated in vacuo. The residue was purified by silica gel column chromatograpy (EtOAc) to give N-(2-(1-phenyl-3-(trifluoromethyl)-1H-pyrazole-4-carboxamido)ethyl)-6-(phenylsulfonyl)nicotinamide (0.06 g, 54%) as colorless crystals: ¹H NMR (400 MHz, CDCl₃) δ 3.64-3.76 (4H, m), 6.72 (1H, br), 7.43 (1H, m), 7.48-7.57 (4H, m), 7.63 (1H, m), 7.71 (2H, d, J=8.0 Hz), 7.92 (1H, m), 8.05 (2H, d, J=8.0 Hz), 8.25 (1H, dd, J=0.8, 8.4 Hz), 8.36 (1H, dd, J=2.0, 8.4 Hz), 8.47 (1H, d, J=0.8 Hz), 9.08 (1H, dd, J=0.8, 2.0 Hz); m/z (APCI pos) 544.1 (100%) (M+H).

### Example A87

### 5-Cyano-N-(2-(1-phenyl-3-(trifluoromethyl)-1H-pyrazole-4-carboxamido)ethyl)-6-(2,2,2-trifluoroethoxy)nicotinamide

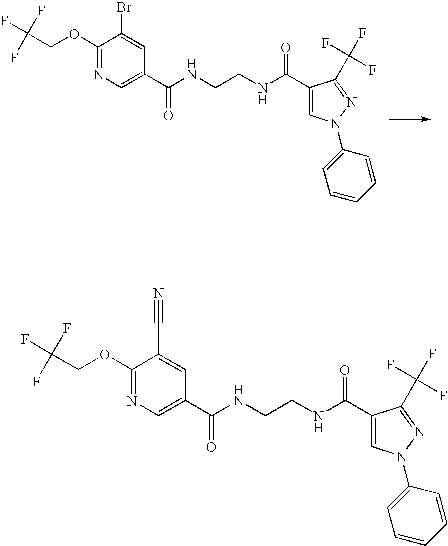

To a solution of Cu(I) CN (0.023 g, 0.258 mmol) in DMF (1 mL) was added 5-bromo-N-(2-(1-phenyl-3-(trifluoromethyl)-1H-pyrazole-4-carboxamido)ethyl)-6-(2,2,2-trifluoroethoxy)nicotinamide (0.100 g, 0.172 mmol) and the solution was heated at reflux (160° C.) overnight. The solution was cooled to room-temperature, diluted with water, extracted with EtOAc, dried, and concentrated. Reverse phase HPLC purification gave 5-cyano-N-(2-(1-phenyl-3-(trifluoromethyl)-1H-pyrazole-4-carboxamido)ethyl)-6-(2,2,2-trifluoroethoxy)nicotinamide (0.045 g, 50%): ¹H NMR (400 MHz, DMSO-d₆) δ 3.44 (br, 4H), 5.22 (q, J=8.8, 17.7 Hz, 2H), 7.47 (t, J=7.4 Hz, 1H), 7.61 (t, J=7.6 Hz, 2H), 7.81 (d, J=7.6 Hz, 2H), 8.53 (br, 1H), 8.73 (d, J=2.3 Hz, 1H), 8.87 (br, 1H), 8.90 (d, J=2.3 Hz, 1H), 9.09 (s, 1H); m/z (APCI pos) 527.1 (M+H).

### Example A88

### 2-Cyano-6-ethoxy-N-(2-(1-phenyl-3-(trifluoromethyl)-1H-pyrazole-4-carboxamido)ethyl)nicotinamide

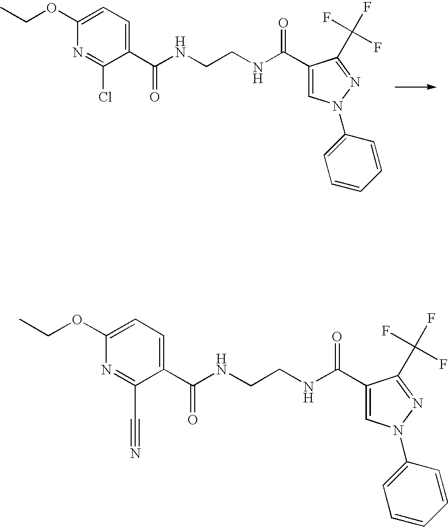

A solution of Cu(I) CN (0.0279 g, 0.311 mmol) in DMF (1 mL) was added to 2-chloro-6-ethoxy-N-(2-(1-phenyl-3-(trifluoromethyl)-1H-pyrazole-4-carboxamido)ethyl)nicotinamide (0.100 g, 0.208 mmol) and the solution was heated at reflux (160° C.) overnight. The solution was cooled and diluted with water, extracted with EtOAc, dried, and concentrated. Flash chromatography on silica gel and recrystallization gave 2-cyano-6-ethoxy-N-(2-(1-phenyl-3-(trifluoromethyl)-1H-pyrazole-4-carboxamido)ethyl)nicotinamide (88 mg, 83%) as a white solid: ¹H NMR (400 MHz, DMSO-d₆) δ 1.38 (t, J=7.0 Hz, 3H), 3.54 (q, J=5.9, 11.9 Hz, 2H), 3.88 (t, J=6.0 Hz, 2H), 4.51 (q, J=7.0, 14.1 Hz, 2H), 7.06 (d, J=8.4 Hz, 1H), 7.47 (t, J=7.5 Hz, 1H), 7.59 (t, J=7.5 Hz, 2H), 7.78 (d, J=8.7 Hz, 2H), 8.09 (d, J=8.4 Hz, 1H), 8.48 (t, J=5.9 Hz, 1H), 8.88 (s, 1H), 9.6 (s, 1H); m/z (APCI pos) 473.1 (M+H).

### Example A89

### 5-Acetyl-N-(2-(1-phenyl-3-(trifluoromethyl)-1H-pyrazole-4-carboxamido)ethyl)-6-(2,2,2-trifluoroethoxy)nicotinamide

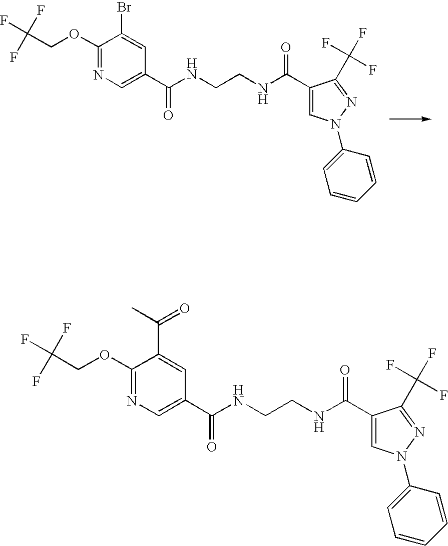

5-Bromo-N-(2-(1-phenyl-3-(trifluoromethyl)-1H-pyrazole-4-carboxamido)ethyl)-6-(2,2,2-trifluoroethoxy)nicotinamide (0.490 g, 0.844 mmol), 1-(vinyloxy)butane (1.09 ml, 8.44 mmol), Pd(OAc)₂ (0.0190 g, 0.0844 mmol), 1,3-bis(diphenyl-phosphino)propane (dppp) (0.0697 g, 0.169 mmol), and potassium carbonate (0.140 g, 1.01 mmol) were diluted in DMF (12.5 mL). Water (3.04 ml, 169 mmol) was added. The mixture was stirred in a sealed tube at 80° C. overnight. The mixture was cooled and diluted with water. The mixture was extracted with EtOAc, dried, and concentrated. Flash chromatography on silica gel (70% EtOAc/hexanes) gave the enol ether which was concentrated. The residue was dissolved in 2N HCl (10 mL) and stirred for 1 hr. The solution was neutralized with sodium bicarbonate and extracted with EtOAc, dried, and concentrated to afford 5-acetyl-N-(2-(1-phenyl-3-(trifluoromethyl)-1H-pyrazole-4-carboxamido)ethyl)-6-(2,2,2-trifluoroethoxy)nicotinamide (0.060 g, 13%) as a white solid: ¹H NMR (400 MHz, DMSO-d₆) δ 2.59 (s, 3H), 3.44 (br, 4H), 5.18 (q, J=8.9, 17.8 Hz, 2H), 7.48 (t, J=7.4H, 1H), 7.61 (t, J=7.6 Hz, 2H), 7.82 (t, J=7.6 Hz, 2H), 8.49 (br, 1H), 8.57 (d, J=2.4 Hz, 1H), 8.84 (d, J=2.4 Hz, 1H), 8.87 (br, 1H), 9.05 (s, 1H); m/z (APCI pos) 544.0 (M+H).

### Example A90

### N2-Methyl-N-5-(2-(1-phenyl-3-(trifluoromethyl)-1H-pyrazole-4-carboxamido)ethyl)pyridine-2,5-dicarboxamide

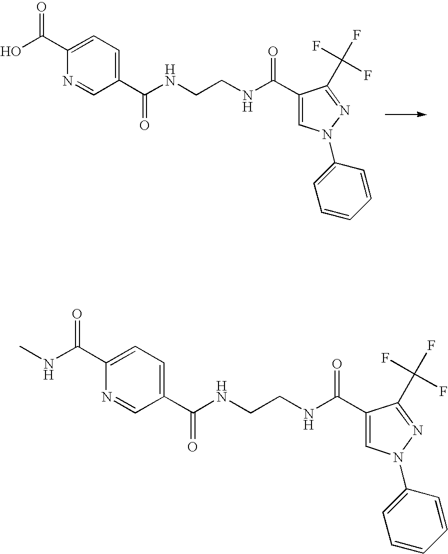

5-((2-(1-Phenyl-3-(trifluoromethyl)-1H-pyrazole-4-carboxamido)ethyl)carbamoyl)picolinic acid (0.200 g, 0.44 mmol), HATU (0.187 g, 0.49 mmol), and methylamine (0.67 ml, 1.34 mmol, 2M in THF) were diluted in THF (5 mL) and the mixture was stirred at room temperature overnight. The solution was quenched with water, extract with DCM, dried, and concentrated. The residue was triturated with EtOAc at reflux. The solid was filtered to provide N2-methyl-N-5-(2-(1-phenyl-3-(trifluoromethyl)-1H-pyrazole-4-carboxamido)ethyl)pyridine-2,5-dicarboxamide (0.077 g, 37%): ¹H NMR (400 MHz, DMSO-d₆) δ 2.83 (d, J=4.9 Hz, 3H), 3.46 (s, 4H), 7.48 (t, J=7.4 Hz, 1H), 7.61 (t, J=7.9 Hz, 2H), 7.80 (g, J=7.6 Hz, 2H), 8.11 (d, J=8.0 Hz, 1H), 8.37 (d, J=8.2 Hz, 1H), 8.51 (s, 1H), 8.88 (d, J=4.9 Hz, 1H), 8.93 (s, 1H), 9.02 (s, 1H), 9.06 (s, 1H); m/z (APCI pos) 461 (M+H)

The compound of the following structure was prepared from 5-((2-(1-phenyl-3-(trifluoromethyl)-1H-pyrazole-4-carboxamido)ethyl)carbamoyl)picolinic acid and dimethylamine, using a similar method to that described above.

| Ex # | Structure | Name | Physical Data |
|---|---|---|---|
| A92 | 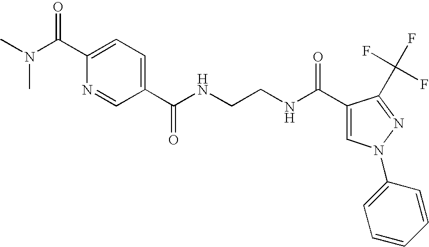 | N2,N2- dimethyl-N5- (2-(1-phenyl- 3- (trifluorometh yl)-1H- pyrazole-4- carboxamido)eth- yl)pyridine- 2,5- dicarboxamide | ¹H NMR (400 MHz, DMSO- d₆) δ 2.91(s, 3 H), 3.02(s, 3 H), 3.45(s, 4 H), 7.48(t, J = 7.4 Hz, 1 H), 7.59-7.65(m, 3 H), 7.81(d, J = 7.6 Hz, 2 H), 8.27-8.30(m, 1 H), 8.50(s, 1 H), 8.87 (s, 1 H), 8.99(d, J = 1.4 Hz, 1 H), 9.06(d, J = 0.8 Hz, 1 H); m/z (APCI pos) 475 (M + H). |

### Example A91

### Methyl 6-ethoxy-3-(2-(1-phenyl-3-(trifluoromethyl)-1H-pyrazole-4-carboxamido)ethylcarbamoyl)picolinate

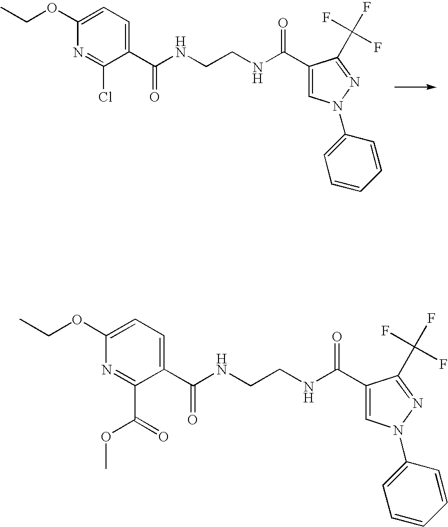

2-Chloro-6-ethoxy-N-(2-(1-phenyl-3-(trifluoromethyl)-1H-pyrazole-4-carboxamido)ethyl)nicotinamide (0.300 g, 0.623 mmol) and triethylamine (0.113 ml, 0.809 mmol) were dissolved in MeOH (3 mL). The solution was degassed with nitrogen for 10 minutes. (R)-(BINAP)PdCl₂ (0.024 g, 0.031 mmol) was added and the solution was purged with nitrogen followed by CO gas (2 times). The vessel was pressurized to 70 psi and heated at 65° C. for 24 hours. The solution was cooled, filtered and concentrated. Flash chromatography on silica gel (100% EtOAc) followed by recrystallization gave methyl 6-ethoxy-3-(2-(1-phenyl-3-(trifluoromethyl)-1H-pyrazole-4-carboxamido)ethylcarbamoyl)picolinate (0.030 g, 10%) as a white solid: ¹H NMR (400 MHz, DMSO-d₆) δ 1.29 (t, J=7.1 Hz, 3H), 3.43 (br, 4H), 3.73 (s, 3H), 4.40 (q, J=7.0, 14.0 Hz, 2H), 6.98 (d, J=8.6 Hz, 1H), 7.46 (t, J=7.4 Hz, 1H), 7.61 (t, J=7.5 Hz, 2H), 7.79-7.84 (m, 2H), 7.96 (d, J=8.5 Hz, 1H), 8.47 (br, 1H), 8.65 (br, 1H), 9.05 (s, 1H); m/z (APCI pos) 506.0 (M+H).

### Example A94

### 6-Cyano-N-(2-(1-phenyl-3-(trifluoromethyl)-1H-pyrazole-4-carboxamido)ethyl)nicotinamide

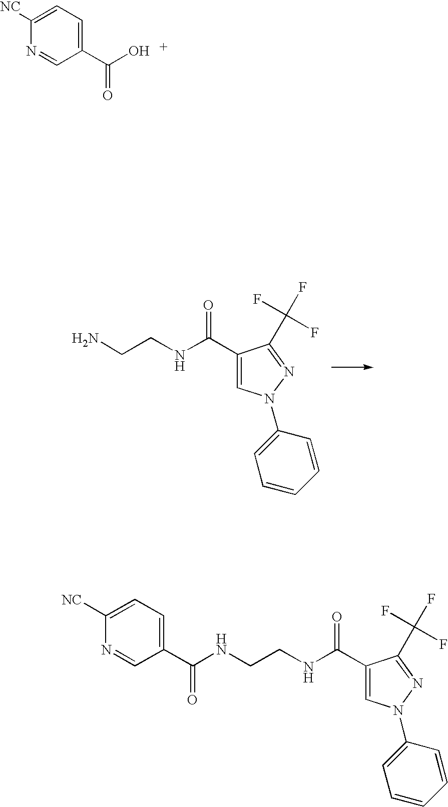

A mixture of N-(2-aminoethyl)-1-phenyl-3-(trifluoromethyl)-1H-pyrazole-4-carboxamide (1.00 g, 3.35 mmol), 6-cyanonicotinic acid (0.55 g, 3.69 mmol), EDAC.HCl (0.84 g, 4.36 mmol), HOBt.H₂O (0.67 g, 4.36 mmol) and CH₃CN (20 mL) was stirred at room temperature for 16 hours. The mixture was diluted with EtOAc, washed with saturated aqueous sodium bicarbonate and brine. The organic layer was then dried and concentrated. The resulting solid was washed with EtOAc and dried to give 6-cyano-N-(2-(1-phenyl-3-(trifluoromethyl)-1H-pyrazole-4-carboxamido)ethyl)nicotinamide (1.00 g, 70%) as a beige solid: ¹H NMR (400 MHz, CDCl₃) δ 3.68-3.82 (4H, m), 6.66 (1H, br), 7.44 (1H, t, J=7.6 Hz), 7.53 (2H, t, J=7.6 Hz), 7.71 (2H, d, J=7.6 Hz), 7.79 (1H, d, J=8.0 Hz), 7.98 (1H, br), 8.31 (1H, dd, J=2.4, 8.0 Hz), 8.47 (1H, s), 9.16 (1H, d, J=2.4 Hz); m/z (APCI pos) 428.9 (25%) (M+H).

### Example A95

### 6-(N′-Hydroxycarbamimidoyl)-N-(2-(1-phenyl-3-(trifluoromethyl)-1H-pyrazole-4-carboxamido)ethyl)nicotinamide

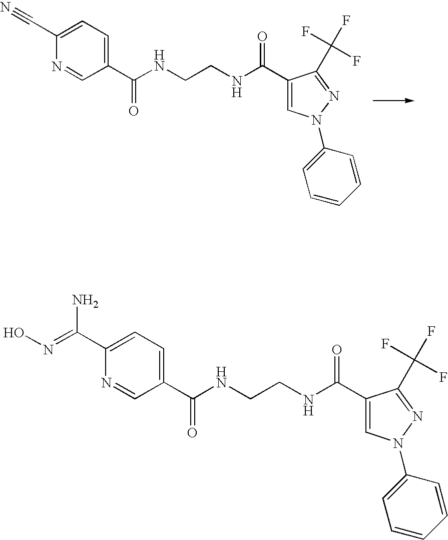

A mixture of 6-cyano-N-(2-(1-phenyl-3-(trifluoromethyl)-1H-pyrazole-4-carboxamido)ethyl)nicotinamide (0.50 g, 1.17 mmol), sodium bicarbonate (0.20 g, 2.3 mmol), hydroxylamine hydrochloride (0.16 g, 2.3 mmol) and EtOH (10 mL) was refluxed for 3 hours. After cooling, the mixture was diluted with EtOAc, washed with water and brine, dried, and concentrated. The residue was recrystallized from EtOAc to give 6-(N′-hydroxycarbamimidoyl)-N-(2-(1-phenyl-3-(trifluoromethyl)-1H-pyrazole-4-carboxamido)ethyl)nicotinamide as colorless crystals (0.35 g, 65%): ¹H NMR (400 MHz, DMSO-d₆) δ 3.45 (4H, s), 5.91 (2H, s), 7.48 (1H, t, J=7.6 Hz), 7.61 (2H, t, J=8.4 Hz), 7.81 (2H, dd, J=0.8, 8.4 Hz), 7.93 (1H, dd, J=0.8, 8.4 Hz), 8.19 (1H, dd, J=2.0, 8.4 Hz), 8.50 (1H, br), 8.82 (1H, br), 8.98 (1H, dd, J=0.8, 2.0 Hz), 9.06 (1H, d, J=0.8 Hz), 10.09 (1H, s); m/z (APCI pos) 462.1 (100%) (M+H).

### Example A96

### 6-(1,2,4-Oxadiazol-3-yl)-N-(2-(1-phenyl-3-(trifluoromethyl)-1H-pyrazole-4-carboxamido)ethyl)nicotinamide

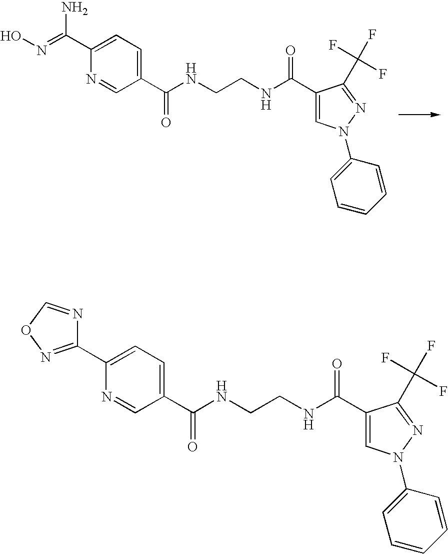

A mixture of 6-(N′-hydroxycarbamimidoyl)-N-(2-(1-phenyl-3-(trifluoromethyl)-1H-pyrazole-4-carboxamido)ethyl)nicotinamide (0.10 g, 0.22 mmol), boron trifluoride diethylether complex (0.028 mL, 0.22 mmol) and trimethyl orthoformate (2 mL) was stirred at 110° C. for 30 minutes. After cooling to room temperature, the mixture was diluted with EtOAc and washed with saturated aqueous sodium bicarbonate and brine. The organic layer was then dried and concentrated in vacuo. The solid was removed by filtration and the filtrate was concentrated in vacuo. The residue was purified by silica gel column chromatography (EtOAc-EtOAc/MeOH=10/1) to afford 6-(1,2,4-oxadiazol-3-yl)-N-(2-(1-phenyl-3-(trifluoromethyl)-1H-pyrazole-4-carboxamido)ethyl)nicotinamide (0.022 g, 22%) as colorless crystals: ¹H NMR (400 MHz, DMSO-d₆) δ 3.47 (4H, s), 7.48 (1H, t, J=7.6 Hz), 7.61 (2H, t, J=7.6 Hz), 7.81 (2H, d, J=7.6 Hz), 8.22 (1H, d, J=8.0 Hz), 8.42 (1H, dd, J=2.0, 8.0 Hz), 8.52 (1H, br), 8.98 (1H, br), 9.07 (1H, s), 9.18 (1H, d, J=2.0 Hz), 9.83 (1H, s); m/z (APCI pos) 472.1 (100%) (M+H).

### Example A97

### tert-Butyl 5-((2-(1-phenyl-3-(trifluoromethyl)-1H-pyrazole-4-carboxamido)ethyl)carbamoyl)pyridin-2-ylcarbamate

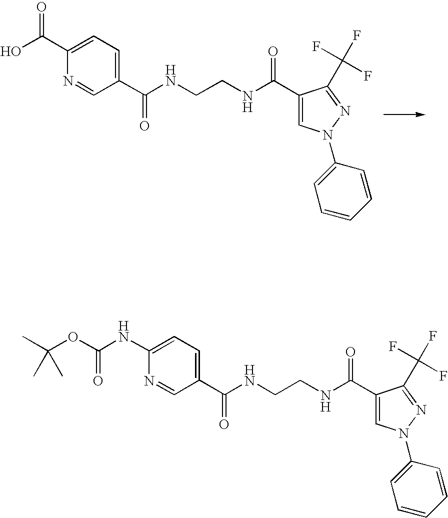

A mixture of 5-((2-(1-phenyl-3-(trifluoromethyl)-1H-pyrazole-4-carboxamido)ethyl)carbamoyl)picolinic acid (1.00 g, 2.24 mmol), diphenylphosphoryl azide (0.52 mL, 2.35 mmol), triethylamine (0.33 mL, 2.35 mmol) and t-BuOH (20 mL) was stirred at 90° C. in a sealed tube for 3 days. After cooling, the mixture was concentrated and recrystallized from EtOAc to give tert-butyl 5-((2-(1-phenyl-3-(trifluoromethyl)-1H-pyrazole-4-carboxamido)ethyl)carbamoyl)pyridin-2-ylcarbamate (0.53 g, 46%) as colorless crystals: ¹H NMR (400 MHz, CDCl₃) δ 1.53 (9H, s), 3.64-3.78 (4H, m), 6.82 (1H, br), 7.24 (1H, br), 7.42 (1H, t, J=7.6 Hz), 7.51 (2H, t, J=7.6 Hz), 7.65 (1H, s), 7.70 (2H, d, J=8.0 Hz), 8.01 (1H, d, J=8.8 Hz), 8.08 (1H, dd, J=2.0, 8.8 Hz), 8.44 (1H, s), 8.73 (1H, d, J=2.0 Hz); m/z (APCI pos) 518.8 (35%) (M+H).

### Example A98

### N-(2-(1-Phenyl-3-(trifluoromethyl)-1H-pyrazole-4-carboxamido)ethyl)-6-(5-(trifluoromethyl)-1,2,4-oxadiazol-3-yl)nicotinamide

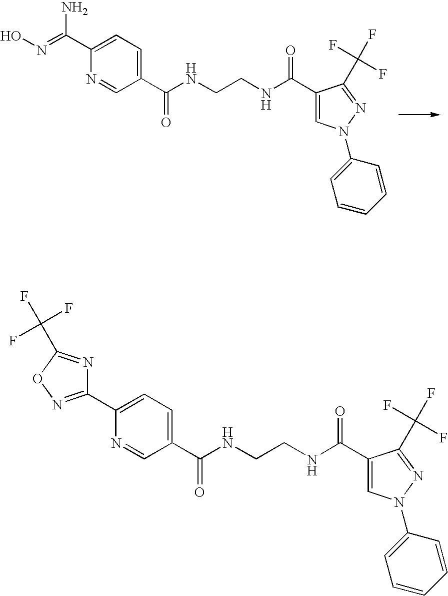

A mixture of 6-(N′-hydroxycarbamimidoyl)-N-(2-(1-phenyl-3-(trifluoromethyl)-1H-pyrazole-4-carboxamido)ethyl)nicotinamide (0.10 g, 0.22 mmol), trifluoroacetic anhydride (0.15 mL, 1.08 mmol) and pyridine (2 mL) was stirred at 100° C. for 6 hours. After cooling to room temperature, the mixture was concentrated in vacuo. The residue was purified by silica gel column chromatography (EtOAc) to afford N-(2-(1-phenyl-3-(trifluoromethyl)-1H-pyrazole-4-carboxamido)ethyl)-6-(5-(trifluoromethyl)-1,2,4-oxadiazol-3-yl)nicotinamide (0.065 g, 56%) as colorless crystals: ¹H NMR (400 MHz, CDCl₃) δ 3.70-3.82 (4H, m), 6.73 (1H, br), 7.43 (1H, m), 7.48-7.56 (2H, m), 7.69-7.75 (2H, m), 7.93 (1H, br), 8.27 (1H, dd, J=0.8, 8.0 Hz), 8.39 (1H, dd, J=2.4, 8.0 Hz), 8.50 (1H, d, J=0.8 Hz), 9.27 (1H, dd, J=0.8, 2.4 Hz); m/z (APCI pos) 540.0 (100%) (M+H).

### Example A99

### 6-Acetamido-N-(2-(1-phenyl-3-(trifluoromethyl)-1H-pyrazole-4-carboxamido)ethyl)nicotinamide

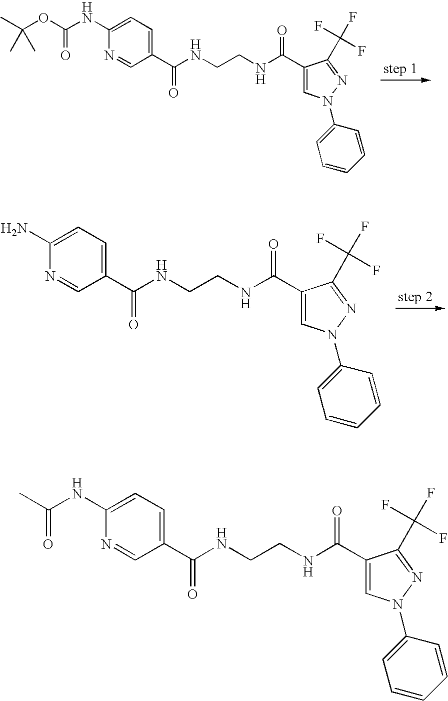

### Step 1

tert-Butyl 5-((2-(1-phenyl-3-(trifluoromethyl)-1H-pyrazole-4-carboxamido)ethyl)carbamoyl)pyridin-2-ylcarbamate (0.450 g, 0.868 mmol) was dissolved in TFA (8 mL). The solution was stirred at room temperature for 2 hours. The solution was concentrated, diluted in EtOAc and washed with saturated aqueous potassium carbonate and brine. The organic layer was then dried, and concentrated to give 6-amino-N-(2-(1-phenyl-3-(trifluoromethyl)-1H-pyrazole-4-carboxamido)ethyl)nicotinamide (0.270 g, 74%) as a white solid: m/z (APCI pos) 419.0 (100%) (M+H).

### Step 2

6-Amino-N-(2-(1-phenyl-3-(trifluoromethyl)-1H-pyrazole-4-carboxamido)ethyl)nicotinamide (0.15 g, 0.35 mmol) in DCM (2 mL) was charged with triethylamine (0.12 ml, 0.84 mmol). The solution was cooled to 0° C. Acetyl chloride (0.03 ml, 0.42 mmol) was added and the solution was warmed to room temperature. The solution was quenched with water, extracted with EtOAc, dried and concentrated. The residue was then diluted in MeOH and 2N NaOH and the mixture was stirred at room temperature for 30 minutes. The solution was extracted with EtOAc, dried, and concentrated to afford 6-acetamido-N-(2-(1-phenyl-3-(trifluoromethyl)-1H-pyrazole-4-carboxamido)ethyl)nicotinamide (0.090 g, 69%): ¹H NMR (400 MHz, DMSO-d₆) δ 2.11 (s, 3H), 3.42 (br, 4H), 7.47 (t, J=7.6 Hz, 1H), 7.61 (t, J=8.4 Hz, 2H), 7.80 (d, J=7.6 Hz, 2H), 8.10-8.20 (m, 2H), 8.49 (s, 1H), 8.65 (s, 1H), 8.76 (d, J=1.6 Hz, 1H), 9.06 (s, 1H), 10.8 (s, 1H); m/z (APCI pos) 461.1 (M+H).

### Example A102

### 2-tert-Butyl-N-(2-(1-phenyl-3-(trifluoromethyl)-1H-pyrazole-4-carboxamido)ethyl)imidazo[1,2-a]pyridine-6-carboxamide

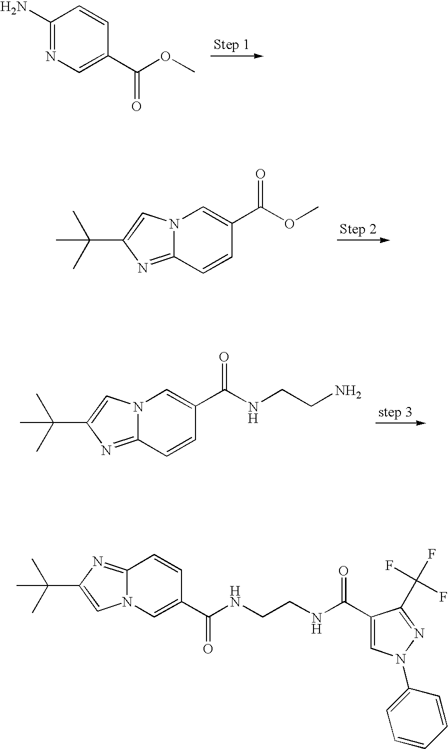

### Step 1

A mixture of methyl 6-aminonicotinate (1.50 g, 9.86 mmol), 1-bromo-3,3-dimethylbutan-2-one (1.77 g, 9.86 mmol), and NaHCO₃ (0.828 g, 9.86 mmol) in MeOH (30 mL) was heated at reflux for 18 hours. The mixture was cooled and concentrated to a residue. The residue was diluted with AcOEt, and the solution was washed with water and brine, dried over Na₂SO₄, and concentrated to a residue. This residue was crystallized from AcOEt/EtOH to afford methyl 2-tert-butylimidazo[1,2-a]pyridine-6-carboxylate (0.690 g, 30%) as a solid: m/z (APCI pos) 233 [M+H].

### Step 2

Methyl 2-tert-butylimidazo[1,2-a]pyridine-6-carboxylate (0.100 g, 0.431 mmol) was dissolved in ethylenediamine (2 mL). The solution was heated at 80° C. for 3 hours. The solution was concentrated and azeotroped with toluene to afford 0.126 g of the crude N-(2-aminoethyl)-2-tert-butylimidazo[1,2-a]pyridine-6-carboxamide. Material was taken on as is: m/z (APCI pos) 261.2 (100%) (M+H)

### Step 3

N-(2-Aminoethyl)-2-tert-butylimidazo[1,2-a]pyridine-6-carboxamide (0.126 g, 0.484 mmol), 1-phenyl-3-(trifluoromethyl)-1H-pyrazole-4-carboxylic acid (0.149 g, 0.581 mmol), HOBt.H₂O (0.0889 g, 0.581 mmol) and EDAC.HCl (0.111 g, 0.581 mmol) were dissolved in DMF (2 mL). DIPEA (0.253 ml, 1.45 mmol) was added last. The reaction was stirred at room temperature overnight. The solution was quenched with water, and extracted with EtOAc. The organic layer was dried, and concentrated. Purification by reverse phase HPLC and recrystallization with EtOAc gave 2-tert-butyl-N-(2-(1-phenyl-3-(trifluoromethyl)-1H-pyrazole-4-carboxamido)ethyl)imidazo[1,2-a]pyridine-6-carboxamide as a white solid (0.037 g, 15%): m/z (APCI pos) 499 (M+H); ¹H NMR (400 MHz, DMSO-d₆) δ 1.32 (s, 9H), 3.44 (s, 4H), 7.46-7.53 (m, 2H), 7.58-7.62 (m, 3H), 7.78 (s, 1H), 7.81 (d, J=7.6 Hz, 2H), 8.51 (br s, 1H), 8.66 (br s, 1H), 9.01 (s, 1H), 9.07 (s, 1H).

### Example A103

### 6-(5-Isopropyl-1,2,4-oxadiazol-3-yl)-N-(2-(1-phenyl-3-(trifluoromethyl)-1H-pyrazole-4-carboxamido)ethyl)nicotinamide

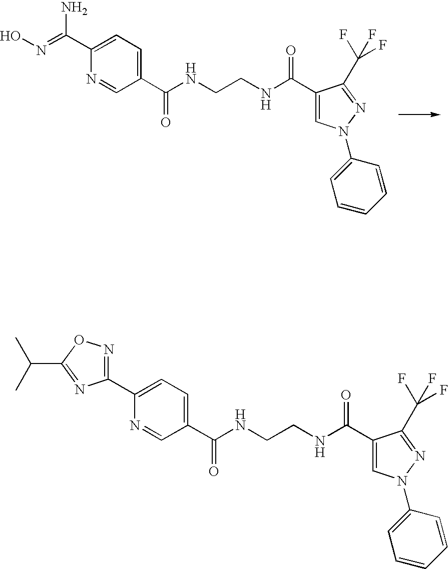

The title compound was prepared from 6-(N′-hydroxycarbamimidoyl)-N-(2-(1-phenyl-3-(trifluoromethyl)-1H-pyrazole-4-carboxamido)ethyl)nicotinamide and isobutyryl chloride using a similar procedure described in Example A98. Colorless crystals. ¹H NMR (400 MHz, CDCl₃) δ 1.49 (6H, d, J=7.2 Hz), 3.34 (1H, m), 3.68-3.80 (4H, m), 6.80 (1H, br), 7.42 (1H, t, J=7.6 Hz), 7.51 (2H, t, J=7.6 Hz), 7.72 (2H, d, J=7.6 Hz), 7.83 (1H, br), 8.22 (1H, d, J=8.0 Hz), 8.33 (1H, dd, J=2.4, 8.0 Hz), 8.50 (1H, s), 9.23 (1H, m); m/z (APCI pos) 514.2 (100%) (M+H).

### Example A104

### 6-Hydroxy-N-(2-(1-phenyl-3-(trifluoromethyl)-1H-pyrazole-4-carboxamido)ethyl)nicotinamide

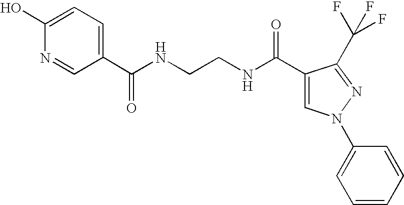

The title compound was obtained in the reaction that afforded the compound of Example A42. Colorless solid. ¹H NMR (400 MHz, DMSO-d₆) δ 3.32-3.40 (4H, m), 6.34 (1H, d, J=9.6 Hz), 7.48 (1H, t, J=7.6 Hz), 7.61 (2H, t, J=7.6 Hz), 7.81 (2H, dd, J=0.8, 8.4 Hz), 7.85 (1H, dd, J=2.4, 9.6 Hz), 7.98 (1H, d, J=2.4 Hz), 8.36 (1H, br), 8.45 (1H, br), 9.05 (1H, d, J=0.8 Hz), 11.95 (1H, s); m/z (APCI pos) 420.0 (100%) (M+H).

### Example A105

### 6-(5-Isopropyl-1,3,4-oxadiazol-2-yl)-N-(2-(1-phenyl-3-(trifluoromethyl)-1H-pyrazole-4-carboxamido)ethyl)nicotinamide

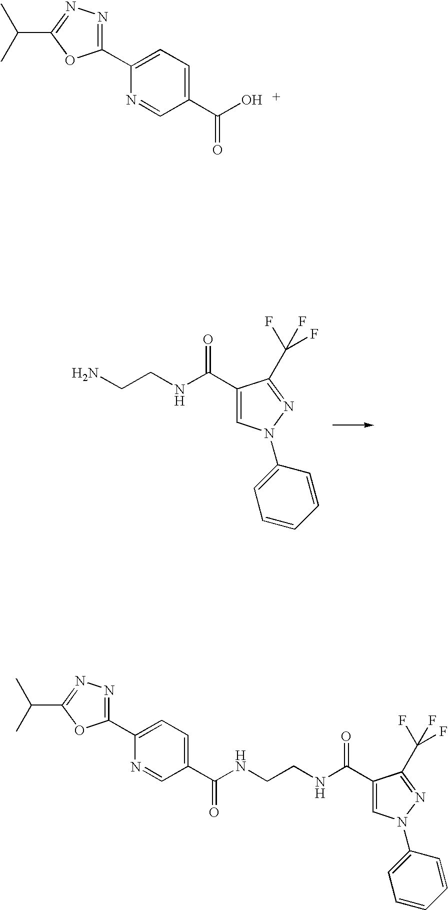

A mixture of 6-(5-isopropyl-1,3,4-oxadiazol-2-yl)nicotinic acid (0.13 g, 0.54 mmol), N-(2-aminoethyl)-1-phenyl-3-(trifluoromethyl)-1H-pyrazole-4-carboxamide (0.18 g, 0.59 mmol), EDAC.HCl (0.15 g, 0.80 mmol), HOBt.H₂O (0.12 g, 0.80 mmol) and triethylamine (0.22 mL, 1.6 mmol) in CH₃CN (10 mL) was stirred for 16 h at room temperature. The mixture was diluted with AcOEt, washed successively with 1N HCl, saturated aqueous NaHCO₃ and brine, dried and concentrated in vacuo. The residue was purified by silica gel column chromatography (AcOEt to AcOEt/MeOH=10/1) and recrystallized from AcOEt to give 6-(5-isopropyl-1,3,4-oxadiazol-2-yl)-N-(2-(1-phenyl-3-(trifluoromethyl)-1H-pyrazole-4-carboxamido)ethyl)nicotinamide (0.14 g, 51%) as colorless crystals. ¹H NMR (400 MHz, CDCl₃) δ 1.49 (6H, d, J=7.2 Hz), 3.30 (1H, m), 3.70-3.82 (4H, m), 6.80 (1H, br), 7.42 (1H, t, J=7.2 Hz), 7.51 (2H, t, J=8.0 Hz), 7.71 (2H, d, J=8.0 Hz), 7.87 (1H, br), 8.26-8.36 (2H, m), 8.50 (1H, s), 9.22 (1H, d, J=1.2 Hz); m/z (APCI pos) 514.2 (100%) (M+H).

### 6-(5-Isopropyl-1,3,4-oxadiazol-2-yl)nicotinic acid

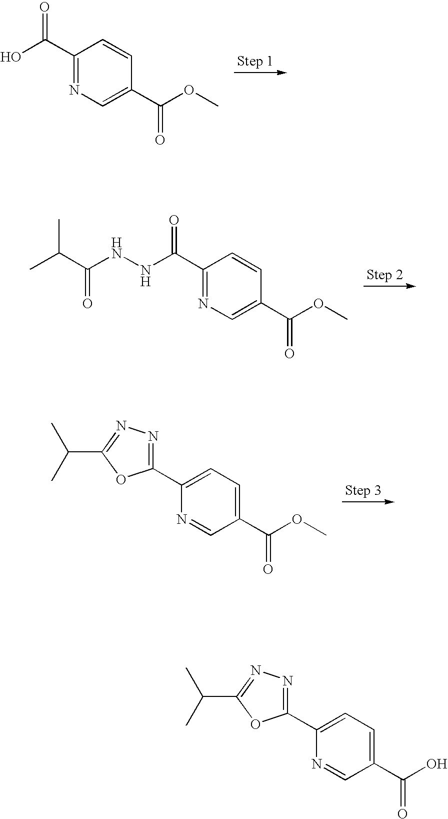

### Step 1

A mixture of 5-(methoxycarbonyl)picolinic acid (0.50 g, 2.62 mmol), isobutyrohydrazide (0.30 g, 2.88 mmol), EDAC.HCl (0.75 g, 3.93 mmol), HOBt.H₂O (0.60 g, 3.93 mmol) and triethylamine (0.73 mL, 5.24 mmol) in CH₃CN (20 mL) was stirred for 16 h at room temperature. The mixture was diluted with AcOEt, washed successively with 1N HCl, saturated aqueous NaHCO₃ and brine, dried and concentrated in vacuo to give methyl 6-(N′-isobutyrylhydrazinocarbonyl)nicotinate (0.51 g, 73%) as yellow solid. ¹H NMR (400 MHz, CDCl₃) δ 1.27 (6H, d, J=6.8 Hz), 2.57 (1H, m), 3.99 (3H, s), 8.22 (1H, d, J=8.0 Hz), 8.38-8.50 (2H, m), 9.17 (1H, d, J=2.0 Hz), 10.29 (1H, s); m/z (APCI pos) 266.0 (100%) (M+H).

### Step 2

A mixture of methyl 6-(N′-isobutyrylhydrazinocarbonyl)nicotinate (0.51 g, 1.92 mmol), phosphorus oxychloride (0.54 mL, 5.77 mmol) and CH₃CN (10 mL) was heated at 80° C. for 16 h. After cooling to room temperature, the mixture was diluted with AcOEt, washed with saturated aqueous NaHCO₃ and brine, dried and concentrated in vacuo. The residue was purified by silica gel column chromatography (hexanes/AcOEt=2/1 to 1/1) to give methyl 6-(5-isopropyl-1,3,4-oxadiazol-2-yl)nicotinate (0.28 g, y. 59%) as pale yellow solid. ¹H NMR (400 MHz, CDCl₃) δ 1.50 (6H, d, J=6.8 Hz), 3.34 (1H, m), 4.01 (3H, s), 8.34 (1H, d, J=8.4 Hz), 8.47 (1H, dd, J=2.0, 8.4 Hz), 9.35 (1H, d, J=2.0 Hz); m/z (APCI pos) 248.2 (100%) (M+H).

### Step 3

A mixture of methyl 6-(5-isopropyl-1,3,4-oxadiazol-2-yl)nicotinate (0.28 g, 1.13 mmol), 1N NaOH (2.3 mL, 2.3 mmol) and MeOH (10 mL) was stirred at 50° C. for 16 h. After cooling to room temperature, the mixture was concentrated in vacuo. The residue was neutralized with 1N HCl and extracted with AcOEt. The AcOEt extract was washed with water and brine, dried and concentrated in vacuo to give 6-(5-isopropyl-1,3,4-oxadiazol-2-yl)nicotinic acid (0.13 g, 48%) as colorless solid. ¹H NMR (400 MHz, CDCl₃) δ 1.51 (6H, d, J=7.2 Hz), 3.36 (1H, m), 8.40 (1H, d, J=8.4 Hz), 8.55 (1H, dd, J=2.0, 8.4 Hz), 9.43 (1H, d, J=2.0 Hz); m/z (APCI pos) 234.2 (M+H).

### Example B1

### N-(2-(4-Ethoxybenzamido)ethyl)-1-(1H-indol-5-yl)-3-(trifluoromethyl)-1H-pyrazole-4-carboxamide

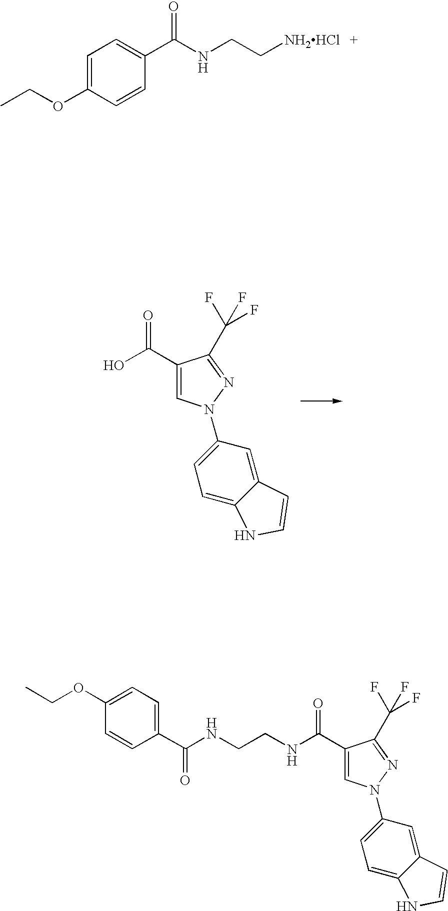

To a 5 ml reacti-vial were added 1-(1H-indol-5-yl)-3-(trifluoromethyl)-1H-pyrazole-4-carboxylic acid (0.162 g, 0.550 mmol), HOBt.H₂O (0.0842 g, 0.550 mmol), and EDAC.HCl (0.105 g, 0.550 mmol). These were dissolved in DMF (1 ml) and N-(2-aminoethyl)-4-ethoxybenzamide hydrochloride (0.122 g, 0.50 mmol) was added followed by DIPEA (0.129 g, 1.00 mmol). The mixture was stirred for 16 hours. The solvent was removed and the residue was dissolved in DCM and washed with water (2×). The combined aqueous layers were back extracted, and the combined organic layers were washed with brine, dried (sodium sulfate), and concentrated in vacuo. The residue was then purified by silica gel column chromatography (3% MeOH/DCM) to give N-(2-(4-ethoxybenzamido)ethyl)-1-(1H-indol-5-yl)-3-(trifluoromethyl)-1H-pyrazole-4-carboxamide (12 mg, 5%) as a solid: ¹H NMR (400 MHz, DMSO-d₆) δ 1.33 (t, 3H, J=7.0 Hz), 3.40 (m, 4H), 4.08 (q, 2H, J=7.0 Hz), 6.57 (m, 1H), 6.97 (d, 2H, J=9.0 Hz), 7.53 (m, 3H), 7.82 (d, 2H, J=9.0 Hz), 7.93 (m, 1H), 8.44 (m, 2H), 8.96 (s, 1H), 11.42 (br, 1H); m/z (APCI pos) 486.0 (10%) (M+H).

Compounds of the following structures were prepared from N-(2-aminoethyl)-4-ethoxybenzamide hydrochloride and the corresponding acids, using a similar method to that described above.

| Ex # | Structure | Name | Physical Data |
|---|---|---|---|
| B2 | 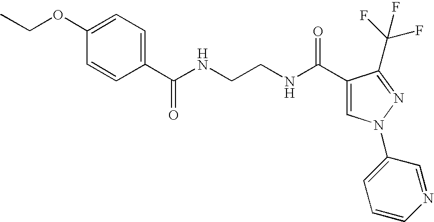 | N-(2-(4- ethoxybenzami do)ethyl)-1- (pyridin-3- yl)-3- (trifluorometh- yl)-1H- pyrazole-4- carboxamide | Solid (39%); ¹H NMR (400 MHz, DMSO-d₆) δ 1.33(t, 3 H, J = 7.0 Hz), 3.41(m, 4 H), 4.08(q, 2 H, J = 7.0 Hz), 6.97(d, 2 H, J = 9.0 Hz), 7.65(m, 1 H), 7.81(d, 2 H, J = 9.0 Hz), 8.23(m, 1 H), 8.43(m, 1 H), 8.51(m, 1 H), 8.68(m, 1 H), 9.06(m, 1 H), 9.13(m, 1 H); m/z (APCI pos) 448.0 (10%) (M + H). |
| B3 | 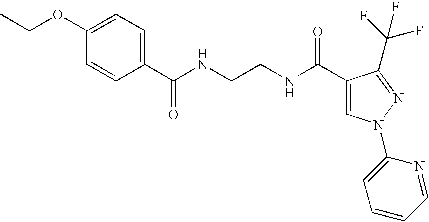 | N-(2-(4- ethoxybenzami do)ethyl)-1- (pyridin-2- yl)-3- (trifluorometh- yl)-1H- pyrazole-4- carboxamide | Solid (30%); ¹H NMR (400 MHz, DMSO-d₆) δ 1.33(t, 3 H, J = 7.0 Hz), 3.40(m, 4 H), 4.08(q, 2 H, J = 7.0 Hz), 6.97(d, 2 H, J = 9.0 Hz), 7.53(m, 1 H), 7.82(d, 2 H, J = 9.0 Hz), 7.99(m, 1 H), 8.10(m, 1 H), 8.43(m, 1 H), 8.58(m, 1 H), 8.66(m, 1 H), 9.38(s, 1 H); m/z (APCI pos) 448.0 (10%) (M + H). |
| B4 | 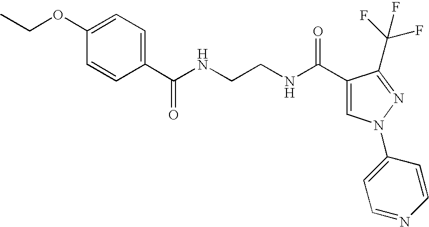 | N-(2-(4- ethoxybenzami do)ethyl)-1- (pyridin-4- yl)-3- (trifluorometh- yl)-1H- pyrazole-4- carboxamide | Solid (55%); ¹H NMR (400 MHz, DMSO-d₆) δ 1.33(t, 3 H, J = 7.0 Hz), 3.41(m, 4 H), 4.08(q, 2 H, J = 7.0 Hz), 6.97(d, 2 H, J = 9.0 Hz), 7.82(d, 2 H, J = 9.0 Hz), 7.85(m, 2 H), 8.43(s, 1 H), 8.55(s, 1 H), 8.78(m, 2 H), 9.26(s, 1 H); m/z (APCI pos) 448.1 (10%) (M + H). |

### Example B5

### N-(2-(4-Ethoxybenzamido)ethyl)-1-(pyrimidin-2-yl)-3-(trifluoromethyl)-1H-pyrazole-4-carboxamide

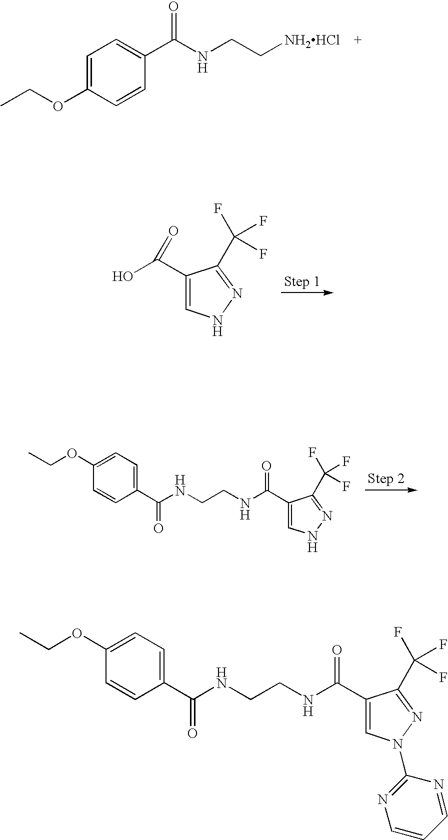

### Step 1

To a 500 ml round-bottom flask were added 3-(trifluoromethyl)-1H-pyrazole-4-carboxylic acid (18.8 g, 104 mmol), HOBt.H₂O (16 g, 104 mmol), and EDAC-HCl (20 g, 104 mmol). These were dissolved in DMF, and N-(2-aminoethyl)-4-ethoxybenzamide hydrochloride (23 g, 95 mmol) was added as a solid. Triethylamine (26 ml, 189 mmol) was then added and the mixture was stirred for 16 hours. The solvent was removed, the residue was dissolved in DCM and the solution was washed with water (2×). The combined aqueous layers were back extracted, and the combined organic layers were washed with brine, dried (sodium sulfate), and concentrated in vacuo. The residue was then purified by silica gel column chromatography (4% MeOH/DCM) and then triturated with ethyl acetate to give N-(2-(4-ethoxybenzamido)ethyl)-3-(trifluoromethyl)-1H-pyrazole-4-carboxamide (12 g, 34%) as a solid: ¹H NMR (CDCl₃) δ 1.34 (t, 3H, J=7.0 Hz), 3.36 (m, 4H), 4.07 (q, 2H, J=7.0 Hz), 6.96 (m, 2H), 7.80 (m, 2H), 8.34 (m, 2H), 8.41 (m, 1H), 13.74 (br, 1H).

### Step 2

According to the procedure of Buchwald et al. (J. Am. Chem. Soc. 2001, 123, 7727-9), to a 50 mL sealed tube were added N-(2-(4-ethoxybenzamido)ethyl)-3-(trifluoromethyl)-1H-pyrazole-4-carboxamide (0.100 g, 0.270 mmol), (1R,2R)—N1,N2-dimethylcyclohexane-1,2-diamine (0.008 g, 0.054 mmol), copper(I) iodide (0.003 g, 0.014 mmol), potassium carbonate (0.075 g, 0.54 mmol), and 2-bromopyrimidine (0.047 g, 0.3 mmol). The mixture was stirred for 16 hours at 11° C., cooled to room temperature, and filtered through celite. The solvent was removed under vacuum and the residue was purified by silica gel column chromatography (9:0.5:0.5 DCM/EtOAc/MeOH) to give N-(2-(4-ethoxybenzamido)ethyl)-1-(pyrimidin-2-yl)-3-(trifluoromethyl)-1H-pyrazole-4-carboxamide (100 mg, 83%) as a solid: ¹H NMR (400 MHz, DMSO-d₆) δ 1.33 (t, 3H, J=7.0 Hz), 3.40 (s, 4H), 4.08 (q, 2H, J=7.0 Hz), 6.97 (d, 2H, J=9.0 Hz), 7.66 (t, 1H, J=5.0 Hz), 7.82 (d, 2H, J=9.0 Hz), 8.44 (s, 1H), 8.69 (s, 1H), 8.98 (d, 2H, J=4.7 Hz), 9.42 (s, 1H); m/z (APCI pos) 449.1 (100%) (M+H).

The compound of the following structure was prepared using a similar method to that described above.

| Ex # | Structure | Name | Physical Data |
|---|---|---|---|
| B6 | 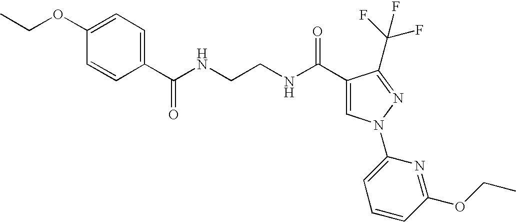 | N-(2-(4- ethoxybenzami do)ethyl)-1- (6- ethoxypyridin- 2-yl)-3- (trifluorometh- yl)-1H- pyrazole-4- carboxamide | Solid (45%); ¹H NMR (400 MHz, DMSO-d₆) δ 1.36(m, 6 H), 3.41(s, 4 H), 4.07(q, 2 H, J = 7.0 Hz), 4.44(q, 2 H, J = 7.0 Hz), 6.91(d, 1 H, J = 8.2 Hz), 6.96 (d, 2 H, J = 8.6 Hz), 7.51(d, 1 H, J = 7.8 Hz), 7.82(d, 2 H, J = 8.6 Hz), 7.95(t, 1 H, 8.20 Hz), 8.47(s, 1 H), 8.69(s, 1 H), 9.27(s, 1 H); m/z (APCI pos) 492.1 (100%) (M + H). |

### Example C1

### (S)-3-(4-Ethoxybenzamido)-2-(1-phenyl-3-(trifluoromethyl)-1H-pyrazole-4-carboxamido)propanoic acid

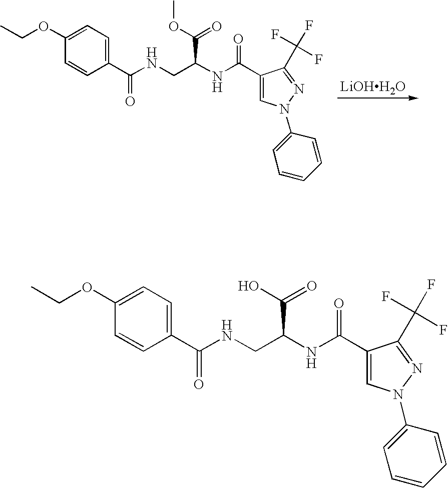

To (S)-methyl 3-(4-ethoxybenzamido)-2-(1-phenyl-3-(trifluoromethyl)-1H-pyrazole-4-carboxamido)propanoate (100 mg, 0.2 mmol) in MeOH (5 mL) was added lithium hydroxyde monohydrate (83 mg, 2 mmol). The mixture was stirred for 48 hours at room temperature and concentrated. Water (10 mL) was added and the suspension was adjusted to acidic with 2N HCl. The aqueous layer was extracted with DCM (5×20 mL). The combined organic layers were dried over MgSO₄ and concentrated to yield (S)-3-(4-ethoxybenzamido)-2-(1-phenyl-3-(trifluoromethyl)-1H-pyrazole-4-carboxamido)propanoic acid as a white solid (65 mg, 67%): ¹H NMR (400 MHz, DMSO-d₆) δ 1.32 (t, 3H, J=7.0 Hz), 3.62 (m, 1H), 3.76 (m, 1H), 4.07 (q, 2H, J=7.0 Hz), 4.62 (m, 1H), 6.96 (m, 2H), 7.49 (m, 1H), 7.62 (m, 2H), 7.75-7.82 (m, 4H), 8.46 (t, 1H, J=5.6 Hz), 8.60 (d, 1H, J=5.6 Hz), 9.14 (s, 1H), 12.80 (s, 1H); m/z (APCI pos) 491.0 (100%) (M+H).

Compounds of the following structures were prepared from the corresponding esters, using a similar method to that described above.

| Ex # | Structure | Name | Physical Data |
|---|---|---|---|
| C6 | 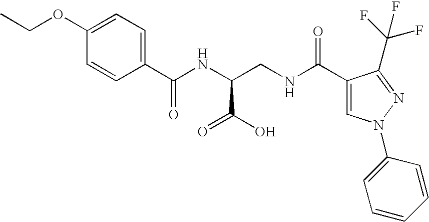 | (S)-2-(4- ethoxybenzamid o)-3-(1- phenyl-3- (trifluorometh yl)-1H- pyrazole-4- carboxamido)pro- panoic acid | White solid (87%); ¹H NMR (400 MHz, CDCl₃) δ 1.40(t, 3 H, J = 7.0 Hz), 3.82(m, 1 H), 3.89(q, 2 H, J = 7.0 Hz), 4.04(m, 1 H), 4.69(m, 1 H), 6.79(d, 2 H), 7.28-7.42(m, 3 H), 7.58(d, 2 H), 7.75(m, 3 H), 8.51(s, 1 H), 8.66(d, 1 H); m/z (APCI pos) 491.0 (30%) (M + H). |
| C17 | 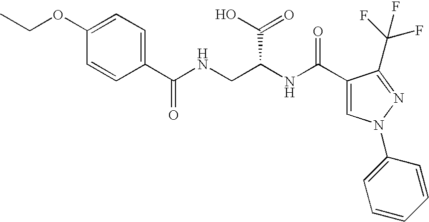 | (R)-3-(4- ethoxybenzamid o)-2-(1- phenyl-3- (trifluorometh yl)-1H- pyrazole-4- carbox amido)propanoic acid | White solid (79%); ¹H NMR(400 MHz, DMSO-d₆) δ 1.32(t, 3 H, J = 7.0 Hz), 3.61(ddd, 1 H, J = 13.3, 7.4, 6.2 Hz), 3.76(dt, 1 H, J = 13.6, 5.8 Hz), 4.06 (q, 2 H, J = 7.0 Hz), 4.62(dt, 1 H, J = 7.8, 5.8 Hz), 6.96(d, 2 H, J = 9.0 Hz), 7.49 (tt, 1 H, J = 7.4, 1.6 Hz), 7.58-7.64(m, 2 H), 7.75-7.85(m, 4 H), 8.46(t, 1 H, J = 5.9 Hz), 8.60(d, 1 H, J = 7.8 Hz), 9.15(s, 1 H), 12.85(br, 1 H); m/z (APCI pos) 491.0 (60%) (M + H). |
| C19 | 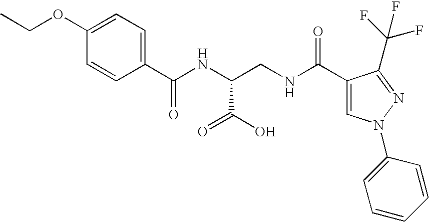 | (R)-2-(4- ethoxybenzamid o)-3-(1- phenyl-3- (trifluorometh yl)-1H- pyrazole-4- carboxamido)pro- panoic acid | White solid (58%); ¹H NMR (400 MHz, DMSO-d₆) δ 1.32(t, 3 H, J = 7.0 Hz), 3.64(ddd, 1 H, J = 13.6, 5.8, 1.9 Hz), 3.81(dt, 1 H, J = 13.6, 5.0 Hz), 4.09(q, 2 H, J = 7.0 Hz), 4.57(dt, 1 H, J = 7.8, 4.7 Hz), 6.98(d, 2 H, J = 9.0 Hz), 7.47(t, 1 H, J = 7.4 Hz), 7.56-7.63(m, 2 H), 7.74-7.88(m, 4 H), 8.55(d, 1 H, J = 7.4 Hz), 8.59(t, 1 H, J = 5.9 Hz), 9.04(s, 1 H), 12.70(br, 1 H); m/z (APCI pos) 491.0 (30%) (M + H). |

### Example C3

### (S)-Methyl 3-(4-ethoxybenzamido)-2-(1-phenyl-3-(trifluoromethyl)-1H-pyrazole-4-carboxamido)propanoate

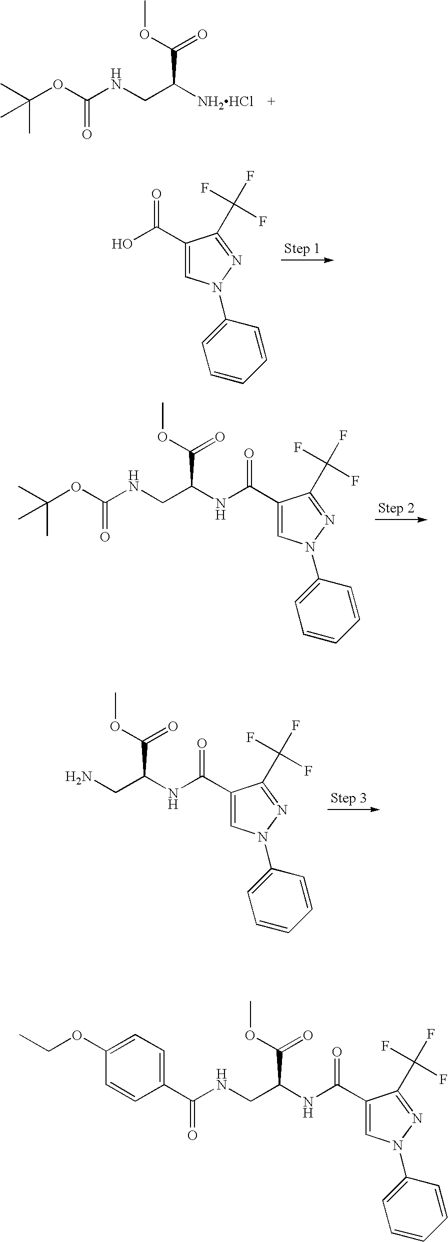

### Step 1

To (S)-methyl 2-amino-3-(tert-butoxycarbonylamino)propanoate hydrochloride (1.0 g, 3.9 mmol) in DCM (50 mL) were added successively 1-phenyl-3-(trifluoromethyl)-1H-pyrazole-4-carboxylic acid (1.0 g, 3.9 mmol), EDAC.HCl (0.9 g, 4.71 mmol), HOBt.H₂O (0.690 g, 5.1 mmol) and triethylamine (0.8 g, 7.9 mmol). The mixture was stirred at room temperature for 12 hours and 2N HCl was added (100 mL). The organic layer was isolated and washed with 10% aqueous K₂CO₃ and brine. The combined organic layers were dried over MgSO₄, concentrated and purified by silica gel column chromatography (DCM/MeOH 99/1). (S)-Methyl 3-(tert-butoxycarbonylamino)-2-(1-phenyl-3-(trifluoromethyl)-1H-pyrazole-4-carboxamido)propanoate was isolated as a white solid (1.2 g, 67%): ¹H NMR (CDCl₃) δ 1.42 (s, 9H), 3.64-3.68 (m, 2H), (s, 3H), 4.74-4.82 (m, 1H), 4.91-4.97 (m, 1H), 7.32-7.38 (m, 1H), 7.39-7.44 (m, 1H), 7.48-7.54 (m, 2H), 7.69-7.72 (m, 2H), 8.44 (br, 1H); m/z (APCI pos) 357.1 (100%) (M+H-Boc).

Compounds of the following structures were prepared from the corresponding Boc-protected diamines and acids, using a similar method to that described above.

| Structure | Name | Physical Data |
|---|---|---|
| 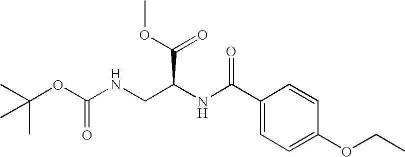 | (S)-methyl 3- (tert- butoxycarbonyl amino)-2-(4- ethoxybenzamid o)propanoate | White solid (60%); ¹H NMR (400 MHz, CDCl₃) δ 1.26(t, 3 H, J = 7.0 Hz), 1.42(s, 9 H), 3.60-3.70(m, 2 H), 3.78(s, 3 H), 4.12(q, 2 H, J = 7.0 Hz), 4.70-4.76(m, 1 H), 6.89-6.94(m, 2 H), 7.70-7.83(m, 2 H); m/z (APCI pos) 267.1 (100%) (M + H − Boc). |
| 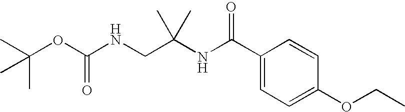 | tert-butyl 2- (4- ethoxybenzamid o)-2- methylpropylcar- bamate | White solid (95%); ¹H NMR (400 MHz, CDCl₃) δ 1.42(t, 3 H, J = 7.0 Hz), 1.47(s, 15 H), 3.26(d, 2 H, J = 6.6 Hz), 4.06(q, 2 H, J = 7.0 Hz), 5.30(t, 1 H, J = 6.6 Hz), 6.85-6.89(m, 2 H), 7.75-7.80(m, 2 H). |
| 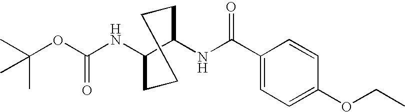 | tert-butyl cis-2-(4- ethoxybenzamid o)cyclohexylcar- bamate | White solid (74%); ¹H NMR (400 MHz, CDCl₃) δ 1.15- 1.45(m, 4 H), 1.32(s, 9 H), 1.42(t, 3 H, J = 7.0 Hz), 1.70-1.82(m, 2 H), 1.99-2.04(m, 1 H), 2.21- 2.29(m, 1 H), 3.45-3.55 (m, 1 H), 3.68-3.78(m, 1 H), 4.06(q, 2 H, J = 7.0 Hz), 4.67(d, 1 H, J = 9.0 Hz), 6.87(m, 2 H), 6.96 (d, 1 H, J = 7.0 Hz), 7.75- 7.81(m, 2 H); m/z (APCI pos) 263.1 (100%) (M − Boc). |
| 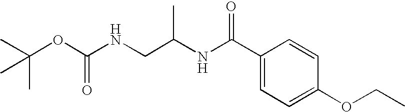 | tert-butyl 2- (4- ethoxybenzamid o)propylcarbamate | White solid (93%); ¹H NMR (400 MHz, CDCl₃) δ 1.23(d, 3 H, J = 6.2 Hz), 1.39(s, 9 H), 1.42(t, 3 H, J = 7.0 Hz), 3.15-3.23(m, 1 H), 3.30-3.40(m, 1 H), 4.06 (q, 2 H, J = 7.0 Hz), 4.14- 4.20(m, 1 H), 5.04(t, 1 H, J = 6.6 Hz), 6.86-6.90(m, 2 H), 6.99(d, 1 H, J = 6.6 Hz), 7.75-7.80(m, 2 H); m/z (APCI pos) 223.1 (100%) (M + H − Boc). |
| 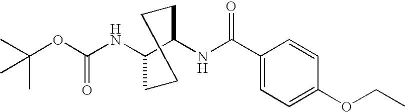 | tert-butyl trans-2-(4- ethoxybenzamid o)cyclohexylcar- bamate | Yellow oil (73%); ¹H NMR (400 MHz, CDCl₃) δ 1.30- 1.80(m, 8 H), 1.41(t, 3 H, J = 7.0 Hz), 1.47(s, 9 H), 2.00-2.10(m, 1 H), 3.94-4.01(m, 1 H), 4.06 (q, 2 H, J = 7.0 Hz), 4.97 (m, 1 H), 6.86-6.90(m, 2 H), 6.74-6.79(m, 2 H); m/z (APCI pos) 263. (10%) (M + H − Boc). |
| 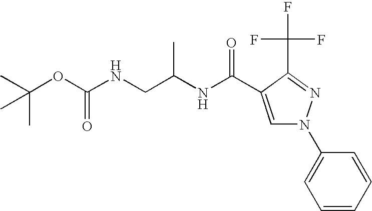 | tert-butyl 2- (1-phenyl-3- (trifluorometh yl)-1H- pyrazole-4- carboxamido)pro- pylcarbamate | White solid (88%); ¹H NMR (400 MHz, CDCl₃) δ 1.25(d, 3 H, J = 6.6 Hz), 1.40(s, 9 H), 3.25-3.30(m, 2 H), 4.16-4.24(m, 1 H), 4.95- 5.01(m, 1 H), 6.74-6.79 (m, 1 H), 7.38-7.43(m, 1 H), 7.47-7.52(m, 2 H), 7.66-7.71(m, 2 H), 8.39 (s, 1 H); m/z (APCI pos) 313.1 (10%) (M + H − Boc). |
| 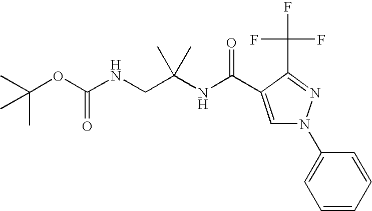 | tert-butyl 2- methyl-2-(1- phenyl-3- (trifluorometh yl)-1H- pyrazole-4- carboxamido)pro- pylcarbamate | White solid (80%); ¹H NMR (400 MHz, CDCl₃) δ 1.45(s, 15 H), 3.30(d, 2 H, J = 6.6 Hz), 5.21(t, 1 H, J = 6.2 Hz), 7.09(br, 1 H), 7.37- 7.42(m, 1 H), 7.46-7.52 (m, 2 H), 7.67-7.71(m, 2 H), 8.37(s, 1 H); m/z (APCI pos) 327.1 (50%) (M + H − Boc). |

### Step 2

To (S)-methyl 3-(tert-butoxycarbonylamino)-2-(1-phenyl-3-(trifluoromethyl)-1H-pyrazole-4-carboxamido)propanoate (1.0 g, 2.2 mmol) was added TFA (10 mL). The mixture was stirred at room temperature for 1 hour and concentrated under vacuum. The residue was dissolved in water (50 mL) and sodium carbonate was added until basic pH. After stirring 1 hour at room temperature, the aqueous layer was extracted with DCM. The combined organic layers were dried over MgSO₄ and concentrated to yield (S)-methyl 3-amino-2-(1-phenyl-3-(trifluoromethyl)-1H-pyrazole-4-carboxamido)propanoate as a white solid (780 mg, quant.): ¹H NMR (CDCl₃) δ 3.17 (dd, 1H, J=13.3, 4.7 Hz), 3.25 (dd, 1H, J=13.3, 3.9 Hz), 3.81 (s, 3H), 4.78 (dt, 1H, J=7.0, 4.3 Hz), 7.17 (br, 1H), 7.39-7.44 (m, 1H), 7.48-7.54 (m, 2H), 7.69-7.73 (m, 2H), 8.49 (s, 1H).

Compounds of the following structures were prepared from the corresponding Boc-protected amines, using a similar method to that described above.

| Structure | Name | Physical Data |
|---|---|---|
| 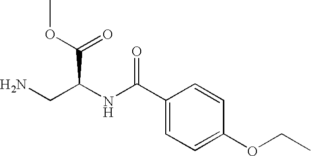 | (S)-methyl 3- amino-2-(4- ethoxybenzamid o)propanoate | Clear paste (quant.); ¹H NMR (400 MHz, DMSO-d₆) δ 1.35(t, 3 H, J = 7.0 Hz), 3.19-3.40(m, 2 H), 3.69 (s, 3 H), 4.10(q, 2 H, J = 7.0 Hz), 4.74-4.80(m, 1 H), 7.00-7.05(m, 2 H), 7.85-7.90(m, 2 H), 8.01 (br, 2 H), 8.20(d, 1 H, J = 8.2 Hz). |
| 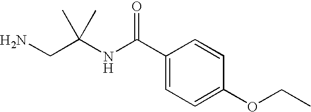 | N-(1-amino-2- methylpropan- 2-yl)-4- ethoxybenzamide | Clear oil (quant.); ¹H NMR (400 MHz, CDCl₃) δ 1.42 (t, 3 H, J = 7.0 Hz), 1.48 (s, 6 H), 3.23-3.29(m, 2 H), 4.04(q, 2 H, J = 7.0 Hz), 6.56(br, 1 H), 6.85- 6.88(m, 2 H), 7.63-7.68 (m, 2 H), 8.44(br, 2 H); m/z (APCI pos) 237.1 (10%) (M + H). |
| 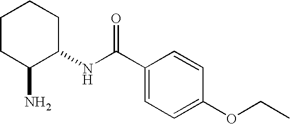 | N-(trans-2- aminocyclohexyl)- 4- ethoxybenzamide | Pink solid (94%); ¹H NMR (400 MHz, CDCl₃) δ 1.00- 1.43(m, 4 H), 1.40(t, 3 H, J = 7.0 Hz), 1.64- 1.74(m, 2 H), 1.96-2.06 (m, 2 H), 3.20(s, 1 H), 3.98-4.05(m, 1 H), 4.00 (q, 2 H, J = 7.0 Hz), 6.77-6.82(m, 2 H), 7.71 (d, 1 H, J = 8.6 Hz), 7.74-7.79(m, 2 H), 7.98 (br, 2 H); m/z (APCI pos) 263.1 (100%) (M + H). |
| 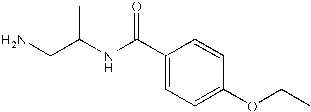 | N-(1- aminopropan-2- yl)-4- ethoxybenzamide | Clear oil (quant.); ¹H NMR (400 MHz, CDCl₃) δ 1.22 (d, 3 H, J = 7.0 Hz), 1.39 (t, 3 H, J = 7.0 Hz), 3.02-3.11(m, 2 H), 3.99 (q, 2 H, J = 7.0 Hz), 4.35-4.43(m,1 H), 6.77- 6.82(m, 2 H), 7.39(d, 1 H, J = 8.6 Hz), 7.64- 7.69(m, 2 H), 8.03(br, 2 H); m/z (APCI pos) 223.1 (100%) (M + H). |
| 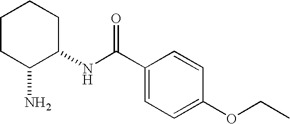 | N-(cis-2- aminocyclohexyl)- 4- ethoxybenzamide | Purple oil (quant.); ¹H NMR (400 MHz, CDCl₃) δ 1.40-1.84(m, 6 H), 1.40 (t, 3 H, J = 7.0 Hz), 3.42-3.47(m, 1 H), 4.00 (q, 2 H, J = 7.0 Hz), 4.40-4.43(m, 1 H), 6.77- 6.82(m, 2 H), 7.14(d, 1 H, J = 8.6 Hz), 7.66- 7.72(m, 2 H), 7.95(br, 2 H); m/z (APCI pos) 263.1 (40%) (M + H). |
| 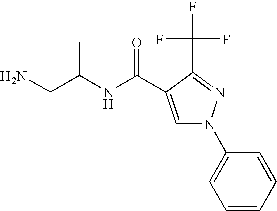 | N-(1- aminopropan-2- yl)-1-phenyl- 3- (trifluorometh yl)-1H- pyrazole-4- carboxamide | White solid (quant.); ¹H NMR (400 MHz, CDCl₃) δ 1.22(d, 3 H, J = 6.6 Hz), 3.10-3.20(m, 2 H), 4.40- 4.50(m,1 H), 6.35(br, 2 H), 7.30-7.43(m, 3 H), 7.47(d, 1 H, J = 7.8 Hz), 7.58-7.63(m, 2 H), 8.67 (s, 1 H); m/z (APCI pos) 313.1 (100%) (M + H). |
| 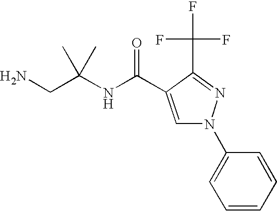 | N-(1-amino-2- methylpropan- 2-yl)-1- phenyl-3- (trifluorometh yl)-1H- pyrazole-4- carboxamide | Clear oil (quant.); ¹H NMR (400 Mhz, CDCl₃) δ 1.46 (s, 6 H), 3.28-3.34(m, 2 H), 5.00(br, 2 H), 6.72 (br, 1 H), 7.35-7.40(m, 1 H), 7.43-7.49(m, 2 H), 7.66-7.70(m, 2 H), 8.60 (s, 1 H). |
| 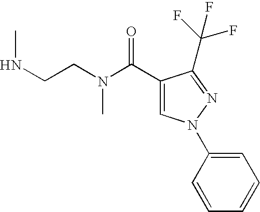 | N-methyl-N-(2- (methylamino)eth- yl)-1- phenyl-3- (trifluorometh yl)-1H- pyrazole-4- carboxamide | ¹H NMR (400 MHz, CDCl₃) δ 1.42(t, 3 H), 2.43(br, 3 H), 2.82(br, 2 H), 3.06 (s, 3 H), 3.57(br, 2 H), 4.05(q, 2 H), 6.88(m, 2 H), 7.38(m, 2 H). |

### Step 3

Following the procedure described for the step 1 in Example C3, (S)-methyl 3-(4-ethoxybenzamido)-2-(1-phenyl-3-(trifluoromethyl)-1H-pyrazole-4-carboxamido)propanoate was obtained as a white solid (1.19 g, 84%) from (S)-methyl 3-amino-2-(1-phenyl-3-(trifluoromethyl)-1H-pyrazole-4-carboxamido)propanoate and 4-ethoxybenzoic acid: ¹H NMR (400 MHz, CDCl₃) δ 1.41 (t, 3H, J=7.0 Hz), 3.80 (t, 3H), 3.95 (t, 2H), 4.04 (q, 2H, J=7.0 Hz), 4.89 (m, 1H), 6.88 (m, 3H), 7.40 (m, 1H), 7.49 (m, 2H), 7.64-7.74 (m, 5H), 8.43 (d, 1H); m/z (APCI pos) 505 (100%) (M+H).

Compounds of the following structures were prepared from the corresponding amines and either 4-ethoxybenzoic acid or 1-phenyl-3-(trifluoromethyl)-1H-pyrazole-4-carboxylic acid, using a similar method to that described above.

| Ex # | Structure | Name | Physcial Data |
|---|---|---|---|
| C2 | 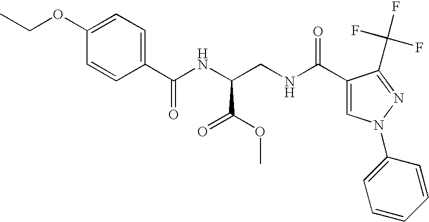 | (S)-methyl 2-(4- ethoxybenzami- do)-3-(1- phenyl-3- (trifluorometh- yl)-1H- pyrazole-4- carboxamido) propanoate | White solid (63%); ¹H NMR (400 MHz, CDCl₃) δ 1.41(t, 3 H, J = 7.0 Hz), 3.77(s, 3 H), 3.86-4.00(m, 2 H), 4.03 (q, 2 H, J = 7.0 Hz), 4.88(m, 1 H), 6.85(m, 2 H), 7.25(t, 1 H), 7.34-7.44(m, 3 H), 7.54-7.62(m, 3 H), 7.26 (m, 2 H), 8.43(s, 1 H); m/z (APCI pos) 505.0 (100%) (M + H). |
| C21 | 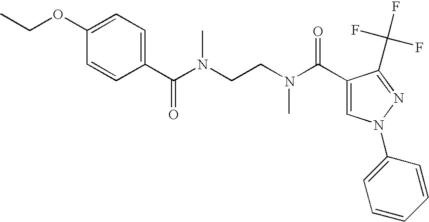 | N-(2-(4- ethoxy-N- methylbenzami- do)ethyl)- N-methyl-1- phenyl-3- (trifluorometh- yl)-1H- pyrazole-4- carboxamide | White solid (14%); ¹H NMR (400 MHz, CDCl₃) δ 1.40(t, 3 H, J = 7.0 Hz), 1.55(s, 3 H), 3.14 (m, 5 H), 3.88(s, 2 H), 4.02(q, 2 H, J = 7.03 Hz), 6.87(m, 2 H), 7.33-7.55(m ,7 H), 7.86 (s, 1 H); m/z (APCI pos) 475.0 (100%) (M + H). |
| C22 | 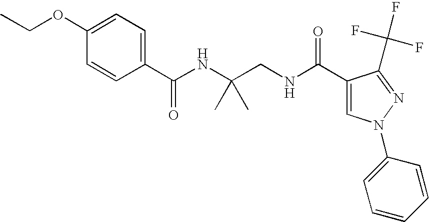 | N-(2-(4- ethoxybenzami- do)-2- methylpropyl)- 1-phenyl- 3- (trifluorometh- yl)-1H- pyrazole-4- carboxamide | White solid (52%); ¹H NMR (400 MHz, DMSO-d₆) δ 1.34(t, 3 H, J = 7.0 Hz), 1.43(s, 6 H), 3.53(d, 2 H, J = 6.2 Hz), 4.07(q, 2 H, J = 7.0 Hz), 6.93(m, 2 H), 7.48(m 1 H), 7.57-7.63 (m, 2 H), 7.76-7.80(m, 2 H), 7.82-7.86(m, 2 H), 7.91(s, 1 H), 8.71(t, 1 H, J = 6.2 Hz), 9.15 (s, 1 H); m/z (APCI pos) 475.0 (100%) (M + H). |
| C23 | 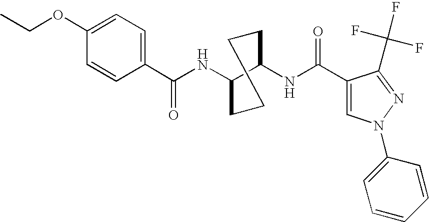 | N-(trans-2- (4- ethoxybenzami- do)cyclohex yl)-1- phenyl-3- (trifluorometh- yl)-1H- pyrazole-4- carboxamide | White solid (11%); ¹H NMR (400 MHz, DMSO-d₆) δ 1.26-1.32(m, 2 H), 1.30 (t, 3 H, J = 7.0 Hz), 1.40-1.50(m, 2 H), 1.71-1.79(m, 2 H), 1.87-1.97(m, 2 H), 3.80-3.95(m, 2 H), 4.03 (q, 2 H, J = 7.0 Hz), 6.85-6.90(m, 2 H), 7.44-7.49(m, 1 H), 7.56-7.63(m, 2 H), 7.67-7.71(m, 2 H), 7.72-7.76(m, 2 H), 8.11 (d, 1 H, J = 8.2 Hz), 8.18(d, 1 H, J = 8.6 Hz), 8.92(s, 1 H); m/z (APCI pos) 501.1 (100%) (M + H). |
| C24 | 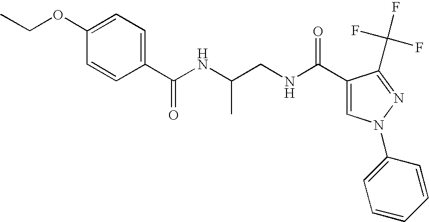 | N-(2-(4- ethoxybenzami- do)propyl)- 1-phenyl-3- (trifluorometh- yl)-1H- pyrazole-4- carboxamide | White solid (44%); ¹H NMR (400 MHz, DMSO-d₆) δ 1.18(d, 3 H, J = 6.6 Hz), 1.34(t, 3 H, J = 7.0 Hz), 3.35-3.41(m, 2 H), 4.07(q, 2 H, J = 7.0 Hz), 4.15-4.25(m, 1 H), 6.94-6.99(m, 2 H), 7.45-7.50(m, 1 H), 7.57-7.64(m, 2 H), 7.78-7.84(m, 4 H), 8.12 (d, 1 H, J = 8.2 Hz), 8.45(t, 1 H, J = 5.9 Hz), 9.05(s, 1 H); m/z (APCI pos) 461.1 (100%) (M + H). |
| C25 | 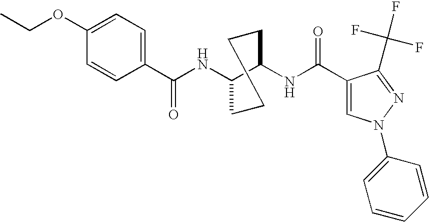 | N-(cis-2-(4- ethoxybenzami- do)cyclohex yl)-1- phenyl-3- (trifluorometh- yl)-1H- pyrazole-4- carboxamide | White solid (30%); ¹H NMR (400 MHz, DMSO-d₆) δ 1.32(t, 3 H, J = 7.0 Hz), 1.36-1.50(m, 2 H), 1.5-1.90(m, 6 H), 4.05(q, 2 H, J = 7.0 Hz), 4.17(m, 1 H), 4.25 (m, 1 H), 6.91-7.00(m, 2 H), 7.44-7.49(m, 1 H), 7.58-7.63(m, 2 H), 7.73-7.79(m, 2 H), 7.82-7.89(m, 4 H), 9.13 (s, 1 H); m/z (APCI pos) 501.1 (100%) (M + H). |
| C26 | 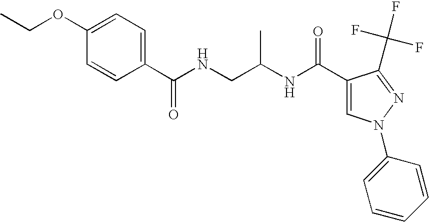 | N-(1-(4- ethoxybenzami- do)propan- 2-yl)-1- phenyl-3- (trifluorometh- yl)-1H- pyrazole-4- carboxamide | White solid (42%); ¹H NMR (400 MHz, DMSO-d₆) δ 1.17(t, 3 H, J = 8.6 Hz), 1.32(t, 3 H, J = 7.0 Hz), 3.34-3.45(m, 2 H), 4.07(q, 2 H, J = 7.0 Hz), 4.15-4.24(m, 1 H), 6.94-6.98(m, 2 H), 7.45-7.51(m, 1 H), 7.57-7.65(m, 2 H), 7.78-7.85(m, 4 H), 8.17 (d, 1 H, J = 8.2 Hz), 8.38(t, 1 H, J = 5.9 Hz), 9.07(s, 1 H); m/z (APCI pos) 461.1 (85%) (M + H). |
| C27 | 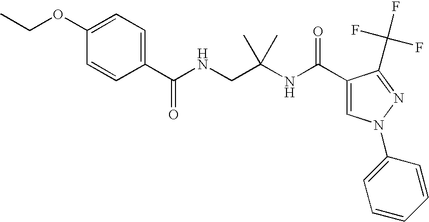 | N-(1-(4- ethoxybenzami- do)-2- methylpropan- 2-yl)-1- phenyl-3- (trifluorometh- yl)-1H- pyrazole-4- carboxamide | White solid (55%); ¹H NMR (400 MHz, DMSO-d₆) δ 1.34(t, 3 H, J = 7.0 Hz), 1.39(s, 6 H), 3.54(d, 2 H, J = 5.9 Hz), 4.08(q, 2 H, J = 7.0 Hz), 6.97-7.01(m, 2 H), 7.45-7.51(m, 1 H), 7.57-7.65(m, 2 H), 7.82-7.86(m, 4 H), 8.05 (s, 1 H), 8.52(t, 1 H, J = 5.9 Hz), 9.01(s, 1 H); m/z (APCI pos) 475.1 (100%) (M + H). |

### Example C5

### (S)—N-(1-(4-Ethoxybenzamido)-3-hydroxypropan-2-yl)-1-phenyl-3-(trifluoromethyl)-1H-pyrazole-4-carboxamide

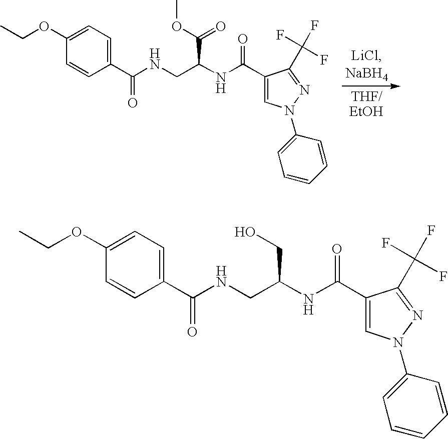

To (S)-methyl 3-(4-ethoxybenzamido)-2-(1-phenyl-3-(trifluoromethyl)-1H-pyrazole-4-carboxamido)propanoate (647 mg, 1.3 mmol) in anhydrous THF/EtOH (2/1, 10 mL) was added lithium chloride (109 mg, 2.6 mmol). Upon dissolution, sodium borohydride (97 mg, 2.6 mmol) was added and the mixture was stirred at room temperature for 18 hours. The mixture was concentrated to dryness, water (50 mL) was added and the pH was brought to 2-3 by addition of 2N HCl. The aqueous layer was extracted with DCM (3×50 mL) and the combined organic layers were dried over MgSO₄. Concentration yielded (S)—N-(1-(4-ethoxybenzamido)-3-hydroxypropan-2-yl)-1-phenyl-3-(trifluoromethyl)-1H-pyrazole-4-carboxamide as a white solid (450 mg, 74%): ¹H NMR (400 MHz, DMSO-d₆) δ 1.32 (t, 3H), 3.40 (m, 1H), 3.45-3.68 (m, 3H), 4.07 (q, 2H), 4.13 (m, 1H), 6.96 (m, 2H), 7.48 (m, 1H), 7.61 (m, 2H), 7.78-7.82 (m, 4H), 8.08 (d, 1H), 8.40 (t, 1H), 9.13 (s, 1H); m/z (APCI pos) 477.1 (20%) (M+H).

Compounds of the following structures were prepared from the corresponding esters, using a similar method to that described above.

| Ex # | Structure | Name | Physical Data |
|---|---|---|---|
| C4 | 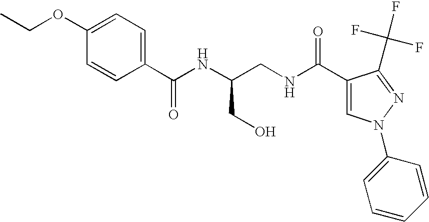 | (S)-N-(2-(4- ethoxybenzami do)-3- hydroxypropyl)- 1-phenyl-3- (trifluorometh- yl)-1H- pyrazole-4- carboxamide | White solid (63%); ¹H NMR (400 MHz, DMSO-d₆) δ 1.33(t, 3 H, J = 7.0 Hz), 3.40-3.60(m, 4 H), 4.07(q, 2 H, J = 7.0 Hz), 4.13(m, 1 H), 4.60(br, 1 H), 6.96 (m, 2 H), 7.48(m, 1 H), 7.61(m, 2 H), 7.78- 7.82(m, 4 H), 8.00(d, 1 H), 8.48(t, 1 H), 9.08(s, 1 H); m/z (APCI pos) 459.2 (100%) (M − H₂O). |
| C16 | 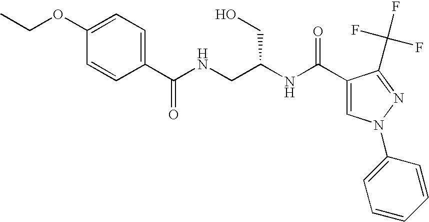 | (R)-N-(3-(4- ethoxybenzami do)-1- hydroxypropan- 2-yl)-1- phenyl-3- (trifluorometh- yl)-1H- pyrazole-4- carboxamide | White solid (88%); ¹H NMR (400 MHz, DMSO-d₆) δ 1.33(t, 3 H, J = 7.0 Hz), 3.35-3.57(m, 4 H), 4.06(q, 2 H, J = 7.0 Hz), 4.10-4.18(m, 1 H), 4.83(t, 1 H, J = 5.8 Hz), 6.96(d, 2 H, J = 9.0 Hz), 7.48 (tt, 1 H, J = 7.4, 1.6 Hz), 7.57-7.64(m, 2 H), 7.76-7.84(m, 4 H), 8.06(d, 1 H, J = 8.2 Hz), 8.38(t, 1 H, J = 5.9 Hz), 9.11(s, 1 H); m/z (APCI pos) 477.0 (20%), 459.0 (100%) (M − H₂O). |
| C20 | 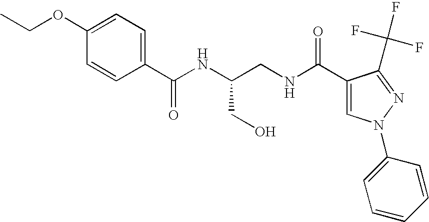 | (R)-N-(2-(4- ethoxybenzami do)-3- hydroxypropyl)- 1-phenyl-3- (trifluorometh- yl)-1H- pyrazole-4- carboxamide | White solid (85%); ¹H NMR (400 MHz, DMSO-d₆) δ 1.33(t, 3 H, J = 7.0 Hz), 3.39-3.60(m, 4 H), 4.07(q, 2 H, J = 7.0 Hz), 4.08-4.16(m, 1 H), 4.79(t, 1 H, J = 5.8 Hz), 6.96(d, 2 H, J = 9.0 Hz), 7.47(tt, 1 H, J = 7.4, 1.5 Hz), 7.56-7.63(m, 2 H), 7.77-7.84(m, 4 H), 7.97(d, 1 H, J = 7.4 Hz), 8.44(t, 1 H, J = 5.9 Hz), 9.04(s, 1 H); m/z (APCI pos) 459.2 (100%) (M + H − H₂O). |

### Example C₇

### 4-Ethoxy-N-(1-(1-phenyl-3-(trifluoromethyl)-1H-pyrazole-4-carbonyl)piperidin-3-yl)benzamide

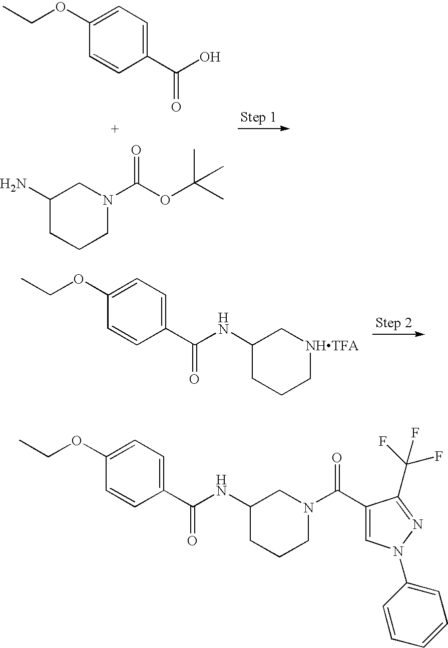

### Step 1

EDAC.HCl (0.1 g, 0.6 mmol), HOBt.H₂O (0.08 g, 0.6 mmol) and 4-ethoxybenzoic acid (0.09 g, 0.6 mmol) were dissolved in DMF (0.5 ml), and tert-butyl 3-aminopiperidine-1-carboxylate (0.1 g, 0.5 mmol) was added. The mixture was stirred for 1 h. The solvent was removed under vacuum, and the residue was placed directly on a silica column. The column was eluted with 10% ether/DCM to give 92 mg of a clear oil. The oil was dissolved in DCM (5 mL), and TFA (5 mL) was added. The mixture was stirred for 15 min and the solvent was removed under vacuum to give 4-ethoxy-N-(piperidin-3-yl)benzamide trifluoroacetate (0.055 g, 0.2 mmol, 30%) as a solid: ¹H NMR (400 MHz, DMSO-d₆) δ 1.34 (t, 3H, J=7.0 Hz), 1.49 (m, 2H), 1.71 (m, 1H), 1.84 (m, 1H), 2.53 (m, 2H), 2.92 (m, 1H), 3.06 (m, 1H), 3.90 (m, 1H), 4.07 (q, 2H, J=7.0 Hz), 6.96 (m, 2H), 7.80 (m, 2H), 8.05 (m, 1H).

Compounds of the following structures were prepared from the corresponding Boc-protected diamines and either 4-ethoxybenzoic acid or 1-phenyl-3-(trifluoromethyl)-1H-pyrazole-4-carboxylic acid, using a similar method to that described above.

| Structure | Name | Physical Data |
|---|---|---|
| 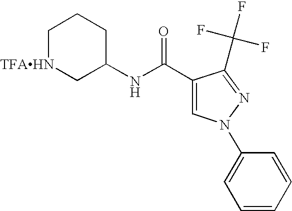 | 1-phenyl-N- (piperidin-3- yl)-3- (trifluorometh yl)-1H- pyrazole-4- carboxamide trifluoroacetate | Solid (58%); m/z (APCI pos) 339.1 (100%) (M + H). |
| 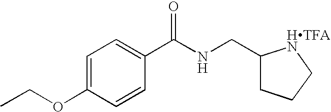 | 4-ethoxy-N- (pyrrolidin-2- ylmethyl)benza mide trifluoroacetate | Solid (33%); ¹H NMR (400 MHz, CDCl₃) δ 1.42(t, 3 H, J = 7.0 Hz), 1.57(m, 1 H), 1.74(m, 1 H), 1.97 (m, 3 H), 3.38(m, 2 H), 3.51(m, 1 H), 4.06(q, 2 H, J = 7.0 Hz), 4.21(m, 1 H), 6.89(m, 2 H), 7.83 (m, 2 H), 8.27(m, 1 H). |
| 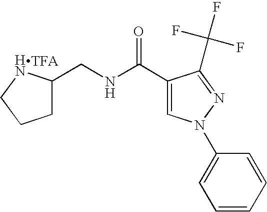 | 1-phenyl-N- (pyrrolidin-2- ylmethyl)-3- (trifluorometh yl)-1H- pyrazole-4- carboxamide trifluoroacetate | Solid (51%); ¹H NMR (400 MHz, CDCl₃) δ 1.71-2.09 (m, 4 H), 3.39(m, 3 H), 3.56(m, 1 H), 4.12(m, 1 H), 7.40(m, 1 H), 7.50 (m, 2 H), 7.71(m ,2 H), 7.99(m, 1 H), 8.37(m, 1 H). |

### Step 2

To a 5 ml reacti-vial were added 1-phenyl-3-(trifluoromethyl)-1H-pyrazole-4-carboxylic acid (0.062 g, 0.24 mmol), EDAC-HCl (0.047 g, 0.24 mmol), and HOBt.H₂O (0.037 g, 0.24 mmol). These were dissolved in DMF and 4-ethoxy-N-(piperidin-3-yl)benzamide trifluoroacetate (0.055 g, 0.22 mmol) and triethylamine (33 μl, 0.24 mmol) were added to this mixture as a solution in DMF (1 mL). The mixture was stirred for 1 hour. The solvent was removed under vacuum and the residue was purified by silica gel column chromatography (1-3% MeOH/DCM) to give a material that was triturated with ether to give 4-ethoxy-N-(1-(1-phenyl-3-(trifluoromethyl)-1H-pyrazole-4-carbonyl)piperidin-3-yl)benzamide (0.052 g, 48%) as a crystalline solid: ¹H NMR (400 MHz, CDCl₃) δ 1.44 (t, 3H, J=7.0 Hz), 1.58 (s, 1H), 1.60-1.90 (m, 3H), 2.14 (br, 1H), 3.19 (br, 0.5H), 3.35 (br, 1H), 3.60 (br, 0.5H), 4.08 (q, 2H, J=7.0 Hz), 4.08-4.25 (m, 2H), 6.02 (br, 0.5H), 6.75 (br, 0.5H), 6.91 (d, 2H, J=8.6 Hz), 7.39 (br, 1H), 7.49 (m, 2H), 7.60-7.90 (m, 4H), 8.05 (br, 0.5H), 8.36 (m, 0.5H); m/z (APCI pos) 487.0 (10%) (M+H).

Compounds of the following structures were prepared from the corresponding amines which were obtained in Step 1 in Example. C7 and either 4-ethoxybenzoic acid or 1-phenyl-3-(trifluoromethyl)-1H-pyrazole-4-carboxylic acid, using a similar method to that described above.

| Ex # | Structure | Name | Physical Data |
|---|---|---|---|
| C8 | 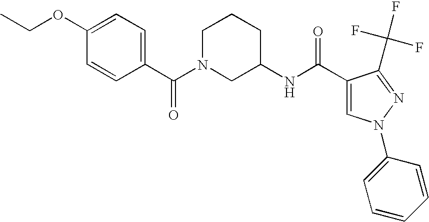 | N-(1-(4- ethoxybenzoyl) piperidin-3- yl)-1-phenyl- 3- (trifluorometh- yl)-1H- pyrazole-4- carboxamide | White solid (48%); ¹H NMR (400 MHz, CDCl₃) δ 1.42(t, 3 H, J = 7.0 Hz), 1.57(s, 1 H), 1.66(br, 1 H), 1.75 (br, 1 H), 1.96(br, 2 H), 3.57-3.75(m, 3 H), 4.05(q, 2 H, J = 7.0 Hz), 4.26(br, 1 H), 6.90(d, 2 H, J = 9.0 Hz), 7.42(m, 3 H), 7.51(m, 2 H), 7.71(m, 2 H), 8.43(m, 1 H); m/z (APCI pos) 487.0 (10%) (M + H). |
| C9 | 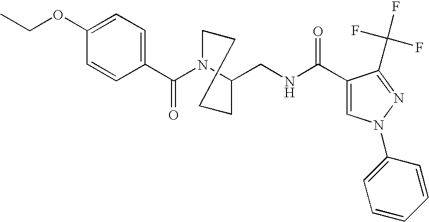 | N-((1-(4- ethoxybenzoyl) piperidin-2- yl)methyl)-1- phenyl-3- (trifluorometh- yl)-1H- pyrazole-4- carboxamide | White solid (10%); ¹H NMR (400 MHz, CDCl₃) δ 1.25(m, 2 H), 1.36(t, 3 H, J = 7.0 Hz), 1.44 (br, 1 H), 1.65-1.78 (m, 4 H), 1.83(m, 2 H), 3.16(0.5 H), 3.5(m, 1 H), 3.76(m, 0.5 H), 3.95(m, 2 H), 4.50(m, 0.5 H), 5.15(m, 0.5 H), 3.95(m, 2 H), 4.50(m, 0.5 H), 5.15(m, 0.5 H), 6.79(d, 2 H, J = 9.0 Hz), 7.06-7.26(m, 5 H), 7.33(d, 2 H, J = 8.59 Hz), 8.41(s, 1 H); m/z (APCI pos) 501.1 (10%) (M + H). |
| C10 | 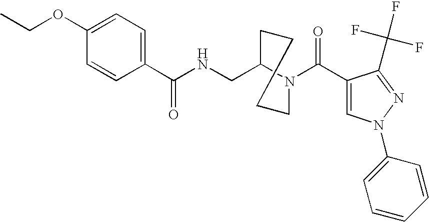 | 4-ethoxy-N- ((1-(1- phenyl-3- (trifluorometh- yl)-1H- pyrazole-4- carbonyl)pipe ridin-2- yl)methyl)ben zamide | White solid (43%); ¹H NMR (400 MHz, CDCl₃) δ 1.27(m, 3 H), 1.40(t, 3 H, J = 7.0 Hz), 1.64- 1.86(m, 5 H), 3.05(m, 1 H), 3.32(m, 1 H), 3.53(m, 1 H), 4.01(q, 2 H, J = 7.0 Hz), 4.40 (m, 0.5 H), 5.15(m, 0.5 H), 6.87(m, 3 H), 7.35-7.57(m, 6 H), 7.78(m, 1 H); m/z (APCI pos) 501.1 (10%) (M + H). |
| C11 | 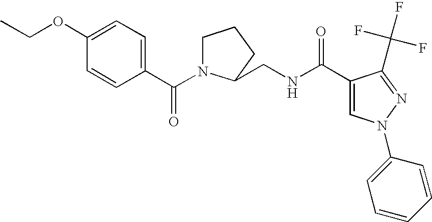 | N-((1-(4- ethoxybenzoyl) pyrrolidin- 2-yl)methyl)- 1-phenyl-3- (trifluorometh- yl)-1H- pyrazole-4- carboxamide | White solid (63%); ¹H NMR (400 MHz, CDCl₃) δ 1.42(t, 3 H, J = 7.0 Hz), 1.78(m, 2 H), 1.96(m, 1 H), 2.24(m, 1 H), 3.59(m, 3 H), 3.78(m, 1 H), 4.05(q, 2 H, J = 7.0 Hz), 4.58 (m, 1 H), 8.89(d, 2 H, J = 8.98 Hz), 7.37(m, 1 H), 7.47(m, 4 H), 7.63(d, 2 H), 7.94(m, 1 H), 8.33(s, 1 H); m/z (APCI pos) 487.0 (10%) (M + H). |
| C12 | 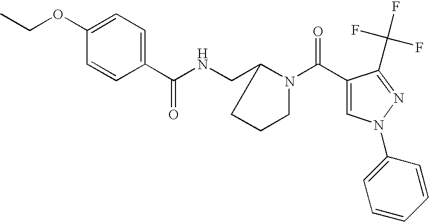 | 4-ethoxy-N- ((1-(1- phenyl-3- (trifluorometh- yl)-1H- pyrazole-4- carbonyl)pyrro- lidin-2- yl)methyl)benz- amide | White solid (76%); ¹H NMR (400 MHz, CDCl₃) δ 1.41(t, 3 H, J = 7.0 Hz), 1.89(m, 2 H), 2.04(m, 1 H), 2.23(m, 1 H), 3.52(m, 3 H), 3.78(m, 1 H), 4.05(q, 2 H, J = 7.0 Hz), 4.63 (m, 1 H), 6.90(d, 2 H, J = 8.6 Hz), 7.41(m, 1 H), 7.50(m, 2 H), 7.67(m, 2 H), 7.83(m, 2 H), 8.04(s, 2 H); m/z (APCI pos) 487.0 (10%) (M + H). |

### Example C14

### N-(2-(4-Ethoxy-N-methylbenzamido)ethyl)-1-phenyl-3-(trifluoromethyl)-1H-pyrazole-4-carboxamide

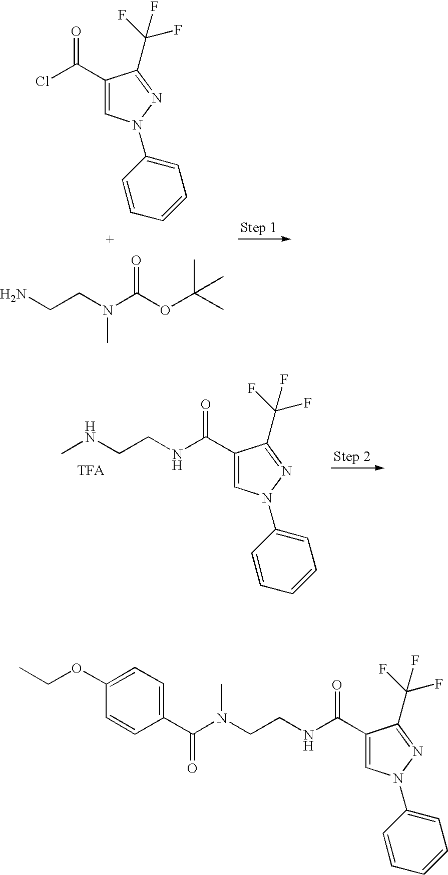

### Step 1

To a solution of tert-butyl 2-aminoethyl(methyl)carbamate (0.174 g, 1.00 mmol) in DCM (5 ml) and saturated aqueous sodium bicarbonate (2 ml) was added 1-phenyl-3-(trifluoromethyl)-1H-pyrazole-4-carbonyl chloride (0.302 g, 1.10 mmol, prepared in the standard way from 1-phenyl-3-(trifluoromethyl)-1H-pyrazole-4-carboxylic acid and oxalyl chloride) as a solution in dichloromethane (1 ml). The mixture was stirred for 1 hour. The layers were separated and the organic layer was dried (sodium sulfate) and the solvent was removed under vacuum to give 405 mg of the Boc protected amine. This was dissolved in DCM (5 ml) and treated with TFA (2 ml) and the mixture was stirred for 1 hour. The solvent was removed under vacuum to give N-(2-(methylamino) ethyl)-1-phenyl-3-(trifluoromethyl)-1H-pyrazole-4-carboxamide trifluoroacetate (0.426 g, 100%) as an oil: ¹H NMR (400 MHz, CDCl₃) δ 2.92 (s, 3H), 3.49 (m, 2H), 3.59 (m, 2H), 7.41 (m, 1H), 7.50 (m, 2H), 7.70 (m, 2H), 8.36 (m, 1H).

The following compound was prepared from tert-butyl 2-aminoethyl(methyl)carbamate and 4-ethoxybenzoyl chloride in the manner described above.

| Structure | Name | Physical Data |
|---|---|---|
| 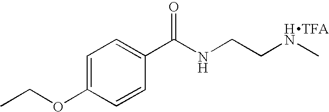 | 4-ethoxy-N-(2- (methylamino)eth- yl)benzamide triluoroacetate | Solid (69%); ¹H NMR (400 MHz, CDCl₃) δ 1.44(m, 3 H), 2.91(s, 3 H), 3.53(4 H), 4.06(q, 2 H, J = 7.03 Hz), 6.89(m, 2 H), 7.75(m, 2 H). |

### Step 2

To a solution of N-(2-(methylamino)ethyl)-1-phenyl-3-(trifluoromethyl)-1H-pyrazole-4-carboxamide trifluoroacetate (0.312 g, 1.00 mmol) in DCM (10 mL) and saturated aqueous sodium bicarbonate (2 ml) was added 4-ethoxybenzoyl chloride (0.203 g, 1.10 mmol) as a solution in dichloromethane (1 ml). The mixture was stirred for 1 hour. The layers were separated, the aqueous layer was back extracted and the combined organic layers were dried (sodium sulfate), and concentrated under vacuum. The resulting residue was triturated with ether/hexanes to give N-(2-(4-ethoxy-N-methylbenzamido)ethyl)-1-phenyl-3-(trifluoromethyl)-1H-pyrazole-4-carboxamide (0.274 g, 60%) as a solid: ¹H NMR (400 MHz, DMSO-d₆) δ 1.32 (t, 3H, J=7.0 Hz), 3.00 (s, 3H), 3.38-3.64 (m, 4H), 4.03 (m, 2H, J=7.0 Hz), 6.90 (s, 2H), 7.33 (s, 2H), 7.48 (m, 1H), 7.61 (m, 2H), 7.80 (m, 2H), 8.51 (br, 1H), 9.01 (s, 1H); m/z (APCI pos) 461.0.0 (10%) (M+H).

The following compound was prepared from 4-ethoxy-N-(2-(methylamino)ethyl)benzamide trifluoroacetate and 1-phenyl-3-(trifluoromethyl)-1H-pyrazole-4-carboxylic acid in the manner described above.

| Ex # | Structure | Name | Physical Data |
|---|---|---|---|
| C13 | 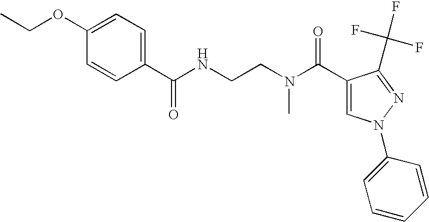 | N-(2-(4- ethoxybenzami do)ethyl)-N- methyl-1- phenyl-3- (trifluorometh- yl)-1H- pyrazole-4- carboxamide | White solid (43%); ¹H NMR (400 MHz, DMSO-d₆) δ 1.34(t, 3 H, J = 7.0 Hz), 3.06(s, 3 H), 3.39-3.53(m, 3 H), 3.64 (m, 1 H), 4.06(q, 2 H, J = 7.0 Hz), 6.95(m, 2 H), 7.46(m, 1 H), 7.57 (m, 2 H), 7.72(m, 1 H), 7.82(m, 3 H), 8.43(s, 1 H), 8.68(s, 0.7 H), 8.87(s, 0.3 H); m/z (APCI pos) 461.0 (10%) (M + H). |

### Example C18

### (R)-Methyl 2-(4-ethoxybenzamido)-3-(1-phenyl-3-(trifluoromethyl)-1H-pyrazole-4-carboxamido)propanoate

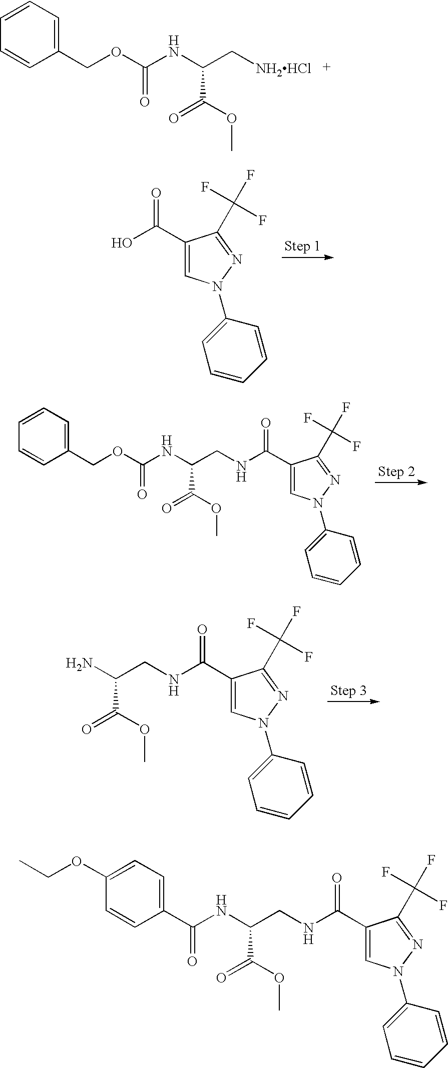

### Step 1

Following the procedure described for the step 1 in Example C3, (R)-methyl 2-(benzyloxycarbonylamino)-3-(1-phenyl-3-(trifluoromethyl)-1H-pyrazole-4-carboxamido)propanoate was obtained as a white solid (4.6 g, 90%) from (R)-methyl 3-amino-2-(benzyloxycarbonyl)propanoate hydrochloride and 1-phenyl-3-(trifluoromethyl)-1H-pyrazole-4-carboxylic acid: ¹H NMR (400 MHz, DMSO-d₆) δ 3.50-3.70 (m, 2H), 3.64 (s, 3H), 4.28-4.35 (m, 1H), 5.05 (s, 2H), 7.25-7.37 (m, 5H), 7.45-7.51 (m, 1H), 7.58-7.63 (m, 2H), 7.70-7.75 (m, 1H), 7.78-7.83 (m, 2H), 8.48 (t, 1H, J=5.9 Hz), 9.03 (s, 1H).

### Step 2

To a solution of (R)-methyl 2-(benzyloxycarbonyl)-3-(1-phenyl-3-(trifluoromethyl)-1H-pyrazole-4-carboxamido)propanoate (4.5 g, 9.2 mmol) in dry THF (300 mL) was added 10% wt palladium on charcoal (dry) (1 g) under argon atmosphere. The mixture was shaken under hydrogen (45 psi) for 4 days at room temperature. The palladium was filtered off and the filtrate was concentrated and purified by silica gel column chromatography (DCM/MeOH=95/5). (R)-Methyl 2-amino-3-(1-phenyl-3-(trifluoromethyl)-1H-pyrazole-4-carboxamido)propanoate was isolated as a white solid (2.57 g, 84%): ¹H NMR (400 MHz, DMSO-d₆) δ 3.17 (s, 1H), 3.18 (s, 1H), 3.40-3.45 (m, 2H), 3.62 (s, 3H), 4.06-4.12 (m, 1H), 7.45-7.50 (m, 1H), 7.58-7.63 (m, 2H), 7.81-7.85 (m, 2H), 8.37 (t, 1H, J=5.9 Hz), 9.09 (s, 1H).

### Step 3

Following the procedure described for the step 1 in Example C3, (R)-methyl 2-(4-ethoxybenzamido)-3-(1-phenyl-3-(trifluoromethyl)-1H-pyrazole-4-carboxamido)propanoate was prepared as white solid (1.35 g, 73%) from (R)-Methyl 2-amino-3-(1-phenyl-3-(trifluoromethyl)-1H-pyrazole-4-carboxamido)propanoate and 4-ethoxbenzoic acid: ¹H NMR (400 MHz, DMSO-d₆) δ 1.34 (t, 3H, J=7.0 Hz), 3.60-3.70 (m, 1H), 3.66 (s, 3H), 3.82 (dt, 1H, J=13.7, 5.8 Hz), 4.09 (q, 2H, J=7.0 Hz), 4.62 (dt, 1H, J=7.4, 5.5 Hz), 7.00 (d, 2H, J=9.0 Hz), 7.48 (tt, 1H, J=7.4, 1.5 Hz), 7.56-7.63 (m, 2H), 7.78-7.88 (m, 4H), 8.61 (t, 1H, J=5.9 Hz), 8.67 (d, 1H, J=7.4 Hz), 9.04 (s, 1H); m/z (APCI pos) 505.0 (100%) (M+H).

The compound of the following structure was prepared from the corresponding amine, using a similar method to that described above.

| Ex # | Structure | Name | Physical Data |
|---|---|---|---|
| C15 | 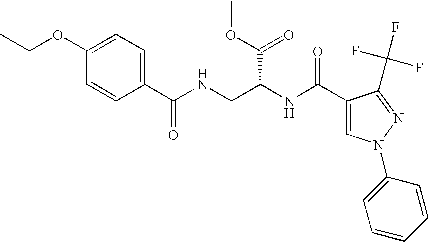 | (R)-methyl 3- (4- ethoxybenzami do)-2-(1- phenyl-3- (trifluorometh- yl)-1H- pyrazole-4- carboxamide)pro- panoate | White solid (76%); ¹H NMR (400 MHz, DMSO-d₆) δ 1.27(t, 3 H, J= 7.0 Hz), 3.51-3.61(m, 1 H), 3.60(s, 3 H), 3.65-3.75 (m, 1 H), 4.02(q, 2 H, J = 7.0 Hz), 4.61(dd, 1 H, J = 13.6, 6.6 Hz), 6.92(d, 2 H, J = 9.0 Hz), 7.44(tt, 1 H, J = 7.4, 1.6 Hz), 7.53-7.61 (m, 2 H), 7.72(d, 2 H, J = 9.0 Hz), 7.75-7.80 (m, 2 H), 8.45(t, 1 H, J = 5.9 Hz), 8.68(d, 1 H, J = 7.4 Hz), 9.08(s, 1 H); m/z (APCI pos) 505.0 (100%) (M + H). |

### Example D1

### N-(2-(4-Ethoxybenzamido)ethyl)-4,6-diphenylpicolinamide

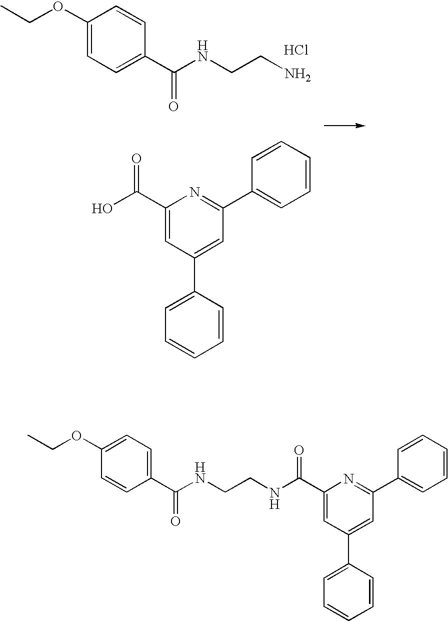

A mixture of N-(2-aminoethyl)-4-ethoxybenzamide hydrochloride (0.16 g, 0.66 mmol), 4,6-diphenylpicolinic acid (0.15 g, 0.55 mmol), triethylamine (0.11 mL, 0.82 mmol), EDAC HCl (0.14 g, 0.71 mmol), HOBt.H₂O (0.11 g, 0.71 mmol) and DMF (6 mL) was stirred at room temperature for 4 h. The mixture was diluted with EtOAc, washed successively with water and brine, dried over MgSO₄, and concentarated in vacuo. The residue was purified by silica gel column chromatography (hexanes/EtOAc=1/1-1/4) to give N-(2-(4-ethoxybenzamido)ethyl)-4,6-diphenylpicolinamide (0.13 g, 49%) as colorless crystals: ¹H NMR (400 MHz, CDCl₃) δ 1.42 (3H, t, J=7.2 Hz), 3.72-3.86 (4H, m), 4.06 (2H, q, J=7.2 Hz), 6.90 (2H, d, J=8.4 Hz), 7.27 (1H, m), 7.44-7.58 (6H, m), 7.73-7.85 (4H, m), 8.04-8.12 (3H, m), 8.40 (1H, s), 8.74 (1H, t, J=5.8 Hz); m/z (APCI pos) 466.1 (30%) (M+H).

### Example D2 and D3

### N-(2-(4-Ethoxybenzamido)ethyl)-4-methyl-1-phenyl-1H-pyrazole-3-carboxamide and

### N-(2-(4-ethoxybenzamido)ethyl)-4-methyl-1-phenyl-1H-pyrazole-5-carboxamide

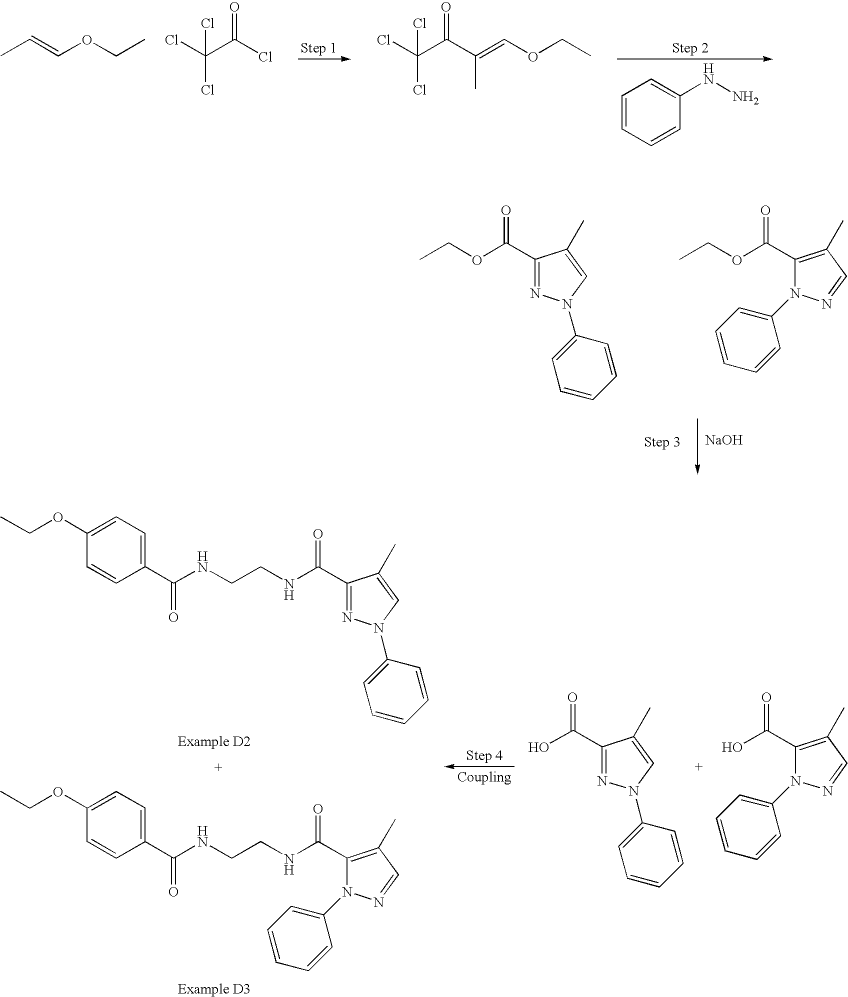

### Step 1

A mixture of (E)-1-ethoxyprop-1-ene (5.00 g, 58.05 mmol) in pyridine (4.685 ml, 58.05 mmol) was added to at −10° C. stirring solution of 2,2,2-trichloroacetyl chloride (10.56 g, 58.05 mmol) in DCM (15 mL) at a rate of 6-10 drops/minute. After the addition was complete, the mixture was stirred at room temperature for 16 hours. The resulting precipitate was filtered and washed with DCM. The filtrate was concentrated to a residue with a bath temperature of 50° C. and the residue was dried on high vacuum overnight to give (E)-1,1,1-trichloro-4-ethoxy-3-methylbut-3-en-2-one (16.14 g, 120% yield). The crude material is used directly in the next step without further purification.

### Step 2

A mixture of (E)-1,1,1-trichloro-4-ethoxy-3-methylbut-3-en-2-one (16.14 g, 69.72 mmol) and 1-phenylhydrazine (9.047 g, 83.66 mmol) in EtOH (70 mL) was heated at 78° C. for 4 hours. The mixture was concentrated to a residue, dissolved in DCM, washed with 1N HCl and water, dried on Na₂SO₄, and concentrated to a residue. The obtained residue was dissolved in minimal DCM and passed through a plug of Silica eluting with 30% EtOAc/hexanes to give 2.1 g of a crude black oil containing a mixture of regio-isomers (ethyl 4-methyl-1-phenyl-1H-pyrazole-3-carboxlate and ethyl 4-methyl-1-phenyl-1H-pyrazole-5-carboxlate). This material was carried directly into the next step.

### Step 3

To a mixture of regio-isomers (ethyl 4-methyl-1-phenyl-1H-pyrazole-3-carboxlate and ethyl 4-methyl-1-phenyl-1H-pyrazole-5-carboxlate) (2.10 g, 9.12 mmol) in EtOH (30 ml) and water (30 ml) was added lithium hydroxide monohydrate (1.15 g, 27.4 mmol) and the mixture was stirred at 50° C. for 90 minutes. The mixture was cooled to room temperature and the solvent was removed. Water and EtOAc were added and the layers were separated. The aqueous layer was acidified with 6N HCl and then extract with DCM. The combined organic layers were dried over Na₂SO₄ and concentrated to give 1.24 g (67% yield) of a mixture of regio-isomers, 4-methyl-1-phenyl-1H-pyrazole-3-carboxylic acid and 4-methyl-1-phenyl-1H-pyrazole-5-carboxylic acid that were used directly in the next step.

### Step 4

The crude mixture of regio-isomers, 4-methyl-1-phenyl-1H-pyrazole-3-carboxylic acid and 4-methyl-1-phenyl-1H-pyrazole-5-carboxylic acid (300 mg, 1.48 mmol), HATU (564 mg, 1.48 mmol), DIPEA (259 ul, 1.48 mmol), and N-(2-aminoethyl)-4-ethoxybenzamide hydrochloride (363 mg, 1.48 mmol) were combined in THF (15 ml) and stirred at room temperature for 6 hr. The mixture was quenched with water (150 ml) and extracted with DCM. The organics were dried over Na₂SO₄, filtered, and concentrated to a residue. The obtained residue was purified by silica gel column chromatography (75% EtOAc/haxanes) to give N-(2-(4-ethoxybenzamido)ethyl)-4-methyl-1-phenyl-1H-pyrazole-3-carboxamide: ¹H NMR (400 MHz, DMSO-d₆) δ 1.33 (t, J=6.9 Hz, 3H), 2.27 (s, 3H), 3.40-3.44 (m, 4H), 4.02-4.10 (m, 2H), 6.97 (d, J=8.8 Hz, 2H), 7.34 (t, J=7.3 Hz, 1H), 7.52 (t, J=7.9 Hz, 2H), 7.82 (d, J=8.8 Hz, 2H), 7.89 (d, J=7.8 Hz, 2H), 8.37 (br, 2H), 8.44 (s, 1H); m/z (APCI pos) 393 (M+H). The other regioisomer was further purified on Prep TLC eluting with 75% EtOAc/hexanes to give N-(2-(4-ethoxybenzamido)ethyl)-4-methyl-1-phenyl-1H-pyrazole-5-carboxamide: ¹H NMR (400 MHz, DMSO-d₆) δ 1.34 (t, J=6.9 Hz, 3H), 2.10 (s, 3H), 3.38-3.39 (m, 4H), 4.05-4.11 (m, 2H), 6.97 (d, J=8.8 Hz, 2H), 7.29-7.33 (m, 1H), 7.38-7.43 (m, 4H), 7.55 (s, 1H), 7.79 (d, J=8.8 Hz, 2H), 8.32 (s, 1H), 8.66 (s, 1H); m/z (APCI pos) 393 (M+H).

### Example D4

### N-(2-(4-Ethoxybenzamido)ethyl)-1-methyl-3-phenyl-1H-pyrazole-5-carboxamide

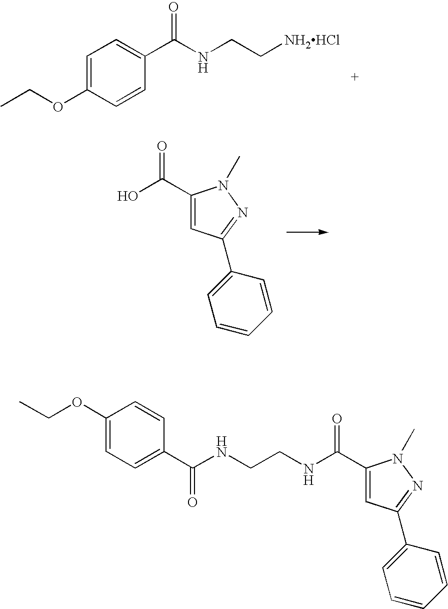

A mixture of HATU (0.5593 g, 1.471 mmol), DIPEA (0.7066 ml, 4.045 mmol), N-(2-aminoethyl)-4-ethoxybenzamide hydrochloride (0.300 g, 1.226 mmol) and 1-methyl-3-phenyl-1H-pyrazole-5-carboxylic acid (0.2479 g, 1.226 mmol) in THF (15 mL) was stirred at room temperature overnight. The mixture was quenched with water (150 ml) and the solid filtered, dried on high vacuum and purified on silica gel by eluting with 75% EtOAc/Hex. The pure material was recrystallized from EtOAc/hexanes to give N-(2-(4-ethoxybenzamido)ethyl)-1-methyl-3-phenyl-1H-pyrazole-5-carboxamide (0.145 g, 30%) as a white solid: ¹H NMR (400 MHz, DMSO-d₆) δ 1.33 (t, J=7.0 Hz, 3H), 3.42 (br, 4H), 4.05-4.10 (m, 5H), 6.97 (d, J=8.8 Hz, 2H), 7.23 (s, 1H), 7.32 (t, J=7.3 Hz, 1H), 7.43 (t, J=7.6 Hz, 2H), 7.74 (d, J=7.3 Hz, 2H), 7.82 (d, J=9.0 Hz, 2H), 8.45 (br, 1H), 8.65 (br, 1H); m/z (APCI pos) 393 (M+H).

Compounds of the following structures were prepared from N-(2-aminoethyl)-4-ethoxybenzamide hydrochloride and the corresponding acids, using a similar method to that described above.

| Ex # | Structure | Name | Physical Data |
|---|---|---|---|
| D5 | 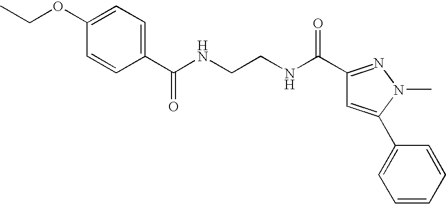 | N-(2-(4- ethoxybenzami do)ethyl)-1- methyl-5- phenyl-1H- pyrazole-3- carboxamide | ¹H NMR (400 MHz, DMSO- d₆) δ 1.34(t, J = 7.0 Hz, 3 H), 3.42(br, 4 H), 3.90(s, 3 H), 4.05-4.10 (m, 2 H), 6.77(s, 1 H), 6.97(d, J = 8.8 Hz, 2 H), 7.45-7.58(m, 5 H), 7.81(d, J = 8.8 Hz, 2 H), 8.32(br, 1 H), 8.39(br, 1 H); m/z (APCI pos) 393 (M + H). |
| D6 | 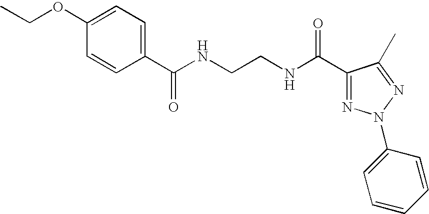 | N-(2-(4- ethoxybenzami do)ethyl)-5- methyl-2- phenyl-1H- 1,2,3- triazole-4- carboxamide | ¹H NMR (400 MHz, DMSO- d₆) δ 1.33(t, J = 6.8 Hz, 3 H), 2.52(s, 3 H), 3.45(s, 4 H), 4.07(q, 2 H), 6.97(d, J = 8.4 Hz, 2 H), 7.46(t, 1 H), 7.59(t, J = 7.6 Hz, 2 H), 7.81(d, J = 8.4 Hz, 2 H), 8.04(Hz, 2 H), 8.44(br, 1 H), 8.67 (br, 1 H); m/z (APCI pos) 394 (M + H). |

### Example S1

### N-(2-(4-Ethoxybenzamido)ethyl)-1-(4-fluorophenyl)-3-(trifluoromethyl)-1H-pyrazole-4-carboxamide

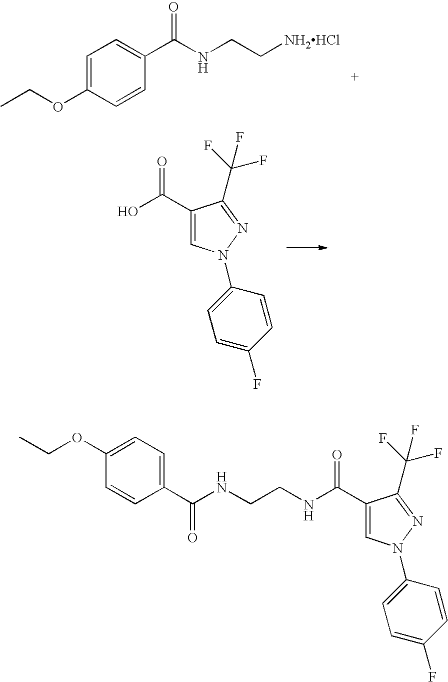

To a 10 ml round-bottomed flask were added EDAC.HCl (0.084 g, 0.44 mmol), HOBt.H₂O (0.067 g, 0.44 mmol), and 1-(4-fluorophenyl)-3-(trifluoromethyl)-1H-pyrazole-4-carboxylic acid (0.100 g, 0.36 mmol). These components were dissolved in DMF (0.8 mL) and N-(2-aminoethyl)-4-ethoxybenzamide hydrochloride (0.098 g, 0.40 mmol) was added, followed by DIPEA (0.13 ml, 0.73 mmol). The mixture was stirred for 16 h. The solvent was removed in vacuo, and the residue was dissolved in DCM (80 ml). The organic layer was washed with water, 10% HCl, and brine (˜20 ml each), dried (sodium sulfate) and filtered. The filtrate was concentrated in vacuo and the residue was purified by silica gel column chromatography (5% MeOH/DCM and 2:1 hexanes/acetone=2/1) to give N-(2-(4-ethoxybenzamido)ethyl)-1-(4-fluorophenyl)-3-(trifluoromethyl)-1H-pyrazole-4-carboxamide (0.037 g, 22%) as a powder: ¹H NMR (400 MHz, DMSO-d₆) δ 1.27 (t, 3H, J=7.0 Hz), 2.51 (m, 4H), 4.25 (q, 2H, J=7.0 Hz), 7.64 (m, 4H), 7.98 (m, 4H), 8.58 (br, 1H), 9.24 (br, 2H); m/z (APCI pos) 465.0 (70%) (M+H)

Compounds of the following structures were made from N-(2-aminoethyl)-4-ethoxybenzamide hydrochloride and the corresponding acids according to the above procedure.

| Ex # | Structure | Name | Physical Data |
|---|---|---|---|
| S2 | 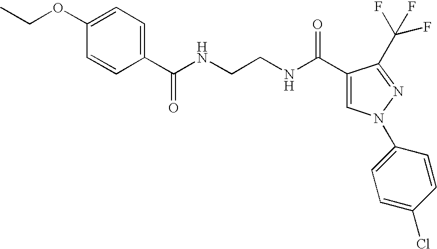 | 1-(4- chlorophenyl)- N-(2-(4- ethoxybenzamido) ethyl)-3- (trifluoromethyl)- 1H- pyrazole-4- carboxamide | Solid (24%); ¹H NMR (400 MHz, DMSO-d₆) δ 1.34 (t, 3H, J = 7.0 Hz), 3.40 (m, 4H), 4.07 (q, 2H, J = 7.0 Hz), 6.97 (m, 2H), 7.68 (m, 2H), 7.83 (m, 4H), 8.43 (m, 1H), 8.48 (m, 1H), 9.08 (m, 1H); m/z (APCI pos) 481.0 (60%) (M + H). |
| S3 | 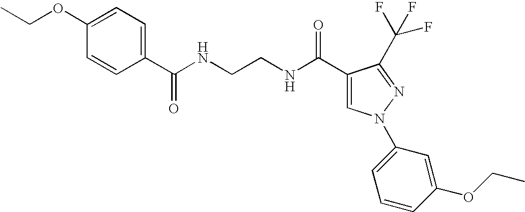 | N-(2-(4- ethoxybenzamido) ethyl)-1- (3- ethoxyphenyl)- 3- trifluoromethyl)- 1H-pyrazole-4- carboxamide | Solid (32%); ¹H NMR (400 MHz, DMSO-d₆) δ 1.35 (m, 6H), 3.40 (m, 4H), 4.10 (m, 4H), 6.96 (m, 2H), 7.02 (m, 1H), 7.35 (m, 2H), 7.49 (t, 1H, J = 7.8 Hz), 7.81 (m, 2H), 8.44 (m, 2H), 9.07 (m, 1H); m/z (APCI pos) 491.0 (20%) (M + H). |
| S4 | 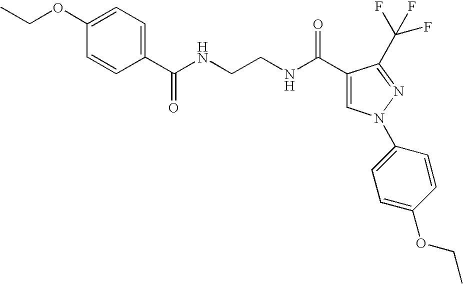 | N-(2-(4- ethoxybenzamido) ethyl)-1- (4- ethoxyphenyl)-3- (trifluoromethyl)- 1H- pyrazole-4- carboxamide | Solid (14%); ¹H NMR (400 MHz, DMSO-d₆) δ 1.34 (m, 6H), 3.39 (m, 4H), 4.09 (m, 4H), 6.96 (m, 2H), 7.12 (m, 2H), 7.68 (m, 2H), 7.80 (m, 2H), 8.93 (br, 1H); m/z (APCI pos) 491.1 (10%) (M + H). |
| S12 | 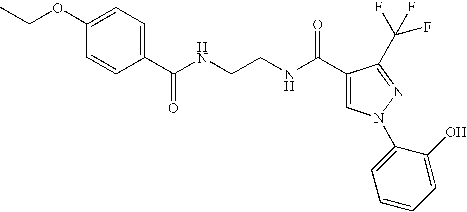 | N-(2-(4- ethoxybenzamido) ethyl)-1-(2- hydroxyphenyl)-3- (trifluoromethyl)-1H- pyrazole-4- carboxamide | Solid (26%); ¹H NMR (400 MHz, DMSO-d₆) δ 1.33 (t, 3H, J = 7.0 Hz), 3.39 (s, 4H), 4.07 (q, 2H, J =7.0 Hz), 6.97 (m, 3H), 7.12 (m, 1H), 7.32 (m, 1H), 7.57 (m, 1H), 7.81 (d, 2H, J = 8.6 Hz), 8.43 (s, 1H), 8.51 (s, 1H), 8.83 (s, 1H), 10.61 (s, 1H); m/z (APCI pos) 463.0 (10%) (M + H). |

### Example S5

### N-(2-(4-Ethoxybenzamido)ethyl)-1-phenyl-5-(trifluoromethyl)-1H-pyrazole-4-carboxamide

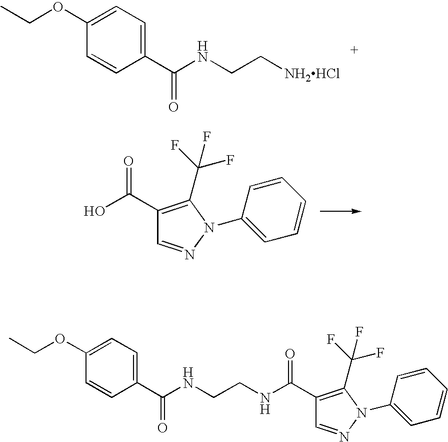

To N-(2-aminoethyl)-4-ethoxybenzamide hydrochloride (382 mg, 1.6 mmol) in DCM (50 mL) were added successively 1-phenyl-5-(trifluoromethyl)-1H-pyrazole-4-carboxylic acid (400 mg, 1.6 mmol), EDAC.HCl (389 mg, 20.3 mmol), HOBt.H₂O (295 mg, 2.2 mmol) and triethylamine (316 mg, 3.1 mmol). The mixture was stirred at room temperature for 12 hours and 2N HCl was added (100 mL). The organic layer was isolated and washed with 10% aqueous potassium carbonate and brine. After concentration and purification by silica gel column chromatography (DCM/MeOH=99/1), N-(2-(4-ethoxybenzamido)ethyl)-1-phenyl-5-(trifluoromethyl)-1H-pyrazole-4-carboxamide was isolated as a white solid (412 mg, 59%): ¹H NMR (400 MHz, DMSO-d₆) δ 1.34 (t, 3H), 3.40 (m, 4H), 4.08 (q, 2H), 6.98 (m, 2H), 7.48-7.62 (m, 5H), 7.82 (d, 2H), 8.12 (s, 1H), 8.42 (t, 1H), 8.66 (t, 1H); m/e (APCI pos) 447.0 (40%) (M+H).

### Example S6

### N-(2-(4-(2-Hydroxyethoxy)benzamido)ethyl)-1-phenyl-3-(trifluoromethyl)-1H-pyrazole-4-carboxamide

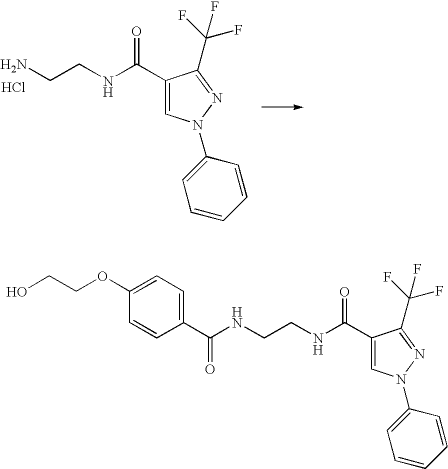

N-(2-Aminoethyl)-1-phenyl-3-(trifluoromethyl)-1H-pyrazole-4-carboxamide hydrochloride (0.20 g, 0.60 mmol), 4-(2-hydroxyethoxy)benzoic acid (0.11 g, 0.60 mmol), and HATU (0.27 g, 0.72 mmol) were suspended in THF (10 mL). DIPEA (0.34 ml, 2.0 mmol) was added and the mixture was stirred at room temperature overnight. The solution was quenched with water (75 mL) and the solid was collected by filtration and dried to provide N-(2-(4-(2-hydroxyethoxy)benzamido)ethyl)-1-phenyl-3-(trifluoromethyl)-1H-pyrazole-4-carboxamide (0.152 g, 55% yield): ¹H NMR (400 MHz, DMSO-d₆) δ 3.41 (br, 4H), 3.71-3.72 (m, 2H), 4.00-4.05 (m, 2H), 4.90 (t, J=5.2 Hz, 1H), 6.99 (d, J=7.6 Hz, 2H), 7.45-7.50 (m, 1H), 7.61 (t, J=7.6 Hz, 2H), 7.80-7.83 (m, 4H), 8.46-8.50 (m, 2H), 9.07 (s, 1H); m/z (APCI pos) 463.0 (100%) (M+H).

Compounds of the following structures were prepared from N-(2-aminoethyl)-1-phenyl-3-(trifluoromethyl)-1H-pyrazole-4-carboxamide hydrochloride and the corresponding acids, using a similar method to that described above. In some cases, compounds were purified by silica gel column chromatography or preparative HPLC. In some cases, compounds were converted to an acid form by a method commonly employed.

| Ex # | Structure | Name | Physical Data |
|---|---|---|---|
| S8 | 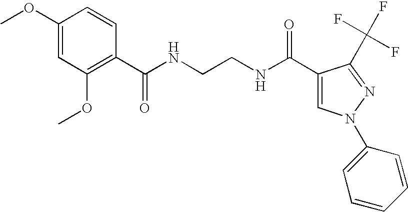 | N-(2-(2,4- dimethoxybenza- mido)ethyl)- 1-phenyl-3- (trifluoromethyl)- 1H- pyrazole-4- carboxamide | 1H NMR (400 MHz, CDCl₃) δ 3.66-3.73 (m, 4H), 3.85 (s, 3H), 3.95 (s, 3H), 6.49 (d, J = 2.4 Hz, 1H), 6.56- 6.59 (m, 1H), 7.32 (br, 1H), 7.39-7.43 (m, 1H), 7.48-7.52 (m, 2H), 7.68-7.71 (m, 2H), 8.15-8.20 (m, 2H), 8.40 (br, 1H); m/z (APCI pos) 462 (M + H). |
| S9 | 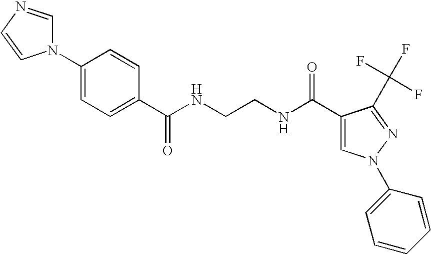 | N-(2-(4-(1H- imidazol-1- yl)benzamido) ethyl)-1- phenyl-3- (trifluoromethyl)- 1H- pyrazole-4- carboxamide | 1H NMR (400 MHz, CDCl₃) δ 3.71-3.77 (m, 4H), 6.79 (s, 1H), 7.24 (s, 1H), 7.33 (s, 1H), 7.33-7.53 (m, 6H), 7.69 (d, J = 7.6 Hz, 2H), 7.71 (s, 1H), 7.92-7.97 (m, 2H), 8.44 (s, 1H); m/z APCI pos) 469 (M + H). |
| S10 | 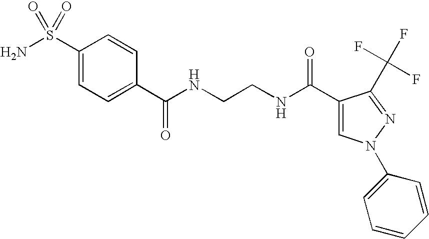 | 4-((2-(1- phenyl-3- (trifluoromethyl)- 1H- pyrazole-4- carboxamido) ethyl)carbamoyl) benzene- sulfonamide | 1H NMR (400 MHz, DMSO- d₆) δ 3.44 (br, 4H), 7.47 (br, 3H), 7.61 (br, 2H), 7.81 (d, J = 7.4 Hz, 2H), 7.89 (d, J = 7.4 Hz, 2H), 8.02 (d, J = 7.8 Hz, 2H), 8.58 (s, 1H), 8.84 (s, 1H), 9.16 (s, 1H); m/z (APCI pos) 482 (M + H). |
| S11 | 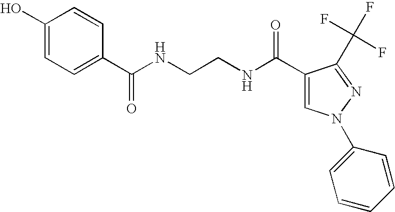 | N-(2-(4- hydroxybenzamido) ethyl)-1- phenyl-3- (trifluoromethyl)- 1H- pyrazole-4- carboxamide | 1H NMR (400 MHz, DMSO- d₆) δ 3.39 (br, 4H), 6.79 (d, J = 8.8 Hz, 2H), 7.48 (t, J = 7.4 Hz, 1H), 7.61 (t, J = 7.9 Hz, 2H), 7.72 (d, J = 8.8 Hz, 2H), 7.81 (d, J = 8.8 Hz, 2H), 8.33 (br, 1H), 8.47 (br, 1H), 9.06 (s, 1H), 9.94 (s, 1H); m/z (APCI pos) 419 (M + H). |
| S13 | 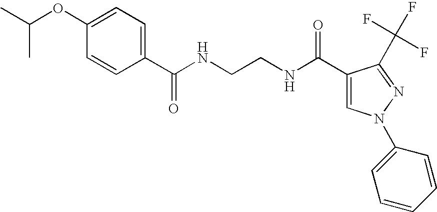 | N-(2-(4- isopropoxybenza- mido)ethyl)- 1-phenyl-3- (trifluoromethyl)- 1H- pyrazole-4- carboxamide | 1H NMR (400 MHz, CDCl₃) δ 1.34 (d, J = 5.3 Hz, 6H), 3.67 (s, 4H), 4.60 (s, br, 1H), 6.88 (d, J = 8.0 Hz, 2H), 7.39-7.42 (m, 1H), 7.47-7.49 (m, 2H), 7.67 (d, J = 7.4 Hz, 2H), 7.75 (d, J = 8.0 Hz, 2H), 8.45 (s, 1H); m/z (APCI pos): 461 (M + H). |
| S14 | 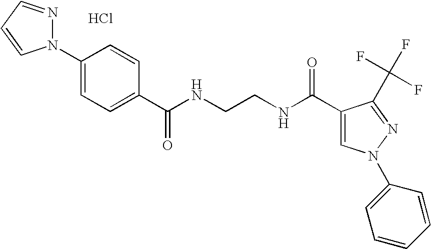 | N-(2-(4-(1H- pyrazol-1- yl)benzamido) ethyl)-1- phenyl-3- (trifluoromethyl)- 1H- pyrazole-4- carboxamide hydrochloride | 1H NMR (400 MHz, CDCl₃) δ 3.72 (s, 4H), 6.50-6.51 (m, 1H), 7.39-7.43 (m, 1H), 7.50 (t, J = 7.8 Hz, 2H), 7.69 (d, J = 7.4 Hz, 2H), 7.76 (d, J = 7.4 Hz, 2H), 7.79 (s, 1H), 7.91 (s, 1H), 7.93 (s, 1H), 7.98 (d, J = 2.4 Hz, 1H), 8.44 (s, 1H); m/z (APCI pos) 469 (M + H). |
| S15 | 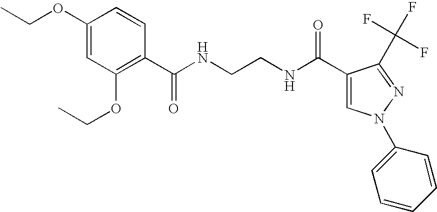 | N-(2-(2,4- diethoxybenza- mido)ethyl)- 1-phenyl-3- (trifluoromethyl)- 1H- pyrazole-4- carboxamide | ¹H NMR (400 MHz, CDCl₃) δ 1.42 (t, J = 6.8 Hz, 3H), 1.50 (t, J = 7.2 Hz, 3H), 3.64-3.72 (m, 4H), 4.06 (q, J = 6.8, 14.0 Hz, 2H), 4.18 (q, J = 6.8, 14.0 Hz, 2H), 6.46 (d, J = 2.4 Hz, 1H), 6.55 (dd, J = 2.4, 9.2 Hz, 1H), 7.40-7.44 (m, 2H), 7.51 (t, J = 7.2 Hz, 2H), 7.70 (d, J = 7.2 Hz, 2H), 8.15 (d, J = 9.2 Hz, 1H), 8.35 (br, 1H), 8.39 (s, 1H); m/z (APCI pos) 491.1 (100%) (M + H). |
| S17 | 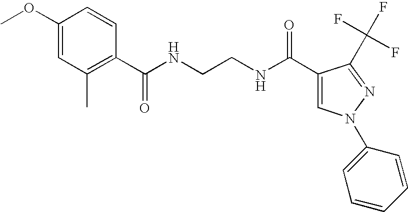 | N-(2-(4- methoxy-2- methylbenza- mido)ethyl)-1- phenyl-3- (trifluoromethyl)- 1H- pyrazole-4- carboxamide | ¹H NMR (400 MHz, CDCl₃) δ 2.43 (s, 3H), 3.68 (t, J = 2.4 Hz, 4H), 3.79 (s, 3H), 6.39 (br, 1H), 6.69-6.72 (m, 2H), 7.08 (br, 1H), 7.34-7.42 (m, 2H), 7.46-7.50 (m, 2H), 7.66 (d, J = 8.0 Hz, 2H), 8.42 (s, 1H); m/z (APCI pos) 447.0, (100%) (M + H). |
| S18 | 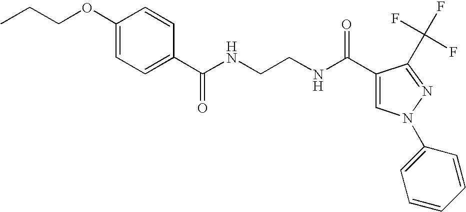 | 1-phenyl-N- (2-(4- propoxybenza- mido)ethyl)-3- (trifluoromethyl)- 1H- pyrazole-4- carboxamide | 1H NMR (400 MHz, DMSO- d₆) δ 0.98 (t, J = 7.4 Hz, 3H), 1.71-1.76 (m, 2H), 3.40-3.41 (m, 4H), 3.98 (t, J = 6.5 Hz, 2H), 6.97 (s, 1H), 6.99 (s, 1H), 7.48 (t, J = 7.4 Hz, 1H), 7.61 (t, J = 8.0 Hz, 2H), 7.80-7.83 (m, 4H), 8.43 (br, 1H), 8.48 (br, 1H), 9.06 (s, 1H); m/z (APCI pos) 461 (M + H). |
| S19 | 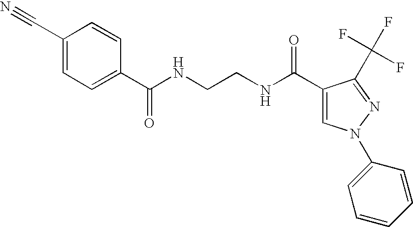 | N-(2-(4- cyanobenzamido) ethyl)-1- phenyl-3- (trifluoromethyl)- 1H- pyrazole-4- carboxamide | 1H NMR (400 MHz, DMSO- d₆) δ 3.44 (br, 4H), 7.48 (t, J = 7.4 Hz, 1H), 7.61 (t, J = 8.0 Hz, 2H), 7.80 (d, J = 7.6 Hz, 2H), 7.95-8.02 (m, 4H), 8.50 (br, 1H), 8.85 (br, 1H), 9.06 (s, 1H); m/z (APCI pos) 428 (M + H). |
| S29 | 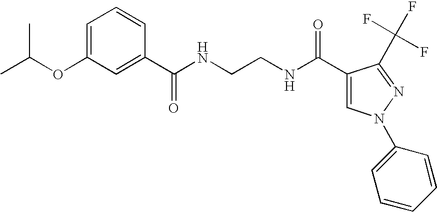 | N-(2-(3- isopropoxybenza- mido)ethyl)- 1-phenyl-3- (triflouromethyl)- 1H- pyrazole-4- carboxamide | 1H NMR (400 MHz, DMSO- d₆) δ 1.25 (d, J = 6.1 Hz, 6H), 3.41-3.42 (m, 4H), 4.61-4.67 (m, 1H), 7.04-7.07 (m, 1H), 7.32-7.41 (m, 3H), 7.48 (t, J = 7.3 Hz, 1H), 7.61 (t, J = 8.0 Hz, 2H), 7.82 (t, J = 7.6 Hz, 2H), 8.48 (s, 1H), 8.55 (s, 1H), 9.06 (s, 1H); m/z (APCI pos) 461 (M + H). |
| S32 | 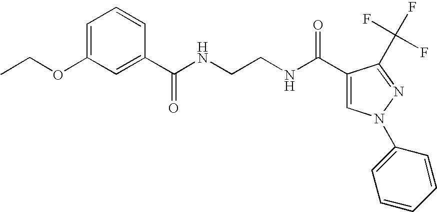 | N-(2-(3- ethoxybenzamido) ethyl)-1- phenyl-3- (trifluoromethyl)- 1H- pyrazole-4- carboxamide | 1H NMR (400 MHz, DMSO- d₆) δ 1.32 (t, J = 6.9 Hz, 3H), 3.42-3.43 (m, 4H), 4.03-4.08 (m, 2H), 7.05-7.08 (m, 1H), 7.34-7.43 (m, 3H), 7.48 (t, J = 7.4 Hz, 1H), 7.61 (t, J = 8.0 Hz, 2H), 7.80 (s, 1H), 7.82 (s, 1H), 8.49 (s, 1H), 8.56 (s, 1H), 9.06 (s, 1H); m/z (APCI pos) 447 (M + H). |
| S33 | 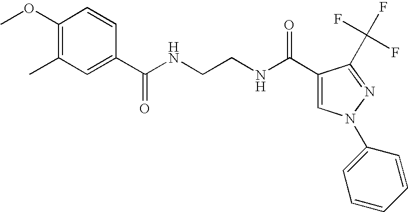 | N-(2-(4- methoxy-3- methylbenzamido) ethyl)-1- phenyl-3- (trifluoromethyl)- 1H- pyrazole-4- carboxamide | 1H NMR (400 MHz, DMSO- d₆) δ 2.17 (s, 3H), 3.40 (s, 4H), 3.83 (s, 3H), 6.99 (d, J = 8.6 Hz, 1H), 7.48 (t, J = 7.4 Hz, 1H), 7.61 (t, J = 7.9 Hz, 2H), 7.68 (s, 1H), 7.71 (d, J = 8.4 Hz, 1H), 7.81 (d, J = 7.8 Hz, 2H), 8.40 (s, 1H), 8.48 (s, 1H), 9.06 (s, 1H); m/z (APCI pos) 447 (M + H). |
| S37 | 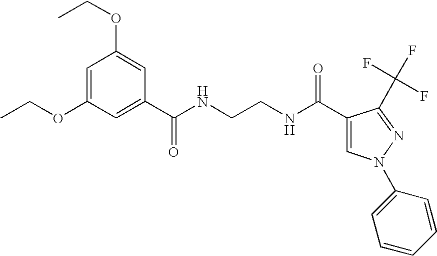 | N-(2-(3,5- diethoxybenza- mido)ethyl)- 1-phenyl-3- (trifluoromethyl)- 1H- pyrazole-4- carboxamide | 1H NMR (400 MHz, DMSO- d₆) δ 1.31 (t, J = 7.0, 6H), 3.40-3.41 (m, 4H), 4.00-4.05 (m, 4H), 6.60 (t, J = 2.2 Hz, 1H), 6.98 (d, J = 2.1 Hz, 2H), 7.48 (t, J = 7.4 Hz, 1H), 7.61 (t, J = 8.0 Hz, 2H), 7.81 (d, J = 7.6 Hz, 2H), 8.48 (br, 1H), 8.52 (br, 1H), 9.06 (s, 1H); m/z (APCI pos) 491 (M + H). |
| S38 | 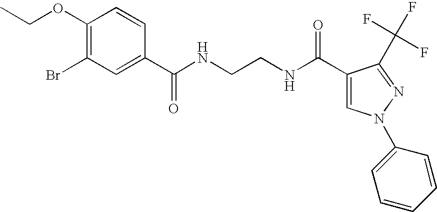 | N-(2-(3- bromo-4- ethoxybenzamido) ethyl)-1- phenyl-3- (trifluoromethyl)- 1H- pyrazole-4- carboxamide | ¹H NmR (400 MHz, DMSO- d₆) δ 1.37 (t, J = 6.9 Hz, 3H), 3.40 (br, 4H), 4.15-4.20 (m, 2H), 7.17 (d, J = 8.8 Hz, 1H), 7.48 (t, J = 7.4 Hz, 1H), 7.59 (t, J = 7.9 Hz, 2H), 7.81 (d, J = 7.8 Hz, 2H), 7.84-7.87 (m, 1H), 8.09 (d, J = 2.1 Hz, 1H), 8.47 (br, 1H), 8.57 (br, 1H), 9.05 (s, 1H); m/z (APCI pos) 525 (M + H). |
| S39 | 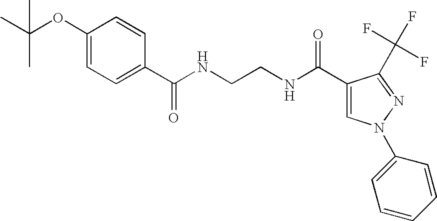 | N-(2-(4-tert- butoxybenzamido) ethyl)-1- phenyl-3- (trifluoromethyl)- 1H- pyrazole-4- carboxamide | ¹H NMR (400 MHz, DMSO- d₆) δ 1.33 (s, 9H), 3.40-3.42 (m, 4H), 7.03 (d, J = 8.6 Hz, 2H), 7.48 (t, J = 7.4 Hz, 1H), 7.61 (t, J = 8.0 Hz, 2H), 7.80 (t, J = 8.3 Hz, 4H), 8.48 (br, 2H), 9.06 (s, 1H); m/z (APCI pos) 475 (M + H). |
| S40 | 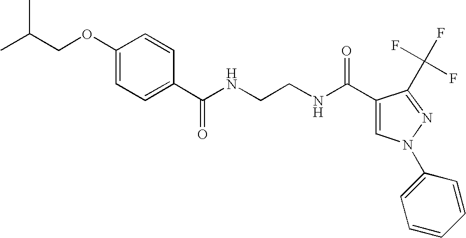 | N-(2-(4- isobutoxybenza- mido)ethyl)- 1-phenyl-3- (trifluoromethyl)- 1H- pyrazole-4- carboxamide | ¹H NMR (400 MHz, DMSO- d₆) δ 0.98 (d, J = 6.8 Hz, 6H), 1.99-2.05 (m, 1H), 3.40 (br, 4H), 3.779 (d, J = 6.6 Hz, 2H), 6.98 (d, J = 8.8 Hz, 2H), 7.48 (t, 7.3 Hz, 1H), 7.61 (t, J = 7.9 Hz, 2H), 7.80-7.83 (m, 4H), 8.43 (br, 1H), 8.48 (br, 1H), 9.06 (s, 1H); m/z (APCI pos) 475 (M + H). |

### Example S16

### N-(2-(4-Ethoxybenzamido)ethyl)-1-(3-hydroxyphenyl)-3-(trifluoromethyl)-1H-pyrazole-4-carboxamide

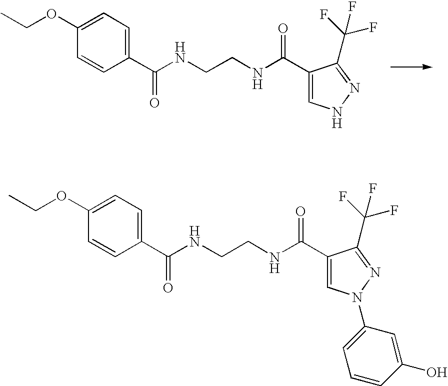

To a 50 mL sealed tube flushed with argon were added copper(I) iodide (0.00257 g, 0.014 mmol), potassium carbonate (0.0784 g, 0.567 mmol), and N-(2-(4-ethoxybenzamido)ethyl)-3-(trifluoromethyl)-1H-pyrazole-4-carboxamide (0.100 g, 0.27 mmol). (1S,2S)—N1,N2-Dimethylcyclohexane-1,2-diamine (0.00768 g, 0.054 mmol) and 3-iodophenol (0.0713 g, 0.32 mmol) were then added, and these components were suspended in toluene (2 mL). The tube was sealed and heated to 110° C. overnight, then cooled to room temperature and the mixture was filtered through celite to remove the solid. The filtrate was concentrated under vacuum and the residue was purified by silicagel column chromatography (10% Et₂O/DCM) to give N-(2-(4-ethoxybenzamido)ethyl)-1-(3-hydroxyphenyl)-3-(trifluoromethyl)-1H-pyrazole-4-carboxamide (0.024 g, 19%) as a solid: ¹H NMR (400 MHz, DMSO-d₆) δ 1.39 (t, 3H, J=7.0 Hz), 3.57 (m, 4H), 4.08 (q, 2H, J=7.0 Hz), 6.85 (m, 1H), 6.95 (d, 2H, J=9.0 Hz), 7.20-7.23 (m, 2H), 7.33 (m, 1H), 7.78 (d, 2H, J=9.0 Hz); m/z (APCI pos) 463.0 (10%) (M+H).

Compound of the following structure was prepared from N-(2-(4-ethoxybenzamido)ethyl)-3-(trifluoromethyl)-1H-pyrazole-4-carboxamide and 3-iodobenzonitrile, using a similar method to that described above.

| Ex # | Structure | Name | Physical Data |
|---|---|---|---|
| S46 | 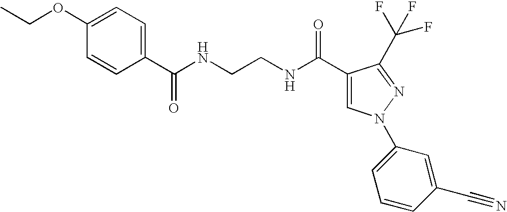 | 1-(3- cyanophenyl)- N-(2-(4- ethoxybenzamido) ethyl)-3- (trifluoromethyl)- 1H- pyrazole-4- carboxamide | Solid (6%); ¹H NMR (400 MHz, DMSO-d₆) δ 1.34 (t, 3H, J = 7.0 Hz), 3.41 (m, 4H), 4.08 (q, 2H, J = 7.0 Hz), 6.97 (d, 2H, J = 9.0 Hz), 7.82 (m, 3H), 7.96 (m, 1H), 8.17 (m, 1), 8.30 (m, 1H), 8.43 (m, 1H), 8.49 (m, 1H), 9.17 (s, 1H); m/z (APCI pos) 472.1 (100%) (M + H). |

### Example S21 and Example S34

### Methyl 2-(4-(2-(4-ethoxybenzamido)ethylcarbamoyl)-3-(trifluoromethyl)-1H-pyrazol-1-yl)benzoate and 2-(4-((2-(4-ethoxybenzamido)ethyl)carbamoyl)-3-(trifluoromethyl)-1H-pyrazol-1-yl)benzoic acid

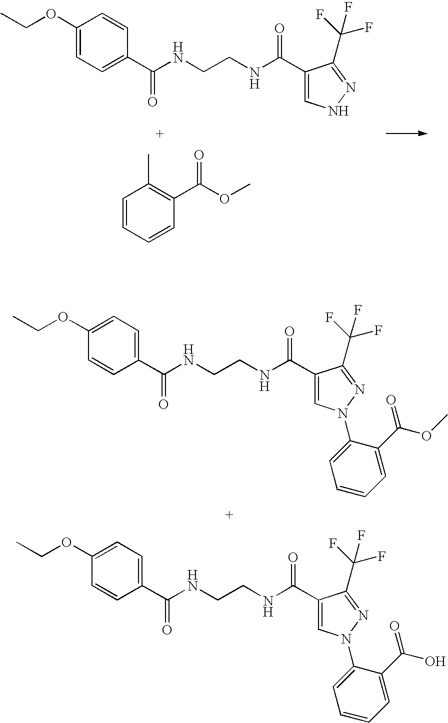

To a 50 mL sealed tube were added N-(2-(4-ethoxybenzamido)ethyl)-3-(trifluoromethyl)-1H-pyrazole-4-carboxamide (0.200 g, 0.540 mmol), (1R,2R)—N1,N2-dimethylcyclohexane-1,2-diamine (0.0154 g, 0.108 mmol), copper(I) iodide (0.00514 g, 0.0270 mmol), potassium carbonate (0.149 g, 1.08 mmol), and methyl 2-iodobenzoate (1.42 g, 5.40 mmol). The mixture was stirred for 16 hours at 110° C., cooled to room temperature, and filtered through celite. The solvent was removed under vacuum and the residue was chromatographed on silica gel (9:0.5:0.5 DCM/EtOAc/MeOH) to give methyl 2-(4-((2-(4-ethoxybenzamido)ethyl)carbamoyl)-3-(trifluoromethyl)-1H-pyrazol-1-yl)benzoate (0.017 g, 6%) as a solid: ¹H NMR (400 MHz, DMSO-d₆) δ 1.42 (t, 3H, J=7.0 Hz), 3.65 (m, 4H), 3.70 (s, 3H), 4.05 (q, 2H, J=7.0 Hz), 6.87 (d, 2H, J=8.6 Hz), 7.20 (s, 1H), 7.33 (s, 1H), 7.43 (m, 1H), 7.56 (m, 1H), 7.63 (m, 1H), 7.75 (d, 2H, J=8.6 Hz), 7.94 (m, 1H), 8.25 (s, 1H); m/z (APCI pos) 505.0 (100%) (M+H).

The column was further eluted with 10% MeOH/DCM to give the corresponding acid analog. This compound was further purified by dissolving in 1N NaOH and washing with EtOAc, followed by acidification of the aqueous layer with 1N HCl, filtering the resulting solid, and drying under vacuum to give 2-(4-((2-(4-ethoxybenzamido)ethyl)carbamoyl)-3-(trifluoromethyl)-1H-pyrazol-1-yl)benzoic acid (40 mg, 15%) as a solid; ¹H NMR (400 MHz, DMSO-d₆) δ 1.33 (t, 3H, J=7.0 Hz), 3.39 (m, 4H), 4.07 (q, 2H, J=7.0 Hz), 6.96 (d, 2H, J=9.0 Hz), 7.66 (m, 2H), 7.79 (m, 3H), 7.91 (m, 1H), 8.45 (m, 2H), 8.71 (s, 1H), 13.26 (s, 1H); m/z (APCI neg) 489.1 (100%) (M+H).

### Example S22

### 1-(2-(2-(Dimethylamino)ethoxy)phenyl)-N-(2-(4-ethoxybenzamido)ethyl)-3-(trifluoromethyl)-1H-pyrazole-4-carboxamide

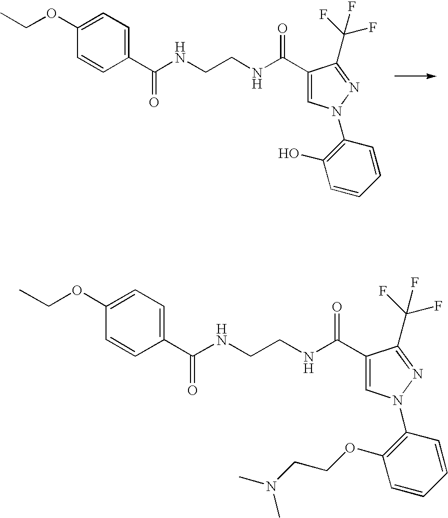

To a solution of N-(2-(4-ethoxybenzamido)ethyl)-1-(2-hydroxyphenyl)-3-(trifluoromethyl)-1H-pyrazole-4-carboxamide (0.050 g, 0.11 mmol), 2-(dimethylamino)ethanol (0.0106 g, 0.12 mmol), and tributylphosphine (0.0405 mL, 0.162 mmol) in THF was added 1,1′-(azodicarbonyl)dipiperidine (ADDP, 0.0409 g, 0.16 mmol). The mixture was stirred for 1 hour. Another 1 equivalent of alcohol, ADDP, and tributylphosphine were added and the mixture was stirred overnight. The solvent was then removed under vacuum, and the residue was purified by silica gel chromatography (9:0.5:0.5 DCM/EtOAc/MeOH) to give 1-(2-(2-(dimethylamino)ethoxy)phenyl)-N-(2-(4-ethoxybenzamido)ethyl)-3-(trifluoromethyl)-1H-pyrazole-4-carboxamide (0.028 g, 48% yield) as a solid after trituration with ether: ¹H NMR (400 MHz, DMSO-d₆) δ 1.33 (t, 3H, J=7.0 Hz), 2.12 (s, 6H), 2.60 (t, 2H, J=5.5 Hz), 3.39 (m, 4H), 4.07 (q, 2H, J=7.0 Hz), 4.19 (t, 2H, J=5.5 Hz), 6.96 (d, 2H, J=9.0 Hz), 7.13 (m, 1H), 7.33 (m, 1H), 7.48 (m, 1H), 7.63 (m, 1H), 7.81 (d, 2H, J=8.6 Hz), 8.41 (m, 2H), 8.82 (s, 1H); m/z (APCI pos) 534.2 (100%) (M+H).

Compounds of the following structures were prepared from the corresponding phenols and alcohols, using a similar method to that described above.

| Ex # | Structure | Name | Physical Data |
|---|---|---|---|
| S23 | 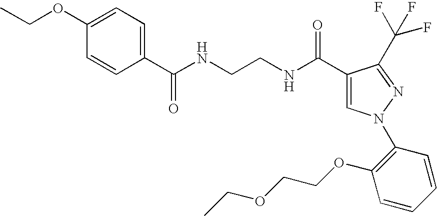 | N-(2-(4- ethoxybenzamido) ethyl)-1-(2- (2- ethoxyethoxy) phenyl)-3- (trifluoromethyl)- 1H- pyrazole-4- carboxamide | Solid (29%); ¹H NMR (400 MHz, CDCl₃) δ 1.19 (t, 3H, J = 7.0 Hz), 1.43 (t, 3H, J = 7.0 Hz), 3.54 (q, 2H, J = 7.0 Hz), 3.69 (m, 4H), 3.78 (m, 2H), 4.07 (q, 2H, J = 7.0 Hz), 4.23 (m, 2H), 6.72 (m, 1H), 6.90 (d, 2H, J = 9.0 Hz), 7.04 (m, 1H), 7.07-7.12 (m, 2H), 7.36 (m, 1H), 7.76 (m, 3H), 8.72 (s, 1H); m/z (APCI pos) 535.1 (10%) (M + H). |
| S25 | 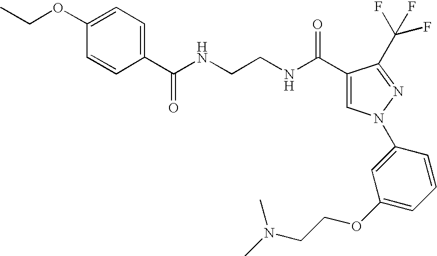 | 1-(3-(2- (dimethylamino) ethoxy)phenyl)- N-(2-(4- ethoxybenzamido) ethyl)-3- (trifluoromethyl)- 1H- pyrazole-4- carboxamide | Solid (42%); ¹H NMR (400 HHz, CDCl₃) δ 1.33 (t, 3H, J = 7.0 Hz), 2.23 (s, 6H), 2.66 (t, 2H, J = 5.9 Hz), 3.40 (m, 4H), 4.08 (q, 2H, J = 7.0 Hz), 4.14 (t, 2H, J = 5.9 Hz), 6.97 (d, 2H, J = 8.6 Hz), 7.05 (m, 1H), 7.38 (m, 2H), 7.49 (m, 1H), 7.81 (d, 2H, J = 9.0 Hz), 8.43 (m, 2H), 9.08 (s, 1H); m/z (APCI pos) 534.1 (100%) (M + H). |
| S26 | 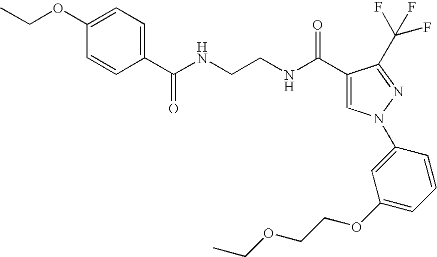 | N-(2-(4- ethoxybenzamido) ethyl)-1-(3- (2- ethoxyethoxy) phenyl)-3- (trifluoromethyl)- 1H- pyrazole-4- carboxamide | Solid (16%); ¹H NMR (400 MHz, CDCl₃) δ 1.24 (t, 3H, J = 7.0 Hz), 1.41 (t, 3H, J = 7.0 Hz), 3.60 (q, 2H, J = 7.0 Hz), 3.64 (m, 4H), 3.80 (m, 2H), 4.04 (q, 2H, J = 7.0 Hz), 4.16 (m, 2H), 6.87 (d, 2H, J = 9.0 Hz), 6.93 (m, 1H), 7.17 (m, 1H), 7.28 (m, 2H), 7.33 (t, 1H, J = 8.2 Hz), 7.42 (m, 1H), 7.76 (d, 2H, J = 9.0 Hz), 8.48 (s, 1H); m/z (APCI pos) 535.1 (100%) (M + H). |

### Example S27 and Example S20

### Ethyl 2-(2-(4-(2-(4-ethoxybenzamido)ethylcarbamoyl)-3-(trifluoromethyl)-1H-pyrazol-1-yl)phenoxy)acetate and 2-(2-(4-(2-(4-ethoxybenzamido)ethylcarbamoyl)-3-(trifluoromethyl)-1H-pyrazol-1-yl)phenoxy)acetic acid

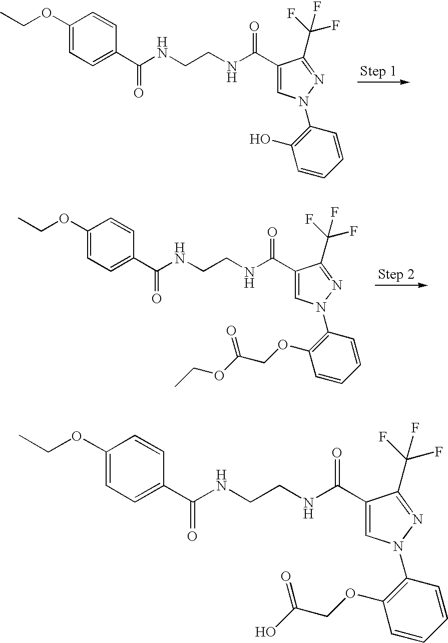

### Step 1

To a mixture of N-(2-(4-ethoxybenzamido)ethyl)-1-(2-hydroxyphenyl)-3-(trifluoromethyl)-1H-pyrazole-4-carboxamide (0.100 g, 0.22 mmol), and potassium carbonate (60 mg, 0.43 mmol) in DMF (1 ml) was added ethyl 2-bromoacetate (0.024 ml, 0.22 mmol). The mixture was stirred for 1 hour then filtered through a 0.45 μM acrodisc syringe filter (Pall Co.). The solvent was removed under vacuum and the residue was purified by silica gel column chromatography (90:5:5 DCM/EtOAc/MeOH −10% 7N ammonia in MeOH/DCM) to give ethyl 2-(2-(4-(2-(4-ethoxybenzamido)ethylcarbamoyl)-3-(trifluoromethyl)-1H-pyrazol-1-yl)phenoxy)acetate (0.089 g, 75%) as a solid: ¹H NMR (400 MHz, CDCl₃) δ 1.28 (t, 3H, J=7.0 Hz), 1.43 (t, 3H, J=7.0 Hz), 3.70 (m, 4H), 4.07 (q, 2H, J=7.0 Hz), 4.25 (q, 2H, J=7.0 Hz), 4.73 (s, 2H), 6.84 (m, 1H), 6.90 (d, 2H, J=8.6 Hz), 6.96 (m, 1H), 7.05 (m, 1H), 7.16 (m, 1H), 7.36 (m, 1H), 7.76 (d, 2H, J=9.0 Hz), 7.86 (m, 1H), 8.98 (s, 1H); m/z (APCI pos) 549.1 (20%) (M+H).

The compound of the following structure was prepared from N-(2-(4-ethoxybenzamido)ethyl)-1-(3-hydroxyphenyl)-3-(trifluoromethyl)-1H-pyrazole-4-carboxamide and ethyl 2-bromoacetate using a similar method to that described above.

| Ex # | Structure | Name | Physical Data |
|---|---|---|---|
| S28 | 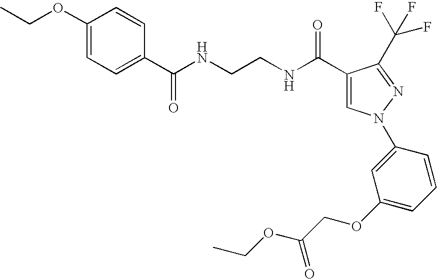 | ethyl 2-(3- (4-(2-(4- ethoxybenzamido) ethylcarba- moyl)-3- (trifluoromethyl)- 1H- pyrazol-1- yl)phenoxy) acetate | Solid (72%); ¹H NMR (400 MHz, CDCl₃) δ 1.31 (t, 3H, J = 7.0 Hz), 1.43 (t, 3H, J = 7.0 Hz), 3.69 (m, 4H), 4.07 (q, 2H, J = 7.0 Hz), 4.29 (q, 2H, J = 7.0 Hz), 4.69 (s, 2H), 6.88-7.00 (m, 4H), 7.27-7.32 (m, 3H), 7.40 (t, 1H, J = 8.2 Hz), 7.76 (d, 2H, J = 9.0 Hz), 8.40 (s, 1H); m/z (APCI pos) 549.0 (50%) (M + H). |

### Step 2

To a solution of ethyl 2-(2-(4-(2-(4-ethoxybenzamido)ethylcarbamoyl)-3-(trifluoromethyl)-1H-pyrazol-1-yl)phenoxy)acetate (0.050 g, 0.091 mmol) in MeOH was added lithium hydroxide monohydrate (0.011 g, 0.27 mmol). The mixture was stirred for 3 hours. The solvent was removed in vacuo and the residue was taken up in water and acidified with 10% HCl to pH ˜4. The solid precipitate was collected and dried under vacuum to give 2-(2-(4-(2-(4-ethoxybenzamido)ethylcarbamoyl)-3-(trifluoromethyl)-1H-pyrazol-1-yl)phenoxy)acetic acid (0.031 g, 65%) as a solid: ¹H NMR (400 MHz, DMSO-d₆) δ 1.33 (t, 3H, J=7.0 Hz), 3.38 (m, 4H), 4.07 (q, 2H, J=7.0 Hz), 4.88 (s, 2H), 6.96 (d, 2H, J=8.6 Hz), 7.15 (m, 1H), 7.22 (m, 1H), 7.46 (m, 1H), 7.65 (m, 1H), 7.81 (d, 2H, J=8.6 Hz), 8.44 (m, 1H), 8.50 (m, 1H), 8.85 (s, 1H); m/z (APCI neg) 519.1 (100%) (M−H).

The compound of the following structure was prepared from ethyl 2-(3-(4-(2-(4-ethoxybenzamido)ethylcarbamoyl)-3-(trifluoromethyl)-1H-pyrazol-1-yl)phenoxy)acetate using a similar method to that described above.

| Ex # | Structure | Name | Physical Data |
|---|---|---|---|
| S24 | 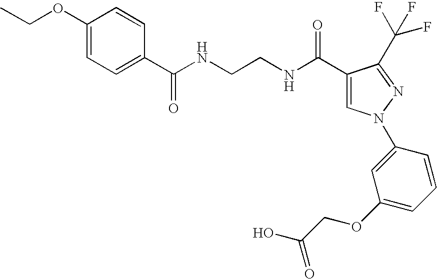 | 2-(3-(4-(2- (4- ethoxybenzamido) ethylcarba- moyl)-3- (trifluoromethyl)- 1H- pyrazol-1- yl)phenoxy) acetic acid | Solid (65%); ¹H NMR (400 MHz, DMSO-d₆) δ 1.33 (t, 3H, J = 7.0 Hz), 3.40 (m, 4H), 4.07 (q, 2H, J = 7.0 Hz), 4.78 (s, 2H), 6.97 (d, 2H, J = 9.0 Hz), 7.02 (m, 1H), 7.38 (m, 2H), 7.50 (t, 1H, J = 8.2 Hz), 7.81 (d, 2H, J = 8.6 Hz), 8.45 (m, 2H), 9.08 (s, 1H); m/z (APCI neg) 519.1 (100%) (M − H). |

### Example S30

### 1-Benzyl-N-(2-(4-ethoxybenzamido)ethyl)-3-(trifluoromethyl)-1H-pyrazole-4-carboxamide

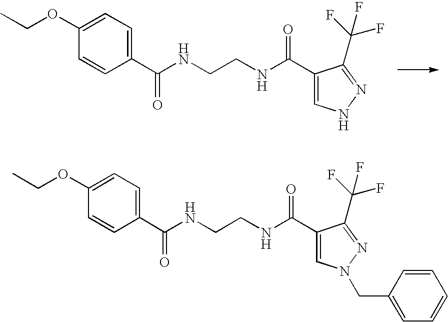

To a solution of N-(2-(4-ethoxybenzamido)ethyl)-3-(trifluoromethyl)-1H-pyrazole-4-carboxamide (0.1 g, 0.27 mmol) in DMF were added benzyl bromide (0.03533 ml, 0.30 mmol) and potassium carbonate (75 mg, 0.54 mmol). The mixture was stirred for 16 hours at room temperature and filtered. The solvent was removed under vacuum, and the residue was purified by silica gel column chromatography (9:0.5:0.5 DCM/EtOAc/MeOH) to give 1-benzyl-N-(2-(4-ethoxybenzamido)ethyl)-3-(trifluoromethyl)-1H-pyrazole-4-carboxamide (0.105 g, 84%) as a solid: ¹H NMR (400 MHz, DMSO-d₆) δ 1.34 (t, 3H, J=7.0 Hz), 3.34 (s, 4H), 4.08 (q, 2H, J=7.0 Hz), 5.45 (s, 2H), 6.96 (d, 2H, J=9.0 Hz), 7.30-7.42 (m, 5H), 7.79 (d, 2H, J=9.0 Hz), 8.38 (m, 3H); m/z (APCI pos) 461.1 (100%) (M+H).

Compounds of the following structures were prepared from N-(2-(4-ethoxybenzamido)ethyl)-3-(trifluoromethyl)-1H-pyrazole-4-carboxamide and the corresponding alkyl halides, using a similar method to that described above.

| Ex # | Structure | Name | Physical Data |
|---|---|---|---|
| S31 | 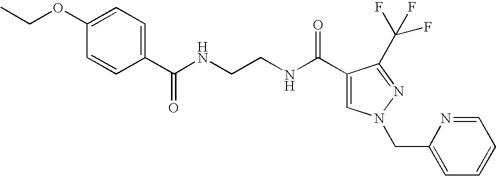 | N-(2-(4- ethoxybenzamido) ethyl)-1- (pyridin-2- ylmethyl)-3- (trifluoromethyl)- 1H- pyrazole-4- carboxamide | Solid (76%); ¹H NMR (400 MHz, DMSO-d₆) δ 1.34 (t, 3H, J = 7.0 Hz), 3.35 (m, 4H), 4.08 (q, 2H, J = 7.0 Hz), 5.55 (s, 2H), 6.96 (d, 2H, J = 9.0 Hz), 7.32 (m, 1H), 7.37 (m, 1H), 7.80 (d, 2H, J = 9.0 Hz), 7.84 (m, 1H), 8.41 (m, 2H), 8.44 (s, 1H), 8.56 (m, 1H); m/z (APCI pos) 462.0 (100%) (M + H). |
| S47 | 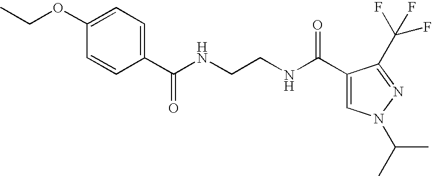 | N-(2-(4- ethoxybenzamido) ethyl)-1- isopropyl-3- (trifluoromethyl)- 1H- pyrazole-4- carboxamide | Solid (13%); ¹H NMR (400 MHz, DMSO-d₆) δ 1.34 (t, 3H, J = 7.0 Hz), 1.44 (d, 6H, J = 6.6 Hz), 3.36 (m, 4H), 4.08 (q, 2H, J = 7.0 Hz), 4.59 (m, 1H), 6.96 (d, 2H, J = 9.0 Hz), 7.80 (d, 2H, J = 9.0 Hz), 8.31 (m, 1H), 8.40 (m, 2H); m/z (APCI pos) 413.1 (100%) (M + H). |
| S48 | 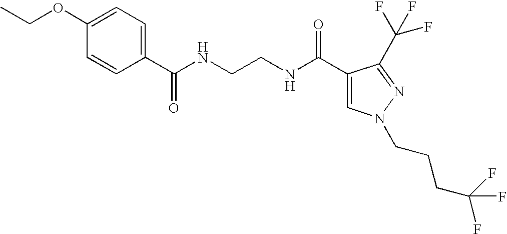 | N-(2-(4- ethoxybenzamido) ethyl)-1- (4,4,4- trifluorobutyl)-3- (trifluoromethyl)- 1H- pyrazole-4- carboxamide | Solid (18%); ¹H NMR (400 MHz, DMSO-d₆) δ 1.34 (t, 3H, J = 7.0 Hz), 2.02 (m, 2H), 2.30 (m, 2H), 3.36 (m, 4H), 4.08 (q, 2H, J = 7.0 Hz), 4.29 (t, 2H, J = 7.0 Hz), 6.96 (d, 2H, J = 9.0 Hz), 7.80 (d, 2H, J = 9.0 Hz), 8.36 (m, 2H), 8.40 (m, 1H); m/z (APCI pos) 481.1 (100%) (M + H). |
| S49 | 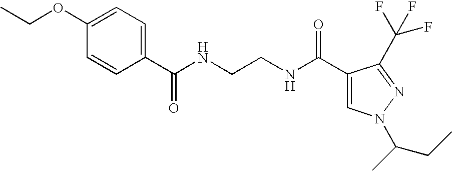 | 1-sec-butyl- N-(2-(4- ethoxybenzamido) ethyl)-3- (trifluoromethyl)- 1H- pyrazole-4- carboxamide | Solid (13%); ¹H NMR (400 MHz, DMSO-d₆) δ 0.74 (t, 3H, J = 7.4 Hz), 1.34 (t, 3H, J = 7.0 Hz), 1.42 (d, 3H, J = 6.64 Hz), 1.77 (m, 2H), 3.36 (m, 4H), 4.08 (q, 2H, J = 7.0 Hz), 4.36 (m, 1H), 6.96 (d, 2H, J = 8.6 Hz), 7.80 (d, 2H, J = 8.6 Hz), 8.33 (m, 1H), 8.37 (s, 1H), 8.41 (m, 1H); m/z (APCI pos) 427.0 (100%) (M + H). |
| S50 | 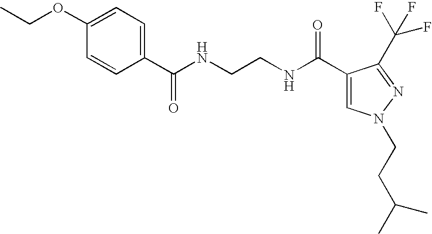 | N-(2-(4- ethoxybenzamido) ethyl)-1- isopentyl-3- (trifluoromethyl)- 1H- pyrazole-4- carboxamide | Solid (34%); ¹H NMR (400 MHz, DMSO-d₆) δ 0.90 (d, 6H, J = 6.6 Hz), 1.33 (t, 3H, J = 7.0 Hz), 1.51 (m, 1H), 1.68 (q, 2H, J = 7.4 Hz), 3.36 (m, 4H), 4.07 (q, 2H, J = 7.0 Hz), 4.21 (t, 2H, J = 7.4 Hz), 6.96 (d, 2H, J = 9.0 Hz), 7.80 (d, 2H, J = 9.0 Hz), 8.33 (m, 1H), 8.34 (s, 1H), 8.40 (m, 1H); m/z (APCI pos) 441.1 (100%) (M + H). |
| S51 | 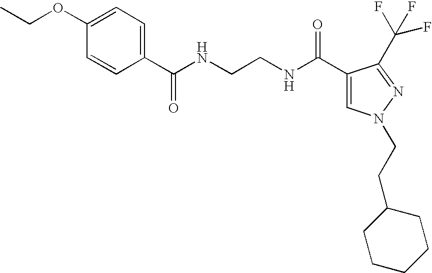 | 1-(2- cyclohexylethyl)- N-(2-(4- ethoxybenzamido) ethyl)-3- (trifluoromethyl)- 1H- pyrazole-4- carboxamide | Solid (36%); ¹H NMR (400 MHz, DMSO-d₆) δ 0.92 (m, 2H), 1.16 (m, 4H), 1.33 (t, 3H, J = 7.0 Hz), 1.56-1.73 (m, 7H), 3.35 (m, 4H), 4.07 (q, 2H, J = 7.0 Hz), 4.21 (t, 2H), 6.96 (d, 2H, J = 8.6 Hz), 7.80 (d, 2H, J = 8.6 Hz), 8.32 (m, 1H), 8.33 (s, 1H), 8.40 (m, 1H); m/z (APCI pos) 481.1 (100%) (M + H). |

### Example S35

### N-(2-(4-Ethoxybenzamido)ethyl)-3-methyl-1-phenyl-1H-pyrazole-4-carboxamide

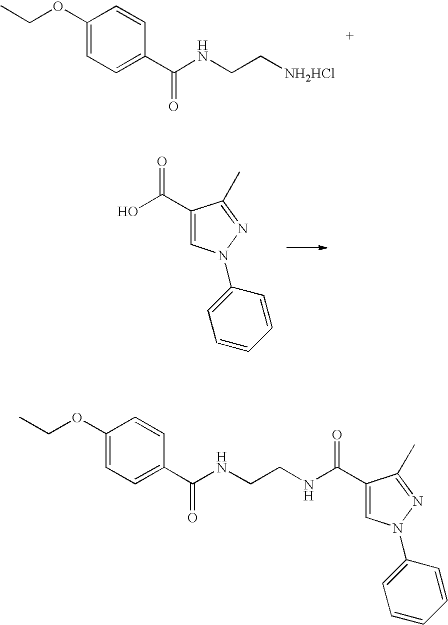

A mixture N-(2-aminoethyl)-4-ethoxybenzamide hydrochloride (200 mg, 0.981 mmol), DIPEA (349 mg, 2.697 mmol), HATU (373 mg, 0.981 mmol), and 3-methyl-1-phenyl-1H-pyrazole-4-carboxylic acid (165 mg, 0.817 mmol) (Bull. Soc. Chim. Fra. 1988, 540-547) in THF (15 mL) was stirred at room temperature overnight. The reaction mixture was quenched by adding water (150 mL). The obtained solid were filtered and washed with water and dried under high vacuum to give N-(2-(4-ethoxybenzamido)ethyl)-3-methyl-1-phenyl-1H-pyrazole-4-carboxamide (146 mg, 43%) as a tan solid: ¹H NMR (400 MHz, DMSO-d₆) δ 1.33 (t, J=6.9 Hz, 3H), 2.43 (s, 3H), 3.39 (s, 4H), 4.05-4.10 (m, 2H), 6.97 (d, J=8.8 Hz, 2H), 7.33 (t, J=7.4 Hz, 1H), 7.52 (t, J=7.9 Hz, 2H), 7.73 (d, J=7.8 Hz, 2H), 7.82 (d, J=8.8 Hz, 2H), 8.12 (s, 1H), 8.44 (s, 1H), 8.83 (s, 1H); m/z (APCI pos) 393 (M+H).

### Example S36

### N-(2-(4-Ethoxybenzamido)ethyl)-1-(2-hydroxycyclohexyl)-3-(trifluoromethyl)-1H-pyrazole-4-carboxamide

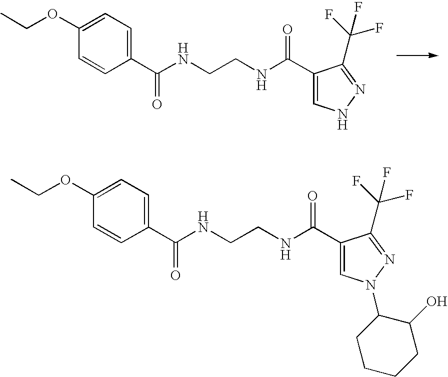

To a solution of N-(2-(4-ethoxybenzamido)ethyl)-3-(trifluoromethyl)-1H-pyrazole-4-carboxamide (0.100 g, 0.27 mmol) in 7-oxa-bicyclo[4.1.0]heptane (0.265 g, 2.70 mmol) was added cesium carbonate (0.0176 g, 0.05 mmol). The mixture was stirred for 16 hours at room temperature, and then heated at 50° C. for 16 hours. The mixture was then diluted with EtOAc and filtered, and the filtrate was concentrated under vacuum. The residue was purified by silica gel column chromatography (9:0.5:0.5 DCM/EtOAc/MeOH) to give N-(2-(4-ethoxybenzamido)ethyl)-1-(2-hydroxycyclohexyl)-3-(trifluoromethyl)-1H-pyrazole-4-carboxamide (0.070 g, 55%) as a solid: ¹H NMR (400 MHz, DMSO-d₆) δ 1.33 (t, 3H, J=7.0 Hz), 1.33 (m, 3H), 1.74 (m, 3H), 1.96 (m, 2H), 3.36 (m, 4H), 3.62 (m, 1H), 3.95 (m, 1H), 4.08 (q, 2H, J=7.0 Hz), 4.97 (d, 1H, J=5.8 Hz), 6.96 (d, 2H, J=8.9 Hz), 7.81 (d, 2H, J=8.9 Hz), 8.32 (m, 2H), 8.41 (m, 1H); m/z (APCI pos) 469.1 (100%) (M+H).

### Example S41

### 1-Cyclopentyl-N-(2-(4-ethoxybenzamido)ethyl)-3-(trifluoromethyl)-1H-pyrazole-4-carboxamide

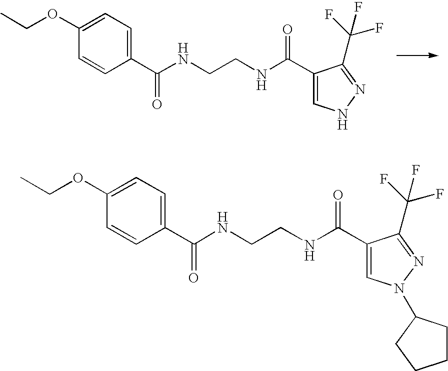

To a solution of N-(2-(4-ethoxybenzamido)ethyl)-3-(trifluoromethyl)-1H-pyrazole-4-carboxamide (0.1 g, 0.27 mmol) in bromocyclopentane (2 ml, excess) was added sodium bicarbonate (0.045 g, 0.54 mmol). The mixture was stirred for 16 hours at 120° C., cooled to room temperature, and filtered. The filtrate was concentrated in vacuo, and the residue was purified by silica gel column chromatography (9:0.5:0.5 DCM/EtOAc/MeOH) and recrystallized from EtOAc to give 1-cyclopentyl-N-(2-(4-ethoxybenzamido)ethyl)-3-(trifluoromethyl)-1H-pyrazole-4-carboxamide (0.011 g, 9%) as a solid: ¹H NMR (400 MHz, DMSO-d₆) δ 1.34 (t, 3H, J=7.0 Hz), 1.67 (m, 2H), 1.78 (m, 2H), 1.90 (m, 2H), 2.12 (m, 2H), 3.35 (m, 4H), 4.08 (q, 2H, J=7.0 Hz), 4.78 (m, 1H), 6.96 (d, 2H, J=8.9 Hz), 7.80 (d, 2H, J=8.9 Hz), 8.31 (s, 1H), 8.39 (s, 1H), 8.41 (s, 1H); m/z (APCI pos) 439.1 (100%) (M+H).

### Example S42

### 1-Cyclohexyl-N-(2-(4-ethoxybenzamido)ethyl)-3-(trifluoromethyl)-1H-pyrazole-4-carboxamide

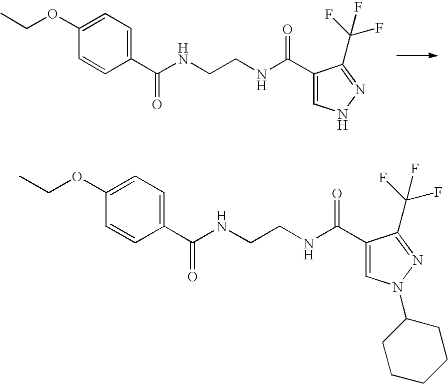

To a solution of N-(2-(4-ethoxybenzamido)ethyl)-3-(trifluoromethyl)-1H-pyrazole-4-carboxamide (0.1 g, 0.27 mmol) in bromocyclohexane (0.44 g, 2.70 mmol) was added potassium carbonate (0.11 g, 0.81 mmol). The mixture was stirred for 16 hours at 120° C. The mixture was diluted with EtOAc and filtered, and the filtrate was concentrated in vacuo. The residue was purified by silica gel column chromatography (9:0.5:0.5 DCM/EtOAc/MeOH) then recrystallized from ethyl acetate to give 1-cyclohexyl-N-(2-(4-ethoxybenzamido)ethyl)-3-(trifluoromethyl)-1H-pyrazole-4-carboxamide (0.030 g, 25%) as a solid: ¹H NMR (400 MHz, DMSO-d₆) δ 1.23 (m, 1H), 1.34 (t, 3H, J=7.0 Hz), 1.43 (m, 2H), 1.65 (m, 2.5H), 1.77-1.90 (m, 2.5H), 2.05 (m, 2H), 3.36 (m, 4H), 4.08 (q, 2H, J=7.0 Hz), 4.24 (m, 1H), 6.96 (d, 2H, J=8.9 Hz), 7.80 (d, 2H, J=8.9 Hz), 8.31 (s, 1H), 8.39 (s, 1H), 8.40 (s, 1H); m/z (APCI pos) 453.1 (100%) (M+H).

### Example S43

### N-(2-(4-Ethoxybenzamido)ethyl)-1-((tetrahydro-2H-pyran-2-yl)methyl)-3-(trifluoromethyl)-1H-pyrazole-4-carboxamide

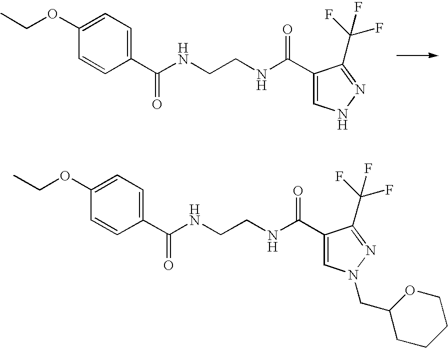

To a solution of N-(2-(4-ethoxybenzamido)ethyl)-3-(trifluoromethyl)-1H-pyrazole-4-carboxamide (0.100 g, 0.27 mmol) in DMF (1 ml) was added NaH (0.0119 g, 0.30 mmol). The mixture was stirred until the gas evolution ceased, about 30 min, and 2-(bromomethyl)-tetrahydro-2H-pyran (0.0346 ml, 0.27 mmol) was added. The mixture was stirred for 16 hours at room temperature, and the mixture was heated at 80° C. for 2 h, then cooled to room temperature. Methanol was added (1 ml), the solvent was removed in vacuo, and the residue was purified by silica gel column chromatography (3% MeOH/DCM) to give N-(2-(4-ethoxybenzamido)ethyl)-1-((tetrahydro-2H-pyran-2-yl)methyl)-3-(trifluoromethyl)-1H-pyrazole-4-carboxamide (0.025 g, 20%) as a solid: ¹H NMR (400 MHz, CDCl₃) δ 1.43 (t, 3H, J=7.0 Hz), 1.52 (m, 3H), 1.61 (m, 1H), 1.86 (m, 1H), 3.36 (m, 1H), 3.66 (m, 6H), 3.94 (m, 1H), 4.05-4.11 (m, 3H), 4.19 (m, 1H), 6.69 (m, 1H), 6.91 (d, 2H, J=8.9 Hz), 6.94 (m, 1H), 7.75 (d, 2H, J=8.9 Hz), 7.98 (m, 1H); m/z (APCI pos) 469.1 (100%) (M+H).

The compounds of the following structures were prepared from N-(2-(4-ethoxybenzamido)ethyl)-3-(trifluoromethyl)-1H-pyrazole-4-carboxamide and the corresponding alkyl halides, using a similar method to that described above.

| Ex # | Structure | Name | Physical Data |
|---|---|---|---|
| S44 | 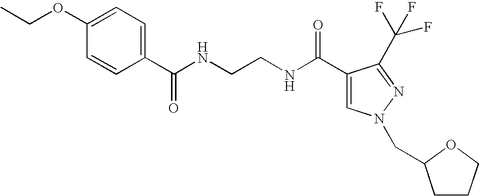 | N-(2-(4- ethoxybenzamido) ethyl)-1- ((tetrahydro- furan-2- yl)methyl)-3- (trifluoromethyl)- 1H- pyrazole-4- carboxamide | Solid (2%); ¹H NMR (400 MHz, CDCl₃) δ 1.43 (t, 3H, J = 7.08 Hz), 1.75- 1.95 (m, 2H), 2.04 (m, 1H), 3.65 (s, 5H), 3.73-3.87 (m, 2H), 4.05-4.16 (m, 3H), 4.22 (m, 1H), 4.32 (m, 1H), 6.76 (s, 1H), 6.91 (d, 2H, J = 8.9 Hz), 6.95 (s, 1H), 7.75 (d, 2H, J = 8.9 Hz), 8.04 (s, 1H); m/z (APCI pos) 455.1 (100%) (M + H). |
| S45 | 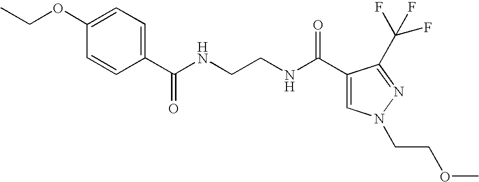 | N-(2-(4- ethoxybenzamido) ethyl)-1- (2-methoxyethyl)-3- (trifluoromethyl)- 1H- pyrazole-4- carboxamide | Solid (0.1%); ¹H NMR (400 MHz, DMSO-d₆) δ 1.34 (t, 3H, J = 7.0 Hz), 3.25 (s, 3H), 3.36 (m, 4H), 3.70 (t, 2H, J = 4.7 Hz), 4.08 (q, 2H, J = 7.0 Hz), 4.36 (t, 2H, J = 4.7 Hz), 6.96 (d, 2H, J = 8.56 Hz), 7.80 (d, 2H, J = 8.9 Hz), 8.32 (s, 1H), 8.39 (m, 2H); m/z (APCI pos) 429.0 (100%) (M + H). |

Ethyl 6-ethoxy-2,4-dimethylnicotinate

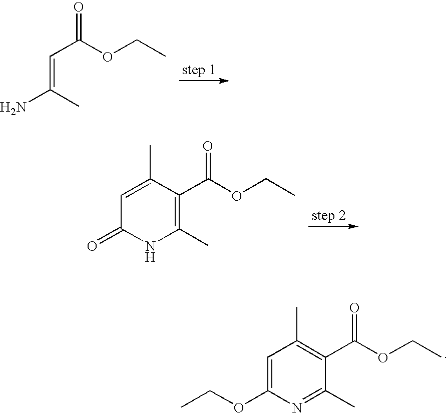

### Step 1

(E)-Ethyl 3-aminobut-2-enoate (2.53 g, 19.6 mmol) in toluene (15 mL) was charged with 4N HCl (10 mL, 40 mmol) in dioxane. The mixture was stirred at 115° C. overnight. The solution was cooled and the solid was filtered off washing with toluene. The filtrate was concentrated and the residue was purified by silica gel column chromatography (5% MeOH/EtOAc) to afford ethyl 2,4-dimethyl-6-oxo-1,6-dihydropyridine-3-carboxylate (1.11 g, 29%) as a white solid: ¹H NMR (400 MHz, CDCl₃) δ 1.37 (t, J=6.8 Hz, 3H), 2.29 (s, 3H), 2.47 (s, 3H), 4.33 (q, J=7.6, 14.4 Hz, 2H), 6.25 (s, 1H); m/z (APCI pos) 196.1 (100%) (M+H).

### Step 2

Ethyl 2,4-dimethyl-6-oxo-1,6-dihydropyridine-3-carboxylate (1.11 g, 5.69 mmol) was dissolved in DMF (5 mL). NaH (0.478 g, 19.9 mmol) was added and mixture was stirred for 30 minutes. Ethyl iodide (4.43 g, 28.4 mmol) was added and mixture was stirred at room temperature for 4 hours. The solution was quenched with water, extracted with EtOAc, dried, and concentrated. Flash chromatography on silica gel (5% EtOAc/hexanes) gave ethyl 6-ethoxy-2,4-dimethylnicotinate as a clear oil (0.69 g, 54%): m/z (APCI pos) 224.1 (100%) (M+H).

### Ethyl 2-chloro-6-ethoxynicotinate

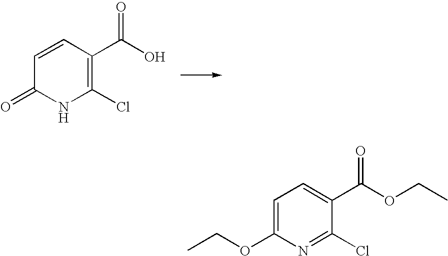

2-Chloro-6-oxo-1,6-dihydropyridine-3-carboxylic acid (3.00 g, 17.3 mmol, prepared according to procedure described in Ger. Offen. (1972) DE 2157289) was dissolved in DMF (10 mL). NaH (1.53 g, 60.5 mmol) was added and the mixture was stirred for 30 minutes. Ethyl iodide (13.5 g, 86.4 mmol) was added and the mixture was stirred at room temperature overnight. The solution was quenched with water and extracted with EtOAc, dried, and concentrated. Flash chromatography on silica gel (5% EtOAc/hexanes) gave ethyl 2-chloro-6-ethoxynicotinate (2.17 g, 55%) as an oil: ¹H NMR (400 MHz, CDCl₃) δ 1.38-1.42 (m, 6H), 4.36-4.41 (m, 4H), 6.66 (d, J=8.0 Hz, 1H), 8.11 (d, J=8.4 Hz, 1H).

### Ethyl 6-ethoxy-2-propylnicotinate

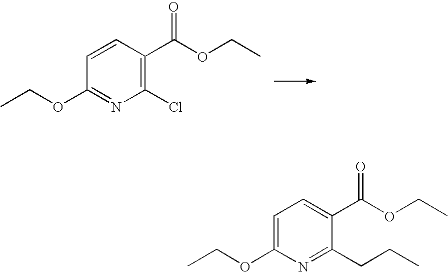

Ethyl 2-chloro-6-ethoxynicotinate (0.50 g, 2.18 mmol) and PdCl₂(dppf) dichloromethane adduct (0.089 g, 0.11 mmol) were dissolved in THF (30 mL) under argon. Propylzinc(II) bromide (10.9 mL, 5.44 mmol, 0.5M in THF) was added and the solution was stirred under argon at 60° C. for 150 minutes. The solution was cooled and filtered through a silica plug rinsing with EtOAc. The solution was concentrated and the residue was purified by silica gel column chromatography (4% EtOAc/hexanes) to give ethyl 6-ethoxy-2-propylnicotinate as an oil (0.378 g, 73%): m/z (APCI pos) 238.1 (100%) (M+H).

### Ethyl 4,6-dipropylnicotinate

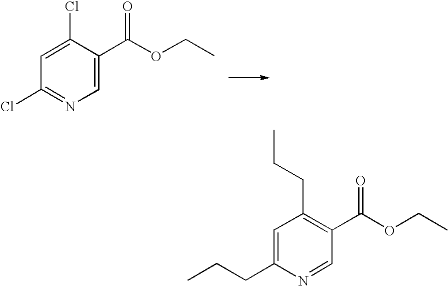

Ethyl 4,6-dichloronicotinate (1.00 g, 4.54 mmol) and PdCl₂(dppf) dichloromethane adduct (0.187 g, 0.227 mmol) were added together in THF (35 mL) under argon. Propylzinc(II) bromide (36.4 ml, 18.2 mmol, 0.5M in THF) was then added. The mixture was stirred under argon at 65° C. for 180 minutes. The solution was filtered through a silica gel plug (rinsing with EtOAc), and dried, and concentrated. Flash chromatography on silica gel (20-40% EtOAc/hexanes) gave ethyl 4,6-dipropylnicotinate (0.490 g, 46%) as a brown oil: m/z (APCI pos) 236.2 (100%) (M+H).

### Ethyl 3-(bis(tert-butoxycarbonyl)amino)-1-phenyl-1H-pyrazole-4-carboxylate

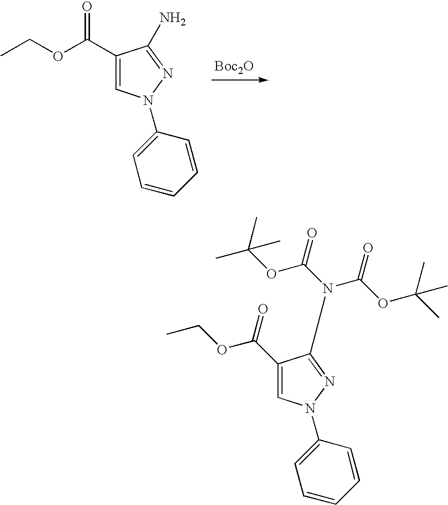

To a mixture of ethyl 3-amino-1-phenyl-1H-pyrazole-4-carboxylate (5.00 g, 21.62 mmol) (J. Het. Chem. 1967, 4, 325) in THF (50 mL) were added triethylamine (6.03 mL, 43.24 mmol), Boc₂O (5.66 g, 25.95 mmol) and 50 mg of DMAP and the mixture was heated at 50° C. for 2 hours and then at room temperature overnight. Water and EtOAc were added and the layers were separated. The organics were washed with water and brine, dried over Na₂SO₄, and concentrated to dryness. The obtained residue was purified on silica gel (20% EtOAc/hexanes) to give ethyl 3-(bis(tert-butoxycarbonyl)amino)-1-phenyl-1H-pyrazole-4-carboxylate (2.11 g, 30%) as a solid: ¹H NMR (400 MHz, CDCl₃) δ 1.35 (t, J=7.1 Hz, 3H), 1.45 (s, 18H), 4.27-4.33 (m, 2H), 7.36 (t, J=7.4 Hz, 1H), 7.48 (t, J=7.9 Hz, 2H), 7.69 (d, J=7.8 Hz, 2H), 8.41 (s, 1H); m/z (APCI pos) 331 (40%) (M+H-Boc).

### Ethyl 3-(dimethylamino)-1-phenyl-1H-pyrazole-4-carboxylate

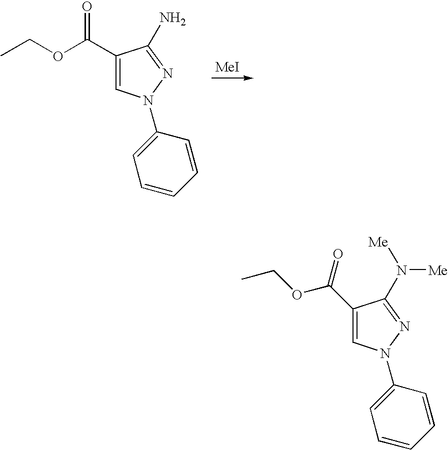

A THF (40 ml) solution of ethyl 3-amino-1-phenyl-1H-pyrazole-4-carboxylate (2.50 g, 10.81 mmol) was cooled in an ice bath and treated with NaH (0.6486 g, 27.03 mmol). This mixture was allowed to stir 30 minutes and then methyl iodide (1.619 ml, 25.95 mmol) was added. The mixture was allowed to warm to room temperature and stirred for 6 hr. The mixture was then quenched with water, dissolved in EtOAc and saturated aqueous sodium bicarbonate. The aqueous layer was extracted with EtOAc, and the organics were combined, washed with water, dried over Na₂SO₄ and concentrated to a residue. The residue was purified by silica gel column chromatography (10% EtOAc/hexanes) to give ethyl 3-(dimethylamino)-1-phenyl-1H-pyrazole-4-carboxylate (0.260 g, 9%): ¹H NMR (400 MHz, CDCl₃) δ 1.37 (t, J=7.1 Hz, 3H), 3.01 (s, 6H), 4.28-4.34 (m, 2H), 7.26 (t, J=7.4 Hz, 1H), 7.43 (t, J=8.0 Hz, 2H), 7.65-7.67 (m, 2H), 8.29 (s, 1H); m/z (APCI pos) 260 (M+H).

### 6-(Ethoxycarbonyl)nicotinic acid

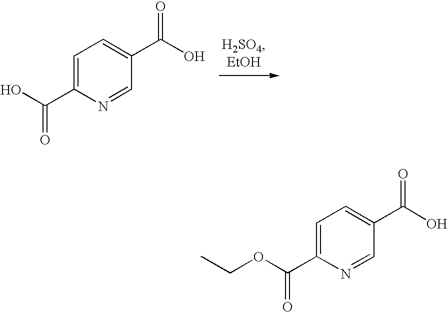

To a mixture of pyridine-2,5-dicarboxylic acid (30.00 g, 179.51 mmol) in ethanol (380 ml, 179.51 mmol) was slowly added sulfuric acid (3.00 ml, 56.28 mmol) and the mixture was heated to reflux for 7 hours. The solution was cooled in an ice bath and diluted with water (400 mL). The resulting suspension was stirred overnight at room temperature. The solid was filtered and washed with water. The solid was triturated in 300 ml of refluxing EtOH for 2 hours and then cooled in an ice bath and the solid was filtered to give 6-(ethoxycarbonyl)nicotinic acid (16.05 g, 46% yield): ¹H NMR (400 MHz, DMSO-d₆) δ 1.29 (t, J=7.0 Hz, 3H), 4.29-4.35 (m, 2H), 8.10 (d, J=8.0 Hz, 1H), 8.39 (d, J=8.2 Hz, 1H), 9.11 (s, 1H).

### Ethyl 3-isopropyl-1-phenyl-1H-pyrazole-4-carboxylate

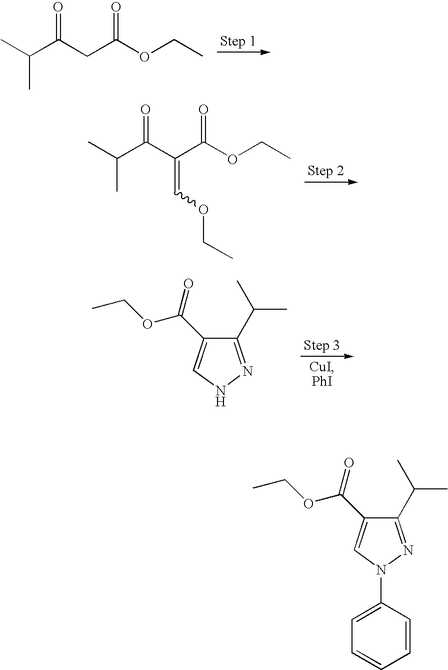

### Step 1

A mixture of ethyl 4-methyl-3-oxopentanoate (25.00 g, 158.0 mmol), triethyl orthoformate (46.84 g, 316.1 mmol) and acetic anhydride (75 ml, 794.9 mmol) was heated to reflux overnight. The volatiles were distilled off at 85° C. under high vacuum. The crude material was taken on to the next step without further purification.

### Step 2

To a mixture of the above crude (E/Z)-ethyl 2-(ethoxymethylene)-4-methyl-3-oxopentanoate (33.86 g, 158.0 mmol) in MeOH (500 ml) was slowly added hydrazine monohydrate (15.33 ml, 316.1 mmol). The mixture was heated to reflux for 3 hours then cooled and concentrated to a residue. EtOAc (250 ml) was added and the organic layer was washed with water and brine, dried over Na₂SO₄, and concentrated to a residue that solidified upon standing to give ethyl 3-isopropyl-1H-pyrazole-4-carboxylate (25.42 g, 88% yield): ¹H NMR (400 MHz, CDCl₃) δ 1.33-1.38 (m, 9H), 3.65-3.75 (m, 1H), 4.28-4.33 (m, 2H), 7.96 (s, 1H), 11.82 (br, 1H); m/z (APCI pos) 183 (M+H).

### Step 3

To a 100 ml sealed tube flushed vigorously with nitrogen were added ethyl 3-isopropyl-1H-pyrazole-4-carboxylate (5.00 g, 27.4 mmol), 1-iodobenzene (3.67 ml, 32.9 mmol), K₂CO₃ (7.96 g, 57.6 mmol), copper(I) iodide (0.261 g, 1.37 mmol), and (1S,2S)—N1,N2-dimethylcyclohexane-1,2-diamine (0.390 g, 2.74 mmol), followed by degassed toluene (25 ml). The mixture was stirred for 24 hours at 110° C. The mixture was cooled to room temperature, and filtered through a short silica pad eluting with 25% EtOAc/hexanes. The filtrate was concentrated under high vacuum to give crude ethyl 3-isopropyl-1-phenyl-1H-pyrazole-4-carboxylate (2.31 g, 33%) that was used without further purification: ¹H NMR (400 MHz, CDCl₃) δ 1.35-1.39 (m, 9H), 3.56-3.63 (m, 1H), 4.29-4.35 (m, 2H), 7.26-7.42 (m, 1H), 7.45 (t, J=8.0 Hz, 2H), 7.70 (d, J=7.4 Hz, 2H), 8.33 (s, 1H); m/z (APCI neg) 259 (100%) (M+H).

### Ethyl 3-methoxy-1-phenyl-1H-pyrazole-4-carboxylate

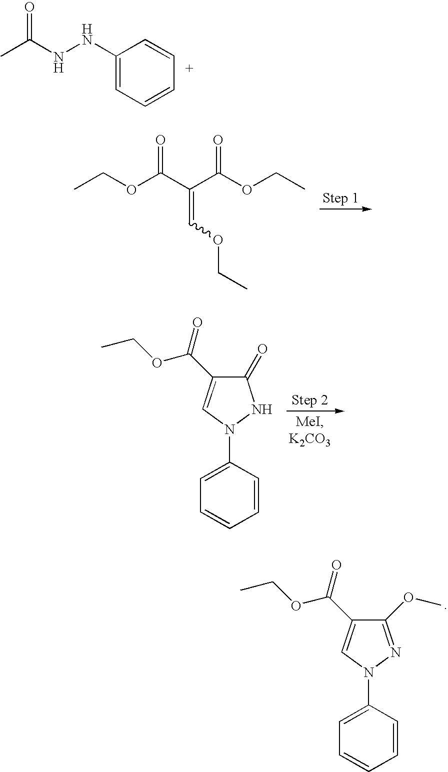

### Step 1

Diethyl 2-(ethoxymethylene)malonate (28.80 g, 133.2 mmol) was added to N′-phenylacetohydrazide (20.00 g, 133.2 mmol) in POCl₃ (200 mL) and the mixture was stirred at 70° C. overnight under nitrogen. The mixture was then cooled and carefully quenched over ice water (500 ml) to give a solution which was cooled to −10° C. The resulting sticky brown solid was filtered, dissolved in DCM and dried on Na₂SO₄ then concentrated. The residue was purified by silica gel column chromatography (25% EtOAc/hexanes) to give ethyl 3-oxo-1-phenyl-2,3-dihydro-1H-pyrazole-4-carboxylate (3.71 g, 12% yield): ¹H NMR (400 MHz, CDCl₃) δ 1.39 (t, J=7.1 Hz, 3H), 4.35-4.41 (m, 2H), 7.31 (t, J=7.4 Hz, 1H), 7.45 (t, J=8.0 Hz, 2H), 7.66 (d, J=7.6 Hz, 2H), 8.12 (s, 1H), 8.18 (s, 1H); m/z (APCI pos) 233 (M+H).

### Step 2

To a mixture of ethyl 3-oxo-1-phenyl-2,3-dihydro-1H-pyrazole-4-carboxylate (0.750 g, 3.229 mmol) in DMF (5 mL) was added potassium carbonate (1.34 g, 9.6 mmol) followed by methyl iodide (0.3 ml, 4.8 mmol) and the mixture was stirred at 40° C. for 3 hours. The mixture was quenched with water and the solids were filtered, washed with water and dried under high vacuum to give ethyl 3-methoxy-1-phenyl-1H-pyrazole-4-carboxylate (0.620 g, 78%): ¹H NMR (400 MHz, CDCl₃) δ 1.36 (t, J=7.1 Hz, 3H), 4.09 (s, 3H), 4.30-4.35 (m, 2H), 7.29 (t, J=7.4 Hz, 1H), 7.45 (t, J=8.0 Hz, 2H), 7.65 (d, J=7.6 Hz, 2H), 8.24 (s, 1H). m/z (APCI pos) 247 (M+H).

The compound of the following structure was prepared from ethyl 3-oxo-1-phenyl-2,3-dihydro-1H-pyrazole-4-carboxylate and benzyl bromide according to the above procedure.

| Structure | Name | Physical Data |
|---|---|---|
| 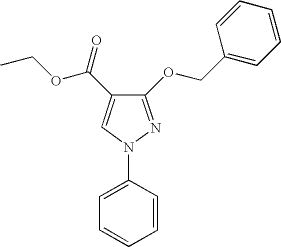 | ethyl 3- (benzyloxy)-1- phenyl-1H- pyrazole-4- carboxylate | ¹H NMR (400 MHz, CDCl₃) δ 1.37 (t, J = 7.1 Hz, 3H), 4.30-4.35 (m, 2H), 5.46 (s, 2H), 7.28-7.33 (m, 2H), 7.38 (t, J = 7.3 Hz, 2H), 7.45 (t, J = 8.0 Hz, 2H), 7.55 (d, J = 7.0 Hz, 2H), 7.65 (d, J = 7.4 Hz, 2H), 8.26 (s, 1H); m/z (APCI pos) 323 (M + H). |

### Methyl 1-methyl-3-phenyl-1H-pyrazole-5-carboxylate and methyl 1-methyl-5-phenyl-1H-pyrazole-3-carboxylate

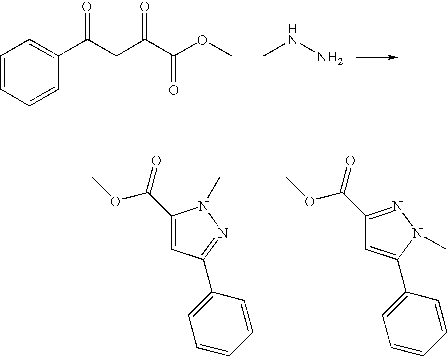

Methyl 2,4-dioxo-4-phenylbutanoate (8.25 g, 40.0 mmol) was dissolved in EtOH (50 mL) and 1-methylhydrazine (1.84 g, 40.0 mmol) was added dropwise. The resulting solution was heated at reflux for 5 hours then cooled to room temperature and concentrated to a residue. The obtained residue was purified by silicagel column chromatography (10% EtOAc/petroleum ether −25% EtOAc/petroleum ether). The first spot to elute was methyl 1-methyl-3-phenyl-1H-pyrazole-5-carboxylate (2.84 g, 33%) as a white solid. The next spot to elute was the regioisomer methyl 1-methyl-5-phenyl-1H-pyrazole-3-carboxylate (1.5 g, 17%) as a yellow oil.

methyl 1-methyl-3-phenyl-1H-pyrazole-5-carboxylate: ¹H NMR (400 MHz, CDCl₃) δ 3.91 (s, 3H), 4.23 (s, 3H), 7.12 (s, 1H), 7.32 (t, J=7.3 Hz, 1H), 7.41 (t, J=7.5 Hz, 2H), 7.79 (d, J=7.0 Hz, 2H); m/z (APCI pos) 217 (100%) (M+H).

methyl 1-methyl-5-phenyl-1H-pyrazole-3-carboxylate: ¹H NMR (400 MHz, CDCl₃) δ 3.95 (s, 3H), 3.96 (s, 3H), 6.86 (s, 1H), 7.41-7.51 (m, 5H); m/z (APCI pos) 217 (100%) (M+H).

### 1-Methyl-3-phenyl-1H-pyrazole-5-carboxylic acid

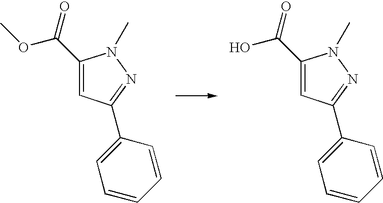

Methyl 1-methyl-3-phenyl-1H-pyrazole-5-carboxylate (1.00 g, 6.23 mmol) was dissolved in EtOH (10 mL) and water (10 mL) then lithium hydroxide monohydrate (0.582 g, 13.87 mmol) was added. The mixture was stirred at 50° C. until complete by HPLC. The mixture was concentrated to a residue, water was added and the mixture was extracted with DCM. The aqueous layer was acidified using 1N HCl and the resulting solid was filtered and dried under high vacuum to give 1-methyl-3-phenyl-1H-pyrazole-5-carboxylic acid (0.887 g, 95%) as a white solid; m/z (APCI pos) 203 (100%) (M+H).

The compound of the following structure was prepared from the corresponding ester using a similar method to that described above.

| Structure | Name | Physical Data |
|---|---|---|
| 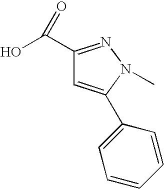 | 1-Methyl-5- phenyl-1H- pyrazole-3- carboxylic acid | m/z (APCI pos) 203 (100%) (M + H). |

### N-(2-Aminoethyl)-1-phenyl-3-(trifluoromethyl)-1H-pyrazole-4-carboxamide

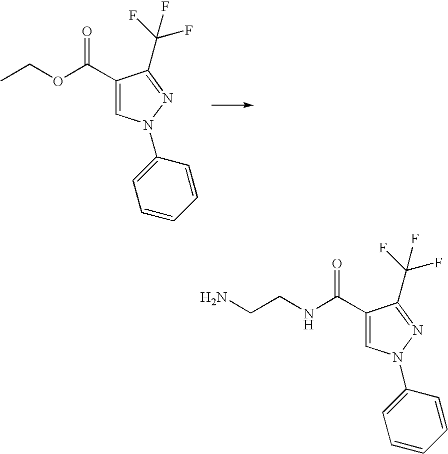

A mixture of ethyl 1-phenyl-3-(trifluoromethyl)-1H-pyrazole-4-carboxylate (6.60 g, 23.2 mmol) and ethylenediamine (50 ml, 23.2 mmol) was heated at reflux for 3 hours. The mixture was concentrated under reduced pressure to a residue. To the residue was added toluene (50 mL) that was concentrate off, twice. The crude solid was dried under high vacuum overnight to give N-(2-aminoethyl)-1-phenyl-3-(trifluoromethyl)-1H-pyrazole-4-carboxamide (6.9 g, 99% yield) as a light tan solid. The desired product may also be isolated as the hydrochloride salt by dissolving in minimal DCM and adding 1N HCl in Et₂O (3-5 molar equivalents) dropwise, filtering the solid and washing with Et₂O. ¹H NMR (Free base analog) (DMSO-d₆) δ 1.83 (br, 2H), 2.68 (t, J=6.3 Hz, 2H), 3.21-3.26 (m, 2H), 7.47 (t, J=7.4 Hz, 1H), 7.60 (t, J=8.0 Hz, 2H), 7.84 (d, J=7.8 Hz, 2H), 8.29-8.30 (m, 1H), 9.11 (s, 1H); m/z (APCI pos) 299 (60%) (M+H).

### N-(2-Aminoethyl)-6-(2,2,2-trifluoroethoxy)nicotinamide

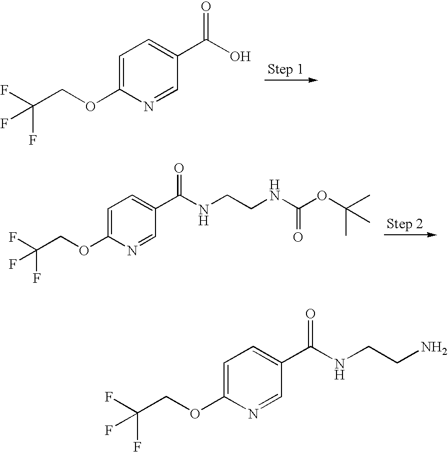

### Step 1

To a solution of 6-(2,2,2-trifluoroethoxy)nicotinic acid (0.507 g, 2.29 mmol), EDAC.HCl (0.483 g, 2.52 mmol), and HOBt.H₂O (0.386 g, 2.52 mmol) in DMF (5 ml) was added tert-butyl 2-aminoethylcarbamate (0.404 g, 2.52 mmol). The solution was stirred for 16 h at room temperature, diluted with EtOAc (100 ml) and washed with water (3×), saturated aqueous sodium bicarbonate, and brine. The aqueous layer was back extracted and the combined organic layers were dried (sodium sulfate), and concentrated in vacuo to give tert-butyl 2-(6-(2,2,2-trifluoroethoxy)nicotinamido)ethylcarbamate (0.700 g, 84%) as a solid after trituration with ether: ¹H NMR (CDCl₃) δ 1.37 (s, 9H), 3.10 (m, 2H), 3.28 (m, 2H), 5.06 (q, 2H, J=8.9 Hz), 6.91 (m, 1H), 7.07 (d, 1H, J=8.6 Hz), 8.19 (m, 1H), 8.54 (m, 1H), 8.65 (m, 1H).

### Step 2

To a solution of tert-butyl 2-(6-(2,2,2-trifluoroethoxy)nicotinamido)ethylcarbamate (0.700 g, 1.93 mmol) in DCM (10 ml) was added TFA (1.48 ml, 19.30 mmol). The mixture was stirred at room temperature for 2 hours then the solvent was removed in vacuo to give the product as the TFA salt, which was an intractable oil that partially crystallized overnight. This material was dissolved in DCM and treated with saturated aqueous sodium bicarbonate. The aqueous layer was extracted multiple times with EtOAc. The combined organic layers were dried (sodium sulfate) and concentrated in vacuo to give N-(2-aminoethyl)-6-(2,2,2-trifluoroethoxy)nicotinamide (0.458 g, 90%) as an oil: ¹H NMR (CDCl₃) δ 3.00 (m, 2H), 3.50 (m, 2H), 5.07 (q, 2H, J=8.9 Hz), 7.11 (m, 1H), 7.84 (br, 2H), 8.22 (m, 1H), 8.69 (m, 1H), 8.72 (m, 1H).

### N-(2-Aminoethyl)-6-(2,2,2-trifluoroethoxy)nicotinamide hydrochloride

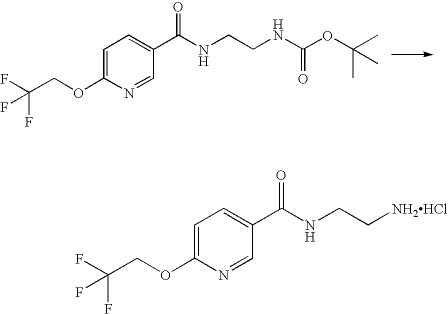

To a solution of tert-butyl 2-(6-(2,2,2-trifluoroethoxy)nicotinamido)ethylcarbamate (8.2 g, 23 mmol) in EtOAc was added hydrogen chloride (17 ml, 68 mmol, 4.0 M in dioxane). The solution was stirred overnight. A white solid was observed in the reaction mixture. The solid was collected by filtration and dried in vacuo to give N-(2-aminoethyl)-6-(2,2,2-trifluoroethoxy)nicotinamide hydrochloride (6.8 g, 100%) as a solid: ¹H NMR (400 MHz, DMSO-d₆) δ 2.99 (q, 2H, J=6.2 Hz), 3.52 (q, 2H, J=6.2 Hz), 5.07 (q, 2H, J=8.9 Hz), 7.09 (m, 1H), 8.03 (br, 3H), 8.28 (m, 1H), 8.75 (m, 1H), 8.89 (m, 1H)

### 1-(Pyridin-2-ylmethyl)-3-(trifluoromethyl)-1H-pyrazole-4-carboxylic acid

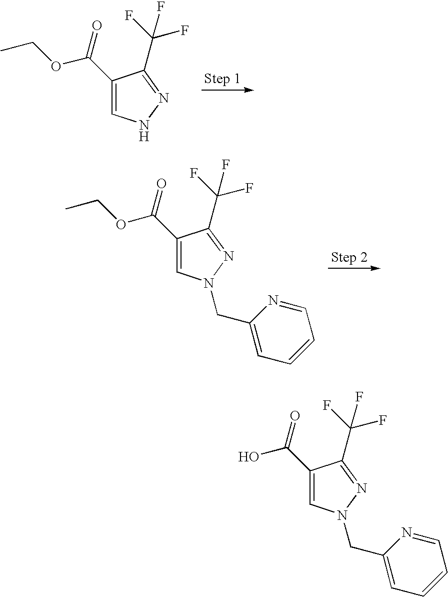

### Step 1

To a solution of ethyl 3-(trifluoromethyl)-1H-pyrazole-4-carboxylate (10.0 g, 48.00 mmol) in DMF were added 2-(bromomethyl)pyridine hydrobromide (13.4 g, 52.80 mmol) and potassium carbonate (13.3 g, 96.1 mmol). The mixture was stirred for 16 hours at room temperature and filtered. The solid was dried under vacuum to give ethyl 1-(pyridin-2-ylmethyl)-3-(trifluoromethyl)-1H-pyrazole-4-carboxylate (12.5 g, 87%) as crystals: ¹H NMR (CDCl₃) δ 1.34 (t, 3H, J=7.0 Hz), 4.32 (q, 2H, J=7.0 Hz), 5.45 (s, 2H), 7.22 (d, 1H, J=7.8 Hz), 7.29 (m, 1H), 7.72 (m, 1H), 8.16 (s, 1H), 8.61 (m, 1H).

### Step 2

To a solution of ethyl 1-(pyridin-2-ylmethyl)-3-(trifluoromethyl)-1H-pyrazole-4-carboxylate (0.273 g, 0.912 mmol) in THF was added 2N NaOH (1.37 ml, 2.74 mmol). Ethanol was added to achieve homogeneity. The mixture was stirred for 4 hours, concentrated under vacuum, and diluted with water. Acidification to pH 3 gave a precipitate, which was filtered and dried under vacuum to give 1-(pyridin-2-ylmethyl)-3-(trifluoromethyl)-1H-pyrazole-4-carboxylic acid (0.203 g, 82%) as a solid: m/z (APCI neg) 226.0 (100%) (M-CO₂)

### 1-(Phenylsulfonyl)-1H-indole-3-carboxylic acid

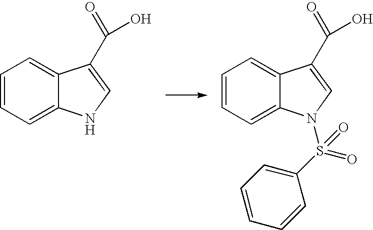

To a solution of 1H-indole-3-carboxylic acid (0.500 g, 3.10 mmol) in DMF at 0° C. was added sodium hydride (0.2606 g, 6.52 mmol). After the gas evolution ceased (30 min), benzenesulfonyl chloride (0.792 ml, 6.20 mmol) was added. The mixture was stirred for 16 hours, then partitioned between 1M H₃PO₄ (aq.) and EtOAc. The organic layer was dried (sodium sulfate) and concentrated in vacuo and the residue was triturated with ether to give 1-(phenylsulfonyl)-1H-indole-3-carboxylic acid (0.254 g, 27%) as a solid: m/z (APCI neg) 256.0 (100%) (M-CO₂).

The compound of the following structure was prepared by reacting 1H-indole-3-carboxylic acid and benzoyl chloride using the procedure outlined above.

| Structure | Name | Physical Data |
|---|---|---|
| 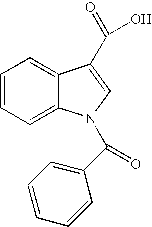 | 1-benzoyl-1H- indole-3- carboxylic acid | Solid (100%); m/z (APCI neg) 220.0 (100%) (M − CO₂). |

### Ethyl 1-(1H-indol-5-yl)-3-(trifluoromethyl)-1H-pyrazole-4-carboxylate

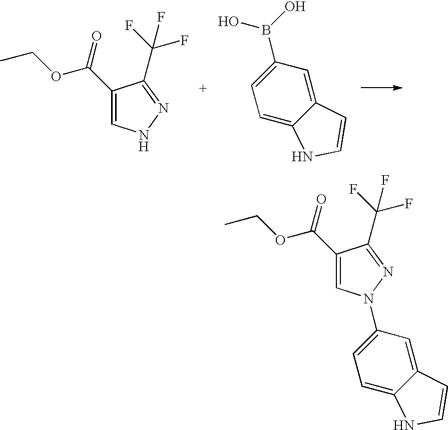

To a solution of ethyl 3-(trifluoromethyl)-1H-pyrazole-4-carboxylate (0.208 g, 1.0 mmol) in DMF were added 1H-indol-5-ylboronic acid (0.322 g, 2.00 mmol), Cu(OAc)₂ (0.136 g, 0.75 mmol), and pyridine (0.162 ml, 2.00 mmol) and the mixture was stirred at room temperature for 3 days. The mixture was diluted with DCM and filtered through a pad of silica gel. The filtrate was concentrated in vacuo and the residue was purified by silica gel column chromatography (3:1 hexanes/EtOAc) to give ethyl 1-(1H-indol-5-yl)-3-(trifluoromethyl)-1H-pyrazole-4-carboxylate (0.313 g, 97%) as a solid: ¹H NMR (CDCl₃) δ 1.39 (t, 3H, J=7.0 Hz), 4.37 (q, 2H, J=7.0 Hz), 6.64 (m, 1H), 7.34 (m, 1H), 7.51 (m, 2H), 7.93 (m, 1H), 8.46 (m, 1H).

Compounds of the following structures were made from ethyl 3-(trifluoromethyl)-1H-pyrazole-4-carboxylate and the corresponding boronic acids according to the above procedure.

| Structure | Name | Physical Data |
|---|---|---|
| 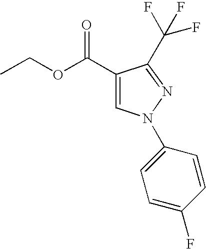 | ethyl 1-(4- fluorophenyl)-3- (trifluoromethyl)- 1H- pyrazole-4- carboxylate | Solid (100%); ¹H NMR (CDCl₃) δ 1.38 (t, 3H, J = 7.0 Hz), 4.37 (q, 2H, J = 7.0 Hz), 7.21 (m, 2H), 7.70 (m, 2H), 8.43 (s, 1H). |
| 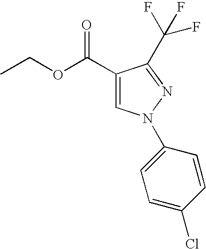 | ethyl 1-(4- chlorophenyl)-3- (trifluoromethyl)- 1H- pyrazole-4- carboxylate | Solid (87%); ¹H NMR (CDCl₃) δ 1.39 (t, 3H, J = 7.0 Hz), 4.37 (q, 2H, J = 7.0 Hz), 7.49 (m, 2H), 7.68 (m, 2H), 8.47 (m, 1H). |
| 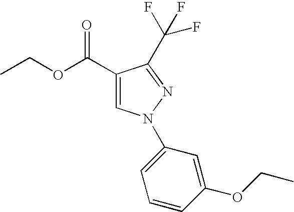 | ethyl 1-(3- ethoxyphenyl)-3- (trifluoromethyl)- 1H- pyrazole-4- carboxylate | Solid (72%); ¹H NMR (CDCl₃) δ 1.38 (t, 3H, J = 7.0 Hz), 1.45 (t, 3H, J = 7.0 Hz), 4.10 (q, 2H, J = 7.0 Hz), 4.36 (q, 2H, J = 7.0 Hz), 6.93 (m, 1H), 7.23 (m, 1H), 7.28 (m, 1H), 7.38 (t, 1H, J = 8.2 Hz), 8.45 (m, 1H). |
| 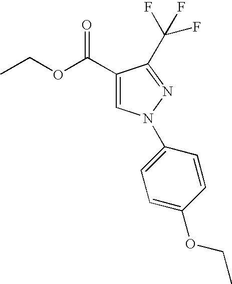 | ethyl 1-(4- ethoxyphenyl)-3- (trifluoromethyl)- 1H- pyrazole-4- carboxylate | Solid (30%); ¹H NMR (CDCl₃) δ 1.38 (t, 3H, J = 7.0 Hz), 1.45 (t, 3H, J = 7.0 Hz), 4.08 (q, 2H, J = 7.0 Hz), 4.36 (q, 2H, J = 7.0 Hz), 6.98 (m, 2H), 7.59 (m, 2H), 8.37 (m, 1H). |

### Ethyl 1-(2-hydroxyphenyl)-3-(trifluoromethyl)-1H-pyrazole-4-carboxylate

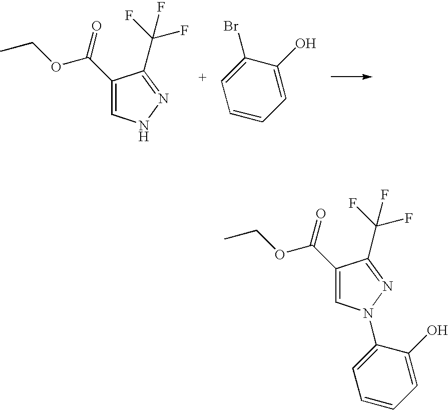

According to the procedure of Buchwald et al. (J. Org. Chem. 2004, 69, 5578), to a 50 mL sealed tube flushed with argon were added copper(I) iodide (0.02288 g, 0.1201 mmol), potassium carbonate (0.6972 g, 5.045 mmol), and ethyl 3-(trifluoromethyl)-1H-pyrazole-4-carboxylate (0.500 g, 2.402 mmol). (1S,2S)—N1,N2-Dimethylcyclohexane-1,2-diamine (0.06834 g, 0.4805 mmol) and 2-bromophenol (0.3343 ml, 2.883 mmol) were then added along with 3 ml of toluene. The tube was sealed and heated to 110° C. overnight, then cooled to room temperature. the mixture was filtered through celite to remove the solid. The filtrate was concentrated under vacuum and the residue was purified by silica gel column chromatography (10% Et₂O/DCM) to give ethyl 1-(2-hydroxyphenyl)-3-(trifluoromethyl)-1H-pyrazole-4-carboxylate (0.660 g, 91%) as a solid: ¹H NMR (CDCl₃) δ 1.40 (t, 3H, J=7.0 Hz), 4.39 (q, 2H, J=7.0 Hz), 7.00 (m, 1H), 7.16 (m, 1H), 7.31 (m, 1H), 7.42 (m, 1H), 8.56 (m, 1H)

Compounds of the following structures were prepared from ethyl 3-(trifluoromethyl)-1H-pyrazole-4-carboxylate and the corresponding bromide or iodide, using a similar method to that described above.

| Structure | Name | Physical Data |
|---|---|---|
| 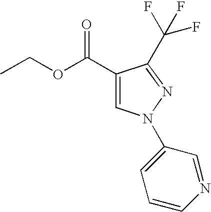 | ethyl 1- (pyridin-3- yl)-3- (trifluoromethyl)- 1H- pyrazole-4- carboxylate | Solid (74%); ¹H NMR (CDCl₃) δ 1.40 (t, 3H, J = 7.4 Hz), 4.38 (q, 2H, J = 7.4 Hz), 7.49 (m, 1H), 8.11 (m, 1H), 8.54 (m, 1H), 8.69 (m, 1H), 9.02 (m, 1H). |
| 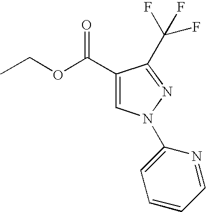 | ethyl 1- (pyridin-2- yl)-3- (trifluoromethyl)- 1H- pyrazole-4- carboxylate | Solid (87%); ¹H NMR (CDCl₃) δ 1.39 (t, 3H, J = 7.4 Hz), 4.37 (q, 2H, J = 7.0 Hz), 7.34 (m, 1H), 7.90 (m, 1H), 8.06 (m, 1H), 8.48 (m, 1H), 9.13 (m, 1H). |
| 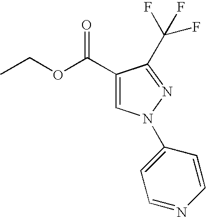 | ethyl 1- (pyridin-4- yl)-3- (trifluoromethyl)- 1H- pyrazole-4- carboxylate | Solid (20%); m/z (APCI pos) 286.1 (100%) (M + H). |
| 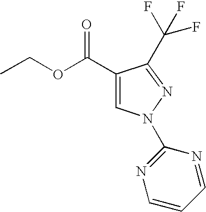 | ethyl 1- (pyrimidin-2- yl)-3- (trifluoromethyl)- 1H- pyrazole-4- carboxylate | Solid (61%); ¹H NMR (CDCl₃) δ 1.40 (t, 3H, J = 7.0 Hz), 4.39 (q, 2H, J = 7.0 Hz), 7.40 (t, 1H, J = 4.7 Hz), 8.86 (d, 2H, J = 4.7 Hz), 9.19 (m, 1H). |

### 1-(1H-Indol-5-yl)-3-(trifluoromethyl)-1H-pyrazole-4-carboxylic acid

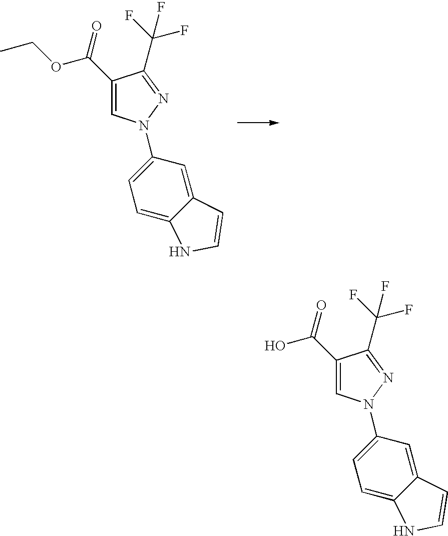

To a solution of ethyl 1-(1H-indol-5-yl)-3-(trifluoromethyl)-1H-pyrazole-4-carboxylate (0.313 g, 0.968 mmol) in EtOH (20 ml) and THF (5 ml) was added 2M NaOH (1.45 ml, 2.90 mmol). The mixture was refluxed until TLC indicated complete consumption of ester, cooled to room temperature, and concentrated in vacuo. The residue was dissolved in water and washed with ether. The aqueous layer was then adjusted to pH 4 with 10% aq. HCl. The solid was collected by filtration and dried under vacuum to give 1-(1H-indol-5-yl)-3-(trifluoromethyl)-1H-pyrazole-4-carboxylic acid (0.220 g, 77%) as a powder: ¹H NMR (400 MHz, CDCl₃) δ 6.55 (m, 1H), 7.52 (m, 2H), 7.62 (m, 1H), 8.06 (m, 1H), 9.08 (s, 1H), 11.39 (br, 1H), 13.10 (br, 1H).

Compounds of the following structures were prepared from the corresponding esters using a similar method to that described above.

| Structure | Name | Physical Data |
|---|---|---|
| 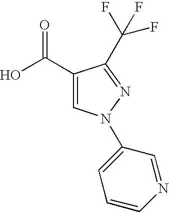 | 1-(pyridin-3- yl)-3- (trifluoromethyl)- 1H- pyrazole-4- carboxylic acid | Solid (96%); ¹H NMR (400 MHz, CDCl₃) δ 7.62 (m, 1H), 8.34 (m, 1H), 8.66 (m, 1H), 9.17 (br, 1H), 9.33 (m, 1H). |
| 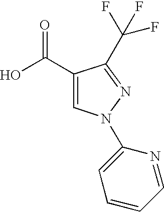 | 1-(pyridin-2- yl)-3- (trifluoromethyl)- 1H- pyrazole-4- carboxylic acid | Solid (95%); ¹H NMR (400 MHz, CDCl₃) δ 7.55 (m, 1H), 7.99 (m, 1H), 8.11 (m, 1H), 8.58 (m, 1H), 9.10 (m, 1H), 13.33 (br, 1H). |
| 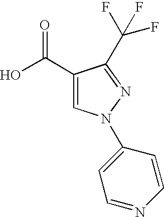 | 1-(pyridin-4- yl)-3- (trifluoromethyl)- 1H- pyrazole-4- carboxylic acid | Solid (70%); ¹H NMR (400 MHz, CDCl₃) δ 8.01 (m, 2H), 8.75 (m, 2H), 9.47 (m, 1H). |
| 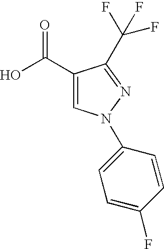 | 1-(4- fluorophenyl)-3- (trifluoromethyl)- 1H- pyrazole-4- carboxylic acid | Solid (78%); ¹H NMR (400 MHz, CDCl₃) δ 7.37 (m, 2H), 7.93 (m, 2H), 9.15 (s, 1H), 13.18 (br, 1H). |
| 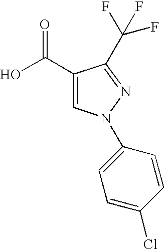 | 1-(4- chlorophenyl)-3- (trifluoromethyl)- 1H- pyrazole-4- carboxylic acid | Solid (29%); ¹H NMR (400 MHz, CDCl₃) δ 7.64 (m, 2H), 7.98 (m, 2H), 9.26 (s, 1H), 13.27 (br, 1H). |
| 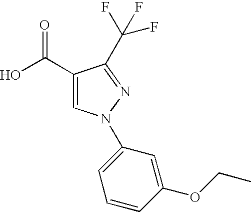 | 1-(3- ethoxyphenyl)-3- (trifluoromethyl)- 1H- pyrazole-4- carboxylic acid | Solid (75%); ¹H NMR (400 MHz, CDCl₃) δ 1.36 (t, 3H, J = 7.0 Hz), 4.13 (q, 2H, J = 7.0 Hz), 7.00 (m, 1H), 7.47 (m, 3H), 9.27 (m, 1H). |
| 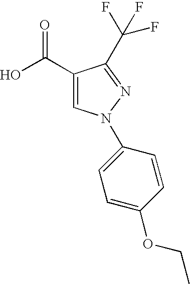 | 1-(4- ethoxyphenyl)-3- (trifluoromethyl)- 1H- pyrazole-4- carboxylic acid | Solid (86%); ¹H NMR (400 MHz, CDCl₃) δ 1.35 (t, 3H, J = 7.0 Hz), 4.09 (q, 2H, J = 7.0 Hz), 7.08 (m, 2H), 7.82 (m, 2H), 9.10 (m, 1H), 13.15 (br, 1H). |
| 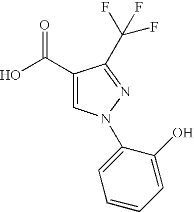 | 1-(2- hydroxyphenyl)-3- (trifluoromethyl)- 1H- pyrazole-4- carboxylic acid | Solid (82%); ¹H NMR (400 MHz, CDCl₃) δ 6.97 (m, 1H), 7.10 (m, 1H), 7.32 (m, 1H), 7.58 (m, 1H), 8.78 (m, 1H), 10.63 (s, 1H), 13.10 (br, 1H). |
| 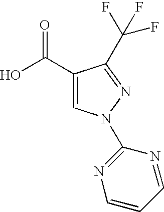 | 1-(pyrimidin- 2-yl)-3- (trifluoromethyl)- 1H- pyrazole-4- carboxylic acid | Solid (97%): m/z 256.9 (APCI neg) (100%) (M − H). |

### N-(2-Aminoethyl)-4-ethoxybenzamide hydrochloride

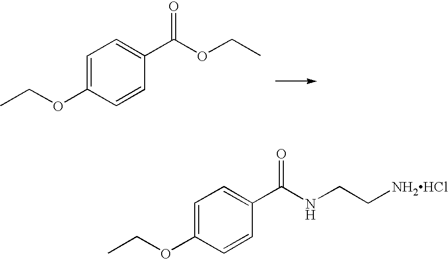

Ethyl 4-ethoxybenzoate (25.40 g, 141.0 mmol) was dissolved in ethylenediamine (200 ml) and the mixture was heated at reflux for 40 hours. The mixture was concentrated under reduced pressure to a residue. The obtained residue was dissolved in DCM (100 mL) and Et₂O (200 mL), and 1N HCl in Et₂O (200 mL) was added. The obtained solid was filtered, washed with DCM and dried under high vacuum to give N-(2-aminoethyl)-4-ethoxybenzamide hydrochloride (31.78 g, 92%) as a tan solid: ¹H NMR (400 MHz, CDCl₃) δ 1.43 (t, J=7.0 Hz, 3H), 2.92 (t, J=5.9 Hz, 2H), 3.49 (t, J=5.9 Hz, 2H), 4.05-4.10 (m, 2H), 6.88-6.92 (m, 2H), 7.74-7.78 (m, 2H); m/z (APCI pos) 209 (M+H).

### 1-Phenyl-3-(trifluoromethyl)-1H-pyrazole-4-carboxylic acid

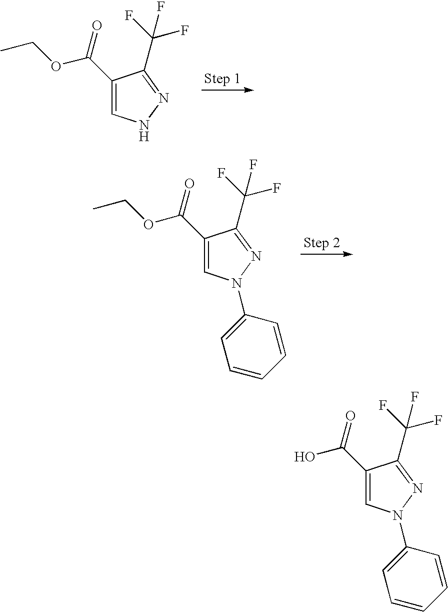

### Step 1

According to the procedure of Buchwald et al. (J. Org. Chem. 2004, 69, 5578), to a 350 mL sealed tube flushed vigorously with nitrogen were added ethyl 3-(trifluoromethyl)-1H-pyrazole-4-carboxylate (20.0 g, 96.1 mmol), 1-iodobenzene (12.9 ml, 115 mmol), potassium carbonate (27.9 g, 202 mmol), copper(I) iodide (0.915 g, 4.80 mmol), and (1S,2S)—N1,N2-dimethylcyclohexane-1,2-diamine (1.37 g, 9.61 mmol), followed by degassed toluene (100 ml). The mixture was stirred for 24 hours at 110° C., cooled to room temperature, and filtered through a short silica pad which was rinsed with toluene and EtOAc thoroughly. The filtrate was concentrated in vacuo to give ethyl 1-phenyl-3-(trifluoromethyl)-1H-pyrazole-4-carboxylate (21 g, 77%) as a solid: ¹H NMR (400 MHz, CDCl₃) δ 1.39 (t, 3H, J=7.0 Hz), 4.37 (q, 2H, J=7.0 Hz), 7.42 (m, 1H), 7.52 (m, 2H), 7.72 (m, 2H), 8.48 (m, 1H).

### Step 2

To a solution of ethyl 1-phenyl-3-(trifluoromethyl)-1H-pyrazole-4-carboxylate (0.800 g, 2.81 mmol) in EtOH (20 ml) and THF (5 ml) was added 2N NaOH (1.69 ml, 8.44 mmol). The mixture was refluxed until TLC indicated completion, cooled to room temperature, and concentrated in vacuo. The residue was dissolved in water and washed with ether. The aqueous layer was then adjusted to pH 4 with 10% HCl. The solid was collected by filtration and dried under vacuum to give 1-phenyl-3-(trifluoromethyl)-1H-pyrazole-4-carboxylic acid (0.560 g, 78%) as a powder: ¹H NMR (400 MHz, DMSO-d₆) δ 7.47 (m, 1H), 7.58 (m, 2H), 7.94 (m, 2H), 9.24 (br, 1H); m/z 254.9 (APCI neg) (100%) (M−H)

### N-(2-(4-Ethoxybenzamido)ethyl)-3-(trifluoromethyl)-1H-pyrazole-4-carboxamide

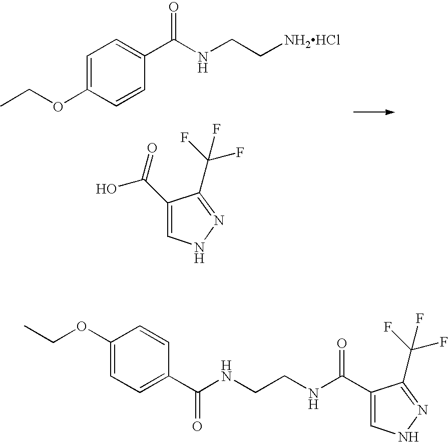

To N-(2-aminoethyl)-4-ethoxybenzamide hydrochloride (1.58 g, 6.44 mmol) in DCM (50 mL) were added successively 3-(trifluoromethyl)-1H-pyrazole-4-carboxylic acid (1.16 g, 6.44 mmol), EDAC.HCl (1.61 g, 8.37 mmol), HOBt.H₂O (1.22 g, 9.02 mmol) and triethylamine (1.3 g, 12.9 mmol). The mixture was stirred at room temperature for 12 hours then concentrated. The residue was purified by chromatography (DCM/MeOH 90/10) to yield N-(2-(4-ethoxybenzamido)ethyl)-3-(trifluoromethyl)-1H-pyrazole-4-carboxamide as a tan solid (1.87 g, 79%): ¹H NMR (400 MHz, DMSO-d₆) δ 1.34 (t, 3H, J=7.0 Hz), 3.37 (m, 4H), 4.07 (q, 2H, J=7.0 Hz), 6.97 (m, 2H), 7.81 (m, 2H), 8.34 (m, 2H), 8.42 (m, 1H); m/z 369.2 (APCI neg)(100%)(M−H).

### Formulation Example 1

### Production of Capsules

| | |
|---|---|
| 1) compound of Example A1 | 30 mg |
| 2) fine cellulose powder | 10 mg |
| 3) lactose | 19 mg |
| 4) magnesium stearate | 1 mg |
| total | 60 mg |

1), 2), 3) and 4) are mixed and filled in gelatin capsules.

### Formulation Example 2

### Production of Tablets

| | |
|---|---|
| 1) compound of Example A1 | 30 g |
| 2) lactose | 50 g |
| 3) corn starch | 15 g |
| 4) carboxymethylcellulose calcium | 44 g |
| 5) magnesium stearate | 1 g |
| total of 1000 tablets | 140 g |

The entire amounts of 1), 2) and 3), and 30 g of 4) are kneaded with water, dried in vacuo and granulated.

The granules are mixed with 14 g of 4) and 1 g of 5) and the mixture is compressed with a tableting machine, whereby 1000 tablets containing 30 mg of compound of Example A1 per tablet are obtained.

### Formulation Example 3

### Production of Capsules

| | |
|---|---|
| 1) compound of Example B1 | 30 mg |
| 2) fine cellulose powder | 10 mg |
| 3) lactose | 19 mg |
| 4) magnesium stearate | 1 mg |
| total | 60 mg |

1), 2), 3) and 4) are mixed and filled in gelatin capsules.

### Formulation Example 4

### Production of Tablets

| | |
|---|---|
| 1) compound of Example B1 | 30 g |
| 2) lactose | 50 g |
| 3) corn starch | 15 g |
| 4) carboxymethylcellulose calcium | 44 g |
| 5) magnesium stearate | 1 g |
| total of 1000 tablets | 140 g |

The entire amounts of 1), 2) and 3), and 30 g of 4) are kneaded with water, dried in vacuo and granulated.

The granules are mixed with 14 g of 4) and 1 g of 5) and the mixture is compressed with a tableting machine, whereby 1000 tablets containing 30 mg of compound of Example B1 per tablet are obtained.

### Formulation Example 5

### Production of Capsules

| | |
|---|---|
| 1) compound of Example C1 | 30 mg |
| 2) fine cellulose powder | 10 mg |
| 3) lactose | 19 mg |
| 4) magnesium stearate | 1 mg |
| total | 60 mg |

1), 2), 3) and 4) are mixed and filled in gelatin capsules.

### Formulation Example 6

### Production of Tablets

| | |
|---|---|
| 1) compound of Example C1 | 30 g |
| 2) lactose | 50 g |
| 3) corn starch | 15 g |
| 4) carboxymethylcellulose calcium | 44 g |
| 5) magnesium stearate | 1 g |
| total of 1000 tablets | 140 g |

The entire amounts of 1), 2) and 3), and 30 g of 4) are kneaded with water, dried in vacuo and granulated.

The granules are mixed with 14 g of 4) and 1 g of 5) and the mixture is compressed with a tableting machine, whereby 1000 tablets containing 30 mg of compound of Example C1 per tablet are obtained.

### Formulation Example 7

### Production of Capsules

| | |
|---|---|
| 1) compound of Example D1 | 30 mg |
| 2) fine cellulose powder | 10 mg |
| 3) lactose | 19 mg |
| 4) magnesium stearate | 1 mg |
| total | 60 mg |

1), 2), 3) and 4) are mixed and filled in gelatin capsules.

### Formulation Example 8

### Production of Tablets

| | |
|---|---|
| 1) compound of Example D1 | 30 g |
| 2) lactose | 50 g |
| 3) corn starch | 15 g |
| 4) carboxymethylcellulose calcium | 44 g |
| 5) magnesium stearate | 1 g |
| total of 1000 tablets | 140 g |

The entire amounts of 1), 2) and 3), and 30 g of 4) are kneaded with water, dried in vacuo and granulated.

The granules are mixed with 14 g of 4) and 1 g of 5) and the mixture is compressed with a tableting machine, whereby 1000 tablets containing 30 mg of compound of Example D1 per tablet are obtained.

### Experimental Example 1

The genetic engineering described below followed the method described in a book (Maniatis et al., Molecular Cloning, Cold Spring Harbor Laboratory (1989)) or a method described in the protocol attached to the reagents.

### (1) Cloning of Human DGAT1 Gene and Preparation of Recombinant Baculovirus

Human DGAT1 gene was cloned by PCR using human adipocyte cDNA (Clontech, QUICK-Clone cDNA, human fat cell, cat# 637220) as a template and, based on the DGAT1 gene information reported by Case, S. et al. (Proc. Natl. Acad. Sci. U.S.A. 95 (22), 13018-13023 (1998)), a nucleotide sequence (245-1711 of Genbank Accession No. NM_{—}012079) encoding DGAT1 was amplified with the following PCR primer. The primer nucleotide sequence is shown below.

DGAT1-U:

(SEQ ID NO: 1)

5′ AATTAAGAATTCATGGGCGACTACAAAGACGATGACGACGGCGACCGC
GGCAGCTCCCGGCGCCGG 3′,
and
DGAT2-L:

(SEQ ID NO: 2)

5′ AATTAAACTAGTTCAGGCCTCTGCCGCTGGGGCCTCATAGTTGAG 3′

The PCR reaction was conducted using a KOD-plus kit (TOYOBO). The obtained PCR product was electrophoresed on agarose gel (1%), the DNA fragment amplified by PCR was recovered from the gel, and then digested with restriction enzymes EcoRI and SpeI. The DNA treated with the restriction enzymes was electrophoresed on agarose gel (1%), and the obtained DNA fragment was recovered and ligated with plasmid pFASTBAC1 (Invitrogen) digested with restriction enzymes EcoRI and SpeI to give expression plasmid pFB-DGAT1. The nucleotide sequence of the inserted fragment was confirmed and found to be identical with the nucleotide sequence of DGAT1 (245-1711 of Genbank Accession No. NM_{—}012079). Furthermore, using BAC-TO-BAC Baculovirus Expression System (Invitrogen), recombinant baculovirus BAC-DGAT1 was prepared.

### (2) Preparation of Microsome of Sf9 Insect Cells Highly Expressing DGAT1 Enzyme

SF9 cells were sown at 1×10⁶ cells/ml on Sf-900II SFM medium (1 L, Invitrogen) containing 10% fetal calf serum (Trace), 50 mg/L gentamicin (Invitrogen) and 0.1% Pluronic F-68 (Invitrogen), and shaking culture was performed using a 2 L volume Erlenmeyer flask at 27° C., 100 rpm. After culturing for 24 hrs, recombinant baculovirus BAC-DGAT1 (6.7 mL) was added, and the mixture was further cultured for 3 days. The culture medium was centrifuged at 2,000 rpm for 5 min to give virus-infected cells. The infected cells were washed with a phosphate buffered saline (Invitrogen), centrifuged under the same conditions, and the cells were preserved at −80° C. The cryopreserved cells were thawed in ice, suspended in buffer A (50 mM Tris buffer (30 mL, pH 7.4) containing 20% glycerol, 0.15 M NaCl) supplemented with Complete Protease Inhibitor (Boehringer), and ruptured 3 times with a Polytron homogenizer (Kinematica) at 20,000 rpm for 30 sec. The Sf9 microsome fractions were obtained by a conventional method and cryopreserved at −80° C. as a DGAT1 high expression Sf9 microsome.

### (3) Determination of DGAT Inhibitory Activity

As a DGAT1 reaction buffer, a solution having a composition of 100 mM Tris-HCl (pH 7.5), 250 mM sucrose, 150 mM MgCl₂, 0.01% bovine serum albumin (BSA) was used. Using this buffer, a given concentration of the test compound and a composition (100 μl) of 25 μM dioleoylglycerol, 25 μM [¹⁴C]-Oleoyl-CoA, 5 μg protein/ml DGAT1 high expression Sf9 microsome, and 1% acetone were subjected to a triglyceride synthesis reaction at 32° C. for 20 min. A mixture of 300 μL of chloroform:methanol (=1:2) was added to the reaction mixture to quench the reaction. The reaction mixture was sufficiently mixed and distilled water (200 μL) was added to partition the mixture between a chloroform layer (lower layer) and an aqueous layer (upper layer). The chloroform layer (50 μL) was spotted on a thin layer chromatography silica gel plate (TLC plate, Merck) and developed with a solvent (n-hexane:diethyl ether:ethyl acetate:acetic acid=255:30:15:0.6). The developed TLC plate was dried, contacted with a BAS imaging plate (manufactured by FUJIFIIM) and measured with BAS2500 (manufactured by FUJIFILM) 16 hr later to numerically show the amount of [¹⁴C]-triglyceride (TG amount) produced during the reaction. The inhibitory rate was calculated by the following formula:

Inhibitory rate (%)=(1−(TG amount with addition of test compound−blank TG amount)/(control TG amount−blank TG amount))×100

The count of the triglyceride produced in the solution reacted without addition of the compound was used as a “control TG amount”, and the count of the triglyceride produced in the solution reacted without addition of the test compound and DGAT1 high expression Sf9 microsome was used as a “blank TG amount”. In addition, the concentration (IC₅₀) of the test compound necessary for inhibiting the triglyceride synthesis by 50% was calculated by PRISM 3.02 (manufactured by GraphPad Software). The inhibitory activity is shown in Table 1.

The inhibitory activity is shown by A<0.0 μM≦B<0.1 μM≦C<1 μM≦D<10 μM according to IC₅₀.

TABLE 1
DGAT inhibitory activity

| Example No. | hDGAT1 IC₅₀ |
|---|---|
| A11 | D |
| A12 | B |
| A15 | C |
| A24 | C |
| A27 | B |
| A33 | A |
| A35 | A |
| A42 | B |
| A43 | B |
| A47 | B |
| A53 | B |
| A55 | C |
| A58 | D |
| A73 | A |
| A76 | C |
| A82 | B |
| A86 | C |
| A87 | C |
| A96 | C |
| A98 | B |
| A99 | D |
| A103 | B |
| B1 | B |
| B2 | D |
| B3 | B |
| B6 | C |
| C11 | D |
| C15 | C |
| C24 | D |
| D1 | D |
| D2 | D |
| D6 | D |

### INDUSTRIAL APPLICABILITY

The compound of the present invention has a DGAT inhibitory activity and is useful for the prophylaxis, treatment or improvement of DGAT-related diseases.

The references cited herein, including patents and patent applications, are hereby incorporated in full by reference, to the extent that they have been disclosed herein.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

This application is based on application No. 60/832,115 filed in USA, the contents of which are incorporated hereinto by reference.

1. A compound represented by formula (Ia): 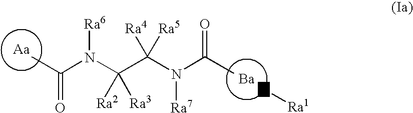
wherein
ring Ba is a 5-membered nitrogen-containing aromatic heterocycle optionally condensed with an aromatic ring, which is optionally further substituted;
Ra¹ is a hydrogen atom or a substituent;
ring Aa is an optionally substituted aromatic heterocycle; and
Ra², Ra³, Ra⁴, Ra⁵, Ra⁶ and Ra⁷ are each independently a hydrogen atom or a substituent;
provided that
1) when ring Ba is pyrazole which is optionally further substituted, then ring Ba does not have optionally substituted tetrahydrofurylmethoxy as a substituent other than Ra¹;
2) when ring Ba is imidazole which is optionally further substituted, then ring Ba does not have optionally substituted quinolyl as a substituent other than Ra¹;
3) when ring Ba is pyrazole which is optionally further substituted, then Ra¹ is not optionally substituted quinolyl; and
4) ring Aa is not the same as ring Ba;
or a salt thereof.

2. The compound of claim 1, wherein ring Ba is pyrazole, benzimidazole, indole or indazole, each of which is optionally further substituted.

3. The compound of claim 1, wherein Ra¹ is a hydrogen atom, an optionally substituted hydrocarbon group, an optionally substituted heterocyclic group or an acyl group.

4. The compound of claim 1, wherein ring Aa is an aromatic heterocycle optionally substituted by 1 to 3 substituents selected from an optionally substituted hydrocarbon group, an optionally substituted heterocyclic group, an optionally substituted hydroxy group, an optionally substituted amino group, an optionally substituted mercapto group, a cyano group, an acyl group and a halogen atom.

5. The compound of claim 1, wherein Ra², Ra³, Ra⁴, Ra⁵, Ra⁶ and Ra⁷ are both hydrogen atoms.

6. A compound represented by formula (Ib): 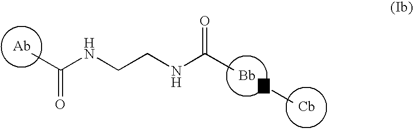
wherein
ring Bb is a 5-membered nitrogen-containing aromatic heterocycle optionally condensed with an aromatic ring, which is optionally further substituted;
ring Cb is an optionally substituted aromatic heterocycle; and
ring Ab is an optionally substituted aromatic hydrocarbon;
provided that when ring Bb is pyrazole which is optionally further substituted, then ring Cb is not optionally substituted quinoline;
or a salt thereof.

7. The compound of claim 6, wherein ring Bb is pyrazole, benzimidazole, indole or indazole, each of which is optionally further substituted.

8. The compound of claim 6, wherein ring Cb is an aromatic heterocycle optionally substituted by 1 to 3 substituents selected from a halogen atom, a hydroxy group, a C₁₋₆ alkyl group and a C₁₋₆ alkoxy group.

9. The compound of claim 6, wherein ring Ab is an aromatic hydrocarbon optionally substituted by 1 to 3 substituents selected from an optionally substituted hydrocarbon group, an optionally substituted heterocyclic group, an optionally substituted hydroxy group, a cyano group, an acyl group and a halogen atom.

10. A compound represented by formula (Ic): 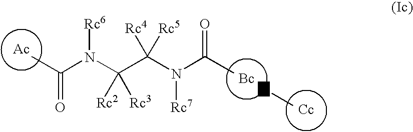
wherein
ring Bc is a 5-membered nitrogen-containing aromatic heterocycle optionally condensed with an aromatic ring, which is optionally further substituted;
ring Cc is an optionally substituted aromatic ring;
ring Ac is an optionally substituted aromatic hydrocarbon; and
Rc², Rc³, Rc⁴, Rc⁵, Rc⁶ and Rc⁷ are each independently a hydrogen atom or a substituent, or any two of Rc², Rc³, Rc⁴, Rc⁵, Rc⁶ and Rc⁷ are optionally bonded to each other to form a non-aromatic ring;
provided that
1) ring Bc is not pyrazol-5-yl and 2H-1,2,3-triazol-4-yl, each of which is optionally further substituted;
2) ring Cc is not optionally substituted quinoline;
3) a compound wherein Rc², Rc³, Rc⁴, Rc⁵, Rc⁶ and Rc⁷ are hydrogen atoms is excluded; and
4) when Rc⁶ and Rc⁷ are bonded, then they do not form piperazine;
or a salt thereof.

11. The compound of claim 10, wherein ring Bc is pyrazole, benzimidazole, indole or indazole, each of which is optionally further substituted.

12. The compound of claim 10, wherein ring Cc is an aromatic hydrocarbon optionally substituted by 1 to 3 substituents selected from a halogen atom, a hydroxy group, a C₁₋₆ alkyl group and a C₁₋₆ alkoxy group.

13. The compound of claim 10, wherein ring Ac is an aromatic hydrocarbon optionally substituted by 1 to 3 substituents selected from an optionally substituted hydrocarbon group, an optionally substituted heterocyclic group, an optionally substituted hydroxy group, a cyano group, an acyl group and a halogen atom.

14. The compound of claim 10, wherein Rc² and Rc³ are each independently a hydrogen atom, an acyl group or an optionally substituted hydrocarbon group, or Rc² or Rc³ is bonded to Rc⁴ or Rc⁵ to form a non-aromatic ring, bonded to Rc⁶ to form a non-aromatic heterocycle, or bonded to Rc⁷ to form a non-aromatic heterocycle.

15. The compound of claim 10, wherein Rc⁴ and Rc⁵ are each independently a hydrogen atom, an acyl group or an optionally substituted hydrocarbon group, or Rc⁴ or Rc⁵ is bonded to Rc² or Rc³ to form a non-aromatic ring, bonded to Rc⁶ to form a non-aromatic heterocycle, or bonded to Rc⁷ to form a non-aromatic heterocycle.

16. The compound of claim 10, wherein Rc⁶ is a hydrogen atom or an optionally substituted hydrocarbon group, or Rc⁶ is bonded to Rc² or Rc³ to form a non-aromatic heterocycle, or bonded to Rc⁴ or Rc⁵ to form a non-aromatic heterocycle.

17. The compound of claim 10, wherein Rc⁷ is a hydrogen atom or an optionally substituted hydrocarbon group, or Rc⁷ is bonded to Rc² or Rc³ to form a non-aromatic heterocycle, or bonded to Rc⁴ or Rc⁵ to form a non-aromatic heterocycle.

18. A compound represented by formula (Id): 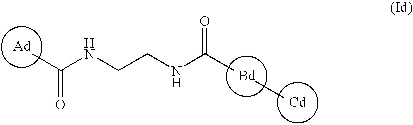
wherein
ring Bd is an aromatic heterocycle which is optionally further substituted;
ring Cd is an optionally substituted aromatic ring; and
ring Ad is an optionally substituted aromatic hydrocarbon;
provided that
1) ring Bd is not pyrazol-4-yl and pyrrol-3-yl, each of which is optionally further substituted;
2) ring Cd is not optionally substituted quinoline;
3) when ring Bd is pyridine or quinoline, each of which is optionally further substituted, then ring Bd has substituent(s) besides ring Cd; and
4) when ring Bd is a 5-membered nitrogen-containing aromatic heterocycle optionally condensed with an aromatic ring, which is optionally further substituted, then ring Bd does not have an optionally substituted aromatic heterocyclic group as a substituent other than ring Cd and ring Cd is an optionally substituted aromatic hydrocarbon;
or a salt thereof.

19. The compound of claim 18, wherein ring Bd is pyridine, pyrazole, triazole or indole, each of which is optionally further substituted.

20. The compound of claim 18, wherein ring Cd is an aromatic hydrocarbon optionally substituted by 1 to 3 substituents selected from a halogen atom, a hydroxy group, a C₁₋₆ alkyl group and a C₁₋₆ alkoxy group.

21. The compound of claim 18, wherein ring Ad is an aromatic hydrocarbon optionally substituted by 1 to 3 substituents selected from an optionally substituted hydrocarbon group, an optionally substituted heterocyclic group, an optionally substituted hydroxy group, a cyano group, an acyl group and a halogen atom.

22. N-(2-(1-phenyl-3-(trifluoromethyl)-1H-pyrazole-4-carboxamido)ethyl)-6-(2,2,2-trifluoroethoxy)nicotinamide;
6-(cyclopropylmethoxy)-N-(2-(1-phenyl-3-(trifluoromethyl)-1H-pyrazole-4-carboxamido)ethyl)nicotinamide;
N-(2-(1-phenyl-3-(trifluoromethyl)-1H-pyrazole-4-carboxamido)ethyl)-6-(3,3,3-trifluoropropoxy)nicotinamide;
6-(2-(ethylsulfonyl)ethoxy)-N-(2-(1-phenyl-3-(trifluoromethyl)-1H-pyrazole-4-carboxamido)ethyl)nicotinamide;
N-(2-(1-phenyl-3-(trifluoromethyl)-1H-pyrazole-4-carboxamido)ethyl)-6-propylnicotinamide;
1-phenyl-N-(2-(6-(2,2,2-trifluoroethoxy)nicotinamido)ethyl)-1H-indole-3-carboxamide;
N-(2-(1-phenyl-3-(trifluoromethyl)-1H-pyrazole-4-carboxamido)ethyl)-6-o-tolylnicotinamide;
1-benzoyl-N-(2-(6-(2,2,2-trifluoroethoxy)nicotinamido)ethyl)-1H-indole-3-carboxamide;
6-(5-isopropyl-1,2,4-oxadiazol-3-yl)-N-(2-(1-phenyl-3-(trifluoromethyl)-1H-pyrazole-4-carboxamido)ethyl)nicotinamide; or
N-(2-(4-ethoxybenzamido)ethyl)-1-(pyridin-2-yl)-3-(trifluoromethyl)-1H-pyrazole-4-carboxamide;
or a salt thereof.

23. A prodrug of the compound of claim 1, 6, 10 or 18.

24. A pharmaceutical agent comprising the compound of claim 1, 6, 10 or 18, or a prodrug thereof.

25. The pharmaceutical agent of claim 24, which is an agent for the prophylaxis or treatment of obesity, hyperlipidemia or diabetes.

26. A DGAT inhibitor comprising the compound of claim 1, 6, 10 or 18, or a prodrug thereof.

27-28. (canceled)

29. A method for the prophylaxis or treatment of obesity, hyperlipidemia or diabetes in a mammal, which comprises administering the compound of claim 1, 6, 10 or 18, or a prodrug thereof to the mammal.

30. A method of inhibiting DGAT in a mammal, which comprises administering the compound of claim 1, 6, 10 or 18, or a prodrug thereof to the mammal.


SEQUENCE LISTING (2 sequences)

SEQ ID NO: 1 - DNA, 66 bp, Artificial
  primer for cloning human DGAT1-U gene
aattaagaattcatgggcgactacaaagacgatgacgacggcgaccgcggcagctcccggcgccgg

SEQ ID NO: 2 - DNA, 45 bp, Artificial
  primer for cloning human DGAT2-L gene
aattaaactagttcaggcctctgccgctggggcctcatagttgag